(19) United States
(12) Patent Application Publication
(10) Pub. No.: US 2002/0119462 A1
(43) Pub. Date: Aug. 29, 2002

(54) Molecular toxicology modeling

(76) Inventors: Donna L. Mendrick, Mount Airy, MD (US); Mark W. Porter, Germantown, MD (US); Kory R. Johnson, Bethesda, MD (US); Arthur L. Castle, Washington, DC (US); Michael R. Elashoff, Germantown, MD (US)

Correspondence Address: MORGAN LEWIS & BOCKIUS LLP, 1111 PENNSYLVANIA AVENUE NW, WASHINGTON, DC, 20004, US

(21) Appl. No.: 09/917,800

(22) Filed: Jul. 31, 2001

Related U.S. Application Data
(60) Provisional application No. 60/222,040, filed on Jul. 31, 2000.
(60) Provisional application No. 60/244,880, filed on Nov. 2, 2000.
(60) Provisional application No. 60/290,029, filed on May 11, 2001.
(60) Provisional application No. 60/290,645, filed on May 15, 2001.
(60) Provisional application No. 60/292,336, filed on May 22, 2001.
(60) Provisional application No. 60/295,798, filed on Jun. 6, 2001.
(60) Provisional application No. 60/297,457, filed on Jun. 13, 2001.
(60) Provisional application No. 60/298,884, filed on Jun. 19, 2001.
(60) Provisional application No. 60/303,459, filed on Jul. 9, 2001.

(51) Int. Cl. C12Q 1/68; G06F 19/00; G01N 33/48; G01N 33/50

(52) U.S. Cl. 435/6; 702/20

(57) ABSTRACT

The present invention is based on the elucidation of the global changes in gene expression and the identification of toxicity markers in tissues or cells exposed to a known toxin. The genes may be used as toxicity markers in drug screening and toxicity assays. The invention includes a database of genes characterized by toxin-induced differential expression that is designed for use with microarrays and other solid-phase probes.

### RELATED APPLICATIONS

This application is related to U.S. Provisional Applications No. 60/222,040, 60/244,880, 60/290,029, 60/290,645, 60/292,336, 60/295,798, 60/297,457, 60/298,884 and 60/303,459, all of which are herein incorporated by reference in their entirety.

### BACKGROUND OF THE INVENTION

The need for methods of assessing the toxic impact of a compound, pharmaceutical agent or environmental pollutant on a cell or living organism has led to the development of procedures which utilize living organisms as biological monitors. The simplest and most convenient of these systems utilize unicellular microorganisms such as yeast and bacteria, since they are most easily maintained and manipulated. Unicellular screening systems also often use easily detectable changes in phenotype to monitor the effect of test compounds on the cell. Unicellular organisms, however, are inadequate models for estimating the potential effects of many compounds on complex multicellular animals, as they do not have the ability to carry out biotransformations to the extent or at levels found in higher organisms.

The biotransformation of chemical compounds by multicellular organisms is a significant factor in determining the overall toxicity of agents to which they are exposed. Accordingly, multicellular screening systems may be preferred or required to detect the toxic effects of compounds. The use of multicellular organisms as toxicology screening tools has been significantly hampered, however, by the lack of convenient screening mechanisms or endpoints, such as those available in yeast or bacterial systems. In addition, previous attempts to produce toxicology prediction systems have failed to provide the necessary modeling information (eg. WO0012760, WO0047761, WO0063435, WO0132928A2, WO0138579A2, and the Affymetrix® Rat Tox Chip.

### SUMMARY OF THE INVENTION

The present invention is based on the elucidation of the global changes in gene expression in tissues or cells exposed to known toxins, in particular hepatotoxins, as compared to unexposed tissues or cells as well as the identification of individual genes that are differentially expressed upon toxin exposure.

In various aspects, the invention includes methods of predicting at least one toxic effect of a compound, predicting the progression of a toxic effect of a compound, and predicting the hepatoxicity of a compound. The invention also includes methods of identifying agents that modulate the onset or progression of a toxic response. Also provided are methods of predicting the cellular pathways that a compound modulates in a cell. The invention includes methods of identifying agents that modulate protein activities.

In a further aspect, the invention provides probes comprising sequences that specifically hybridize to genes in Tables 1-3. Also provided are solid supports comprising at least two of the previously mentioned probes. The invention also includes a computer system that has a database containing information identifying the expression level in a tissue or cell sample exposed to a hepatotoxin of a set of genes comprising at least two genes in Tables 1-3.

### DETAILED DESCRIPTION

Many biological functions are accomplished by altering the expression of various genes through transcriptional (e.g. through control of initiation, provision of RNA precursors, RNA processing, etc.) and/or translational control. For example, fundamental biological processes such as cell cycle, cell differentiation and cell death are often characterized by the variations in the expression levels of groups of genes.

Changes in gene expression are also associated with the effects of various chemicals, drugs, toxins, pharmaceutical agents and pollutants on an organism or cells. For example, the lack of sufficient expression of functional tumor suppressor genes and/or the over expression of oncogene/protooncogenes after exposure to an agent could lead to tumorgenesis or hyperplastic growth of cells (Marshall, Cell, 64: 313-326 (1991); Weinberg, Science, 254:1138-1146 (1991)). Thus, changes in the expression levels of particular genes (e.g. oncogenes or tumor suppressors) may serve as signposts for the presence and progression of toxicity or other cellular responses to exposure to a particular compound.

Monitoring changes in gene expression may also provide certain advantages during drug screening and development. Often drugs are screened for the ability to interact with a major target without regard to other effects the drugs have on cells. These cellular effects may cause toxicity in the whole animal, which prevents the development and clinical use of the potential drug.

The present inventors have examined tissue from animals exposed to the known hepatotoxins which induce detrimental liver effects, to identify global changes in gene expression induced by these compounds. These global changes in gene expression, which can be detected by the production of expression profiles, provide useful toxicity markers that can be used to monitor toxicity and/or toxicity progression by a test compound. Some of these markers may also be used to monitor or detect various disease or physiological states, disease progression, drug efficacy and drug metabolism.

Identification of Toxicity Markers

To evaluate and identify gene expression changes that are predictive of toxicity, studies using selected compounds with well characterized toxicity have been conducted by the present inventors to catalogue altered gene expression during exposure in vivo and in vitro. In the present study, amitryptiline, alpha-naphthylisothiocyante (ANIT), acetaminophen, carbon tetrachloride, cyproterone acetate (CPA), diclofenac, 17α-ethinylestradiol, indomethacin, valproate and WY-14643 were selected as a known hepatotoxins.

The pathogenesis of acute CCl₄- induced hepatotoxicity follows a well-characterized course in humans and experimental animals resulting in centrilobular necrosis and steatosis, followed by hepatic regeneration and tissue repair. Severity of the hepatocellular injury is also dose-dependent and may be affected by species, age, gender and diet.

Differences in susceptibility to CCl₄ hepatotoxicity are primarily related to the ability of the animal model to metabolize CCl₄ to reactive intermediates. CCl₄-induced hepatotoxicity is dependent on CCl₄ bioactivation to trichloromethyl free radicals by cytochrome P450 enzymes (CYP2E1), localized primarily in centrizonal hepatocytes. Formation of the free radicals leads to membrane lipid peroxidation and protein denaturation resulting in hepatocellular damage or death.

The onset of hepatic injury is rapid following acute administration of CCl₄ to male rats. Morphologic studies have shown cytoplasmic accumulation of lipids in hepatocytes within 1 to 3 hours of dosing, and by 5 to 6 hours, focal necrosis and hydropic swelling of hepatocytes are evident. Centrilobular necrosis and inflammatory infiltration peak by 24 to 48 hours post dose. The onset of recovery is also evident within this time frame by increased DNA synthesis and the appearance of mitotic figures. Removal of necrotic debris begins by 48 hours and is usually completed by one week, with full restoration of the liver by 14 days.

Increases in serum transaminase levels also parallel CCl₄-induced hepatic histopathology. In male Sprague Dawley (SD) rats, alanine aminotrasferase (ALT) and aspartate aminotransferase (AST) levels increase within 3 hours of CCl₄ administration (0.1, 1,2, 3, 4 mL/kg, ip; 2.5 mL/kg, po) and reach peak levels (approximately 5-10 fold increases) within 48 hours post dose. Significant increases in serum α-glutathione s-transferase (α-GST) levels have also been detected as early as 2 hours after CCl₄ administration (25 μL/kg, po) to male SD rats.

At the molecular level, induction of the growth-related proto-oncogenes, c-fos and c-jun, is reportedly the earliest event detected in an acute model of CCl₄-induced hepatotoxicity (Schiaffonato et al. (1997) Liver 17:183-191). Expression of these early-immediate response genes has been detected within 30 minutes of a single dose of CCl₄ to mice (0.05-1.5 mL/kg, ip) and by 1 to 2 hours post dose in rats (2 mL/kg, po; 5 mL/kg,po) (Schiaffonato et al. (1997) Liver 17:183-191 and Hong et al.(1997) Yonsei Medical. J. 38:167-177). Similarly, hepatic c-myc gene expression is increased by 1 hour following an acute dose of CCl₄ to male SD rats (5 mL/kg, po) (Hong et al.). Expression of these genes following exposure to CCl₄ is rapid and transient. Peak hepatic mRNA levels for c-fos, c-jun, and c-myc, after acute administration of CCl₄ have been reported at 1 to 2 hours, 3 hours, and 1 hour post dose, respectively.

The expression of tumor necrosis factor-α (TNF-α) is also increased in the livers of rodents exposed to CCl₄, and TNF-α has been implicated in initiation of the hepatic repair process. Pre-treatment with anti-TNF-α antibodies has been shown to prevent CCl₄-mediated increases in c-jun and c-fos gene expression, whereas administration of TNF-αinduced rapid expression of these genes (Brucicoleri et al.(1997) Hepatol. 25:133-141). Up-regulation of transforming growth factor-β (TGF-β) and transforming growth factor receptors (TBRI-III) later in the repair process (24 and 48 hours after CCl₄ administration) suggests that TGF-β may play a role in limiting the regenerative response by induction of apoptosis (Gras1-Kraupp et al. (1998) Hepatol. 28:717-7126).

Acetaminophen is a widely used analgesic that at supratherapeutic doses can be metabolized to N-acetyl-p-benzoquinone imine (NAPQI) which causes hepatic and renal failure. At the molecular level, until the present invention little was known about the effects of acetominophen.

Amitriptyline is a commonly used antidepressant, although it is recognized to have toxic effects on the liver (Physicians Desk Reference, 47^{th} ed., Medical Economics Co., Inc., 1993; Balkin, U.S. Pat. No. 5,656,284). Nevertheless, amitriptyline's beneficial effects on depression, as well as on sleep and dyspepsia (H. Mertz et al., Am J Gastroenterol 93(2):160-165, 1998), migraines (E. Beubler, Wien Med Wochenschr 144(5-6):100-101, 1994), arterial hypertension (T. Bobkiewicz et al., Arch Immunol Ther Exp (Warsz) 23(4):543-547, 1975) and premature ejaculation (Smith et al., U.S. Pat. No. 5,923,341) mandate its continued use.

Differences in susceptibility to amitriptyline toxicity are considered related to differential metabolism. Amitriptyline-induced hepatotoxicity is primarily mediated by development of cholestasis, the condition caused by the failure of the liver to secrete bile, resulting in accumulation in blood plasma of substances normally secreted into bile-bilirubin and bile salts. Cholestasis is also characterized by liver cell necrosis and bile duct obstruction, which leads to increased pressure on the lumenal side of the canalicular membrane and release of enzymes (alkaline phosphatase, 5′-nucleotidase, gammaglutamyl transpeptidase) normally localized on the canalicular membrane. These enzymes also begin to accumulate in the plasma. Typical symptoms of cholestasis are general malaise, weakness, nausea, anorexia and severe pruritis (Cecil Textbook of Medicine, 20^{th} ed., part XII, pp. 772-773, 805-808, J. C. Bennett and F. Plum Eds., W. B. Saunders Co., Philadelphia, 1996).

The effects of amitriptyline or phenobarbital (PB) on phospholipid metabolism in rat liver have been studied. In one study, male Sprague-Dawley rats received amitriptyline orally in one dose of 600 mg/kg. PB was given intraperitonially (IP) at a dosage of 80 mg/kg. Animals were sacrificed by decapitation at 6, 12, 18, and 24 hr. The phospholipid level in liver was measured by enzymatic assay and by gas chromatography-mass spectrometry. Both agents caused an increase in the microsomal phosphatidylcholine content. Levels of glycerophosphate acyltransferase (GAT) and phosphatidate cytidylyltransferase (PCT) were slightly affected by amitriptyline but were significantly affected by PB. Levels of phosphatidate phosphohydrolase (PPH) and choline phosphotransferase (CPT) were significantly altered by amitriptyline and by PB (K. Hoshi et al., “Effect of amitriptyline or phenobarbital on the activities of the enzymes involved in rat liver,” Chem Pharm Bull 38:3446-3448, 1990).

In another experiment, amitriptyline was given orally to male Sprague-Dawley rats (4-5 weeks old) in a single dose of 600 mg/kg. The animals were sacrificed 12 or 24 hours later. This caused a marked increase in δ-aminolevulinic acid (δ-ALA) activity at both time points. Total heme and cytochrome b5 levels were increased but cytochrome P450 (CYP450) content remained the same. The authors concluded that hepatic heme synthesis is increased through prolonged induction of 8-ALA but this may be accounted for by the increases in cytochrome b5 and total heme and not by the CYP450 content (K. Hoshi et al., “Acute effect of amitriptyline, phenobarbital or cobaltous chloride on δ-aminolevulinic acid synthetase, heme oxygenase and microsomal heme content and drug metabolism in rat liver”, Jpn J Pharmacol 50:289-293, 1989).

Amitriptyline can cause hypersensititivity syndrome, a specific severe idiosyncratic reaction characterized by skin, liver, joint and haematological abnormalities (H. J. Milionis et al., Postgrad Med 76(896):361-363, 2000). Amitriptyline has also been shown to cause drug-induced hepatitis, resulting in liver peroxisomes with impaired catalase function (D. De Creaemer et al., Hepatology 14(5):811-817, 1991). The peroxisomes are larger in number, but smaller in size and deformed in shape. Using cultured hepatocytes, the cytotoxicity of amitriptyline was examined and compared to other psychotropic drugs (U. A. Boelsterli et al., Cell Biol Toxicol 3(3):231-250, 1987). The effects observed were release of lactate dehydrogenase from the cytosol, as well as impairment of biosynthesis and secretion of proteins, bile acids and glycolipids.

Aromatic and aliphatic isothiocyanates are commonly used soil fumigants and pesticides (E. Shaaya et al., Pesticide Science 44(3):249-253, 1995; T. Cairns et al., J Assoc Official Analytical Chemists 71(3):547-550, 1988). These compounds are also environmental hazards, however, because they remain as toxic residues in plants, either in their original or in a metabolized form (M. S. Cerny et al, J Agricultural and Food Chemistry 44(12):3835-3839, 1996) and because they are released from the soil into the surrounding air (J. Gan et al., J Agricutural and Food Chemistry 46(3):986-990, 1998). Alpha-naphthylthiourea, an amino-substituted form of ANIT, is a known rodenticide whose principal toxic effects are pulmonary edema and pleural effusion, resulting from the action of this compound on pulmonary capillaries. Microsomes from lung and liver release atomic sulfur (Goodman and Gilman's The Pharmacological Basis of Therapeutics, 9^{th} ed., chapter 67, p. 1690, J. G. Hardman et al. Eds., McGraw-Hill, New York, N.Y., 1996).

In one study in rats, ANIT (80 mg/kg) was dissolved in olive oil and given orally to male Wistar rats (180-320g). All animals were fasted for 24 hours before ANIT treatment, and blood and bile excretion were analyzed 24 hours later. Levels of total bilirubin, alkaline phosphatase, serum glutamic oxaloacetic transaminase and serum glutamic pyruvic transaminase were found to be significantly increased, while ANIT reduced total bile flow, all of which are indications of severe biliary dysfunction. This model is used to induce cholestasis with jaundice because the injury is reproducible and dose-dependent. ANIT is metabolized by microsomal enzymes, and a metabolite plays a fundamental role in its toxicity (M. Tanaka et al., “The inhibitory effect of SA3443, a novel cyclic disulfide compound, on alpha-naphthyl isothiocyanate-induced intrahepatic cholestasis in rats,” Clinical and Experimental Pharmacology and Physiology 20:543-547, 1993).

ANIT fails to produce extensive necrosis, but has been found to produce inflammation and edema in the portal tract of the liver (T. J. Maziasa et al., “The differential effects of hepatotoxicants on the sulfation pathway in rats,” Toxicol Appl Pharmacol 110:365-373, 1991). Livers treated with ANIT are significantly heavier than control-treated counterparts and serum levels of alanine aminotransferase (ALT), gamma-glutamyl transpeptidase (γ-GTP), total bilirubin, lipid peroxide and total bile acids showed significant increases (Anonymous, “An association between lipid peroxidation and α-naphthylisothiocyanate-induced liver injury in rats,” Toxicol Lett 105:103-110, 2000).

ANIT-induced hepatotoxicity may also be characterized by cholangiolitic hepatitis and bile duct damage. Acute hepatotoxicity caused by ANIT in rats is manifested as neutrophil-dependent necrosis of bile duct epithelial cells (BDECs) and hepatic parenchymal cells. These changes mirror the cholangiolitic hepatitis found in humans (D. A. Hill, Toxicol Sci 47:118-125, 1999).

Exposure to ANIT also causes liver injury by the development of cholestasis, the condition caused by failure to secrete bile, resulting in accumulation in blood plasma of substances normally secreted into bile, such as bilirubin and bile salts. Cholestasis is also characterized by liver cell necrosis, including bile duct epithelial cell necrosis, and bile duct obstruction, which leads to increased pressure on the lumenal side of the canalicular membrane, decreased canalicular flow and release of enzymes normally localized on the canalicular membrane (alkaline phosphatase, 5′-nucleotidase, gammaglutamyl transpeptidase). These enzymes also begin to accumulate in the plasma. Typical symptoms of cholestasis are general malaise, weakness, nausea, anorexia and severe pruritis (Cecil Textbook of Medicine, 20^{th} ed., part XII, pp. 772-773, 805-808, J. C. Bennett and F. Plum Eds., W. B. Saunders Co., Philadelphia, 1996 and D. C. Kossor et al., “Temporal relationship of changes in hepatobiliary function and morphology in rats following α-naphthylisothiocyanate (ANIT) administration,” Toxicol Appl Pharmacol 119:108-114, 1993).

ANIT-induced cholestatis is also characterized by abnormal serum levels of alanine aminotransferase, aspartic acid aminotransferase and total bilirubin. In addition, hepatic lipid peroxidation is increased, and the membrane fluidity of microsomes is decreased. Histological changes include an infiltration of polymorphonuclear neutrophils and elevated number of apoptotic hepatocytes (J. R. Calvo et al., J Cell Biochem 80(4):461-470, 2001). Other known hepatotoxic effects of exposure to ANIT include a damaged antioxidant defense system, decreased activities of superoxide dismutase and catalase (Y. Ohta et al.

Toxicology 139(3):265-275, 1999), and the release of several proteases from the infiltrated neutrophils, alanine aminotransferase, cathepsin G, elastase, which mediate hepatocyte killing (D. A. Hill et al., Toxicol Appl Pharmacol 148(1):169-175, 1998).

Indomethacin is a non-steroidal antiinflammatory, antipyretic and analgesic drug commonly used to treat rheumatoid arthritis, osteoarthritis, ankylosing spondylitis, gout and a type of severe, chronic cluster headache characterized by many daily occurrences and jabbing pain. This drug acts as a potent inhibitor of prostaglandin synthesis; it inhibits the cyclooxygenase enzyme necessary for the conversion of arachidonic acid to prostaglandins (PDR 47th ed., Medical Economics Co., Inc., Montvale, N.J., 1993; Goodman & Gilman's The Pharmalogical Basis of Therapeutics 9th ed., J. G. Hardman et al. Eds., McGraw Hill, New York, 1996, pp. 1074-1075, 1089-1095; Cecil Textbook of Medicine, 20^{th} ed., part XII, pp. 772-773, 805-808, J. C. Bennett and F. Plum Eds., W. B. Saunders Co., Philadelphia, 1996).

The most frequent adverse effects of indomethacin treatment are gastrointestinal disturbances, usually mild dyspepsia, although more severe conditions, such as bleeding, ulcers and perforations can occur. Hepatic involvement is uncommon, although some fatal cases of hepatitis and jaundice have been reported. Renal toxicity can also result, particularly after long-term administration. Renal papillary necrosis has been observed in rats, and interstitial nephritis with hematuria, proteinuria and nephrotic syndrome have been reported in humans. Patients suffering from renal dysfunction risk developing a reduction in renal blood flow, because renal prostaglandins play an important role in renal perfusion.

In rats, although indomethacin produces more adverse effects in the gastrointestinal tract than in the liver, it has been shown to induce changes in hepatocytic cytochrome P450. In one study, no widespread changes in the liver were observed, but a mild, focal, centrilobular response was noted. Serum levels of albumin and total protein were significantly reduced, while the serum level of urea was increased. No changes in creatinine or aspartate aminotransferase (AST) levels were observed (M. Falzon et al., “Comparative effects of indomethacin on hepatic enzymes and histology and on serum indices of liver and kidney function in the rat,” Br J exp Path 66:527-534, 1985). In another rat study, a single dose of indomethacin has been shown to reduce liver and renal microsomal enzymes, including CYP450, within 24 hours. Histopathological changes were not monitored, although there were lesions in the GI tract. The effects on the liver seemed to be waning by 48 hours (M. E. Fracasso et al., “Indomethacin induced hepatic alterations in mono-oxygenase system and faecal clostridium perfringens enterotoxin in the rat,” Agents Actions 31:313-316, 1990).

A study of hepatocytes, in which the relative toxicity of five nonsteroidal antiinflammatory agents was compared, showed that indomethacin was more toxic than the others. Levels of lactate dehydrogenase release and urea, as well as viability and morphology, were examined. Cells exposed to high levels of indomethacin showed cellular necrosis, nuclear pleomorphism, swollen mitochondria, fewer microvilli, smooth endoplasmic reticulum proliferation and cytoplasmic vacuolation (E. M. Sorensen et al., “Relative toxicities of several nonsteroidal antiinflammatory compounds in primary cultures of rat hepatocytes,” J Toxicol Environ Health 16(3-4);425-440, 1985). 17a-ethinylestradiol, a synthetic estrogen, is a component of oral contraceptives, often combined with the progestational compound norethindrone. It is also used in post-menopausal estrogen replacement therapy (PDR 47^{th} ed., pp. 2415-2420, Medical Economics Co., Inc., Montvale, N.J., 1993; Goodman & Gilman's The Pharmalogical Basis of Therapeutics 9^{th} ed., pp. 1419-1422, J. G. Hardman et al. Eds., McGraw Hill, New York, 1996).

The most frequent adverse effects of 17α-ethinylestradiol usage are increased risks of cardiovascular disease: myocardial infarction, thromboembolism, vascular disease and high blood pressure, and of changes in carbohydrate metabolism, in particular, glucose intolerance and impaired insulin secretion. There is also an increased risk of developing benign hepatic neoplasia, although the incidence of this disease is very low. Because this drug decreases the rate of liver metabolism, it is cleared slowly from the liver, and carcinogenic effects, such as tumor growth, may result.

In a recent study, 17α-ethinylestradiol was shown to cause a reversible intrahepatic cholestasis in male rats, mainly by reducing the bile-salt-independent fraction of bile flow (BSIF) (N. R. Koopen et al., “Impaired activity of the bile canalicular organic anion transporter (Mrp2/cmoat) is not the main cause of ethinylestradiol-induced cholestasis in the rat,” Hepatology 27:537-545, 1998). Plasma levels of bilirubin, bile salts, aspartate aminotransferase (AST) and alanine aminotransferase (ALT) in this study were not changed. This study also showed that 17α-ethinylestradiol produced a decrease in plasma cholesterol and plasma triglyceride levels, but an increase in the weight of the liver after 3 days of drug administration, along with a decrease in bile flow. Further results from this study are as follows. The activities of the liver enzymes leucine aminopeptidase and alkaline phosphatase initially showed significant increases, but enzyme levels decreased after 3 days. Bilirubin output increased, although glutathione (GSH) output decreased. The increased secretion of bilirubin into the bile without affecting the plasma level suggests that the increased bilirubin production must be related to an increased degradation of heme from heme-containing proteins. Similar results were obtained in another experiment (G. Bouchard et al., “Influence of oral treatment with ursodeoxycholic and tauroursodeoxycholic acids on estrogen-induced cholestasis in rats: effects on bile formation and liver plasma membranes,” Liver 13:193-202, 1993) in which the livers were also examined by light and electron microscopy. Despite the effects of the drug, visible changes in liver tissue were not observed.

In another study of male rats, cholestasis was induced by daily subcutaneous injections of 17α-ethinylestradiol for five days. Cholestasis was assessed by measuring the bile flow rate. Rats allowed to recover for five days after the end of drug treatment showed normal bile flow rates (Y. Hamada et al., “Hormone-induced bile flow and hepatobiliary calcium fluxes are attenuated in the perfused liver of rats made cholestatic with ethynylestradiol in vivo and with phalloidin in vitro,” Hepatology 21:1455-1464, 1995).

An experiment with male and female rats (X. Mayol, “Ethinyl estradiol-induced cell proliferation in rat liver. Involvement of specific populations of hepatocytes,” Carcinogenesis 13:2381-2388, 1992) found that 17a-ethinylestradiol induced acute liver hyperplasia (increase in mitotic index and BrdU staining) after two days of treatment, although growth regression occurred within the first few days of treatment. With long-term treatment, lasting hyperplasia was again observed after three to six months of administration of the drug. Apoptosis increased around day 3 and returned to normal by one week. Additional experiments in this same study showed that proliferating hepatocytes were predominantly located around a periportal zone of vacuolated hepatocytes, which were also induced by the treatment. Chronic induced activation was characterized by flow cytometry on hepatocytes isolated from male rats, and ploidy analysis of hepatocyte cell suspensions showed a considerably increased proportion of diploid hepatocytes. These diploid cells were the most susceptible to drug-induced proliferation. The results from this study support the theory that cell target populations exist that respond to the effects of tumor promoters. The susceptibility of the diploid hepatocytes to proliferation during treatment may explain, at least in part, the behavior of 17α-ethinylestradiol as a tumor promoter in the liver.

Wy-14643, a tumor-inducing compound that acts in the liver, has been used to study the genetic profile of cells during the various stages of carcinogenic development, with a view toward developing strategies for detecting, diagnosing and treating cancers (J. C. Rockett et al., “Use of suppression-PCR subtractive hybridisation to identify genes that demonstrate altered expression in male rat and guinea pig livers following exposure to Wy-14,643, a peroxisome proliferator and non-genotoxic hepatocarcinogen,” Toxicology 144(1-3):13-29, 2000). In contrast to other carcinogens, Wy-14643 does not mutate DNA directly. Instead, it acts on the peroxisome proliferator activated receptor-alpha (PPARalpha), as well as on other signaling pathways that regulate growth (T. E. Johnson et al., “Peroxisome proliferators and fatty acids negatively regulate liver X receptor-mediated activity and sterol biosynthesis,” J Steroid Biochem Mol Biol. 77(1):59-71, 2001). The effect is elevated and sustained cell replication, accompanied by a decrease in apoptosis (I. Rusyn et al., “Expression of base excision repair enzymes in rat and mouse liver is induced by peroxisome proliferators and is dependent upon carcinogenic potency,” Carcinogenesis 21(12):2141-2145, 2000). These authors (Rusyn et al) noted an increase in the expression of enzymes that repair DNA by base excision, but no increased expression of enzymes that do not repair oxidative damage to DNA. In a study on rodents, Johnson et al. noted that Wy-14643 inhibited liver-X-receptor-mediated transcription in a dose-dependent manner, as well as de novo sterol synthesis.

In experiments with mouse liver cells (J. M. Peters et al., “Role of peroxisome proliferator-activated receptor alpha in altered cell cycle regulation in mouse liver,” Carcinogenesis 19(11):1989-1994, 1998), exposure to Wy-14643 produced increased levels of acyl CoA oxidase and proteins involved in cell proliferation: CDK-1, 2 and 4, PCNA and c-myc. Elevated levels may be caused by accelerated transcription that is mediated directly or indirectly by PPARalpha. It is likely that the carcinogenic properties of peroxisome proliferators are due to the PPARalpha-dependent changes in levels of cell cycle regulatory proteins.

Another study on rodents (B. J. Keller et al., “Several nongenotoxic carcinogens uncouple mitochondrial oxidative phosphorylation,” Biochim Biophys Acta 1102(2):237-244, 1992) showed that Wy-14643 was capable of uncoupling oxidative phosphorylation in rat liver mitochondria. Rates of urea synthesis from ammonia and bile flow, two energy-dependent processes, were reduced, indicating that the energy supply for these processes was disrupted as a result of cellular exposure to the toxin.

Wy-14643 has also been shown to activate nuclear factor kappaB, NADPH oxidase and superoxide production in Kupffer cells (I. Rusyn et al., “Oxidants from nicotinamide adenine dinucleotide phosphate oxidase are involved in triggering cell proliferation in the liver due to peroxisome proliferators,” Cancer Res 60(17):4798-4803, 2000). NADPH oxidase is known to induce mitogens, which cause proliferation of liver cells.

CPA is a potent androgen antagonist and has been used to treat acne, male pattern baldness, precocious puberty, and prostatic hyperplasia and carcinoma (Goodman & Gilman's The Pharmacological Basis of Therapeutics 9^{th} ed., p. 1453, J. G. Hardman et al., Eds., McGraw Hill, New York, 1996). Additionally, CPA has been used clinically in hormone replacement therapy (HRT). CPA is useful in HRT as it protects the endometrium, decreases menopausal symptoms, and lessens osteoporotic fracture risk (H. P. Schneider, “The role of antiandrogens in hormone replacement therapy,” Climacteric 3 (Suppl. 2): 21-27, 2000).

Although CPA has numerous clinical applications, it is tumorigenic, mitogenic, and mutagenic. CPA has been used to treat patients with adenocarcinoma of the prostate, however in two documented cases (A. G. Macdonald and J. D. Bissett, “Avascular necrosis of the femoral head in patients with prostate cancer treated with cyproterone acetate and radiotherapy,” Clin Oncol 13: 135-137, 2001), patients developed femoral head avascular necrosis following CPA treatment. In one study (O. Krebs et al., “The DNA damaging drug cyproterone acetate causes gene mutations and induces glutathione-S-transferase P in the liver of female Big Blue transgenic F344 rats,” Carcinogenesis 19(2): 241-245, 1998), Big Blue transgenic F344 rats were giving varying doses of CPA. As the dose of CPA increased, so did the mutation frequency, but a threshold dose was not determined. Another study (S. Werner et al., “Formation of DNA adducts by cyproterone acetate and some structural analogues in primary cultures of human hepatocytes,” Mutat Res 395(2-3): 179-187, 1997), showed that CPA caused the formation of DNA adducts in primary cultures of human hepatocytes. The authors suggest that the genotoxicity associated with CPA may be due to the double bond in position 6-7 of the steroid.

In additional experiments with rats (P. Kasper and L. Mueller, “Time-related induction of DNA repair synthesis in rat hepatocytes following in vivo treatment with cyproterone acetate,” Carcinogenesis 17(10): 2271-2274, 1996), CPA was shown to induce unscheduled DNA synthesis in vitro. After a single oral dose of 100 mg CPA/kg body weight, continuous DNA repair activity was observed after 16 hours. Furthermore, CPA increased the occurrence of S phase cells, which corroborated the mitogenic potential of CPA in rat liver.

CPA has also been shown to produce cirrhosis (B. Z. Garty et al., “Cirrhosis in a child with hypothalamic syndrome and central precocious puberty treated with cyproterone acetate,” Eur J Pediatr 158(5): 367-370, 1999). A child, who had been treated with CPA for over 4 years for hypothalamic syndrome and precocious puberty, developed cirrhosis. Even though the medication was discontinued, the child eventually succumbed to sepsis and multiorgan failure four years later.

In one study on rat liver treated with CPA (W. Bursch et al., “Expression of clusterin (testosterone-repressed prostate message-2) mRNA during growth and regeneration of rat liver,” Arch Toxicol 69(4): 253-258, 1995), the expression of clusterin, a marker for apoptosis, was examined and measured by Northern and slot blot analysis. Bursch et al. showed that post-CPA administration, the clusterin mRNA concentration level increased. Moreover, in situ hybridization demonstrated that clusterin was expressed in all hepatocytes, therefore it is not limited to cells in the process of death by apoptosis.

Diclofenac, a non-steroidal anti-inflammatory drug, has been frequently administered to patients suffering from rheumatoid arthritis, osteoarthritis, and ankylosing spondylitis. Following oral administration, diclofenac is rapidly absorbed and then metabolized in the liver by cytochrome P450 isozyme of the CYC2C subfamily (Goodman & Gilman's The Pharmacological Basis of Therapeutics 9th ed., p. 637, J. G. Hardman et al., Eds., McGraw Hill, New York, 1996). In addition, diclofenac has been applied topically to treat pain due to corneal damage (D. G. Jayamanne et al., “The effectiveness of topical diclofenac in relieving discomfort following traumatic corneal abrasions,” Eye 11 (Pt. 1): 79-83, 1997; D. I. Dornic et al., “Topical diclofenac sodium in the management of anesthetic abuse keratopathy,” Am J. Ophthalmol 125(5): 719-721, 1998).

Although diclofenac has numerous clinical applications, adverse side-effects have been associated with the drug. In one study, out of 16 patients suffering from corneal complications associated with diclofenac use, 6 experienced corneal or scleral melts, three experienced ulceration, and two experienced severe keratopathy (A. C. Guidera et al., “Keratitis, ulceration, and perforation associated with topical nonsteroidal anti-inflammatory drugs,” Ophthalmology 108(5): 936-944, 2001). Another report described a term newborn who had premature closure of the ductus arteriosus as a result of maternal treatment with diclofenac (M. Zenker et al., “Severe pulmonary hypertension in a neonate caused by premature closure of the ductus arteriosus following maternal treatment with diclofenac: a case report,” J Perinat Med 26(3): 231-234, 1998). Although it was only two weeks prior to delivery, the newborn had severe pulmonary hypertension and required treatment for 22 days of high doses of inhaled nitric oxide.

Another study investigated 180 cases of patients who had reported adverse reactions to diclofenac to the Food and Drug Administration (A. T. Banks et al., “Diclofenac-associated hepatoxicity: analysis of 180 cases reported to the Food and Drug Administration as adverse reactions,” Hepatology 22(3): 820-827, 1995). Of the 180 reported cases, the most common symptom was jaundice (75% of the symptomatic patients). Liver sections were taken and analyzed, and hepatic injury was apparent one month after drug treatment. An additional report showed that a patient developed severe hepatitis five weeks after beginning diclofenac treatment for osteoarthritis (A. Bhogaraju et al., “Diclofenac-associated hepatitis,” South Med J 92(7): 711-713, 1999). Within a few months following the cessation of diclofenac treatment there was complete restoration of liver functions.

In one study on diclofenac-treated Wistar rats (P. E. Ebong et al., “Effects of aspirin (acetylsalicylic acid) and Cataflam (potassium diclofenac) on some biochemical parameters in rats,” Afr J Med Med Sci 27(3-4): 243-246, 1998), diclofenac treatment induced an increase in serum chemistry levels of alanine aminotransferase, aspartate aminotransferase, methaemoglobin, and total and conjugated bilirubin. Additionally, diclofenac enhanced the activity of alkaline phosphatase and 5′nucleotidase. Another study showed that humans given diclofenac had elevated levels of hepatic transaminases and serum creatine when compared to the control group (F. McKenna et al., “Celecoxib versus diclofenac in the management of osteoarthritis of the knee,” Scand J Rheumatol 30(1): 11-18,, 2001).

Toxicity Prediction and Modeling

The genes and gene expression information, as well as the portfolios and subsets of the genes provided in Tables 1-3, may be used to predict at least one toxic effect, including the hepatotoxicity of a test or unknown compound. As used, herein, at least one toxic effect includes, but is not limited to, a detrimental change in the physiological status of a cell or organism. The response may be, but is not required to be, associated with a particular pathology, such as tissue necrosis. Accordingly, the toxic effect includes effects at the molecular and cellular level. Hepatotoxicity is an effect as used herein and includes but is not limited to the pathologies of liver necrosis, hepatitis, fatty liver and protein adduct formation.

In general, assays to predict the toxicity or hepatotoxicity of a test agent (or compound or multi-component composition) comprise the steps of exposing a cell population to the test compound, assaying or measuring the level of relative or absolute gene expression of one or more of the genes in Tables 1-3 and comparing the identified expression level(s) to the expression levels disclosed in the Tables and database(s) disclosed herein. Assays may include the measurement of the expression levels of about 2, 3, 4, 5, 6, 7, 8, 9, 10, 15, 20, 25, 30, 50, 75, 100 or more genes from Tables 1-3.

In the methods of the invention, the gene expression level for a gene or genes induced by the test agent, compound or compositions may be comparable to the levels found in the Tables or databases disclosed herein if the expression level varies within a factor of about 2, about 1.5 or about 1.0 fold. In some cases, the expression levels are comparable if the agent induces a change in the expression of a gene in the same direction (e.g., up or down) as a reference toxin.

The cell population that is exposed to the test agent, compound or composition may be exposed in vitro or in vivo. For instance, cultured or freshly isolated hepatocytes, in particular rat hepatocytes, may be exposed to the agent under standard laboratory and cell culture conditions. In another assay format, in vivo exposure may be accomplished by administration of the agent to a living animal, for instance a laboratory rat.

Procedures for designing and conducting toxicity tests in in vitro and in vivo systems are well known, and are described in many texts on the subject, such as Loomis et al. Loomis's Esstentials of Toxicology, 4th Ed. (Academic Press, New York, 1996); Echobichon, The Basics of Toxicity Testing (CRC Press, Boca Raton, 1992); Frazier, editor, In Vitro Toxicity Testing (Marcel Dekker, New York, 1992); and the like.

In in vitro toxicity testing, two groups of test organisms are usually employed: One group serves as a control and the other group receives the test compound in a single dose (for acute toxicity tests) or a regimen of doses (for prolonged or chronic toxicity tests). Since in some cases, the extraction of tissue as called for in the methods of the invention requires sacrificing the test animal, both the control group and the group receiving compound must be large enough to permit removal of animals for sampling tissues, if it is desired to observe the dynamics of gene expression through the duration of an experiment.

In setting up a toxicity study, extensive guidance is provided in the literature for selecting the appropriate test organism for the compound being tested, route of administration. dose ranges, and the like. Water or physiological saline (0.9% NaCl in water) is the solute of choice for the test compound since these solvents permit administration by a variety of routes. When this is not possible because of solubility limitations, vegetable oils such as corn oil or organic solvents such as propylene glycol may be used.

Regardless of the route of administration, the volume required to administer a given dose is limited by the size of the animal that is used. It is desirable to keep the volume of each dose uniform within and between groups of animals. When rats or mice are used, the volume administered by the oral route generally should not exceed 0.005 ml per gram of animal. Even when aqueous or physiological saline solutions are used for parenteral injection the volumes that are tolerated are limited, although such solutions are ordinarily thought of as being innocuous. The intravenous LD₅₀ of distilled water in the mouse is approximately 0.044 ml per gram and that of isotonic saline is 0.068 ml per gram of mouse. In some instances, the route of administration to the test animal should be the same as, or as similar as possible to, the route of administration of the compound to man for therapeutic purposes.

When a compound is to be administered by inhalation, special techniques for generating test atmospheres are necessary. The methods usually involve aerosolization or nebulization of fluids containing the compound. If the agent to be tested is a fluid that has an appreciable vapor pressure, it may be administered by passing air through the solution under controlled temperature conditions. Under these conditions, dose is estimated from the volume of air inhaled per unit time, the temperature of the solution, and the vapor pressure of the agent involved. Gases are metered from reservoirs. When particles of a solution are to be administered, unless the particle size is less than about 2 μm the particles will not reach the terminal alveolar sacs in the lungs. A variety of apparatuses and chambers are available to perform studies for detecting effects of irritant or other toxic endpoints when they are administered by inhalation. The preferred method of administering an agent to animals is via the oral route, either by intubation or by incorporating the agent in the feed.

When the agent is exposed to cells in vitro or in cell culture, the cell population to be exposed to the agent may be divided into two or more subpopulations, for instance, by dividing the population into two or more identical aliquots. In some prefered embodiments of the methods of the invention, the cells to be exposed to the agent are derived from liver tissue. For instance, cultured or freshly isolated rat hepatocytes may be used.

The methods of the invention may be used to generally predict at least one toxic response, and as described in the Examples, may be used to predict the likelihood that a compound or test agent will induce various specifc liver pathologies such as liver necrosis, fatty liver disease, protein adduct formation or hepatitis. The methods of the invention may also be used to determine the similarity of a toxic response to one or more individual compounds. In addition, the methods of the invention may be used to predict or elucidate the potential cellular pathways influenced, induced or modulated by the compound or test agent due to the similarity of the expression profile compared to the profile induced by a known toxin (see Tables 3A-3S).

Diagnostic Uses for the Toxicity Markers

As described above, the genes and gene expression information or portfolios of the genes with their expression information as provided in Tables 1-3 may be used as diagnostic markers for the prediction or identification of the physiological state of tissue or cell sample that has been exposed to a compound or to identify or predict the toxic effects of a compound or agent. For instance, a tissue sample such as a sample of peripheral blood cells or some other easily obtainable tissue sample may be assayed by any of the methods described above, and the expression levels from a gene or genes from Tables 1-3 may be compared to the expression levels found in tissues or cells exposed to the toxins described herein. These methods may result in the diagnosis of a physiological state in the cell or may be used to identify the potential toxicity of a compound, for instance a new or unknown compound or agent. The comparison of expression data, as well as available sequence or other information may be done by researcher or diagnostician or may be done with the aid of a computer and databases as described below.

In another format, the levels of a gene(s) of Tables 1-3, its encoded protein(s), or any metabolite produced by the encoded protein may be monitored or detected in a sample, such as a bodily tissue or fluid sample to identify or diagnose a physiological state of an organism. Such samples may include any tissue or fluid sample, including urine, blood and easily obtainable cells such as peripheral lymphocytes.

Use of the Markers for Monitoring Toxicity Progression

As described above, the genes and gene expression information provided in Tables 1-3 may also be used as markers for the monitoring of toxicity progression, such as that found after initial exposure to a drug, drug candidate, toxin, pollutant, etc. For instance, a tissue or cell sample may be assayed by any of the methods described above, and the expression levels from a gene or genes from Tables 1-3 may be compared to the expression levels found in tissue or cells exposed to the hepatotoxins described herein. The comparison of the expression data, as well as available sequence or other information may be done by researcher or diagnostician or may be done with the aid of a computer and databases.

Use of the Toxicity Markers for Drug Screening

According to the present invention, the genes identified in Tables 1-3 may be used as markers or drug targets to evaluate the effects of a candidate drug, chemical compound or other agent on a cell or tissue sample. The genes may also be used as drug targets to screen for agents that modulate their expression and/or activity. In various formats, a candidate drug or agent can be screened for the ability to simulate the transcription or expression of a given marker or markers or to down-regulate or counteract the transcription or expression of a marker or markers. According to the present invention, one can also compare the specificity of a drug's effects by looking at the number of markers which the drug induces and comparing them. More specific drugs will have less transcriptional targets. Similar sets of markers identified for two drugs may indicate a similarity of effects.

Assays to monitor the expression of a marker or markers as defined in Tables 1-3 may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell.

In one assay format, gene chips containing probes to one, tow or more genes from Tables 1-3 may be used to directly monitor or detect changes in gene expression in the treated or exposed cell. Cell lines, tissues or other samples are first exposed to a test agent and in some instances, a known toxin, and the detected expression levels of one or more, or preferably 2 or more of the genes of Tables 1-3 are compared to the expression levels of those same genes exposed to a known toxin alone. Compounds that modulate the expression patterns of the known toxin(s) would be expected to modulate potential toxic physiological effects in vivo. The genes in Tables 1-3 are particularly appropriate marks in these assays as they are differentially expressed in cells upon exposure to a known hepatotoxin.

In another format, cell lines that contain reporter gene fusions between the open reading frame and/or the transcriptional regulatory regions of a gene in Tables 1-3 and any assayable fusion partner may be prepared. Numerous assayable fusion partners are known and readily available including the firefly luciferase gene and the gene encoding chloramphenicol acetyltransferase (Alam et al. (1990) Anal. Biochem. 188:245-254). Cell lines containing the reporter gene fusions are then exposed to the agent to be tested under appropriate conditions and time. Differential expression of the reporter gene between samples exposed to the agent and control samples identifies agents which modulate the expression of the nucleic acid.

Additional assay formats may be used to monitor the ability of the agent to modulate the expression of a gene identified in Tables 1-3. For instance, as described above, mRNA expression may be monitored directly by hybridization of probes to the nucleic acids of the invention. Cell lines are exposed to the agent to be tested under appropriate conditions and time and total RNA or mRNA is isolated by standard procedures such those disclosed in Sambrook et al. (Molecular Cloning: A Laboratory Manual, 2nd Ed. Cold Spring Harbor Laboratory Press, 1989).

In another assay format, cells or cell lines are first identified which express the gene products of the invention physiologically. Cell and/or cell lines so identified would be expected to comprise the necessary cellular machinery such that the fidelity of modulation of the transcriptional apparatus is maintained with regard to exogenous contact of agent with appropriate surface transduction mechanisms and/or the cytosolic cascades. Further, such cells or cell lines may be transduced or transfected with an expression vehicle (e.g., a plasmid or viral vector) construct comprising an operable non-translated 5′-promoter containing end of the structural gene encoding the gene products of Tables 1-3 fused to one or more antigenic fragments or other detectable markers, which are peculiar to the instant gene products, wherein said fragments are under the transcriptional control of said promoter and are expressed as polypeptides whose molecular weight can be distinguished from the naturally occurring polypeptides or may further comprise an immunologically distinct or other detectable tag. Such a process is well known in the art (see Maniatis).

Cells or cell lines transduced or transfected as outlined above are then contacted with agents under appropriate conditions; for example, the agent comprises a pharmaceutically acceptable excipient and is contacted with cells comprised in an aqueous physiological buffer such as phosphate buffered saline (PBS) at physiological pH, Eagles balanced salt solution (BSS) at physiological pH, PBS or BSS comprising serum or conditioned media comprising PBS or BSS and/or serum incubated at 37° C. Said conditions may be modulated as deemed necessary by one of skill in the art. Subsequent to contacting the cells with the agent, said cells are disrupted and the polypeptides of the lysate are fractionated such that a polypeptide fraction is pooled and contacted with an antibody to be further processed by immunological assay (e.g. ELISA, immunoprecipitation or Western blot). The pool of proteins isolated from the “agent-contacted” sample is then compared with the control samples (no exposure and exposure to a known toxin) where only the excipient is contacted with the cells and an increase or decrease in the immunologically generated signal from the “agent-contacted” sample compared to the control is used to distinguish the effectiveness and/or toxic effects of the agent.

Another embodiment of the present invention provides methods for identifying agents that modulate at least one activity of a protein(s) encoded by the genes in Tables 1-3. Such methods or assays may utilize any means of monitoring or detecting the desired activity.

In one format, the relative amounts of a protein (Tables 1-3) between a cell population that has been exposed to the agent to be tested compared to an unexposed control cell population and a cell population exposed to a known toxin may be assayed. In this format, probes such as specific antibodies are used to monitor the differential expression of the protein in the different cell populations. Cell lines or populations are exposed to the agent to be tested under appropriate conditions and time. Cellular lysates may be prepared from the exposed cell line or population and a control, unexposed cell line or population. The cellular lysates are then analyzed with the probe, such as a specific antibody.

Agents that are assayed in the above methods can be randomly selected or rationally selected or designed. As used herein, an agent is said to be randomly selected when the agent is chosen randomly without considering the specific sequences involved in the association of the a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a nonrandom basis which takes into account the sequence of the target site and/or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site.

The agents of the present invention can be, as examples, peptides, small molecules, vitamin derivatives, as well as carbohydrates. Dominant negative proteins, DNAs encoding these proteins, antibodies to these proteins, peptide fragments of these proteins or mimics of these proteins may be introduced into cells to affect function. “Mimic” used herein refers to the modification of a region or several regions of a peptide molecule to provide a structure chemically different from the parent peptide but topographically and functionally similar to the parent peptide (see Grant GA. in: Meyers (ed.) Molecular Biology and Biotechnology (New York, VCH Publishers, 1995), pp. 659-664). A skilled artisan can readily recognize that there is no limit as to the structural nature of the agents of the present invention.

Nucleic Acid Assay Formats

The genes identified as being differentially expressed upon exposure to a known hepatotoxin (Tables 1-3) may be used in a variety of nucleic acid detection assays to detect or quantititate the expression level of a gene or multiple genes in a given sample. The genes described in Tables 1-3 may also be used in combination with one or more additional genes whose differential expression is associate with toxicity in a cell or tissue. In preferred embodiments, the genes in Tables 1-3 may be combined with one or more of the genes described in related application Nos .60/222,040, 60/244,880, 60/290,029, 60/290,645, 60/292,336, 60/295,798, 60/297,457, 60/298,884 and 60/303,459, all of which are incorporated by reference on page 1 of this application.

Any assay format to detect gene expression may be used. For example, traditional Northern blotting, dot or slot blot, nuclease protection, primer directed amplification, RT-PCR, semi- or quantitative PCR, branched-chain DNA and differential display methods may be used for detecting gene expression levels. Those methods are useful for some embodiments of the invention. In cases where smaller numbers of genes are detected, amplification based assays may be most efficient. Methods and assays of the invention, however, may be most efficiently designed with hybridization-based methods for detecting the expression of a large number of genes.

Any hybridization assay format may be used, including solution-based and solid support-based assay formats. Solid supports containing oligonucleotide probes for differentially expressed genes of the invention can be filters, polyvinyl chloride dishes, particles, beads, microparticles or silicon or glass based chips, etc. Such chips, wafers and hybridization methods are widely available, for example, those disclosed by Beattie (WO 95/11755).

Any solid surface to which oligonucleotides can be bound, either directly or indirectly, either covalently or non-covalently, can be used. A preferred solid support is a high density array or DNA chip. These contain a particular oligonucleotide probe in a predetermined location on the array. Each predetermined location may contain more than one molecule of the probe, but each molecule within the predetermined location has an identical sequence. Such predetermined locations are termed features. There may be, for example, from 2, 10, 100, 1000 to 10,000, 100,000 or 400,000 of such features on a single solid support. The solid support, or the area within which the probes are attached may be on the order of about a square centimeter. Probes corresponding to the genes of Tables 1-3 or from the related applications described above may be attached to single or multiple solid support structures, e.g., the probes may be attached to a single chip or to multiple chips to comprise a chip set.

Oligonucleotide probe arrays for expression monitoring can be made and used according to any techniques known in the art (see for example, Lockhart et al., Nat. Biotechnol. (1996) 14, 1675-1680; McGall et al., Proc. Nat. Acad. Sci. USA (1996) 93, 13555-13460). Such probe arrays may contain at least two or more oligonucleotides that are complementary to or hybridize to two or more of the genes described in Tables 1-3. For instance, such arrays may contain oligonucleotides that are complementary or hybridize to at least 2, 3, 4, 5, 6, 7, 8, 9, 10, 20, 30, 50, 70, 100 or more the genes described herein. Preferred arrays contain all or nearly all of the genes listed in Tables 1-3, or individually, the gene sets of Tables 3A-3S. In a preferred embodiment, arrays are constructed that contain oligonucleotides to detect all or nearly all of the genes in any one of or all of Tables 1-3 on a single solid support substrate, such as a chip.

The sequences of the expression marker genes of Tables 1-3 are in the public databases. Table 1 provides the GenBank Accession Number for each of the sequences (see www.ncbi.nlm.nih.gov/). The sequences of the genes in GenBank are expressly herein incorporated by reference in their entirety as of the filing date of this application, as are related sequences, for instance, sequences from the same gene of different lengths, variant sequences, polymorphic sequences, genomic sequences of the genes and related sequences from different species, including the human counterparts, where appropriate. These sequences may be used in the methods of the invention or may be used to produce the probes and arrays of the invention. In some embodiments, the genes in Tables 1-3 that correspond to the genes or fragments previously associated with a toxic response may be excluded from the Tables.

As described above, in addition to the sequences of the GenBank Accessions Numbers disclosed in the Tables 1-3 , sequences such as naturally occurring variant or polymorphic sequences may be used in the methods and compositions of the invention. For instance, expression levels of various allelic or homologous forms of a gene disclosed in the Tables 1-3 may be assayed. Any and all nucleotide variations that do not alter the functional activity of a gene listed in the Tables 1-3, including all naturally occurring allelic variants of the genes herein disclosed, may be used in the methods and to make the compositions (e.g., arrays) of the invention.

Probes based on the sequences of the genes described above may be prepared by any commonly available method. Oligonucleotide probes for screening or assaying a tissue or cell sample are preferably of sufficient length to specifically hybridize only to appropriate, complementary genes or transcripts. Typically the oligonucleotide probes will be at least 10, 12, 14, 16, 18, 20 or 25 nucleotides in length. In some cases, longer probes of at least 30, 40, or 50 nucleotides will be desirable.

As used herein, oligonucleotide sequences that are complementary to one or more of the genes described in Tables 1-3 refer to oligonucleotides that are capable of hybridizing under stringent conditions to at least part of the nucleotide sequences of said genes. Such hybridizable oligonucleotides will typically exhibit at least about 75% sequence identity at the nucleotide level to said genes, preferably about 80% or 85% sequence identity or more preferably about 90% or 95% or more sequence identity to said genes. “Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals may also be produced by intrinsic fluorescence of the array components themselves. A single background signal can be calculated for the entire array, or a different background signal may be calculated for each target nucleic acid. In a preferred embodiment, background is calculated as the average hybridization signal intensity for the lowest 5% to 10% of the probes in the array, or, where a different background signal is calculated for each target gene, for the lowest 5% to 10% of the probes for each gene. Of course, one of skill in the art will appreciate that where the probes to a particular gene hybridize well and thus appear to be specifically binding to a target sequence, they should not be used in a background signal calculation. Alternatively, background may be calculated as the average hybridization signal intensity produced by hybridization to probes that are not complementary to any sequence found in the sample (e.g. probes directed to nucleic acids of the opposite sense or to genes not found in the sample such as bacterial genes where the sample is mammalian nucleic acids). Background can also be calculated as the average signal intensity produced by regions of the array that lack any probes at all.

The phrase “hybridizing specifically to” refers to the binding, duplexing, or hybridizing of a molecule substantially to or only to a particular nucleotide sequence or sequences under stringent conditions when that sequence is present in a complex mixture (e.g., total cellular) DNA or RNA.

Assays and methods of the invention may utilize available formats to simultaneously screen at least about 100, preferably about 1000, more preferably about 10,000 and most preferably about 1,000,000 different nucleic acid hybridizations.

As used herein a “probe” is defined as a nucleic acid, capable of binding to a target nucleic acid of complementary sequence through one or more types of chemical bonds, usually through complementary base pairing, usually through hydrogen bond formation. As used herein, a probe may include natural (i.e., A, G, U, C, or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages.

The term “perfect match probe” refers to a probe that has a sequence that is perfectly complementary to a particular target sequence. The test probe is typically perfectly complementary to a portion (subsequence) of the target sequence. The perfect match (PM) probe can be a “test probe”, a “normalization control” probe, an expression level control probe and the like. A perfect match control or perfect match probe is, however, distinguished from a “mismatch control” or “mismatch probe.” The terms “mismatch control” or “mismatch probe” refer to a probe whose sequence is deliberately selected not to be perfectly complementary to a particular target sequence. For each mismatch (MM) control in a high-density array there typically exists a corresponding perfect match (PM) probe that is perfectly complementary to the same particular target sequence. The mismatch may comprise one or more bases.

While the mismatch(s) may be located anywhere in the mismatch probe, terminal mismatches are less desirable as a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions.

The term “stringent conditions” refers to conditions under which a probe will hybridize to its target subsequence, but with only insubstantial hybridization to other sequences or to other sequences such that the difference may be identified. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH.

Typically, stringent conditions will be those in which the salt concentration is at least about 0.01 to 1.0 M Na+ion concentration (or other salts) at pH 7.0 to 8.3 and the temperature is at least about 30° C. for short probes (e.g., 10 to 50 nucleotides). Stringent conditions may also be achieved with the addition of destabilizing agents such as formamide.

The “percentage of sequence identity” or “sequence identity” is determined by comparing two optimally aligned sequences or subsequences over a comparison window or span, wherein the portion of the polynucleotide sequence in the comparison window may optionally comprise additions or deletions (i.e., gaps) as compared to the reference sequence (which does not comprise additions or deletions) for optimal alignment of the two sequences. The percentage is calculated by determining the number of positions at which the identical submit (e.g. nucleic acid base or amino acid residue) occurs in both sequences to yield the number of matched positions, dividing the number of matched positions by the total number of positions in the window of comparison and multiplying the result by 100 to yield the percentage of sequence identity. Percentage sequence identity when calculated using the programs GAP or BESTFIT (see below) is calculated using default gap weights.

Probe Design

One of skill in the art will appreciate that an enormous number of array designs are suitable for the practice of this invention. The high density array will typically include a number of test probes that specifically hybridize to the sequences of interest. Probes may be produced from any region of the genes identified in the Tables and the attached representative sequence listing. In instances where the gene reference in the Tables is an EST, probes may be designed from that sequence or from other regions of the corresponding full-length transcript that may be available in any of the sequence databases, such as those herein described. See WO99/32660 for methods of producing probes for a given gene or genes. In addition, any available software may be used to produce specific probe sequences, including, for instance, software available from Molecular Biology Insights, Olympus Optical Co. and Biosoft International. In a preferred embodiment, the array will also include one or more control probes.

High density array chips of the invention include “test probes.” Test probes may be oligonucleotides that range from about 5 to about 500, or about 7 to about 50 nucleotides, more preferably from about 10 to about 40 nucleotides and most preferably from about 15 to about 35 nucleotides in length. In other particularly preferred embodiments, the probes are 20 or 25 nucleotides in length. In another preferred embodiment, test probes are double or single strand DNA sequences. DNA sequences are isolated or cloned from natural sources or amplified from natural sources using native nucleic acid as templates. These probes have sequences complementary to particular subsequences of the genes whose expression they are designed to detect. Thus, the test probes are capable of specifically hybridizing to the target nucleic acid they are to detect.

In addition to test probes that bind the target nucleic acid(s) of interest, the high density array can contain a number of control probes. The control probes may fall into three categories referred to herein as 1) normalization controls; 2) expression level controls; and 3) mismatch controls.

Normalization controls are oligonucleotide or other nucleic acid probes that are complementary to labeled reference oligonucleotides or other nucleic acid sequences that are added to the nucleic acid sample to be screened. The signals obtained from the normalization controls after hybridization provide a control for variations in hybridization conditions, label intensity, “reading” efficiency and other factors that may cause the signal of a perfect hybridization to vary between arrays. In a preferred embodiment, signals (e.g., fluorescence intensity) read from all other probes in the array are divided by the signal (e.g., fluorescence intensity) from the control probes thereby normalizing the measurements.

Virtually any probe may serve as a normalization control. However, it is recognized that hybridization efficiency varies with base composition and probe length. Preferred normalization probes are selected to reflect the average length of the other probes present in the array, however, they can be selected to cover a range of lengths. The normalization control(s) can also be selected to reflect the (average) base composition of the other probes in the array, however in a preferred embodiment, only one or a few probes are used and they are selected such that they hybridize well (i.e., no secondary structure) and do not match any target-specific probes.

Expression level controls are probes that hybridize specifically with constitutively expressed genes in the biological sample. Virtually any constitutively expressed gene provides a suitable target for expression level controls. Typically expression level control probes have sequences complementary to subsequences of constitutively expressed “housekeeping genes” including, but not limited to the actin gene, the transferrin receptor gene, the GAPDH gene, and the like.

Mismatch controls may also be provided for the probes to the target genes, for expression level controls or for normalization controls. Mismatch controls are oligonucleotide probes or other nucleic acid probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent) Preferred mismatch probes contain a central mismatch. Thus, for example, where a probe is a 20 mer, a corresponding mismatch probe will have the identical sequence except for a single base mismatch (e.g., substituting a G, a C or a T for an A) at any of positions 6 through 14 (the central mismatch).

Mismatch probes thus provide a control for non-specific binding or cross hybridization to a nucleic acid in the sample other than the target to which the probe is directed. For example, if the target is present the perfect match probes should be consistently brighter than the mismatch probes. In addition, if all central mismatches are present, the mismatch probes can be used to detect a mutation, for instance, a mutation of a gene in the accompanying Tables 1-3 . The difference in intensity between the perfect match and the mismatch probe provides a good measure of the concentration of the hybridized material.

Nucleic Acid Samples

Cell or tissue samples may be exposed to the test agent in vitro or in vivo. When cultured cells or tissues are used, appropriate mammalian liver extracts may also be added with the test agent to evaluate agents that may require biotransformation to exhibit toxicity. In a preferred format, primary isolates of animal or human hepatocytes which already express the appropriate complement of drug-metabolizing enzymes may be exposed to the test agent without the addition of mammalian liver extracts.

The genes which are assayed according to the present invention are typically in the form of mRNA or reverse transcribed mRNA. The genes may be cloned or not. The genes may be amplified or not. The cloning and/or amplification do not appear to bias the representation of genes within a population. In some assays, it may be preferable, however, to use polyA+RNA as a source, as it can be used with less processing steps.

As is apparent to one of ordinary skill in the art, nucleic acid samples used in the methods and assays of the invention may be prepared by any available method or process. Methods of isolating total mRNA are well known to those of skill in the art. For example, methods of isolation and purification of nucleic acids are described in detail in Chapter 3 of

Laboratory Techniques in Biochemistry and Molecular Biology: Hybridization With Nucleic Acid Probes, Part I Theory and Nucleic Acid Preparation, P. Tijssen, Ed., Elsevier, N.Y. (1993). Such samples include RNA samples, but also include cDNA synthesized from a mRNA sample isolated from a cell or tissue of interest. Such samples also include DNA amplified from the cDNA, and RNA transcribed from the amplified DNA. One of skill in the art would appreciate that it is desirable to inhibit or destroy RNase present in homogenates before homogenates are used.

Biological samples may be of any biological tissue or fluid or cells from any organism as well as cells raised in vitro, such as cell lines and tissue culture cells. Frequently the sample will be a tissue or cell sample that has been exposed to a compound, agent, drug, pharmaceutical composition, potential environmental pollutant or other composition. In some formats, the sample will be a “clinical sample” which is a sample derived from a patient. Typical clinical samples include, but are not limited to, sputum, blood, blood-cells (e.g., white cells), tissue or fine needle biopsy samples, urine, peritoneal fluid, and pleural fluid, or cells therefrom.

Biological samples may also include sections of tissues, such as frozen sections or formalin fixed sections taken for histological purposes.

Forming High Density Arrays

Methods of forming high density arrays of oligonucleotides with a minimal number of synthetic steps are known. The oligonucleotide analogue array can be synthesized on a single or on multiple solid substrates by a variety of methods, including, but not limited to, light-directed chemical coupling, and mechanically directed coupling. See Pirrung, U.S. Pat. No. 5,143,854.

In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′ photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In addition to the foregoing, additional methods which can be used to generate an array of oligonucleotides on a single substrate are described in PCT Publication Nos. WO93/09668 and WO01/23614. High density nucleic acid arrays can also be fabricated by depositing premade or natural nucleic acids in predetermined positions. Synthesized or natural nucleic acids are deposited on specific locations of a substrate by light directed targeting and oligonucleotide directed targeting. Another embodiment uses a dispenser that moves from region to region to deposit nucleic acids in specific spots.

Hybridization

Nucleic acid hybridization simply involves contacting a probe and target nucleic acid under conditions where the probe and its complementary target can form stable hybrid duplexes through complementary base pairing. See WO99/32660. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization tolerates fewer mismatches. One of skill in the art will appreciate that hybridization conditions may be selected to provide any degree of stringency.

In a preferred embodiment, hybridization is performed at low stringency, in this case in 6X SSPET at 37° C. (0.005% Triton X-100), to ensure hybridization and then subsequent washes are performed at higher stringency (e.g., I×SSPET at 37° C.) to eliminate mismatched hybrid duplexes. Successive washes may be performed at increasingly higher stringency (e.g., down to as low as 0.25×SSPET at 37° C. to 50° C.) until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

Signal Detection

The hybridized nucleic acids are typically detected by detecting one or more labels attached to the sample nucleic acids. The labels may be incorporated by any of a number of means well known to those of skill in the art. See WO99/32660.

Databases

The present invention includes relational databases containing sequence information, for instance, for the genes of Tables 1-3, as well as gene expression information from tissue or cells exposed to various standard toxins, such as those herein described (see Table 3A-3S). Databases may also contain information associated with a given sequence or tissue sample such as descriptive information about the gene associated with the sequence information (see Table 1), or descriptive information concerning the clinical status of the tissue sample, or the animal from which the sample was derived. The database may be designed to include different parts, for instance a sequence database and a gene expression database. Methods for the configuration and construction of such databases are widely available, for instance, see U.S. Pat. No. 5,953,727, which is herein incorporated by reference in its entirety.

The databases of the invention may be linked to an outside or external database such as GenBank (www.ncbi.nlm.nih.gov/entrez.index.html); KEGG (www.genome.ad.jp/kegg); SPAD (www.grt.kyushu-u.ac.jp/spad/index.html); HUGO (www.gene. ucl.ac.uk/hugo); Swiss-Prot (www.expasy.ch.sprot); Prosite (www. expasy.ch/tools/scnpsitl.html); OMIM (www.ncbi.nlm.nih.gov/omim); GDB (www.gdb.org); and GeneCard (bioinformatics.weizmann.ac.il/cards). In a preferred embodiment, as described in Tables 1-3, the external database is GenBank and the associated databases maintained by the National Center for Biotechnology Information (NCBI) (www.ncbi.nlm.nih.gov).

Any appropriate computer platform may be used to perform the necessary comparisons between sequence information, gene expression information and any other information in the database or information provided as an input. For example, a large number of computer workstations are available from a variety of manufacturers, such has those available from Silicon Graphics. Client/server environments, database servers and networks are also widely available and appropriate platforms for the databases of the invention.

The databases of the invention may be used to produce, among other things, electronic Northerns that allow the user to determine the cell type or tissue in which a given gene is expressed and to allow determination of the abundance or expression level of a given gene in a particular tissue or cell.

The databases of the invention may also be used to present information identifying the expression level in a tissue or cell of a set of genes comprising one or more of the genes in Tables 1-3, comprising the step of comparing the expression level of at least one gene in Tables 1-3 in a cell or tissue exposed to a test agent to the level of expression of the gene in the database. Such methods may be used to predict the toxic potential of a given compound by comparing the level of expression of a gene or genes in Tables 1-3 from a tissue or cell sample exposed to the test agent to the expression levels found in a control tissue or cell samples exposed to a standard toxin or hepatotoxin such as those herein described. Such methods may also be used in the drug or agent screening assays as described below.

Kits

The invention further includes kits combining, in different combinations, high-density oligonucleotide arrays, reagents for use with the arrays, protein reagents encoded by the genes of the Tables, signal detection and array-processing instruments, gene expression databases and analysis and database management software described above. The kits may be used, for example, to predict or model the toxic response of a test compound, to monitor the progression of hepatic disease states, to identify genes that show promise as new drug targets and to screen known and newly designed drugs as discussed above.

The databases packaged with the kits are a compilation of expression patterns from human or laboratory animal genes and gene fragments (corresponding to the genes of Tables 1-3). In particular, the database software and packaged information include the expression results of Tables 1-3 that can be used to predict toxicity of a test agent by comparing the expression levels of the genes of Tables 1-3 induced by the test agent to the expression levels presented in Tables 3A-3S. In another format, database and software information may be provided in a remote electronic format, such as a website, the address of which may be packaged in the kit.

The kits may used in the pharmaceutical industry, where the need for early drug testing is strong due to the high costs associated with drug development, but where bioinformatics, in particular gene expression informatics, is still lacking. These kits will reduce the costs, time and risks associated with traditional new drug screening using cell cultures and laboratory animals. The results of large-scale drug screening of pre-grouped patient populations, pharmacogenomics testing, can also be applied to select drugs with greater efficacy and fewer side-effects. The kits may also be used by smaller biotechnology companies and research institutes who do not have the facilities for performing such large-scale testing themselves.

Databases and software designed for use with use with microarrays is discussed in Balaban et al., U.S. Pat. Nos. 6,229,911, a computer-implemented method for managing information, stored as indexed Tables 1-3 , collected from small or large numbers of microarrays, and U.S. Pat. No. 6,185,561, a computer-based method with data mining capability for collecting gene expression level data, adding additional attributes and reformatting the data to produce answers to various queries. Chee et al., U.S. Pat. No. 5,974,164, disclose a software-based method for identifying mutations in a nucleic acid sequence based on differences in probe fluorescence intensities between wild type and mutant sequences that hybridize to reference sequences.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### EXAMPLES

### Example 1

### Identification of Toxicity Markers

The hepatotoxins amitryptiline, ANIT, acetaminophen, carbon tetrachloride, CPA, diclofenac, estradiol, indomethacin, valproate, WY-14643 and control compositions were administered to male Sprague-Dawley rats at various time points using adminstration diluents, protocols and dosing regimes as previously described in the art and previously described in the priority applications discussed above.

After adminstration, the dosed animals were observed and tissues were collected as described below:

Observation of Animals

1. Clinical Observations Twice daily—mortality and moribundity check. Cage Side Observations—skin and fur, eyes and mucous membrane, respiratory system, circulatory system, autonomic and central nervous system, somatomotor pattern, and behavior pattern.

Potential signs of toxicity, including tremors, convulsions, salivation, diarrhea, lethargy, coma or other atypical behavior or appearance, were recorded as they occurred and included a time of onset, degree, and duration.

2. Physical Examinations Prior to randomization, prior to initial treatment, and prior to sacrifice.

3. Body Weights Prior to randomization, prior to initial treatment, and prior to sacrifice.

Clinical Pathology

1. Frequency Prior to necropsy.

2. Number of animals All surviving animals.

3. Bleeding Procedure Blood was obtained by puncture of the orbital sinus while under 70% CO₂/30% O₂ anesthesia.

4. Collection of Blood Samples Approximately 0.5 mL of blood was collected into EDTA tubes for evaluation of hematology parameters.

Approximately 1 mL of blood was collected into serum separator tubes for clinical chemistry analysis.

Approximately 200 uL of plasma was obtained and frozen at ^{˜}−80° C. for test compound/metabolite estimation.

An additional 2 mL of blood was collected into a 15 mL conical polypropylene vial to which ^{˜}3 mL of Trizol was immediately added. The contents were immediately mixed with a vortex and by repeated inversion. The tubes were frozen in liquid nitrogen and stored at ^{˜}−80° C.

Termination Procedures

Terminal Sacrifice

Approximately 1 and 3 and 6 and 24 and 48 hours and 5-7 days after the initial dose, rats were weighed, physically examined, sacrificed by decapitation, and exsanguinated. The animals were necropsied within approximately five minutes of sacrifice. Separate sterile, disposable instruments were used for each animal, with the exception of bone cutters, which were used to open the skull cap. The bone cutters were dipped in disinfectant solution between animals.

Necropsies were conducted on each animal following procedures approved by board-certified pathologists.

Animals not surviving until terminal sacrifice were discarded without necropsy (following euthanasia by carbon dioxide asphyxiation, if moribund). The approximate time of death for moribund or found dead animals was recorded.

Postmortem Procedures

Fresh and sterile disposable instruments were used to collect tissues. Gloves were worn at all times when handling tissues or vials. All tissues were collected and frozen within approximately 5 minutes of the animal's death. The liver sections and kidneys were frozen within approximately 3-5 minutes of the animal's death. The time of euthanasia, an interim time point at freezing of liver sections and kidneys, and time at completion of necropsy were recorded. Tissues were stored at approximately −80° C. or preserved in 10% neutral buffered formalin.

Tissue Collection and Processing

Liver

1. Right medial lobe—snap frozen in liquid nitrogen and stored at −80° C.

2. Left medial lobe—Preserved in 10% neutral-buffered formalin (NBF) and evaluated for gross and microscopic pathology.

3. Left lateral lobe—snap frozen in liquid nitrogen and stored at ˜−80° C.

Heart

A sagittal cross-section containing portions of the two atria and of the two ventricles was preserved in 10% NBF. The remaining heart was frozen in liquid nitrogen and stored at ˜−80° C.

Kidneys (both)

1. Left—Hemi-dissected; half was preserved in 10% NBF and the remaining half was frozen in liquid nitrogen and stored at −80° C.

2. Right—Hemi-dissected; half was preserved in 10% NBF and the remaining half was frozen in liquid nitrogen and stored at ˜−80° C.

Testes (Both)

A sagittal cross-section of each testis was preserved in 10% NBF. The remaining testes were frozen together in liquid nitrogen and stored at ˜−80° C.

Brain (Whole)

A cross-section of the cerebral hemispheres and of the diencephalon was preserved in 10% NBF, and the rest of the brain was frozen in liquid nitrogen and stored at ˜−80° C.

Microarray sample preparation was conducted with minor modifications, following the protocols set forth in the Affymetrix GeneChip Expression Analysis Manual. Frozen tissue was ground to a powder using a Spex Certiprep 6800 Freezer Mill. Total RNA was extracted with Trizol (GibcoBRL) utilizing the manufacturer's protocol. The total RNA yield for each sample was 200-500 μg per 300 mg tissue weight. mRNA was isolated using the Oligotex mRNA Midi kit (Qiagen) followed by ethanol precipitation. Double stranded cDNA was generated from mRNA using the SuperScript Choice system (GibcoBRL). First strand cDNA synthesis was primed with a T7-(dT24) oligonucleotide. The CDNA was phenol-chloroform extracted and ethanol precipitated to a final concentration of 1 μg/ml. From 2 μg of cDNA, cRNA was synthesized using Ambion's T7 MegaScript in vitro Transcription Kit.

To biotin label the cRNA, nucleotides Bio-11-CTP and Bio-16-UTP (Enzo Diagnostics) were added to the reaction. Following a 37° C. incubation for six hours, impurities were removed from the labeled cRNA following the RNeasy Mini kit protocol (Qiagen). cRNA was fragmented (fragmentation buffer consisting of 200 mM Tris-acetate, pH 8.1, 500 mM KOAc, 150 mM MgOAc) for thirty-five minutes at 94° C. Following the Affymetrix protocol, 55 μg of fragmented cRNA was hybridized on the Affymetrix rat array set for twenty-four hours at 60 rpm in a 45° C. hybridization oven. The chips were washed and stained with Streptavidin Phycoerythrin (SAPE) (Molecular Probes) in Affymetrix fluidics stations. To amplify staining, SAPE solution was added twice with an anti-streptavidin biotinylated antibody (Vector Laboratories) staining step in between. Hybridization to the probe arrays was detected by fluorometric scanning (Hewlett Packard Gene Array Scanner). Data was analyzed using Affymetrix GeneChip^{□} version 3.0 and Expression Data Mining (EDMT) software (version 1.0), GeneExpress2000, and S-Plus.

Table 1 discloses those genes that are differentially expressed upon exposure to the named toxins and their corresponding GenBank Accession and Sequence Identification numbers, the identities of the metabolic pathways in which the genes function, the gene names if known, and the unigene cluster titles. The comparison code represents the various toxicity or liver pathology state that each gene is able to discriminate as well as the individual toxin type associated with each gene. The codes are defined in Table 2. The GLGC ID is the internal Gene Logic identification number.

Table 2 defines the comparison codes used in Table 1 .

Tables 3A-3S disclose the summary statistics for each of the comparisons performed. Each gene is identified by its Gene Logic identification number and can be cross-referenced to a gene name and representative SEQ ID NO. in Table 1. The group mean (eg. toxicity group) is the mean signal intensity as normalized for the various chip parameters in the samples that are being assayed for in the particular comparison. The non-group (eg. non-toxicity group) mean represents the mean signal intensity as normalized for the various chip parameters in the samples that are not being assayed for in the particular comparison. The mean values are derived from Average Difference (AveDiff) values for a particular gene, averaged across the corresponding samples. Each individual Average Difference value is calculated by integrating the intensity information from multiple probe pairs that are tiled for a particular fragment. The normalization algorithm used to calculate the AveDiff is based on the observation that the expression intensity values from a single chip experiment have different distributions, depending on whether small or large expression values are considered. Small values, which are assumed to be mostly noise, are approximately normally distributed with mean zero, while larger values roughly obey a log-normal distribution; that is, their logarithms are normally distributed with some nonzero mean.

The normalization process computes separate scale factors for “non-expressors” (small values) and “expressors” (large ones). The inputs to the algorithm are pre-normalized Average Difference values, which are already scaled to set the trimmed mean equal to 100. The algorithm computes the standard deviation SD noise of the negative values, which are assumed to come from non-expressors. It then multiplies all negative values, as well as all positive values less than 2.0* SD noise, by a scale factor proportional to 1/SD noise.

Values greater than 2.0* SD noise are assumed to come from expressors. For these values, the standard deviation SD log (signal) of the logarithms is calculated. The logarithms are then multiplied by a scale factor proportional to 1/SD log (signal) and exponentiated. The resulting values are then multiplied by another scale factor, chosen so there will be no discontinuity in the normalized values from unscaled values on either side of 2.0* SD noise. Some AveDiff values may be negative due to the general noise involved in nucleic acid hybridization experiments. Although many conclusions can be made corresponding to a negative value on the GeneChip platform, it is difficult to assess the meaning behind the negative value for individual fragments. Our observations show that, although negative values are observed at times within the predictive gene set, these values reflect a real biological phenomenon that is highly reproducible across all the samples from which the measurement was taken. For this reason, those genes that exhibit a negative value are included in the predictive set. It should be noted that other platforms of gene expression measurement may be able to resolve the negative numbers for the corresponding genes. The predictive ability of each of those genes should extend across platforms, however. Each mean value is accompanied by the standard deviation for the mean. LDA is the linear discriminant analysis that measures the ability of each gene to predict whether or not a sample is toxic. The LDA score is calculated by the following steps:

Calculation of a Discriminant Score.

Let X₁ represent the AveDiff values for a given gene across the Group 1 samples, i=1 . . . n.

Let Y₁ represent the AveDiff values for a given gene across the Group 2 samples, i=1 . . . t.

The calculations proceed as follows:

1. Calculate mean and standard deviation for Xᵢ's and Yᵢ's, and denote these by m_{X}, m^{Y}, s_{X},s_{Y}.

2. For all X₁'s and Yᵢ's, evaluate the function f(z)=((1/s_{Y})*exp(−0.5*((z-m_{Y})/s_{Y})²))/(((1/s_{Y})*exp(−0.5*((z-m_{Y})/s_{Y})²))+((1/s_{X})*exp(−0.5*((z-m_{X})/s_{X})²))).

3. The number of correct predictions, say P, is then the number of Yᵢ's such that f(Yᵢ)>0.5 plus the number of Xᵢ's such that f(Xᵢ)<0.5.

4. The discriminant score is then P/(n+t)

Linear discriminant analysis uses both the individual measurements of each gene and the calculated measurements of all combinations of genes to classify samples. For each gene a weight is derived from the mean and standard deviation of the tox and nontox groups. Every gene is multiplied by a weight and the sum of these values results in a collective discriminate score. This discriminant score is then compared against collective centroids of the tox and nontox groups. These centroids are the average of all tox and nontox samples respectively. Therefore, each gene contributes to the overall prediction. This contribution is dependent on weights that are large positive or negative numbers if the relative distances between the tox and nontox samples for that gene are large and small numbers if the relative distances are small. The discriminant score for each unknown sample and centroid values can be used to calculate a probability between zero and one as to which group the unknown sample belongs.

### Example 2

### General Toxicity Modeling

Samples were selected for grouping into tox-responding and non-tox-responding groups by examining each study individually with PCA to determine which treatments had an observable response. Only groups where confidence of their tox-responding and non-tox-responding status was established were included in building a general tox model.

Two general types of models were built for general toxicity determination. One model used information from the expression patterns of each gene individually and then combined all the information using linear weights for each gene. The second type determined orthogonal vectors describing all the expression information collectively and used these composite vectors to predict toxicity.

Over 500 linear discriminant models were generated to describe toxic and non-toxic samples. The top 10, 25, 50 and 100 discriminant genes were used to determine toxicity by calculating each gene's contribution with homo and heteroscedastic treatment of variance and inclusion or exclusion of mutual information between genes. Prediction of samples within the database exceeded 90% for most models. In addition, models were built by sequential use of two, five, ten, twenty five, and fifty genes, starting with the best discriminators and proceeding to the worst discriminators without replication. All discriminating genes and/or ESTs had at least 70% discriminate ability, which was previously determined to be significant via randomization experiments. It was determined that combinations of genes generally provided a better predictive ability then individual genes and that the more genes used the better predictive ability. It was also determined that combining the worst fifty discriminating genes provided better prediction than the best single gene and that many combinations of two or more genes provided better prediction than the best individual gene. Although the preferred embodiment includes fifty or more genes, many pairings or greater combinations of genes can work better than individual genes. All combinations of two or more genes from the selected list may be used to predict toxicity. These combinations could be selected by pairing in an ordered, agglomerate, divisive, or random approach. Further, as yet undetermined genes could be combined with individual or combination of genes described here to increase predictive ability. However, the genes described here may contribute most of the predictive ability of any such undetermined combinations.

The second approach used has been described in U.S. Provisional Application 60/______, using this approach all 527 genes and/or EST were used to predict toxic from non-toxic samples with greater than 94% accuracy when 15 components are used. Although using the first fifteen components provided a preferred model, other variations of this method can provide adequate predictive ability. These include selective inclusion of components via agglomerate, divisive, or random approaches or extraction of loading and combining them in ordered, agglomerate, divisive, or random approaches. Also the use of these composite variables in logistic regression to determine classification of samples can also be accomplished with linear discriminate analysis, neural or Bayesian networks, or other forms of regression and classification based on categorical or continual dependent and independent variables.

### Example 3

### Modeling Methods

The above modeling methods provide broad approaches of combining the expression of genes to predict sample toxicity. One method uses each variable individually and weights them; the other combines variables as a composite measure and adds weights to them after combination into a new variable. One could also provide no weight in a simple voting method or determine weights in a supervised or unsupervised method using agglomerate, divisive, or random approaches. All or selected combinations of genes may be combined in ordered, agglomerate, or divisive, supervised or unsupervised clustering algorithms with unknown samples for classification. Any form of correlation matrix may also be used to classify unknown samples. The spread of the group distribution and discriminate score alone provide enough information to enable a skilled person to generate all of the above types of models with accuracy that can exceed discriminate ability of individual genes. Some examples of methods that could be used individually or in combination after transformation of data types include but are not limited to: Discriminant Analysis, Multiple Discriminant Analysis, logistic regression, multiple regression analysis, linear regression analysis, conjoint analysis, canonical correlation, hierarchical cluster analysis, k-means cluster analysis, self-organizing maps, multidimensional scaling, structural equation modeling, support vector machine determined boundaries, factor analysis, neural networks, bayesian classifications, and resampling methods.

### Example 4

### Grouping of Individual compound and Pathology Classes

Samples were grouped into individual pathology classes based on known toxicological responses and observed clinical chemical and pathology measurements or into early and late phases of observable toxicity within a compound (Tables 3A-3S). The top 10, 25, 50, 100 genes based on individual discriminate scores were used in a model to ensure that combination of genes provided a better prediction than individual genes. As described above, all combinations of two or more genes from this list could potentially provide better prediction than individual genes when selected in any order or by ordered, agglomerate, divisive, or random approaches. In addition, combining these genes with other genes could provide better predictive ability, but most of this predictive ability would come from the genes listed here.

Samples may be considered toxic if they score positive in any pathological or individual compound class represented here or in any modeling method mentioned under general toxicology models based on combination of individual time and dose grouping of individual toxic compounds obtainable from the data. The pathological groupings and early and late phase models are preferred examples of all obtainable combinations of sample time and dose points. Most logical groupings with one or more genes and one or more sample dose and time points should produce better predictions of general toxicity, pathological specific toxicity, or similarity to known toxicant than individual genes.

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

TABLE 1
Document Number 1650775

| | | | | | | |
|---|---|---|---|---|---|---|
| | | Nucleo- | | | | |
| | | tide | | | | |
| | Compar- | Se- | | | | |
| GLGC | ison | quence | GenBank | | | |
| ID | Code | ID | Acc ID | Pathways | Known Gene Name | Unigene Cluster Title |

| | | | | | | |
|---|---|---|---|---|---|---|
| 19 | N | 1729 | NM_017258 | | B-cell translocation gene 1, | B-cell translocation gene 1, anti- |
| | | | | | anti-proliferative | proliferative |
| 20 | L, N | 1729 | NM_017258 | | B-cell translocation gene 1, | B-cell translocation gene 1, anti- |
| | | | | | anti-proliferative | prolifeative |
| 43 | E, P | 1698 | NM_022287 | Glycosaminoglycan | HMm: alpha L-iduronidase | Rattus norvegicus sulfate anion |
| | | | | degradation | | transporter (sat-1) mRNA, complete |
| | | | | | | cds |
| 55 | O | 1535 | NM_012511 | Oxidative | ATPase, Cu++ transporting, | ATPase, Cu++ transporting, beta |
| | | | | phosphorylation | beta polypeptide (same as | polypeptide (same as Wilson disease) |
| | | | | | Wilson disease) | |
| 64 | H | 1620 | NM_016991 | | Adrenergic, alpha 1B-, receptor | Adrenergic, alpha 1B-, receptor |
| 72 | F | 1420 | M57263 | | Hsp: PROTEIN-GLUTAMINE | Rat protein-glutamine gamma- |
| | | | | | GAMMA- | glutamyltransferase mRNA, complete |
| | | | | | GLUTAMYL- | cds |
| | | | | | TRANSFERASE K | |
| 90 | E | 1454 | U20796 | | | Rattus norvegicus nuclear receptor |
| | | | | | | Rev-ErbA-beta mRNA, partial cds |
| 134 | A | 1346 | D87839 | Alanine and aspartate | HHs: 4-aminobutyrate | Rattus norvegicus mRNA for beta- |
| | | | | metabolism, Butanoate | aminotransferase | alanine oxogultarate aminotrans- |
| | | | | metabolism, Glutamate | | ferase, complete cds |
| | | | | metabolism, Propanoate | | |
| | | | | metabolism, beta-Alanine | | |
| | | | | metabolism | | |
| 135 | A | 1346 | D87839 | Alanine and aspartate | HHs: 4-aminobutyrate | Rattus norvegicus mRNA for beta- |
| | | | | metabolism, Butanoate | aminotransferase | alanine oxoglutarate aminotrans- |
| | | | | metabolism, Glutamate | | ferase, complete cds |
| | | | | metabolism, Propanoate | | |
| | | | | metabolism, beta-Alanine | | |
| | | | | metabolism | | |
| 155 | P, Q | 1712 | NM_022849 | | crp-ductin | Rattus norvegicus ebnerin mRNA, |
| | | | | | | complete cds |
| 155 | P | 1712 | NM_022849 | | crp-ductin | Rattus norvegicus ebnerin mRNA, |
| | | | | | | compelete cds |
| 164 | H | 538 | AI010480 | Citrate cycle (TCA cycle), | Malate dehydrogenase 2 NAD | Rat mRNA for mitochondrial malate |
| | | | | Glyoxylate and | (mitochondrial) | dehydrogenase (EC 1.1.1.37) |
| | | | | dicarboxylate metabolism | | |
| | | | | Pyruvate metabolism | | |
| 228 | D | 1452 | U20194 | | | Rattus norvegicus complement |
| | | | | | | C8 beta (C8b) mRNA, partial cds |
| 291 | O | 1538 | NM_012522 | Glycine, serine and | Cystathionine beta synthase | Cystathionine beta synthase |
| | | | | threonine metabolism, | | |
| | | | | Methionine metabolism, | | |
| | | | | Selenoamino acid | | |
| | | | | metabolism | | |
| 330 | R | 1251 | AI235460 | | | Rattus norvegicus synapse-associated |
| | | | | | | protein 102 mRNA, complete cds |
| 347 | J | 1443 | U01914 | | | Rattus norvegicus AKAP95 mRNA, |
| | | | | | | partial cds |
| 351 | A | 1720 | NM_024127 | | HHs: growth arrest and DNA- | Rattus norvegicus GADD45 mNRA, |
| | | | | | damage-inducible, alpha | complete cds |
| 352 | A, J | 1720 | NM_024127 | | HHs: growth arrest and DNA- | Rattus norvegicus GADD45 mRNA, |
| | | | | | damage-inducible, alpha | complete cds |
| 353 | A, B, C, J | 1720 | NM_024127 | | HHs: growth arrest and DNA- | Rattus norvegicus GADD45 mRNA, |
| | | | | | damage-inducible, alpha | complete cds |
| 354 | A, J, Q | 1720 | NM_024127 | | HHs: growth arrest amd DNA- | Rattus norvegicus GADD45 mRNA, |
| | | | | | damage-inducible, alpha | complete cds |
| 355 | N | 1600 | NM_013086 | | CAMP responsive element | CAMP responsive element modulator |
| | | | | | modulator, transcriptional | |
| | | | | | repressor CREM | |
| 356 | N | 1658 | NM_017334 | | CAMP responsive element | CAMP responsive element modulator |
| | | | | | modular | |
| 360 | R | 1728 | NM_012894 | | RNA editing deaminase of | RNA editing deaminase of glutamate |
| | | | | | glutamate receptors | receptors |
| 372 | F, M | 1482 | U94708 | | | Rattus norvegicus prostaglandin E |
| | | | | | | receptor EP2 subtype mRNA, |
| | | | | | | complete cds |
| 373 | P | 1578 | NM_012833 | | Canalicular multispecific | Canalicular mustispecific organic |
| | | | | | organic anion transporter | anion transporter |
| 384 | O | 1457 | U25137 | | | Rattus norvegicus alternatively |
| | | | | | | spliced signal transducer an regulator |
| | | | | | | of transcription 5a2 (STAT5a2) |
| | | | | | | mRNA, partial cds |
| 396 | M | 1464 | U49694 | | Hsp: CYTOSOLIC ACYL | Rattus norvegicus brain cytosolic acyl |
| | | | | | COENZYME A | coenzyme A thioester hydrolase |
| | | | | | THIOESTER HYDROLASE | mRNA, complete cds |
| 397 | S | 1614 | NM_013214 | | acyl-CoA hydrolase | Rattus norvegicus brain cytosolic acyl |
| | | | | | | coenzyme A thioester hydrolase |
| | | | | | | mRNA complete cds, acyl-CoA |
| | | | | | | hydrolase |
| 402 | N | 1734 | NM_022403 | Tryptophan metabolism | HHs: tryptophan 2, | Rat tryptophan-2, 3-dioxygenase |
| | | | | | 3-dioxygenase | mRNA complete cds |
| 466 | L | 1517 | X81395 | | Hsp: LIVER CARBOXYL- | R. norvegicus mRNA for pl |
| | | | | | ESTERASE 3 PRECURSOR | 5.5 esterase (ES-3) |
| 475 | F | 1224 | AI233828 | | | ESTs, Moderately similar to |
| | | | | | | LYSOMOMAL ALPHA- |
| | | | | | | MANNOSIDASE PRECURSOR |
| | | | | | | [M. musculus] |
| 488 | F | 1350 | E00717 | Fatty acid metabolism, | Cytochrome P450, subfamily I | Cytochrome P450, subfamily I |
| | | | | Tryptophan metabolism | (aromatic compound-inducible), | (aromatic compund-inducible), |
| | | | | | member A1 (C6, form c) | member A1 (C6, form c) |
| 489 | F | 1540 | NM_012540 | Fatty acid metabolism, | Cytochrome P450, Subfamily I | Cytochrome P450, subfamily I |
| | | | | Tryptophan metabolism | (aromatic compound-inducible), | (aromatic compund-inducible), |
| | | | | | member A1 (C6, form c) | member A1 (C6, form c) |
| 494 | G | 1581 | NM-012880 | | Superoxide dimutase 3 | Superoxide dimutase 3 |
| 498 | C | 402 | AA956278 | | | ESTs |
| 556 | A, E | 1575 | NM_012803 | | Protein C | Protein C |
| 563 | M | 1536 | NM_012516 | | Complement component 4 | Complement component 4 binding |
| | | | | | binding protein, alpha | protein, alpha |
| 573 | A | 1169 | AI232087 | | | R. norvegicus mRNA for |
| | | | | | | (S)-2-hydroxy acid oxidase |
| 574 | H, I | 1682 | NM_019905 | | calpactin I heavy chain | R. norvegicus mRNa for |
| | | | | | | (S)-2-hydroxy acid oxidase, |
| | | | | | | Rattus norvegicus clone |
| | | | | | | BB. 1. 4. 1 unknown Glu-Pro |
| | | | | | | dipeptide repeat protein mRNA, |
| | | | | | | complete cds, calpactin I heavy chain |
| 633 | A, G | 1146 | AI231127 | | | ESTs |
| 634 | P | 1381 | K01932 | Glutathione metabolism | Hsp: GLUTATHIONE S- | Rat liver glutathione S-transferase Yc |
| | | | | | TRANSFERASE YC-1 | subunit mRNA, complete cds |
| 635 | P | 1515 | X78848 | | | Rat liver glutathione S-transferase Yc |
| | | | | | | subunit mRNA, complete cds |
| 650 | J | 1607 | NM_013134 | Sterol biosynthesis | 3-hydroxy-3-methylglutaryl- | 3-hydroxy-3-methylglutaryl- |
| | | | | | Coenzyme A reductase | Coenzyme A reductase |
| 651 | J | 1607 | NM_013134 | Sterol biosynthesis | 3-hydroxy-3-methylglutaryl- | 3-hydroxy-3-methylglutaryl- |
| | | | | | Coenzyme A reductase | Coenzyme A reductase |
| 671 | B | 1445 | U04808 | | | Rattus norvegicus Sprague-Dawley |
| | | | | | | putative G-protein coupled receptor |
| | | | | | | (GCR) mRNA, complete cds |
| 672 | O | 1492 | X13722 | | Low density lipoprotein receptor | Rat mRNA for LDL-receptor |
| 682 | P | 1627 | NM_017051 | | Superoxide dimutase 2, | Superoxide dimutase 2, mitochondrial |
| | | | | | mitochondrial | |
| 699 | M, P | 1465 | U55765 | | | Rattus norvegicus RASP1-mRNA, |
| | | | | | | complete cds |
| 729 | O | 1429 | M95762 | | | Rattus norvegicus GABA transporter |
| | | | | | | GAT-2 mRNA, complete cds |
| 761 | A | 41 | AA817685 | | | Rattus norvegicus mRNA for |
| | | | | | | cytochrome b5 |
| 794 | A, D, | 1472 | U68168 | Tryptophan metabolism | HHs: kynureninase | Rattus norvegicus L-kynurenine |
| | E, G | | | | (L-kynurenine) hydrolase) | hydrolase mRNA, compled cds |
| 809 | J | 1451 | U17035 | | | Rattus norvegicus interferon |
| | | | | | | inducible protein 10 (IP-10) mRNA, |
| | | | | | | complete cds |
| 811 | A | 1342 | D63704 | Pantothenate and CoA | HHs: dihydropyrimidinase | Rat mRNA for dihydrophyrimidinase, |
| | | | | biosynthesis, Pyrimidine | | complete cds |
| | | | | metabolism, beta-Alanine | | |
| | | | | metabolism | | |
| 812 | A | 1342 | D63704 | Pantothenate and CoA | HHs: dihydropydropyrimidinase | EST, Highly similar to DPYS_RAT |
| | | | | biosynthesis, Pyrimidine | | DIHYDROPYRIMIDINASE |
| | | | | metabolism, beta-Alanine | | [R. norvegicus], Rat mRNA for |
| | | | | metabolism | | dihydropyrimidinase, complete cds |
| 820 | E | 238 | AA892395 | Fructose and mannose | Aldolase B, fructose- | Aldolase B, fructose-biophosphate |
| | | | | metabolism, Glycolysis/ | biophosphate | |
| | | | | Gluconeogenesis, Pentose | | |
| | | | | phosphate cycle | | |
| 825 | A | 381 | AA946108 | | | Rattus norvegicus laminin-5 alpha 3 |
| | | | | | | chain mRNA, complete cds |
| 851 | A | 1721 | NM_024132 | | fatty acid amide hydrolase | Rattus norvegicus fatty acid amide |
| | | | | | | hydrolase mRNA. complete cds |
| 906 | K | 1480 | U83112 | | | Rattus norvegicus INS-1 winged |
| | | | | | | helix mRNA, complete cds |
| 912 | A | 1467 | U59184 | | BcI2-associated X protein | BcI2-asspciated X protein |
| 923 | A, J | 1632 | NM_017076 | | Tumor-associated glycoprotein | Tumor-associated glycoprotein pE4 |
| 945 | P | 1349 | D88666 | | pE4 | Rattus norvegicus mRNA for PS-PLA1, |
| | | | | | | complete cds |
| 955 | M | 1471 | U67138 | | | Rattus norvegicus PSD-95/SAP90- |
| | | | | | | associated protein-2 mRNA, complete |
| | | | | | | cds |
| 958 | I, Q | 1591 | NM_012977 | | Lectin, galactose binding, | Lectin, galactose binding, soluble 9 |
| | | | | | soluble 9 (Galectin-9) | (Galectin-9) |
| 961 | A | 1573 | NM_012796 | Glutathione metabolism | Glutathione S-transferase 1 | Glutathione S-transferase 1 (theta) |
| | | | | | (theta) | |
| 1007 | A | 1589 | NM_012942 | Bile acid biosynthesis | Cytochrom P450 (cholesterol | Cytochrom P450 (cholesterol |
| | | | | | hydroxylase 7 alpha) | hydroxylase 7 alpha) |
| 1037 | I | 1500 | X57523 | | Transporter 1, ABC (ATP | R. norvegicus mtp 1 mRNA |
| | | | | | binding cassette) | |
| 1099 | A | 1678 | NM_019303 | | Cytochrome P450, | Cytochrome P450, subfamily IIF, |
| | | | | | subfamily IIF, polypeptide 1 | polypeptide 1 |
| 1114 | N | 586 | AI029917 | | | Rattus norvegicus neuron-specific |
| | | | | | | enolase (NSF) mRNA, complete cds |
| 1126 | A, I | 1143 | AI231007 | | | Rattus norvegicus cca1 mRNA, |
| | | | | | | complete cds |
| 1141 | E, Q | 1505 | X59601 | | | Rat mRNA for plectin |
| 1169 | E, H | 1008 | A1177161 | | | Rattus norvegicus NF-E2-related factor |
| | | | | | | 2 mNRA, complete cds |
| 1173 | A | 1661 | NM_019184 | Fatty acid metabolism, | Cytochrome P450, | Cytochrome P450, subfamily IIC |
| | | | | | subfamily IIC | |
| | | | | Tryptophan metabolism | (mephenytoin 4-hydroxylase) | (mephenytoin 4-hydroxylase) |
| 1174 | N | 1661 | NM_019184 | Fatty acid metabolism, | Cytochrome P450, | Cytochrome P450, subfamily IIC |
| | | | | Tryptophan metabolism | subfamily IIC (mephenytoin | (mephenytoin 4-hydroxylase) |
| | | | | | 4-hydroxylase) | |
| 1175 | A, E, M | 1661 | NM_019184 | Fatty acid metabolism, | Cytochrome P450, | Cytochrome P450, subfamily IIC |
| | | | | Tryptophan metabolism | subfamily IIC (mephenytoin | (mephenytoin 4-hydroxylase) |
| | | | | | 4-hydroxylase) | |
| 1183 | J | 485 | AF013144 | | Hsp: DUAL SPECIFICITY | Rattus norvegicus MAP-kinase |
| | | | | | PROTEIN PHOSPHATASE 5 | phosplatase (cpg21) mRNA, complete |
| | | | | | | cds |
| 1221 | B, F, Q | 1326 | D11445 | | | Rattus norvegicus mRNA for gro, |
| | | | | | | complete cds |
| 1223 | E | 1423 | M75281 | | | Rat cystatain S (CysS) gene, complete |
| | | | | | | cds |
| 1246 | A | 1569 | NM_012770 | Purine metabolism | Guanylate cyclase, soluble, beta | Guanylate cyclase, soluble, beta 2 (GTP |
| | | | | | 2 (GTP pyrophosphate-lyase) | pyrophosphate-lyase) |
| 1258 | I | 1611 | NM_013185 | | Hemopoietic cell tyrosine kinase | Hemopoietic cell tyrosine kinase |
| 1271 | Q | 1384 | L07073 | | | Rat clathrin-associated adaptor protein |
| | | | | | | homolog (p47A) mRNA, complete cds |
| 1279 | F | 1477 | U75916 | | | Rattus norvegicus zonula occludens 2 |
| | | | | | | protein (ZO-2) mRNA, partial cds |
| 1305 | J | 1636 | NM_017127 | Glycerolipid metabolism | choline kinase | choline kinase |
| 1306 | J | 1636 | NM_017127 | Glycerolipid metabolism | choline kinase | choline kinase |
| 1394 | G | 1461 | U37099 | | | Rattus norvegicus GTP-binding protein |
| | | | | | | (rab 3C) mRNA, complete cds |
| 1399 | C, D, G | 1623 | NM_017006 | Glutathione metabolism, | Glucose-6-phosphate | Gluscose-6-phosphate dehydrogenase |
| | | | | Pentose phosphate cycle | dehydrogenase | |
| 1409 | A | 560 | AI012802 | Pyruvate metabolism | HHs: hydroxyacyl gluathione | Rattus norvegicus round spermatid |
| | | | | | hydrolase | protein RSP29 gene, complete cds |
| 1411 | C, D | 920 | AI172075 | | | ESTs |
| 1426 | Q | 1528 | Z48225 | | | R. norvegicus mRNA for protein |
| | | | | | | synthesis initiation factor eIF-2B delta |
| | | | | | | subunit |
| 1430 | M | 1542 | NM_012545 | Histidine metabolism, | Dopa decarboxylase (aromatic | Dopa decarboxylase (aromatic L-amino |
| | | | | Phenylalanine metabolism, | L-amino acid decarboxylase) | acid decarboxylase) |
| | | | | Tryptophan metabolism, | | |
| | | | | Tyrosine metabolism | | |
| 1447 | F | 1651 | NM_017281 | | proteasome (prosome, | proteasome (prosome, macropain) |
| | | | | | macropain) subunit, alpha type 4 | subunit, alpha type 4 |
| 1460 | C, D | 1439 | S76054 | | Keratin 8 | Keratin 8 |
| 1475 | J | 1386 | L16764 | | Heat shock protein 70-1, S100 | Rattus norvegicus S100A1 gene, Rattus |
| | | | | | calcium binding protein A1 | norvegicus heat shock protein 70 |
| | | | | | | (HSP70) mNRA, complete cds |
| 1478 | A | 1566 | NM_012744 | Alanine and aspartate | Pyruvate carboxylase | Pyruvate carboxylase |
| | | | | metabolism, Citrate cycle | | |
| | | | | (TCA cycle), Pyruvate | | |
| | | | | metabolism | | |
| 1479 | A, G, K | 1566 | NM_012744 | Alanine and aspartate | Pyruvate carboxylase | Pyruvate carboxylase |
| | | | | metabolism, Citrate cycle | | |
| | | | | (TCA cycle), Pyruvate | | |
| | | | | metabolism | | |
| 1501 | A, C, F, H | 690 | AI072634 | | | Rattus norvegicus cytokeratin-18 |
| | | | | | | mRNA, partial cds |
| 1507 | B, Q | 1105 | AI229235 | | | ESTs |
| 1510 | Q | 1646 | NM_017224 | | organic cationic transporter- | organic cationic transporter-like 1 |
| | | | | | like 1 | |
| 1514 | B | 1559 | NM_012678 | | Tropomycin 4 | Tropomycin 4 |
| 1520 | H | 1659 | NM_019165 | | interleukin 18 | interleukin 18 |
| 1521 | B, Q | 1601 | NM_013091 | | Tumor necrosis factor receptor | Tumor necrosis factor receptor |
| 1529 | A, G | 1599 | NM_013082 | | Ryudocan/syndec 2 | Ryudocan/syndec 2 |
| 1531 | A | 1655 | NM_017300 | Bile acid biosynthesis, | bile acid-Coenzyme A | bile acid-Coenzyme A dehydrogenase: |
| | | | | Taurine and hypotaurine | dehydrogenase: amino acid | amino n-acyltransferase |
| | | | | metabolism | n-acyltransferase | |
| 1538 | E | 493 | AF039890 | | Leucine arylaminopeptidase 1 | Rat kidney Zn-peptidase amino- |
| | | | | | | peptidase N mRNA, complete cds |
| 1542 | G, H | 1643 | NM_017193 | | kynurenine aminotransferase II | kynuremine aminotransferase II |
| 1551 | K | 1633 | NM_017084 | Glycine, serine and | Glycine methyltransferase | Glycine methyltransferase |
| | | | | threonine metabolism | | |
| 1554 | I | 625 | AI045440 | | Sialophorin (gpL115, | Sialoporin (gpL115, leukosianin, |
| | | | | | leukosianin, CD43) | CD43) |
| 1561 | A, M, O | 1621 | NM_016995 | | Complement component | Complement component 4 binding |
| | | | | | 4 binding protein, beta | protein, beta |
| 1562 | F, G | 267 | AA893552 | | | Rattus norvegicus kallistatin mRNA, |
| | | | | | | complete cds |
| 1571 | I | 1446 | U05014 | | | Rattus norvegicus Sprague/Dawley |
| | | | | | | PHAS-I mRNA, complete cds |
| 1572 | Q | 1046 | AI178828 | | | Rattus norvegicus Sprague/Dawley |
| | | | | | | PHAS-I mRNA, complete cds |
| 1579 | R | 1512 | X73411 | | | Rat small nuclear ribonucleoparticle- |
| | | | | | | associated protein (snRNP) mRNA, |
| | | | | | | complete cds, clone Sm51 |
| 1583 | A | 1448 | U07201 | Alanine and aspartate | Asparagine synthetase | Asparagine synthetase |
| | | | | metabolism, Nitrogen | | |
| | | | | metabolism | | |
| 1598 | C, J | 1722 | NM_024134 | | DNA-damage inducible | Rattus norvegicus GADD153 mNRA, |
| | | | | | transcript 3 | complete cds |
| 1610 | C | 1703 | NM_022509 | | | Rattus norvegicus survival |
| | | | | | | motor neuron (smn), RNA, |
| | | | | | | complete cds |
| 1625 | I | 1588 | NM_012924 | | Cell surface glycoprotein CD44 | Cell surface glycoprotein CD44 |
| | | | | | (hyaluronate binding protein) | (hyaluronate binding protein) |
| 1641 | E | 1354 | E03428 | | Peptidylglycine alpha-amidating | Peptidylglycin alpha-amidating |
| | | | | | monooxygenase | momooxygenase |
| 1644 | G | 208 | AA891068 | | Peptidylglycine alpha-amidating | Peptidylglycine alpha-amidating |
| | | | | | monooxygenase | monooxygenase |
| 1653 | G | 1222 | AI233806 | | Peptidylglycine alpha-amidating | Peptidylglycine alpha-amidating |
| | | | | | monooxygenase | monooxygenase |
| 1661 | B, E | 1459 | U26397 | Inositol phosphate | HHs: inositol polyphosphate-4- | Rattus norvegicus inositol |
| | | | | metabolism | phosphatase, type I, 107kD | polyphosphate 4-phosphatase |
| | | | | | | mRNA, complete cds |
| 1690 | A, E | 46 | AA817829 | | | ESTs, Highly similar to MEK binding |
| | | | | | | partner 1 [M. musculus] |
| 1700 | P | 1486 | X03369 | | tubulin, beta 2 | ESTs, Highly similar to TBB1_RAT |
| | | | | | | TUBULIN BETA CHAIN |
| | | | | | | [R. norvegicus], Rat mRNA for beta- |
| | | | | | | tubulin T beta 15 |
| 1727 | C, J | 482 | AF001417 | | | Rattus norvegicus zinc finger protein |
| | | | | | | mRNA, complete cds |
| 1728 | E, S | 1332 | D16479 | Bile acid biosynthesis, | HHs:hydroxyacyl-Coenzyme A | Rat mRNA for mitochondrial |
| | | | | Fatty acid biosynthesis | dehydrogenase/3-ketoacyl- | long-chain 3 ketoacyl-CoA thiolase |
| | | | | (path 2), Fatty acid | Coenzyme A thiolase/enoyl- | beta-subunit of mitochondrial |
| | | | | metabolism, | Coenzyme A hydratase | trifunctional protein, complete dds |
| | | | | Phenylalanine | (trifunctional protein), | |
| | | | | metabolism, | beta subunit | |
| | | | | Valine, leucine and | | |
| | | | | isoleucine degradation | | |
| 1749 | K | 1657 | NM_017327 | | GTP-binding protein | GTP-binding protein |
| 1753 | A | 1462 | U39208 | Prostaglandin and | HHs:cytochrome P450, | Rattus norvegicus cytochrome P450 4F6 |
| | | | | leukotriene metabolism | subfamily IVF, polypeptide 2 | (CYP4F6) mRNA, complete cds |
| 1777 | P | 1586 | NM_012918 | | Calcium channel alpha 1A | Calcium channel alpha 1A |
| 1795 | B, K, Q | 1392 | L24207 | | Cytochrome P450, | Cytochrome P450, subfamily IIIA, |
| | | | | | subfamily IIA, polypeptide 3 | polypeptide 3 |
| 1796 | B, K | 1392 | L24207 | | Cytochrome P450, | Cytochrome P450, subfamily IIIA, |
| | | | | | subfamily IIA, polypeptide 3 | polypeptide 3 |
| 1802 | H | 47 | AA817841 | | | ESTs |
| 1805 | N | 508 | AI007824 | | | Rattus rattus guanine nucleotide- |
| | | | | | | releasing protein (mss4) mRNA, |
| | | | | | | complete cds |
| 1809 | F | 391 | AA946503 | | | Rat mRNA for alpha-2u globulin- |
| | | | | | | related protein |
| 1841 | C, N | 1555 | NM_012637 | | Protein-tyrosine phosphatase | Protein-tyrosine phosphatase |
| 1843 | N, Q | 1555 | NM_012637 | | Protein-tyrosine phosphatase | Protein-tyrosine phosphatase |
| 1844 | A, N | 1555 | NM_012637 | | Protein-tyrosine phosphatase | ESTs,Protein-tyrosine phosphatase |
| 1854 | M | 1382 | K02814 | | K-kininogen, differential | K-kininogen, differential splicing leads |
| | | | | | splicing leads to HMW Kngk, | to HMW Kngk,T-kininogen |
| | | | | | T-kininogen | |
| 1858 | S | 1524 | Y09333 | | acyl-CoA thioesterase 1, | R. norvegicus mRNA for mitochondrial |
| | | | | | cytosolic | very-long-chain acyl-CoA |
| | | | | | | thioesterase, Rattus norvegicus mRNA |
| | | | | | | for acyl-CoA hydrolase, complete cds |
| 1877 | A | 1513 | X74593 | Fructose and mannose | Sorbitol dehydrogenase | Sorbitol dehydrogenase |
| | | | | metabolism | | |
| 1884 | L | 1340 | D50695 | | | Rattus norvegicus mRNA for |
| | | | | | | proteasomal ATPase (tat-binding |
| | | | | | | protein7), complete cds |
| 1893 | P | 1495 | X51529 | Glycerolipid metabolism, | phospholipase A2, group IIA | Rattus norvegicus mRNA for |
| | | | | Phospholipid degradation, | (platelets, synovial fluid) | phospholipase A2 precursor, complete |
| | | | | Prostaglandin and | | cds |
| | | | | leukotriene metabolism | | |
| 1900 | A, B, L | 48 | AA817849 | | | ESTs |
| 1901 | L | 48 | AA817849 | | | ESTs |
| 1903 | L | 1013 | AI177377 | | | ESTs |
| 1919 | H | 815 | AI137856 | | P450 (cytochrome) | Rat NADPH-cytochrome P-450 |
| | | | | | oxidoreductase | oxidoreductase mRNA, complete cds |
| 1920 | H | 1397 | M10068 | | P450 (cytochrome) | Rat NADPH-cytochrome P-450 |
| | | | | | oxidoreductase | oxidoreductase mRNA, complete cds |
| 1921 | H | 1351 | E01524 | | P450 (cytochrome) | Rat NADPH-cytochrome P-450 |
| | | | | | oxidoreductase | oxidoreductase mRNA, complete cds |
| 1929 | A | 1449 | U10357 | | Hsp:[PYRUVATE | Rattus norvegicus pyruvate |
| | | | | | DEHYDROGENASE(LIPO- | dehydrogenase kinase 2 subunit p45 |
| | | | | | AMIDE)] KINASE | (PDK2) mRNA, complete cds |
| | | | | | ISOZYME 2, MITO- | |
| | | | | | CHONDRIAL PRECURSOR | |
| 1930 | L | 410 | AA957202 | | | Rattus norvegicus pyruvate |
| | | | | | | dehydrogenase kinase 2 subunit p45 |
| | | | | | | (PDK2) mRNA, complete cds |
| 1957 | K | 1628 | NM_017060 | | Hras-revertant gene 107 | Hras-revertant gene 107 |
| 1995 | N | 492 | AF038870 | Glycine, serine and | HMm:betaine-homocysteine | Rattus norvegicus betaine homocysteine |
| | | | | threonine metabolism, | methyltransferase | methyltransferase (BHMT) mRNA, |
| | | | | Methionine metabolism | | complete cds |
| 2006 | E | 1716 | NM_022936 | | | R. norvegicus mRNA for cytosolic |
| | | | | | | expoxide hydrolase |
| 2011 | P | 1610 | NM_013173 | | Solute carrier family 11 | Solute carrier family 11 member 2 |
| | | | | | member 2 (natural resistance- | (natural resistance-associated |
| | | | | | associated macrophage | macrophage protein 2) |
| | | | | | protein 2) | |
| 2012 | P | 1610 | NM_013173 | | Solute carrier family 11 | Solute carrier family 11 member 2 |
| | | | | | member 2 (natural resistance- | (natural resistance-associated |
| | | | | | associated macrophage | macrophage protein 2) |
| | | | | | protein 2) | |
| 2013 | P | 1610 | NM_013173 | | Solute carrier family 11 | Solute carrier family 11 member 2 |
| | | | | | member 2 (natural resistance- | (natural resistance-associated |
| | | | | | associated macrophage | macrophage protein 2) |
| | | | | | protein 2) | |
| 2042 | Q, R | 721 | AI101921 | | | ESTs |
| 2043 | E, H | 1125 | AI230171 | | | ESTs |
| 2049 | J | 417 | AA963369 | | | ESTs |
| 2051 | S | 418 | AA963372 | | | ESTs |
| 2065 | I | 1084 | AI227769 | | | ESTs |
| 2101 | R | 565 | AI013667 | | | ESTs |
| 2111 | A | 750 | AI103550 | | | Rattus norvegicus CDK102 mRNA |
| 2113 | S | 423 | AA964275 | | | ESTs, Weakly similar to AF077030_1 |
| | | | | | | hypothetical 43.2 kDa protein |
| | | | | | | [H. sapiens] |
| 2117 | R | 324 | AA925961 | | | Rattus norvegicus Na—K—Cl |
| | | | | | | cotransporter (Nkcc1) mRNA, complete |
| | | | | | | cds |
| 2153 | E | 1475 | U75404 | | | ESTs |
| 2154 | R | 1223 | AI233818 | | | ESTs |
| 2164 | A | 781 | AI111413 | | | ESTs |
| 2190 | S | 420 | AA964004 | | | ESTs |
| 2196 | A | 776 | AI105243 | | | ESTs |
| 2216 | R | 912 | AI171745 | | | ESTs |
| 2264 | A | 821 | AI144741 | | | ESTs |
| 2280 | H | 421 | AA964139 | | | EST |
| 2292 | E | 714 | AI101362 | | | ESTs |
| 2310 | M | 587 | AI029969 | | | ESTs |
| 2326 | L | 432 | AA964892 | | | ESTs, Highly similar to |
| | | | | | | CA14_MOUSE COLLAGEN |
| | | | | | | ALPHA 1(IV) CHAIN PRECURSOR |
| | | | | | | [M. musculus] |
| 2335 | A | 424 | AA964302 | | | ESTs |
| 2339 | E | 1162 | AI231798 | | | ESTs |
| 2342 | E | 425 | AA964336 | | | EST |
| 2350 | D | 426 | AA964368 | | | ESTs, Highly similar to TGT_HUMAN |
| | | | | | | QUEUINE TRNA- |
| | | | | | | RIBOSYLTRANSFERASE |
| | | | | | | [H. sapiens] |
| 2354 | L | 454 | AA997763 | | | ESTs, Highly similar to hypothetical |
| | | | | | | protein [H. sapiens] |
| 2359 | N | 998 | AI177029 | | | ESTs, Highly similar to JU0227 |
| | | | | | | protein-tyrosine kinase |
| | | | | | | [M. musculus] |
| 2368 | N | 504 | AF095741 | | | Rattus norvegicus MG87 mRNA, |
| | | | | | | complete cds |
| 2372 | A, L | 1130 | AI230373 | | | ESTs |
| 2373 | O | 428 | AA964455 | | | ESTs |
| 2383 | A, E | 429 | AA964514 | | | ESTs |
| 2457 | S | 431 | AA964752 | | | EST |
| 2484 | A, O | 761 | AI104675 | | | ESTs |
| 2505 | A, G | 1549 | NM_012597 | Glycerolipid | Lipase, hepatic | Lipase, hepatic |
| | | | | metabolism | | |
| 2506 | E | 524 | AI009341 | | | ESTs |
| 2532 | A | 975 | AI176590 | | | ESTs |
| 2536 | A | 978 | AI176616 | | | ESTs |
| 2555 | B, C, Q | 1590 | NM_012967 | | Intercellular adhesion | Intercellular adhesion molecule 1 |
| | | | | | molecule 1 | |
| 2569 | A, C, | 435 | AA965122 | | | ESTs |
| | F, K, R | | | | | |
| 2576 | A | 226 | AA891884 | | | ESTs |
| 2587 | G | 1170 | AI232103 | | | ESTs |
| 2594 | L | 1241 | AI234843 | | | ESTs, Moderately similar to Similarity |
| | | | | | | to Yeast LPG22P protein |
| | | | | | | [C. elegans] |
| 2615 | C, J | 1109 | AI229318 | | | ESTs |
| 2628 | J | 1551 | NM_012603 | | Avian myelocytomatosis | Avian myelocytomatosis viral (v-myc) |
| | | | | | viral (v-myc) oncogene homolog | oncogene homolog |
| 2629 | J | 1551 | NM_012603 | | Avian myelocytomatosis | Avian myelocytomatosis viral (v-myc) |
| | | | | | viral (v-myc) oncogene homolog | oncogene homolog |
| 2655 | B, N, Q | 343 | AA943886 | | | Rattus norvegicus protein kinase SNK |
| | | | | | | (Snk) mRNA, complete cds |
| 2667 | G | 1568 | NM_012766 | | Tocopherol transfer protein | Tocopherol transfer protein alpha |
| | | | | | alpha | |
| 2691 | R | 434 | AA965075 | | | ESTs |
| 2696 | A | 1737 | NM_022515 | | | R. norvegicus (Sprague Dawley) mRNA |
| | | | | | | for ribosomal protein L24 |
| 2727 | H | 252 | AA892918 | | | ESTs |
| 2736 | Q | 1537 | NM_012519 | | Ca++/calmodulin-dependent | Ca++/calmodulin-dependent protein |
| | | | | | protein kinase II, delta subunit | kinase II, delta subunit |
| 2744 | I | 1347 | D87991 | | | ESTs, Highly similar to UGTrel1 |
| | | | | | | [M. musculus] |
| 2757 | L | 456 | AA997851 | | | ESTs |
| 2762 | A | 350 | AA944165 | | | ESTs, Highly similar to C10 |
| | | | | | | [M. musculus] |
| 2763 | E | 1173 | AI232269 | | | ESTs |
| 2781 | I | 50 | AA817925 | | | ESTs |
| 2788 | J | 939 | AI175513 | | | Rattus norvegicus mRNA for phocein |
| | | | | | | protein |
| 2799 | A | 568 | AI013778 | | | ESTs |
| 2801 | F | 1345 | D85435 | | | Rattus norvegicus mRNA for protein |
| | | | | | | kinase C delta-bindig protein, complete |
| | | | | | | cds |
| 2802 | F | 1345 | D85435 | | | Rattus norvegicus mRNA for protein |
| | | | | | | kinase C delta-bindig protein, complete |
| | | | | | | cds |
| 2803 | L | 437 | AA996451 | | | ESTs |
| 2813 | S | 365 | AA945052 | Butanoate metabolism, | HMm:3-hydroxy-3- | R.norvegicus mRNA for 3-hydroxy-3- |
| | | | | Synthesis and degradation | methylglutaryl | methylglutaryl CoA lyase |
| | | | | of ketone bodies, Valine, | Coenzyme A lyase | |
| | | | | leucine and isoleucine | | |
| | | | | degradation | | |
| 2818 | C, D, F | 1055 | AI179144 | | | ESTs |
| 2838 | D | 655 | AI070511 | | | ESTs, Highly similar to G7A |
| | | | | | | [M. musculus] |
| 2853 | I | 1579 | NM_012838 | | Cystatin beta | Cystatin beta |
| 2854 | I | 1579 | NM_012838 | | Cystatin beta | Cystatin beta |
| 2868 | E | 1171 | AI232209 | | | ESTs |
| 2897 | C, D | 51 | AA818039 | | | ESTs |
| 2901 | A | 603 | AI043752 | | | ESTs |
| 2905 | A, B | 438 | AA996727 | | | ESTs |
| 2911 | A | 597 | AI030835 | | | ESTs |
| 2915 | R | 439 | AA996782 | | | ESTs |
| 2932 | R | 1204 | AI233288 | | | ESTs |
| 2933 | E | 1665 | NM_019204 | | | ESTs, Highly similar to beta-site APP |
| | | | | | | cleaving enzyme [R. norvegicus] |
| 2938 | C | 440 | AA996883 | | | ESTs |
| 2993 | A | 971 | AI176492 | | | ESTs, Highly similar to AF1886297₁₃ 1 |
| | | | | | | TGF-beta receptor binding protein |
| | | | | | | [M. musculus] |
| 3023 | G | 885 | AI170795 | | | ESTs |
| 3062 | D | 468 | AA998857 | | | EST, Weakly similar to CBPB_RAT |
| | | | | | | CARBOXYPEPTIDASE B |
| | | | | | | PRECURSOR [R. norvegicus] |
| 3073 | A, E, O | 1213 | AI233494 | | | ESTs |
| 3074 | A, E, O | 1213 | AI233494 | | | ESTs |
| 3075 | A, O | 1213 | AI233494 | | | ESTs |
| 3080 | H | 242 | AA892553 | | HHs:signal transducer and | Rattus norvegicus signal transducer and |
| | | | | | activator of transcription 1, | activator of transcription 1 (Stat1) |
| | | | | | 91kD | mRNA, complete cds |
| 3091 | E | 1260 | AI236027 | | | ESTs |
| 3099 | S | 1113 | AI229680 | Oxidative | HHs:NADH dehydrogenase | ESTs, Highly similar to |
| | | | | phosphorylation, | (ubiquinone) Fe—S protein | NADH:ubiquinone oxidoreductase |
| | | | | Ubiquinone | 3 (30kD) (NADH-coenzyme | NDUFS3 subunit [H. sapiens] |
| | | | | biosynthesis | Q reductase) | |
| 3121 | A, B, E | 510 | AI008160 | | | ESTs, Moderately similar to |
| | | | | | | AF151841_1 CGI-83 protein |
| | | | | | | [H. sapiens] |
| 3131 | A | 256 | AA893032 | | | ESTs |
| 3138 | I | 1047 | AI178850 | | | ESTs |
| 3139 | J | 540 | AI010618 | | | ESTs |
| 3143 | E, H | 1180 | AI232408 | | | ESTs |
| 3145 | A | 444 | AA997237 | | | EST |
| 3175 | S | 447 | AA997414 | | | ESTs |
| 3189 | A | 448 | AA997438 | | | ESTs, Moderately similar to LDL |
| | | | | | | receptor member LR3 [M. musculus] |
| 3203 | C | 1624 | NM_017039 | | Protein phosphatase 2 | Protein phosphatase 2 (formerly 2A), |
| | | | | | (formerly 2A), catalytic subunit, | catalytic subunit, alpha isoform |
| | | | | | alpha isoform | |
| 3207 | A | 449 | AA997466 | | | ESTs |
| 3219 | E | 767 | AI105065 | | | ESTs, Highly similar to |
| | | | | | | PNAD_MOUSE PROTEIN |
| | | | | | | N-TERMINAL ASPARAGINE |
| | | | | | | AMIDOHYDROLASE [M. musculus] |
| 3233 | L | 53 | AA818105 | | | ESTs, Moderately similar to Unknown |
| | | | | | | gene product [H. sapiens] |
| 3250 | M | 455 | AA997765 | | | Rattus norvegicus fibrillin-1 mRNA, |
| | | | | | | complete cds |
| 3253 | F | 1652 | NM_017282 | | proteasome (prosome, | proteasome (prosome, macropain) |
| | | | | | macropain) subunit, alpha type 5 | subunit, alpha type 5 |
| 3260 | S | 571 | AI013875 | | | ESTs |
| 3266 | L | 915 | AI171948 | | | ESTs |
| 3279 | S | 747 | AI103224 | | | ESTs, Weakly similar to putative short- |
| | | | | | | chain dehydrogenase/reductase |
| | | | | | | [R. norvegicus] |
| 3280 | C | 1083 | AI227699 | | | ESTs |
| 3292 | M, N | 1325 | D00753 | | | Rat mRNA for contrapsin-like protease |
| | | | | | | inhibitor related protein (CPi-26) |
| 3365 | A, B | 518 | AI008919 | | | ESTs |
| 3381 | K | 254 | AA892993 | | | ESTs |
| 3418 | A, C, D | 936 | AI175475 | | | ESTs, Highly similar to NHPX_RAT |
| | | | | | | NHP2/RS6 FAMILY PROTEIN |
| | | | | | | YEL026W HOMOLOG [R. norvegicus] |
| 3430 | J | 1441 | S85184 | | Cathepsin L | Cathepsin L |
| 3439 | S | 255 | AA893000 | | | ESTs, Highly similar to KIAA0564 |
| | | | | | | protein [H. sapiens] |
| 3452 | M, N | 452 | AA997721 | | | Rattus norvegicus orphan chemokine |
| | | | | | | receptor mRNA, complete cds |
| 3486 | H | 869 | AI170313 | | | ESTs |
| 3504 | A, B | 760 | AI104659 | | | Rattus norvegicus mRNA for R-RCD1, |
| | | | | | | complete cds |
| 3510 | K | 963 | AI176423 | | | ETSs, Highly similar to ZO1_MOUSE |
| | | | | | | TIGHT JUNCTION PROTEIN ZO-1 |
| | | | | | | [M. musculus] |
| 3513 | S | 1639 | NM_017177 | Glycerolipid metabolism | choline/ethanolamine | choline/ethanolamine kinase |
| | | | | | kinase | |
| 3549 | H, I | 1385 | L11319 | | | Rat signal peptidase mRNA, complete |
| | | | | | | cds |
| 3558 | S | 463 | AA998461 | | | EST |
| 3570 | O | 464 | AA998510 | | | ESTs, Weakly similar to RET1_RAT |
| | | | | | | RETINOL-BINDING PROTEIN I, |
| | | | | | | CELLULAR [R. norvegicus] |
| 3587 | J | 1078 | AI180253 | | | ESTs |
| 3617 | N | 1259 | AI236021 | | | Rattus norvegicus gene for |
| | | | | | | hepatocarcinogenesis-related |
| | | | | | | transcription factor (HTF), complete cds |
| 3626 | P | 950 | AI176031 | | | ESTs, Weakly similar to JC1450 |
| | | | | | | fibroblast growth factor receptor 4 - rat |
| | | | | | | [R. norvegicus] |
| 3631 | S | 302 | AA924460 | | | ESTs, Highly similar to Opa-interacting |
| | | | | | | protein OIP2 [H. sapiens] |
| 3660 | B | 467 | AA998833 | | | ESTs |
| 3708 | M | 469 | AA999060 | | | EST |
| 3710 | B, Q | 470 | AA999064 | | | ESTs |
| 3713 | A, N | 791 | AI112571 | | | ESTs |
| 3720 | S | 471 | AA999138 | | | ESTs |
| 3722 | N | 457 | AA997979 | | | ESTs |
| 3730 | N | 460 | AA998234 | | | EST |
| 3743 | S | 1335 | D30666 | | | Rat mRNA for brain acyl-CoA |
| | | | | | | synthetase II, complete cds |
| 3749 | P | 461 | AA998276 | | | EST |
| 3776 | Q | 1679 | NM_019354 | | Uncoupling protein 2, | Uncoupling protein 2, mitochondrial |
| 3803 | L, R | 884 | AI170773 | | mitochondrial | Rattus norvegicus 250 kDa estrous- |
| | | | | | | specific protein mRNA, partial cds |
| 3816 | J | 1219 | AI233729 | | | ESTs, Highly similar to |
| | | | | | | PSD5_HUMAN 26S PROTEASOME |
| | | | | | | SUBUNIT S5B [H. sapiens] |
| 3822 | A | 288 | AA900863 | | | ESTs, Weakly similar to nuclear RNA |
| | | | | | | helicase [R. norvegicus] |
| 3823 | A | 1196 | AI233147 | | | ESTs, Weakly similar to nuclear RNA |
| | | | | | | helicase [R. norvegicus] |
| 3831 | C, J | 1525 | Y12635 | Oxidative | HMm:ATPase, H+ transporting, | R. norvegicus mRNA for vacuolar |
| | | | | phosphorylation | lysosomal (vacuolar proton | adenosine triphosphatase subunit B |
| | | | | | pump), beta 56/58 kDa, | |
| | | | | | isoform 2 | |
| 3846 | O | 658 | AI070895 | | | ESTs, Weakly similar to similar to acyl- |
| | | | | | | CoA dehydrogenases and epoxide |
| | | | | | | hydrolases [C. elegans] |
| 3849 | A | 567 | AI013745 | | | ESTs, Moderately similar to CGI-147 |
| | | | | | | protein [H. sapiens] |
| 3916 | A, F | 865 | AI169947 | | | ESTs |
| 3917 | B | 1194 | AI232970 | | | ESTs |
| 3929 | O | 270 | AA894233 | | | ESTs |
| 3934 | A | 544 | AI011510 | | | ESTs |
| 3959 | A | 292 | AA901338 | | | ESTs, Highly similar to |
| | | | | | | IF2B_HUMAN |
| | | | | | | EUKARYOTIC TRANSLATION |
| | | | | | | INITIATION FACTOR 2 BETA |
| | | | | | | SUBUNIT [H. sapiens] |
| 3969 | A | 1001 | AI177055 | | | ESTs |
| 3972 | Q | 300 | AA924307 | | | ESTs |
| 3976 | E | 61 | AA818264 | | | ESTs, Weakly similar to similar to |
| | | | | | | GTPase-activating proteins [H. sapiens] |
| 3981 | A | 554 | AI012235 | | | ESTs |
| 3995 | A | 545 | AI011678 | | | ESTs |
| 4017 | A | 63 | AA818287 | | | ESTs |
| 4026 | B, Q | 1225 | AI233835 | | | ESTs |
| 4048 | I | 139 | AA851814 | | | Rattus norvegicus osteoactivin mRNA, |
| | | | | | | complete cds |
| 4049 | I | 784 | AI112012 | | | Rattus norvegicus osteoactivin mRNA, |
| | | | | | | complete cds |
| 4082 | O | 624 | AI045256 | | | ESTs |
| 4084 | A | 512 | AI008504 | | | ESTs |
| 4092 | L | 1095 | AI228723 | Glycolysis/ | HHs:phosphoglycerate mutase 1 | R. norvegicus phosphoglycerate mutase |
| | | | | Gluconeogenesis | (brain) | B isozyme (PGAM) mRNA, complete |
| | | | | | | cds |
| 4097 | I | 1037 | AI178635 | | | ESTs |
| 4119 | J | 720 | AI101901 | | | ESTs |
| 4127 | H | 1057 | AI179206 | | | ESTs |
| 4143 | A | 786 | AI112107 | | | ESTs |
| 4157 | E | 525 | AI009481 | | | ESTs, Weakly similar to putative |
| | | | | | | [C. elegans] |
| 4168 | E | 527 | AI009654 | | | ESTs |
| 4178 | I | 170 | AA859536 | | | ESTs |
| 4179 | A, C, E, R | 1132 | AI230431 | | | ESTs |
| 4193 | A, C, D, | 923 | AI172274 | | | ESTs, Weakly similar to I37195 AU- |
| | E, F, I | | | | | specific RNA-binding protein/enoyl- |
| | | | | | | CoA hydratase [H. sapiens] |
| 4199 | G | 1425 | M83143 | | Sialyltransferase 1 | Rat beta-galactoside-alpha 2,6- |
| | | | | | (beta-glactoside alpha- | sialyltransferase mRNA |
| | | | | | 2,6-sialytransferase) | |
| 4207 | F | 371 | AA945591 | | | ESTs, Weakly similar to JC5105 |
| | | | | | | stromal cell-derived factor 2 - mouse |
| | | | | | | [M. musculus] |
| 4224 | G | 1415 | M31322 | | | Rat sperm membrane protein (YWK-II) |
| | | | | | | mRNA, 3′ end |
| 4231 | R | 1159 | AI231763 | | | Rattus norvegicus late gestation lung 2 |
| | | | | | | protein (Lgl2) mRNA, complete cds |
| 4234 | H | 1685 | NM_021577 | | | Rattus norvegicus mRNA for AIF-C1, |
| | | | | | | complete cds |
| 4250 | B | 76 | AA818700 | | | ESTs |
| 4271 | S | 321 | AA925603 | | | ESTs, Moderately similar to |
| | | | | | | AF153605_1 androgen induced protein |
| | | | | | | [H. sapiens] |
| 4272 | S | 1152 | AI231309 | | | ESTs, Moderately similar to |
| | | | | | | AF153605_1 androgen induced protein |
| | | | | | | [H. sapiens] |
| 4281 | A, G | 1663 | NM_019192 | | selenoprotein P, plasma, 1 | selenoprotein P, plasma, 1 |
| 4290 | S | 1323 | AJ224120 | | | Rattus norvegicus peroxisomal |
| | | | | | | membrane protein Pmp26p (Peroxin-11) |
| 4291 | A, H | 79 | AA818741 | | | ESTs |
| 4312 | K | 480 | AB010635 | | | Rattus norvegicus mRNA for |
| | | | | | | carboxylesterase precursor, complete |
| | | | | | | cds |
| 4314 | G, M | 483 | AF010597 | | | Rattus norvegicus bile salt export pump |
| | | | | | | (spgp) mRNA, complete cds |
| 4318 | F | 474 | AB005900 | | | Rattus norvegicus mRNA for |
| | | | | | | endothelial receptor for oxidized |
| | | | | | | low-density lipoprotein, complete cds |
| 4327 | I | 498 | AF063447 | | | Rattus norvegicus nuclear RNA helicase |
| | | | | | | mRNA, complete cds |
| 4330 | A, C, D, E | 80 | AA818747 | | | Rattus norvegicus stromal cell-derived |
| | | | | | | factor-1 gamma mRNA, complete cds |
| 4348 | E | 874 | AI170447 | | | Rattus norvegicus mRNA for |
| | | | | | | norepinephrine transporter b (rNETb), |
| | | | | | | complete cds |
| 4360 | A | 1358 | H31813 | | | ESTs |
| 4371 | E | 295 | AA924196 | | | ESTs |
| 4426 | I | 3 | AA685974 | | | ESTs |
| 4438 | S | 2 | AA684919 | | | ESTs |
| 4440 | A, O | 1189 | AI232643 | | | ESTs |
| 4473 | A | 229 | AA891965 | | | ESTs |
| 4504 | Q | 1725 | NM_024159 | | | Rattus norvegicus DOC-2 p59 isoform |
| | | | | | | mRNA, complete cds |
| 4520 | O | 751 | AI103694 | Oxidative phosphorylation, | HHs:NADH dehydrogenase | ESTs, Moderately similar to NADH- |
| | | | | Ubiquinone biosynthesis | (ubiquinone) 1 alpha | ubiquinone oxidoreductase subunit Cl- |
| | | | | | subcomplex, 2 (8kD, B8) | B8 [H. sapiens] |
| 4553 | A, C | 999 | AI177038 | | | ESTs |
| 4576 | K | 1049 | AI178872 | | | ESTs |
| 4588 | K | 477 | AB009636 | | | Rattus norvegicus mRNA for |
| | | | | | | phosphoinositide 3-kinase, complete cds |
| 4592 | C, D | 1680 | NM_019356 | | eukaryotic translation initiation | eukaryotic translation initiation factor 2, |
| | | | | | factor 2, subunit 1 (alpha) | subunit 1 (alpha) |
| 4610 | E | 1075 | AI179991 | | | ESTs |
| 4650 | G | 718 | AI101582 | | | ESTs |
| 4670 | A, N | 1217 | AI233714 | | | ESTs |
| 4674 | O | 279 | AA899847 | | | EST |
| 4679 | L | 585 | AI029847 | | | ESTs, Highly similar to IRF3_MOUSE |
| | | | | | | INTERFERON REGULATORY |
| | | | | | | FACTOR 3 [M. musculus] |
| 4719 | A | 1087 | AI228265 | | | ESTs |
| 4725 | L | 282 | AA900290 | | | ESTs |
| 4759 | E | 285 | AA900553 | | | ESTs |
| 4781 | C, D | 1228 | AI233925 | | | ESTs |
| 4856 | I | 752 | AI103708 | | | ESTs |
| 4868 | A | 882 | AI170763 | | | ESTs |
| 4892 | P | 611 | AI044292 | | | ESTs |
| 4914 | A | 785 | AI112086 | | | ESTs |
| 4929 | E | 296 | AA924236 | | | EST |
| 4931 | S | 297 | AA924261 | | | ESTs, Moderately similar to unknown |
| | | | | | | [H. sapiens] |
| 4933 | A, E, P | 299 | AA924301 | | | EST |
| 4937 | A, L | 1294 | AI237189 | | | ESTs |
| 4940 | S | 1738 | NM_022526 | | | Rattus norvegicus rap7a mRNA, |
| | | | | | | complete cds |
| 4944 | A, F | 301 | AA924405 | | | ESTs, Moderately similar to |
| | | | | | | NO56_HUMAN NUCLEOLAR |
| | | | | | | PROTEIN NOP56 [H. sapiens] |
| 4951 | A | 519 | AI009026 | | | ESTs |
| 4952 | C, J | 86 | AA818907 | | | ESTs |
| 4969 | M | 795 | AI113008 | | | ESTs, Moderately similar to |
| | | | | | | megakaryocyte stimulating factor |
| | | | | | | [H. sapiens] |
| 5008 | A, C | 88 | AA818921 | | | ESTs |
| 5018 | L | 306 | AA924767 | | | EST |
| 5020 | E | 307 | AA924768 | | | ESTs, Weakly similar to MRJ |
| | | | | | | [M. musculus] |
| 5027 | A | 308 | AA924793 | | | ESTs |
| 5038 | E | 846 | AI169239 | | | ESTs |
| 5046 | A, L | 1303 | AI237855 | | | ESTs |
| 5052 | R | 1270 | AI236302 | | | ESTs, Weakly similar to TTHY_RAT |
| | | | | | | TRANSTHYRETIN PRECURSOR |
| | | | | | | [R. norvegicus] |
| 5059 | Q | 1288 | AI236947 | | | ESTs |
| 5091 | E | 699 | AI073092 | | | ESTs |
| 5110 | E, M | 317 | AA925274 | | | ESTs |
| 5111 | E | 397 | AA955729 | | | EST,ESTs |
| 5175 | A | 90 | AA818951 | Glycolysis/ | Pyruvate kinase, muscle | Pyruvate kinase, muscle |
| | | | | Gluconeogenesis, Purine | | |
| | | | | metabolism, Pyruvate | | |
| | | | | metabolism | | |
| 5219 | A | 322 | AA925807 | | | ESTs |
| 5235 | F | 829 | AI145569 | | | ESTs, Moderately similar to |
| | | | | | | BcDNA.GH02974 [D. melanogaster] |
| 5291 | M | 1190 | AI232700 | | | ESTs |
| 5331 | I | 91 | AA818996 | Aminoacyl-tRNA | HHs:glutaminyl-tRNA | ESTs, Moderately similar to |
| | | | | biosynthesis, Glutamate | synthetase | SYQ_HUMAN GLUTAMINYL- |
| | | | | metabolism | | TRNA SYNTHETASE [H. sapiens] |
| 5339 | E, M | 911 | AI171727 | Nicotinate and nicotinamide | HMm:nicotinamide | ESTs, Weakly similar to PNMT |
| | | | | metabolism | N-methyltransferase | [R. norvegicus] |
| 5381 | R | 1038 | AI178734 | | | ESTs |
| 5384 | A, B, F | 207 | AA891041 | | | ESTs |
| 5434 | E | 1380 | K01878 | | Proopoimelanocortin, | Rat proopiomelanocortin (POMC) gene |
| | | | | | beta (endorphin,beta) | |
| 5437 | F | 407 | AA956910 | | | ESTs |
| 5461 | A | 613 | AI044338 | | | EST |
| 5464 | B, O | 614 | AI044345 | | | ESTs, Highly similar to AF172275_1 |
| | | | | | | FUS2 [M. musculus] |
| 5489 | C, J | 914 | AI171795 | | | ESTs |
| 5492 | G | 1336 | D38061 | Androgen and estrogen | UDP-glucuronosyltrans- | ESTs,UDP-glucuronosyltrnasferase 1 |
| | | | | metabolism, Pentose and | ferase 1 family member 1 | family, member 1 |
| | | | | glucuronate | | |
| | | | | interconversions, Porphyrin | | |
| | | | | and chlorophyll | | |
| | | | | metabolism, Starch | | |
| | | | | and sucrose metabolism | | |
| 5493 | G, O | 1433 | S56936 | Androgen and estrogen | UDP-glucuronosyltrans- | ESTs,UDP-glucuronosyltransferase 1 |
| | | | | metabolism, Pentose and | ferase 1 family member 1 | family, member 1 |
| | | | | glucuronate | | |
| | | | | interconversions, Porphyrin | | |
| | | | | and chlorophyll | | |
| | | | | metabolism, | | |
| | | | | Starch and sucrose | | |
| | | | | metabolism | | |
| 5504 | D | 1165 | AI231805 | | | ESTs, Weakly similar to |
| | | | | | | NUML_MOUSE |
| | | | | | | NADH-UBIQUINONE |
| | | | | | | OXIDOREDUCTASE MLRQ |
| | | | | | | SUBUNIT [M. musculus] |
| 5518 | S | 617 | AI044550 | | | EST |
| 5565 | S | 377 | AA945879 | | | ESTs |
| 5602 | S | 1187 | AI232611 | | | ESTs, Weakly similar to mitochondrial |
| | | | | | | very-long-chain acyl-CoA thioesterase |
| | | | | | | [R. norvegicus] |
| 5608 | R | 93 | AA819041 | | | ESTs |
| 5616 | M, S | 1731 | NM_019143 | | Fibronectin 1 | Fibronectin 1 |
| 5622 | A | 1731 | NM_019143 | | Fibronectin 1 | Fibronectin 1 |
| 5687 | P | 705 | AI101006 | | | ESTs |
| 5696 | L | 621 | AI045116 | | | ESTs |
| 5733 | C | 1424 | M81855 | | P-glycoprotein 2/multidrug | P-glycoprotein/multidrug resistance 1 |
| | | | | | resistance 1b,P-glycoprotein/ | |
| | | | | | multidrug resistance 1 | |
| 5740 | L | 680 | AI072092 | | | ESTs, Moderately similar to |
| | | | | | | DYNC_HUMAN DYNACTIN, 50 KD |
| | | | | | | ISOFORM [H. sapiens] |
| 5748 | A | 1650 | NM_017279 | | proteasome (prosome, | proteasome (prosome, macropain) |
| | | | | | macropain) subunit, | subunit, alpha type 2 |
| | | | | | alpha type 2 | |
| 5749 | A, H | 1650 | NM_017279 | | proteasome (prosome, | proteasome (prosome, macropain) |
| | | | | | subunit, alpha type 2 | macropain) subunit, |
| | | | | | alpha type 2 | |
| 5754 | L, R | 133 | AA850738 | | | ESTs |
| 5780 | C, D | 1019 | AI177869 | | | ESTs, Weakly similar to DRAL |
| | | | | | | [R. norvegicus] |
| 5794 | C | 1212 | AI233480 | | | ESTs |
| 5795 | E | 626 | AI045441 | | | ESTs |
| 5813 | A | 1026 | AI178231 | | | ESTs |
| 5820 | J | 1285 | AI236771 | | | ESTs |
| 5824 | K | 627 | AI045555 | | | EST |
| 5863 | A | 95 | AA819111 | | | ESTs |
| 5867 | A, C, D | 158 | AA858953 | Alanine and aspartate | HHs:asparaginyl-tRNA | ESTs, Highly similar to |
| | | | | | | SYN_HUMAN |
| | | | | metabolism, Aminoacyl- | synthetase | ASPARAGINYL-TRNA |
| | | | | tRNA biosynthesis | | SYNTHETASE, CYTOPLASMIC |
| | | | | | | [H. sapiens] |
| 5885 | I | 1322 | AJ223184 | | | Rattus norvegicus mRNA for DORA |
| | | | | | | protein |
| 5887 | S | 1053 | AI179099 | | vanin 1 | ESTs, Moderately similar to Vanin-1 |
| | | | | | | [M. musculus] |
| 5899 | A, D, F | 867 | AI170038 | | | ESTs |
| 5920 | G | 843 | AI169163 | | | ESTs |
| 5923 | A | 65 | AA818355 | | | ESTs |
| 5926 | C | 1017 | AI177638 | | | ESTs, Moderately similar to M phase |
| | | | | | | phosphoprotein 10 [H. sapiens] |
| 5930 | E | 42 | AA817688 | | | ESTs |
| 5932 | J | 756 | AI104254 | | | ESTs |
| 5934 | A, F | 43 | AA817695 | | | ESTs, Highly similar to 2008147C |
| | | | | | | protein RAKd [R. norvegicus] |
| 5937 | J | 908 | AI171684 | | | ESTs |
| 5943 | A | 1005 | AI177105 | | | ESTs |
| 5953 | H | 893 | AI171231 | | | Rattus norvegicus amino acid |
| | | | | | | transporter system A (ATA2) mRNA, |
| | | | | | | complete cds |
| 5966 | H | 89 | AA818947 | | | ESTs |
| 5993 | R | 820 | AI144612 | | | ESTs |
| 5998 | G | 1317 | AI639501 | | | ESTs |
| 6003 | E | 54 | AA818107 | | | ESTs |
| 6007 | A | 55 | AA818123 | | | ESTs |
| 6012 | D | 56 | AA818139 | | | ESTs |
| 6013 | N | 1634 | NM_017096 | | C-reactive protein | C-reactive protein |
| 6015 | A, O | 57 | AA818158 | | | ESTs |
| 6016 | A, C, D | 58 | AA818163 | | | EST |
| 6017 | A | 1676 | NM_019292 | Nitrogen metabolism | carbonic anhydrase 3 | carbonic anhydrase 3 |
| 6018 | E, N | 96 | AA819140 | Nitrogen metabolism | carbonic anhydrase 3 | carbonic anhydrase 3 |
| 6026 | E | 59 | AA818211 | | | EST |
| 6032 | E | 60 | AA818258 | | | ESTs |
| 6033 | A | 1195 | AI233081 | | | ESTs |
| 6037 | A | 64 | AA818288 | | | ESTs |
| 6039 | D | 330 | AA942716 | | | ESTs, Highly similar to HN1 |
| | | | | | | [M. musculus] |
| 6060 | A, O | 77 | AA818702 | | | ESTs |
| 6066 | E | 83 | AA818781 | | | ESTs |
| 6072 | A, B, E, F | 1093 | AI228630 | | | ESTs, Weakly similar to Similarity to |
| | | | | | | litosperm LEC14B protein [C. elegans] |
| 6085 | C | 916 | AI171990 | | | ESTs, Moderately similar to axonemal |
| | | | | | | dynein heavy chain [H. sapiens] |
| 6101 | R | 881 | AI170752 | | | ESTs |
| 6132 | A, C, D | 94 | AA819055 | | | EST |
| 6143 | A, C | 771 | AI105167 | | | ESTs, Moderately similar to selenium- |
| | | | | | | binding protein [H. sapiens] |
| 6151 | G | 98 | AA819199 | | | EST |
| 6153 | G | 203 | AA875531 | | | Rattus norvegicus pro-alpha-2(I) |
| | | | | | | collagen (col1a2) mRNA, complete cds |
| 6155 | G | 715 | AI101443 | | | Rattus norvegicus pro-alpha-2(I) |
| | | | | | | collagen (col1a2) mRNA, complete cds |
| 6188 | E | 82 | AA818774 | | | ESTs |
| 6189 | B, E, G | 1023 | AI178027 | | | ESTs, Weakly similar to GTP_RAT |
| | | | | | | GLUTATHIONE S-TRANSFERASE P |
| | | | | | | [R. norvegicus] |
| 6190 | A | 107 | AA819812 | | | ESTs |
| 6193 | I | 1161 | A1231797 | | | ESTs |
| 6198 | M | 109 | AA819840 | | | ESTs |
| 6200 | P | 110 | AA819853 | | HHs:lymphotoxin beta (TNF | ESTs, Highly similar to |
| | | | | | superfamily, member 3) | TNFC_MOUSE |
| | | | | | | LYMPHOTOXIN-BETA [M. musculus] |
| 6213 | N | 726 | AI102190 | | | ESTs |
| 6222 | N | 68 | AA818474 | | | ESTs |
| 6226 | A | 70 | AA818521 | | | ESTs |
| 6236 | B, E, P | 75 | AA818627 | | | EST, Moderately similar to ISI1_RAT |
| | | | | | | INSULIN-INDUCED PROTEIN 1 |
| | | | | | | [R. norvegicus] |
| 6272 | L | 875 | AI170617 | | | ESTs, Weakly similar to B39066 |
| | | | | | | proline-rich protein 15 - |
| | | | | | | rat [R. norvegicus] |
| 6291 | H | 822 | AI144797 | | | ESTs |
| 6292 | S | 422 | AA964181 | | | ESTs |
| 6295 | N | 103 | AA819672 | | | EST |
| 6321 | A, J | 712 | AI101256 | | | ESTs, Weakly similar to AIF-C1 |
| | | | | | | [R. norvegicus] |
| 6322 | A | 85 | AA818801 | | | EST |
| 6330 | H | 873 | AI170426 | | | ESTs |
| 6366 | A, E, H | 152 | AA858716 | | | Rattus norvegicus mRNA for signal |
| | | | | | | peptidase 21kDa subunit, complete cds |
| 6380 | A, C, D | 153 | AA858758 | | | ESTs, Weakly similar to dJ413H6.1.1 |
| | | | | | | [H. sapiens] |
| 6409 | E | 156 | AA858910 | | | ESTs |
| 6410 | A | 157 | AA858926 | | | ESTs |
| 6431 | K, P | 159 | AA859085 | | | EST |
| 6439 | S | 636 | AI058436 | | | ESTs |
| 6440 | R | 160 | AA859130 | | | ESTs |
| 6443 | A | 161 | AA859150 | | | ESTs |
| 6473 | A | 1002 | AI177091 | | | ESTs |
| 6477 | N | 1371 | J00735 | | Fibrinogen, gamma polypeptide | Fibrinogen, gamma polypeptide |
| 6479 | K | 860 | AI169690 | | Fibrinogen, gamma polypeptide | Fibrinogen, gamma polypeptide |
| 6532 | B, Q | 1232 | AI234105 | | | ESTs |
| 6533 | E | 155 | AA858852 | | | ESTs, Moderately similar to |
| | | | | | | hypothetical protein |
| | | | | | | [H. sapiens] |
| 6541 | O | 740 | AI102905 | | | ESTs |
| 6549 | O | 949 | AI176002 | Folate biosynthesis | Folylpolyglutamate synthase | ESTs, Highly similar to S65755 |
| | | | | | | tetrahydrofolylpolyglutamate synthase |
| | | | | | | [M. musculus] |
| 6553 | S | 594 | AI030271 | | | ESTs |
| 6554 | A | 505 | AF097723 | | | Rattus norvegicus liver annexin-like |
| | | | | | | protein (LAL) mRNA, complete cds |
| 6582 | L | 910 | AI171726 | | | ESTs, Weakly similar to ESR1_RAT |
| | | | | | | ESTROGEN RECEPTOR |
| | | | | | | [R. norvegicus] |
| 6585 | F | 1695 | NM_022266 | | | Rattus norvegicus mRNA |
| | | | | | | for connective tissue growth |
| | | | | | | factor, complete cds |
| 6604 | A, O | 1104 | AI229192 | | | ESTs |
| 6613 | A, F | 117 | AA848758 | Butanoate metabolism, | HMm:hydroxylacyl- | Rattus norvegicus L-3-hydroxyacyl- |
| | | | | Fatty acid biosynthesis | Coenzyme A | CoA dehydrogenase precursor (HAD) |
| | | | | (path 2), Fatty acid | dehydrogenase | mRNA, complete cds; nuclear gene for |
| | | | | metabolism, | | mitochondrial product |
| | | | | Lysine degradation, | | |
| | | | | Tryptophan metabolism, | | |
| | | | | Valine, leucine and | | |
| | | | | isoleucine degradation | | |
| 6615 | A | 335 | AA942889 | | | ESTs, Weakly similar to putative |
| | | | | | | type III alcohol dehydrogenase |
| | | | | | | [D. melanogaster] |
| 6632 | A | 1246 | AI235277 | | | ESTs |
| 6633 | A, N | 1098 | AI228931 | | | ESTs |
| 6640 | A | 716 | AI101500 | | | ESTs |
| 6667 | K | 905 | AI171646 | | | ESTs |
| 6673 | E | 612 | AI044325 | | | Rattus norvegicus mRNA for |
| | | | | | | N-cadherin, complete cds |
| 6676 | L | 143 | AA851967 | | | ESTs |
| 6677 | S | 542 | AI011471 | | | ESTs |
| 6682 | A | 1168 | AI232065 | | | ESTs |
| 6686 | R | 952 | AI176130 | | | ESTs |
| 6761 | A | 513 | AI008699 | | | ESTs, Highly similar to methyl-CpG |
| | | | | | | binding domain-containing |
| | | | | | | protein MBD3 [M. musculus] |
| 6789 | O, R | 459 | AA998207 | | | ESTs |
| 6796 | C | 735 | AI102753 | | | ESTs |
| 6798 | E | 857 | AI169619 | | | ESTs |
| 6801 | A, E, K | 536 | AI010316 | | | ESTs |
| 6804 | E | 509 | AI007877 | | | ESTs |
| 6814 | E | 717 | AI101534 | | | EST, Rattus norvegicus Mdk mRNA for |
| | | | | | | midkine, complete cds |
| 6820 | A, D | 1133 | AI230439 | | | ESTs |
| 6821 | E, L | 990 | AI176841 | | | ESTs |
| 6824 | A, C, D, | 104 | AA819709 | | | ESTs |
| | F, I | | | | | |
| 6825 | A, B, Q, S | 631 | AI045972 | | | ESTs |
| 6855 | A, L | 899 | AI171370 | | | ESTs |
| 6861 | H, R | 995 | AI176970 | | | ESTs |
| 6879 | I | 907 | AI171674 | | | ESTs |
| 6892 | J | 33 | AA800551 | | | Rattus norvegicus DnaJ-like protein |
| | | | | | | (RDJ1) mRNA, complete cds |
| 6911 | D | 1343 | D85035 | Pantothenate and CoA | HHs:dihydropyrimidine | Rattus norvegicus mRNA for |
| | | | | biosynthesis, Pyrimidine | dehydrogenase | dihydropyrimidine dehydrogenase, |
| | | | | metabolism , beta-Alanine | | complete cds |
| | | | | metabolism | | |
| 6919 | N | 537 | AI010461 | | | ESTs |
| 6975 | O | 953 | AI176229 | | | ESTs |
| 7003 | A, L | 593 | AI030259 | | | ESTs, Weakly similar to Dreg-2 protein |
| | | | | | | [D. melanogaster] |
| 7036 | C, J | 1164 | AI231801 | | | ESTs, Weakly similar to TERA_RAT |
| | | | | | | TRANSITIONAL ENDOPLASMIC |
| | | | | | | RETICULUM ATPASE [R. norvegicus] |
| 7056 | B, M | 543 | AI011503 | | | ESTs |
| 7062 | A | 1533 | NM_012495 | Fructose and mannose | Aldolase A, | Aldolase A, fructose-biphosphate |
| | | | | metabolism, Glycolysis/ | fructose-biphosphate | |
| | | | | Gluconeogenesis, Pentose | | |
| | | | | phosphate cycle | | |
| 7063 | A, C, D | 1533 | NM_012495 | Fructose and mannose | Aldolase A, | Aldolase A, fructose-biphosphate |
| | | | | metabolism, Glycolysis/ | fructose-biphosphate | |
| | | | | Gluconeogenesis, Pentose | | |
| | | | | phosphate cycle | | |
| 7064 | A, C | 1533 | NM_012495 | Fructose and mannose | Aldolase A, | Aldolase A, fructose-biphosphate |
| | | | | metabolism, Glycolysis/ | fructose-biphosphate | |
| | | | | Gluconeogenesis, Pentose | | |
| | | | | phosphate cycle | | |
| 7111 | R | 108 | AA819816 | | | ESTs |
| 7113 | A | 868 | AI170260 | | | ESTs |
| 7122 | Q | 809 | AI137468 | | | ESTs |
| 7161 | C | 1209 | AI233407 | | | ESTs |
| 7176 | Q | 1306 | AI639029 | | | ESTs |
| 7196 | P | 1585 | NM_012904 | | Annexin 1 (p35) (Lipocortin 1) | Annexin 1 (p35) (Lipocortin 1) |
| 7199 | C, D | 562 | AI013044 | | | ESTs |
| 7225 | M | 564 | AI013657 | | | ESTs |
| 7243 | A, C | 1218 | AI233717 | | | ESTs |
| 7262 | D, L | 946 | AI175833 | | | ESTs |
| 7271 | C | 1115 | AI229739 | | | ESTs |
| 7295 | S | 572 | AI013876 | | | ESTs |
| 7299 | A | 573 | AI013911 | | | ESTs, Weakly similar to CIRP |
| | | | | | | [R. norvegicus] |
| 7301 | J | 111 | AA819854 | | | ESTs |
| 7352 | A | 577 | AI028973 | | | ESTs, Weakly similar to AF165892_1 |
| | | | | | | RNA-binding protein SiahBP |
| | | | | | | [R. norvegicus] |
| 7362 | L | 578 | AI029026 | | | ESTs |
| 7403 | C, D | 579 | AI029212 | | | EST |
| 7414 | C, D | 813 | AI137586 | | | ESTs, Highly similar to |
| | | | | | | IMB3_HUMAN |
| | | | | | | IMPORTIN BETA-3 SUBUNIT |
| | | | | | | [H. sapiens] |
| 7420 | S | 580 | AI029291 | | | ESTs, Highly similar to ClpX-like |
| | | | | | | protein [H. sapiens] |
| 7451 | E, N | 581 | AI029450 | | | ESTs, Moderately similar to |
| | | | | | | SYEP_HUMAN |
| | | | | | | MULTIFUNCTIONAL AMINOACYL- |
| | | | | | | TRNA SYNTHETASE |
| | | | | | | [H. sapiens] |
| 7497 | O | 849 | AI169302 | Sphingophospholipid | HMm:sphingomyelin | ESTs, Moderately similar to |
| | | | | biosynthesis | phosphodiesterase 1, acid | sphingomyelin phosphodiesterase 1, |
| | | | | | lysosomal | acid lysosomal [H. sapiens] |
| 7517 | S | 582 | AI029709 | | | ESTs |
| 7528 | H | 749 | AI103548 | | | ESTs, Highly similar to AF115778_1 |
| | | | | | | short coiled coil protein SCOCO |
| | | | | | | [M. musculus] |
| 7531 | A | 1298 | AI237614 | | | ESTs |
| 7537 | E | 584 | AI029829 | | | ESTs |
| 7552 | E, G, I | 629 | AI045802 | | | EST |
| 7582 | A | 588 | AI029996 | | | ESTs |
| 7584 | O | 601 | AI043724 | | | ESTs |
| 7586 | L | 589 | AI030024 | | | ESTs |
| 7602 | I | 1320 | AJ001929 | | | Rattus norvegicus mRNA for |
| | | | | | | of CBP-50 protein |
| 7617 | A | 591 | AI030170 | | | ESTs |
| 7665 | F | 596 | AI030668 | | | ESTs |
| 7681 | A | 595 | AI030449 | | | ESTs, Moderately similar to |
| | | | | | | methyltransferase related protein |
| | | | | | | [M. musculus] |
| 7684 | O | 592 | AI030242 | | | ESTs |
| 7690 | I | 1700 | NM_0222 | | | Rattus norvegicus uroguanylin mRNA, |
| | | | | | | complete cds |
| 7697 | A, M | 992 | AI176942 | | | ESTs |
| 7743 | P | 651 | AI070233 | | | ESTs |
| 7784 | A | 1570 | NM_012789 | | Dipeptidyl peptidase 4 | Dipeptidyl peptidase 4 |
| 7785 | A, C | 1570 | NM_012789 | | Dipeptidyl peptidase 4 | Dipeptidyl peptidase 4 |
| 7806 | J | 67 | AA818421 | | | ESTs |
| 7858 | M, P | 599 | AI043654 | | | EST |
| 7868 | A | 711 | AI101229 | | | ESTs |
| 7887 | C, D | 823 | AI144832 | Aminoacyl-tRNA | HHs:arginyl-tRNA | ESTs, Moderately similar to |
| | | | | biosynthesis, Arginine | synthetase | SYR_HUMAN ARGINYL-TRNA |
| | | | | and proline metabolism | | SYNTHETASE [H. sapiens] |
| 7888 | A, C, D | 1215 | AI233583 | Aminoacyl-tRNA | HHs:arginyl-tRNA | ESTs, Moderately similar to |
| | | | | biosynthesis, Arginine | synthetase | SYR_HUMAN ARGINYL-TRNA |
| | | | | and proline metabolism | | SYNTHETASE [H. sapiens] |
| 7892 | F | 1102 | AI229172 | | | ESTs, Weakly similar to FIBA_RAT |
| | | | | | | FIBRINOGEN ALPHA/ALPHA- |
| | | | | | | E CHAIN PRECURSOR |
| | | | | | | [R. norvegicus] |
| 7893 | A | 604 | AI043761 | | | EST |
| 7903 | A, E, F | 605 | AI043805 | | | ESTs |
| 7916 | E | 606 | AI043855 | Sterol biosynthesis | HMm:sterol-C5-desaturase | ESTs, Highly similar to sterol-C5- |
| | | | | | (fungal ERG3, | desaturase [M. musculus] |
| | | | | | delta-5-desaturase) | |
| | | | | | homolog (S. | |
| | | | | | cerevisae) | |
| 7918 | A | 1069 | AI179750 | | | ESTs |
| 7927 | A, H, O | 831 | AI145931 | Aminosugars metabolism | HHs:UDP-N- | R. norvegicusmRNA for UDP-N- |
| | | | | | acetylglucosamine-2- | acetyl-D glucosamine-2-epimerase |
| | | | | | epimerase/ | |
| | | | | | N-acetylmannosamine | |
| | | | | | kinase | |
| 7935 | C | 607 | AI043945 | Porphyrin and chlorophyll | HMm:ferrochelatase | ESTs |
| | | | | metabolism | | |
| 7936 | A | 202 | AA875495 | | | ESTs |
| 7967 | L | 1124 | AI230134 | Purine metabolism | HHs:adenylate cyclase 9 | ESTs |
| 8017 | P | 633 | AI058341 | | | EST, Weakly similar to putative integral |
| | | | | | | membrane transport protein |
| | | | | | | [R. norvegicus] |
| 8053 | K | 932 | AI175033 | | | ESTs |
| 8054 | R | 1099 | AI228959 | | | ESTs |
| 8079 | B, M, Q | 637 | AI058581 | | | ESTs |
| 8107 | G | 1318 | AI639534 | | | ESTs, Moderately similar to |
| | | | | | | PROP_MOUSE PROPERDIN |
| | | | | | | [M. musculus] |
| 8124 | E | 742 | AI103071 | | Protein tyrosine phosphatase, | ESTs |
| | | | | | gamma (provisional HGM11 | |
| | | | | | symbol) | |
| 8152 | I | 1478 | U77038 | | HMm:hemopoietic cell | Rattus norvegicus protein-tyrosine |
| | | | | | phosphatase | phosphatase (SHP-1) mRNA, complete |
| | | | | | | cds |
| 8173 | E | 450 | AA997699 | | | ESTs |
| 8177 | S | 638 | AI058603 | | | ESTs |
| 8215 | L | 909 | AI171692 | | | Rat ferritin light chain subunit, |
| | | | | | | mRNA, Rattus norvegicus kynurenine |
| | | | | | | aminotransferase/glutamine |
| | | | | | | transaminase K (Kat) gene, complete |
| | | | | | | cds |
| 8273 | P | 765 | AI104908 | | | ESTs |
| 8274 | B | 641 | AI059270 | | | EST, Weakly similar to hypothetical |
| | | | | | | protein [H. sapiens] |
| 8310 | P | 1048 | AI178868 | | | ESTs |
| 8314 | J | 642 | AI059386 | | | ESTs |
| 8315 | S | 643 | AI059389 | Alanine and aspartate | HMm:adenylosuccinate | ESTs, Highly similar to |
| | | | | metabolism, Purine | synthetase 1, muscle | PUA1_MOUSE |
| | | | | metabolism | | ADENYLOSUCCINATE |
| | | | | | | SYNTHETASE, MUSCLE ISOZYME |
| | | | | | | [M. musculus] |
| 8317 | A, E | 234 | AA892234 | Glutathione metabolism | HHs:microsomal | ESTs, Moderately similar to |
| | | | | | glutathione | microsomal glutathione S-transferase 3 |
| | | | | | [H. sapiens] S-transferase 3 | |
| 8356 | G | 645 | AI059543 | | | EST |
| 8387 | A | 962 | AI176365 | | | ESTs |
| 8477 | A | 1056 | AI179167 | | | ESTs |
| 8515 | N | 127 | AA849917 | | | ESTs |
| 8522 | M, P | 647 | AI060071 | | | ESTs |
| 8549 | A, F, H | 1216 | AI233639 | | | ESTs |
| 8592 | G | 1364 | H33491 | | | Rattus norvegicus sterol delta 8- |
| | | | | | | isomerase (RSI) mRNA, complete cds |
| 8597 | B, H | 72 | AA818593 | | | Rattus norvegicus phosphatidate |
| | | | | | | phosphohydrolase type 2 mRNA, |
| | | | | | | complete cds |
| 8600 | A | 640 | AI058956 | | | ESTs |
| 8630 | A | 529 | AI009677 | | | ESTs |
| 8661 | J | 73 | AA818604 | | Heat shock protein 70-1 | Rattus norvegicus heat shock protein 70 |
| | | | | | | (HSP70) mRNA, complete cds |
| 8662 | J | 115 | AA848563 | | Heat shock protein 70-1 | Rattus norvegicus heat shock protein 70 |
| | | | | | | (HSP70) mRNA, complete cds |
| 8663 | J | 1527 | Z27118 | | Heat shock protein 70-1 | Rattus norvegicus heat shock protein 70 |
| | | | | | | (HSP70) mRNA, complete cds |
| 8664 | J | 1530 | Z75029 | | Heat shock protein 70-1 | ESTs, Rattus norvegicus heat shock |
| | | | | | | protein 70 (HSP70) mRNA, complete |
| | | | | | | cds |
| 8665 | J | 675 | AI071965 | | Heat shock protein 70-1 | ESTs, Rattus norvegicus heat shock |
| | | | | | | protein 70 (HSP70) mRNA, complete |
| | | | | | | cds |
| 8692 | A | 610 | AI044247 | | | ESTs, Weakly similar to putative |
| | | | | | | peroxisomal 2,4-dienoyl-CoA reductase |
| | | | | | | [R. norvegicus] |
| 8700 | E, M | 634 | AI058388 | | | ESTs |
| 8709 | R | 1185 | AI232534 | | | ESTs, Weakly similar to DnaJ |
| | | | | | | homolog 2 [R. norvegicus] |
| 8715 | N | 648 | AI069920 | | | ESTs |
| 8728 | R | 74 | AA818615 | | | ESTs |
| 8730 | H | 1028 | AI178483 | | | ESTs |
| 8735 | H | 697 | AI073047 | | | Rattus norvegicus clone Pr2 unknown |
| | | | | | | mRNA |
| 8766 | A | 549 | AI012085 | | | ESTs, Weakly similar to thyroid |
| | | | | | | hormone responsive protein |
| | | | | | | [R. norvegicus] |
| 8820 | S | 650 | AI070152 | | | ESTs |
| 8829 | A | 1567 | NM_012749 | | Nucleolin | Nucleolin |
| 8864 | P | 652 | AI070319 | | | ESTs |
| 8872 | G, K | 134 | AA851050 | | | ESTs |
| 8880 | A | 824 | AI144936 | | | ESTs |
| 8886 | D | 1221 | AI233766 | | | ESTs, Highly similar to Ki antigen |
| | | | | | | [M. musculus] |
| 8905 | K | 790 | AI112511 | | | ESTs |
| 8928 | I | 212 | AA891221 | | | ESTs |
| 8946 | A | 656 | AI070611 | | | ESTs |
| 8984 | J | 1735 | NM_022539 | | Hsp:METHIONINE | Rattus norvegicus initiation factor 2 |
| | | | | | AMINOPEPTIDASE 2 | associated 67 kDa protein (p67) mRNA, |
| | | | | | | complete cds |
| 8993 | R | 948 | AI175997 | | | ESTs |
| 9012 | A | 657 | AI070879 | | | EST |
| 9015 | K | 1239 | AI234810 | | | ESTs |
| 9016 | A, B, C, | 659 | AI070903 | | | EST |
| | D, E | | | | | |
| 9053 | A | 249 | AA892861 | | | ESTs |
| 9063 | A | 1197 | AI233162 | | | ESTs |
| 9072 | G | 942 | AI175635 | | | ESTs |
| 9079 | P | 667 | AI071251 | | | ESTs |
| 9128 | L | 903 | AI171611 | | | ESTs |
| 9148 | B | 516 | AI008813 | | | ESTs |
| 9164 | H | 1565 | NM_012726 | | Spinocerebellar ataxia type 1 | ESTs |
| 9166 | E | 807 | AI137406 | | | ESTs |
| 9170 | E | 993 | AI176947 | | | ESTs |
| 9181 | C, D | 1071 | AI179870 | | | ESTs |
| 9190 | H | 702 | AI100835 | | | ESTs |
| 9191 | A | 681 | AI072107 | | | EST, Weakly similar to PE2R_RAT |
| | | | | | | 20-ALPHA-HYDROXYSTEROID |
| | | | | | | DEHYDROGENASE [R. norvegicus] |
| 9192 | E | 805 | AI137345 | | | ESTs |
| 9223 | Q | 1417 | M36151 | | | Rat MHC class II RT1.B beta gene, |
| | | | | | | encoding cell surface glycoprotein beta |
| | | | | | | chain, Rat mRNA for MHC class II |
| | | | | | | antigen RT1.B-1 beta-chain, Rattus |
| | | | | | | norvegicus MHC class II antigen RT1.B |
| | | | | | | beta chain mRNA, partial cds |
| 9245 | A | 684 | AI072778 | | | ESTs |
| 9267 | Q | 685 | AI072384 | | | ESTs, Moderately similar to human |
| | | | | | | formiminotransferase cyclodeaminase |
| | | | | | | [H. sapiens] |
| 9326 | A | 799 | AI136514 | | | ESTs, Moderately similar to SPIN |
| | | | | | | [H. sapiens ] |
| 9331 | A, C, D | 689 | AI072633 | | | ESTs |
| 9336 | A | 691 | AI072643 | | | ESTs |
| 9372 | S | 692 | AI072712 | | | ESTs |
| 9373 | S | 802 | AI136714 | | | ESTs |
| 9374 | R | 854 | AI169557 | | | ESTs Highly similar to |
| | | | | | | CDN6_MOUSE |
| | | | | | | CYCLIN-DEPENDENT KINASE 6 |
| | | | | | | INHIBITOR [M. musculus] |
| 9399 | A | 693 | AI072812 | | | ESTs |
| 9402 | O, R | 101 | AA819383 | | | ESTs |
| 9423 | S | 1556 | NM_012649 | | Ryudocan/syndecan 4 | Ryudocan/syndecan 4 |
| 9424 | N | 1556 | NM_012649 | | Ryudocan/syndecan 4 | Ryudocan/syndecan 4 |
| 9425 | A | 27 | AA800059 | | Ryudocan/syndecan 4 | Ryudocan/syndecan 4 |
| 9432 | E | 695 | AI072914 | | | EST |
| 9475 | A, O | 698 | AI073059 | | | ESTs |
| 9486 | L | 69 | AA818490 | | | ESTs |
| 9541 | A | 1704 | NM_022542 | | | Rat rhoB gene mRNA, complete cds |
| 9572 | R | 660 | AI071162 | | | ESTs |
| 9583 | A | 664 | AI071185 | | | ESTs |
| 9595 | B, E, Q | 800 | AI136630 | | | ESTs |
| 9598 | F | 1365 | H33832 | | | ESTs |
| 9603 | E | 666 | AI071227 | | | ESTs |
| 9621 | O | 937 | AI175486 | | ribosomal protein S7 | Rat PRRHIS8 mRNA for ribosomal |
| | | | | | | protein S8 |
| 9627 | A | 840 | AI169401 | | | ESTs |
| 9635 | N | 676 | AI071967 | | | ESTs, Weakly simiar to |
| | | | | | | Y281_HUMAN |
| | | | | | | HYPOTHETICAL PROTIEN |
| | | | | | | KIAA0281 [H. sapiens] |
| 9668 | K | 669 | AI071538 | | | ESTs |
| 9674 | L | 1044 | AI178784 | | | ESTs |
| 9697 | K | 671 | AI071642 | | | EST |
| 9712 | B, E | 988 | AI176836 | | | ESTs. Weakly similar to F25H5.6 |
| | | | | | | [C. elegans] |
| 9754 | A | 788 | AI112194 | | | ESTs |
| 9766 | R | 672 | AI071858 | | | ESTS |
| 9775 | L | 124 | AA849767 | | | Rattus norvegicus brain-enriched SH3- |
| | | | | | | domain protein mRNA, complete cds |
| 9784 | C | 710 | AI101226 | | | ESTs |
| 9796 | C | 677 | AI071990 | | | Rattus norvegicus pEachy mRNA, |
| | | | | | | complete cds |
| 9800 | R | 678 | AI072014 | | | ESTs, Weakly similar to AF165892_1 |
| | | | | | | RNA-binding protein SiahBP |
| | | | | | | [R. norvegicus] |
| 9826 | A, M | 228 | AA891950 | | | ESTs |
| 9889 | A | 618 | AI044621 | | | EST |
| 9905 | A, G | 221 | AA891774 | | | ESTs |
| 9925 | S | 620 | AI044925 | | | ESTs |
| 9969 | K | 622 | AI045195 | | | EST |
| 9977 | M | 623 | AI045253 | | | EST |
| 10002 | K | 816 | AI137988 | | | ESTs, Highly similar to myosin X |
| | | | | | | [M. musculus |
| 10016 | F, I | 1673 | NM_019289 | | Action-related | Actin-related protein complex 1b |
| | | | | | complex 1b | |
| 10019 | J | 1043 | AI178756 | | | ESTs |
| 10093 | G | 639 | AI058746 | | | EST |
| 10109 | A | 1502 | X58465 | | Ribosomal protein S5 | Ribsomal protein S5 |
| 10176 | A | 102 | AA819530 | | | Rattus norvegicus E-septin long form |
| | | | | | | mRNA complete cds |
| 10184 | E | 1363 | H33426 | | | ESTs |
| 10187 | F | 985 | AI176781 | | | ESTs |
| 10200 | L | 644 | AI059444 | | | ESTs |
| 10248 | A | 1574 | NM_012797 | | Inhibitor of DNA | Inhibitor of DNA binding |
| | | | | | binding 1, helix-loop | 1, helix-loop-helix protein |
| | | | | | -helix protein (splice | (splice varition) |
| | | | | | variation) | |
| 10306 | I | 506 | AF100470 | | | Rattus norvegicus SERP1 mRNA, |
| | | | | | | completed cds |
| 10378 | F | 1205 | AI233300 | | Complement | ESTs, Moderately similar to |
| | | | | | component 5 | CO5_HUMAN COMPLEMENT C5 |
| | | | | | | PRECURSOR [H. sapiens] |
| 10394 | R | 337 | AA943564 | | | ESTs |
| 10509 | A | 1696 | NM_022268 | Starch and sucrose | HHs: phosphorylase, | [R. norvegicus ] gene for glycogen |
| | | | | metabolism | glycogen; liver (Hers | phosphorylase (liver type) |
| | | | | | disease, glycogen | |
| | | | | | storage disease type | |
| | | | | | VI) | |
| 10533 | S | 635 | AI058430 | | | ESTs, highly similar to HG17_RAT |
| | | | | | | NONHISTONE CHROMOSOMAL |
| | | | | | | PROTEIN HMG-17 [R. norvegicus] |
| 10540 | O | 269 | AA894027 | | | EST |
| 10544 | A, B | 1341 | D63411 | | | Rattus norvegicus outer |
| | | | | | | mitochondrial membrane receptor |
| | | | | | | rTOM20 mRNA, complete cds |
| 10545 | A | 1455 | U21871 | | | Rattus norvegicus outer |
| | | | | | | mitochondrial membrane receptor |
| | | | | | | rTOM20 mRNA, complete cds |
| 10549 | C, D, E | 39 | AA801255 | | | ESTs |
| 10593 | R | 876 | AI170673 | | | ESTs |
| 10594 | E | 704 | AI100878 | | | ESTs, Highly similar to EST00098 |
| | | | | | | protein [H. sapiens] |
| 10611 | O | 1018 | AI177790 | | | ESTs |
| 10667 | N | 1273 | AI236366 | | | Rattus norvegicus RNA-binding protein |
| | | | | | | SiahBP mRNA, partial cds |
| 10790 | F, M | 602 | AI043728 | | | EST |
| 10879 | A, N | 687 | AI072476 | | | ESTs |
| 10984 | A, P | 842 | AI169156 | | | ESTs, Weakly similar to HP33 |
| | | | | | | [R. norvegicus] |
| 11021 | A, N | 106 | AA819767 | | | ESTs |
| 11039 | G | 1705 | NM_022543 | | | Rattus norvegicus steriod sensitive gene |
| | | | | | | 1 protein (SSG-1) mRNA, complete cds |
| 11048 | E | 668 | AI071456 | | | EST, Moderately similar to |
| | | | | | | AF099186_1EH domain-containing |
| | | | | | | protein EHD1 [M. musculus] |
| 11125 | L | 673 | AI071867 | | | ESTs, Highly similar to |
| | | | | | | phosphatidylserine synthase-2 |
| | | | | | | [M. musculus] |
| 11127 | E | 674 | AI071868 | | | EST |
| 11152 | G | 1629 | NM_017073 | Aminoacyl-tRNA | Glutamine synthetase | Glutamine synthetase |
| | | | | biosynthesis, Arginine | (glutamete- | (glutamate-ammonia |
| | | | | and proline metabolism, | ammonia ligase) | ligase) |
| | | | | Glutamate metabolism, | | |
| | | | | Nitrogen metabolism, | | |
| | | | | Porphyrin and chlorophyll | | |
| | | | | metabolism | | |
| 11153 | G | 1629 | NM_017073 | Aminoacyl-tRNA | Glutamine syntheta | Glutamine syntheta glutamate- |
| | | | | biosynthesis, Arginine and | glutamate-ammonia ligase) | ammonia ligase) |
| | | | | proline metabolism, | | |
| | | | | Glutamate metabolism, | | |
| | | | | Nitrogen metabolism, | | |
| | | | | Porphyrin and chlorophyll | | |
| | | | | metabolism | | |
| 11157 | A, E | 1184 | AI232494 | | | ESTs |
| 11166 | A | 40 | AA801346 | | | ESTs, Highly similar to |
| | | | | | | KIAA0315 [H. sapiens ] |
| 11172 | P | 338 | AA943730 | | | ESTs, Weakly similar to TISB_RAT |
| | | | | | | TIS11B PROTEIN [R. norvegicus] |
| 11174 | E | 333 | AA942745 | | | ESTs |
| 11179 | A, H | 783 | AI111559 | | | ESTs |
| 11205 | A, G | 919 | AI172057 | | | ESTs |
| 11215 | E | 49 | AA817921 | | | ESTs, Moderately similar to weak |
| | | | | | | similarity to Arabidopsis thaliana |
| | | | | | | ubiguitin-like protein 8 [C. elegans] |
| 11227 | 0 | 541 | AI010660 | | | ESTs |
| 11228 | A | 739 | AI102871 | | | ESTs |
| 11235 | D | 1068 | AI179709 | | | ESTs, Weakly similar to similar to |
| | | | | | | C. elegans hypothetical protein |
| | | | | | | CET01H8.1, CEC05C12.3, |
| | | | | | | CEF54D1.5. |
| | | | | | | similar to trp and trp-like proteins |
| | | | | | | [H. sapiens] |
| 11280 | R | 808 | AI137420 | | | ESTs, Moderately similar to hepatoma- |
| | | | | | | derived growth factor [M. musculus] |
| 11315 | R | 892 | AI171229 | | | ESTs, Moderately similar to imogen 44 |
| | | | | | | [M. musculus] |
| 11322 | E | 526 | AI009492 | | | ESTs, Highly similar to Unknown |
| | | | | | | [H. sapiens] |
| 11331 | C | 828 | AI145556 | | | ESTs |
| 11336 | R | 388 | AA946441 | | | ESTs |
| 11354 | R | 833 | AI146215 | | | ESTs |
| 11357 | A | 835 | AI146237 | | | ESTs |
| 11403 | A, D, L | 889 | AI171088 | Arginine and proline | HMm: spermidine synthase | ESTs, Highly similar to |
| | | | | | | SPEE_MOUSE |
| | | | | metabolism, Selenoamino | | SPERMIDINE SYNTHASE |
| | | | | acid metabolism, Urea | | [M. musculus] |
| | | | | cycle and metabolism of | | |
| | | | | amino groups, beta- | | |
| | | | | Alanine metabolism | | |
| 11404 | A, C, D, L | 1291 | AI237002 | Arginine and proline | HMm: spermidine synthase | ESTs, Highly similar to |
| | | | | | | SPEE_MOUSE |
| | | | | metabolism, Selenoamino | | SPERMIDINE SYNTHASE |
| | | | | acid metabolism, Urea | | [M. musculus] |
| | | | | cycle and metabolism of | | |
| | | | | amino groups, beta-Alanine | | |
| | | | | metabolism | | |
| 11422 | Q | 26 | AA799812 | | | ESTs, Moderately similar to |
| | | | | | | PTN3_HUMAN PROTEIN |
| | | | | | | TYROSINE PHOSPHATASE, |
| | | | | | | NON-RECEPTOR |
| | | | | | | TYPE 3 [H. sapiens] |
| 11423 | B, H, Q | 26 | AA799812 | | | ESTs, Moderately similar to |
| | | | | | | PTN3_HUMAN PROTEIN |
| | | | | | | TYROSINE PHOSPHATASE, |
| | | | | | | NON-RECEPTOR TYPE 3 |
| | | | | | | [H. sapiens] |
| 11426 | H | 896 | AI171305 | | | ESTs, Moderately similar to |
| | | | | | | PTN3_HUMAN PROTEIN |
| | | | | | | TYROSINE PHOSPHATASE, |
| | | | | | | NON-RECEPTOR TYPE 3 |
| | | | | | | [H. sapiens] |
| 11429 | A, G | 862 | AI169706 | | | ESTs |
| 11438 | E | 922 | AI172189 | | | ESTs |
| 11465 | O | 1263 | AI236084 | | | ESTs, Moderately similar to |
| | | | | | | 41BB_MOUSE 4-1BB LIGAND |
| | | | | | | RECEPTOR PRECURSOR |
| | | | | | | [M. musculus] |
| 11483 | J | 487 | AF020618 | | | ESTs, Moderately similar to progression |
| | | | | | | elevated gene 3 protein |
| | | | | | | [R. norvegicus], Rattus norvegicus |
| | | | | | | progression elevated gene 3 protein |
| | | | | | | mRNA, complete cds |
| 11485 | E | 1248 | AI235348 | | | ESTs, Highly similar to nuclear |
| | | | | | | transcriptional repressor Mph1 |
| | | | | | | [M. musculus] |
| 11492 | A | 770 | AI105145 | | | ESTs |
| 11493 | J | 1356 | H31287 | | | ESTs, Weakly similar to putative |
| | | | | | | serine/threonine protein kinase MAK-V |
| | | | | | | [M. musculus] |
| 11494 | J | 1356 | H31287 | | | ESTs, Weakly similar to putative |
| | | | | | | serine/threonine protein kinase MAK-V |
| | | | | | | [M. musculus] |
| 11495 | J | 991 | AI176901 | | | ESTs, Weakly similar to putative |
| | | | | | | serine/threonine protein kinase MAK-V |
| | | | | | | [M. musculus] |
| 11504 | A, B | 906 | AI171652 | | | ESTs |
| 11520 | A | 443 | AA997068 | | | ESTs, Weakly similar to CAG6_RAT |
| | | | | | | CMP-N-ACETYLNEURANMINATE- |
| | | | | | | BETA-1,4-GALACTOSIDE ALPHA- |
| | | | | | | 2,3-SIALYLTRANSFERASE |
| | | | | | | [R. norvegicus] |
| 11527 | A, C, R | 1108 | AI229307 | | | ESTs |
| 11536 | A | 984 | AI176739 | | | ESTs |
| 11561 | C | 1200 | AI233182 | | | ESTs |
| 11563 | A | 728 | AI102560 | | | ESTs |
| 11576 | A | 832 | AI146177 | | | ESTs |
| 11590 | E | 78 | AA818721 | | | ESTs, Moderately similar to S65785 |
| | | | | | | mel-13a protein-mouse |
| | | | | | | [M. musculus] |
| 11596 | M | 665 | AI071194 | | | ESTs |
| 11608 | F | 172 | AA859633 | | | ESTs |
| 11619 | L | 701 | AI100769 | | | ESTs |
| 11623 | E | 930 | AI172471 | | | ESTs, Highly similar to small |
| | | | | | | EDRK-rich factor 2 [M. musculus] |
| 11625 | R | 708 | AI101167 | | | ESTs, Weakly similar to ARL5_RAT |
| | | | | | | ADP-RIBOSYLATION FACTOR- |
| | | | | | | LIKE PROTEINS 5 [R. norvegicus] |
| 11635 | A, G | 173 | AA859645 | | | ESTs |
| 11644 | K, O | 1247 | AI235282 | | | ESTs |
| 11645 | F, M | 725 | AI102093 | | | ESTs, Weakly similar to B39066 |
| | | | | | | proline-rich protein 15-rat |
| | | | | | | [R. norvegicus] |
| 11660 | C, D | 1050 | AI178944 | | | ESTs, Highly similar to AF167573_1 |
| | | | | | | protein methyltransferase [M. musculus] |
| 11691 | A, E | 327 | AA926193 | | | Rattus norvegicus mRNA for |
| | | | | | | Sulfotransferase K2 |
| 11693 | A, C, D, | 836 | AI168953 | | | Rattus norvegicus mRNA for |
| | E, K | | | | | Sulfotransferase K2 |
| 11700 | E | 557 | AI012574 | | | ESTs |
| 11720 | B, O, Q | 1174 | AI232273 | | | ESTs, Highly similar to RNA cyclase |
| | | | | | | homolog [H. sapiens] |
| 11724 | K | 736 | AI102812 | | | ESTs |
| 11731 | P | 1544 | NM_012561 | | Follistatin | Follistatin |
| 11742 | A, E | 713 | AI101262 | | | ESTs |
| 11745 | A | 475 | AB006450 | | translocator of inner | translocator of inner mitochondrial |
| | | | | | mitochondrial membrane | membrane 17 kDa, a |
| | | | | | 17 kDa, a | |
| 11821 | 0 | 653 | AI070350 | | | ESTs, Weakly similar to DP1_MOUSE |
| | | | | | | POLYPOSIS LOCUS PROTEIN 1 |
| | | | | | | HOMOLOG [M. musculus] |
| 11830 | N | 1052 | AI179093 | | | ESTs |
| 11840 | N | 1526 | Y15068 | | | Rattus norvegicus mRNA for |
| | | | | | | Hsp70/Hsp90 organizing protein |
| 11850 | G | 1431 | R46985 | | | [R. norvegicus] mRNA for ribosomal |
| | | | | | | protein L10a |
| 11876 | L | 522 | AI009321 | | | ESTs |
| 11893 | B | 1139 | AI230951 | | | ESTs |
| 11904 | B, F, M, | 1344 | D85183 | | Brain immunoglobulin like | Brain immunoglobulin like |
| | Q | | | | protein with tyrosine-base | protein with tyrosine-base |
| | | | | | activation motifs, Protein | activation motifs, Protein |
| | | | | | tyrosine phosphatase, non- | tyrosine phosphatase, non- |
| | | | | | receptor type substrate 1 | receptor type substrate 1 |
| | | | | | (SHP substrate 1) | (SHP substrate 1) |
| 11940 | F, H | 209 | AA891108 | | | ESTs |
| 11959 | A | 217 | AA891735 | | | ESTs |
| 11960 | K | 220 | AA891740 | | | ESTs, Weakly similar to EPOR_RAT |
| | | | | | | ERYTHROPOIETIN RECEPTOR |
| | | | | | | PRECURSOR [R. norvegicus] |
| 11974 | B | 363 | AA944958 | | | ESTs |
| 12058 | R | 1393 | L25387 | Fructose and mannose | Hsp: | ESTs, Highly similar to K6PP_RAT 6- |
| | | | | metabolism, Galactose | 6-PHOSPHOFRUCTOKINASE, | PHOSPHOFRUCTOKINASE, TYPE C |
| | | | | metabolism, Glycolysis / | TYPE C | [R. norvegicus] |
| | | | | Gluconeogenesis, | | |
| | | | | Pentose phosphate cycle | | |
| 12064 | A | 32 | AA800429 | | | ESTs |
| 12087 | A | 1683 | NM_020082 | | ribonuclease 4 | ribonuclease 4 |
| 12120 | 0 | 121 | AA849365 | | | ESTs |
| 12155 | K | 1370 | J00728 | Fatty acid metabolism, | cytochrome P450, 2b19 | cytochrome P450, 2b19 |
| | | | | Tryptophan metabolism | | |
| 12156 | B, G, K | 1378 | K00996 | Fatty acid metabolism, | cytochrome P450, 2b19 | cytochrome P450, 2b19 |
| | | | | Tryptophan metabolism | | |
| 12157 | K | 1379 | K01721 | Fatty acid metabolism, | cytochrome P450, 2b19 | cytochrome P450, 2b19 |
| | | | | Tryptophan metabolism | | |
| 12158 | K | 1383 | L00320 | Fatty acid metabolism, | cytochrome P450, 2b19 | cytochrome P450, 2b19 |
| | | | | Tryptophan metabolism | | |
| 12160 | A, K | 66 | AA818412 | Fatty acid metabolism, | cytochrome P450, 2b19 | cytochrome P450, 2b19 |
| | | | | Tryptophan metabolism | | |
| 12185 | E | 890 | AI171094 | | | ESTs, Weakly similar to Cys2/His2 zinc |
| | | | | | | finger protein [R. norvegicus] |
| 12198 | R | 273 | AA899195 | | | Rattus norvegicus replication factor C |
| | | | | | | subunit 2 (RFC2) mRNA, partial cds |
| 12203 | L | 274 | AA899256 | | | ESTs, Weakly similar to translation |
| | | | | | | initiation factor [M. musculus] |
| 12215 | E, S | 696 | AI072959 | | | ESTs, Moderately similar to |
| | | | | | | monoglyceride lipase [M. musculus] |
| 12216 | A | 1106 | AI229240 | | | ESTs |
| 12277 | M, P | 342 | AA943800 | | | ESTs |
| 12306 | A, E, N | 360 | AA944898 | | | ESTs |
| 12312 | A | 263 | AA893453 | | | ESTs |
| 12314 | G | 372 | AA945596 | | | ESTs, Moderately similar to LECT2 |
| | | | | | | precursor [H. sapiens] |
| 12317 | E, R | 1237 | AI234361 | | | ESTs |
| 12331 | A | 389 | AA946466 | | | ESTs, Weakly similar to cytoplasmic |
| | | | | | | aminopeptidase P [R. norvegicus] |
| 12332 | A | 389 | AA946466 | | | ESTs, Weakly similar to cytoplasmic |
| | | | | | | aminopeptidase P [R. norvegicus] |
| 12361 | O | 433 | AA965031 | | | ESTs |
| 12375 | L | 798 | AI136478 | | | ESTs, Highly similar to p116Rip |
| | | | | | | [M. musculus] |
| 12450 | A, P | 755 | AI103955 | | | ESTs, Weakly similar to predicted using |
| | | | | | | Genefinder [C. elegans] |
| 12463 | Q | 1191 | AI232706 | | | ESTs |
| 12467 | S | 1193 | AI232924 | | | ESTs |
| 12471 | A | 413 | AA957433 | | | ESTs |
| 12551 | I | 1122 | AI230056 | | | ESTs |
| 12577 | F, M | 779 | AI111344 | | | Rattus norvegicus cyclin H mRNA, |
| | | | | | | complete cds |
| 12585 | O | 380 | AA946034 | | | ESTs, Highly similar to |
| | | | | | | AF151803_1 CGI 45 protein |
| | | | | | | [H. sapiens] |
| 12587 | A | 1120 | AI229979 | | | ESTs |
| 12613 | I | 1357 | H31620 | | | ESTs, Highly similar to hypothetical |
| | | | | | | protein [H. sapiens] |
| 12614 | C, D, R | 933 | AI175294 | | | ESTs |
| 12625 | R | 458 | AA998029 | | | ESTs |
| 12655 | A, O | 1226 | AI233836 | | | ESTs |
| 12694 | A | 416 | AA957906 | | | ESTs |
| 12714 | P | 533 | AI010050 | | | ESTs, Weakly similar to |
| | | | | | | LIS1_MOUSE |
| | | | | | | PLATELET-ACTIVATING FACTOR |
| | | | | | | ACETYLHYDROLASE IB ALPHA |
| | | | | | | SUBUNIT [R. norvegicus] |
| 12746 | O | 548 | AI011809 | | | ESTs |
| 12844 | N | 679 | AI072054 | | | ESTs |
| 12848 | A, G | 251 | AA892916 | | | ESTs, Weakly similar to hemomucin |
| | | | | | | [D. melanogaster] |
| 12857 | N | 694 | AI072866 | | | ESTs |
| 12880 | E | 782 | AI111558 | | | ESTs |
| 12928 | B, F, R | 396 | AA955564 | | | ESTs |
| 12946 | A, N | 1088 | AI228291 | | | ESTs |
| 12956 | L | 1296 | AI237580 | | | ESTs |
| 12964 | N | 1267 | AI236227 | | | ESTs |
| 12965 | C | 792 | AI112926 | | | ESTs |
| 12969 | J | 794 | AI112969 | | | ESTs |
| 12999 | C | 956 | AI176276 | Aminosugars | HHs:UDP-N-acteylglucosamine | ESTs |
| | | | | metabolism | pyrophosphorylase 1 | |
| 13045 | M | 801 | AI136702 | | | ESTs |
| 13055 | E | 1054 | AI179100 | | | ESTs, Highly similar to potential |
| | | | | | | membrane protein C14orf1 [H. sapiens] |
| 13088 | A, F, G | 266 | AA893495 | | | ESTs, Highly similar to CBG_RAT |
| | | | | | | CORTICOSTEROID-BINDING |
| | | | | | | GLOBULIN PRECURSOR |
| | | | | | | [R. norvegicus] |
| 13092 | O | 1158 | AI231547 | | HMm:FK506 binding | ESTs, Weakly similar to PPP5_RAT |
| | | | | | protein 4 (59 kDa) | SERINE/THREONINE PROTEIN |
| | | | | | | PHOSPHATASE 5 [R. norvegicus] |
| 13093 | B, O | 552 | AI012177 | | HMm:FK506 binding | ESTs, Weakly similar to PPP5_RAT |
| | | | | | protein 4 (59kDa) | SERINE/THREONINE PROTEIN |
| | | | | | | PHOSPHATASE 5 [R. norvegicus] |
| 13166 | A, R | 1039 | AI178736 | | | ESTs |
| 13175 | E | 965 | AI176465 | | | ESTs |
| 13203 | A, C | 1096 | AI228728 | | | ESTs |
| 13229 | O | 154 | AA858760 | | | ESTs |
| 13251 | C, D, R | 1059 | AI179264 | | | ESTs, Moderately similar to LZIP-1 and |
| | | | | | | LZIP-2 [M. musculus] |
| 13265 | J | 719 | AI101708 | | | ESTs |
| 13283 | A | 1598 | NM_013078 | Arginine and proline | Ornithine | Ornithine carbamoyltransferase |
| | | | | metabolism, Urea cycle and | carbamoyltransferase | |
| | | | | metabolism of amino | | |
| | | | | groups | | |
| 13294 | D | 1220 | AI233731 | | | ESTs, Weakly similar to TCPA_RAT |
| | | | | | | T-COMPLEX PROTEIN 1, ALPHA |
| | | | | | | SUBUNIT [R. norvegicus] |
| 13332 | B, Q | 257 | AA893080 | | | ESTs |
| 13351 | A, H | 62 | AA818271 | | | ESTs |
| 13353 | M, N | 938 | AI175508 | | | ESTs |
| 13458 | C, D, I | 934 | AI175338 | | | ESTs |
| 13467 | C | 817 | AI138034 | Sphingoglycolipid | HHs:UDP-glucose | Rattus norvegicus UDP- |
| | | | | metabolism | ceramide | glucose:ceramide glycosyltransferase |
| | | | | | glucosyltransferase | mRNA, complete cds |
| 13501 | R | 957 | AI176284 | | | ESTs |
| 13534 | E | 382 | AA946187 | | | ESTs |
| 13557 | B, E, L, N | 367 | AA945090 | | | ESTs |
| 13568 | H | 28 | AA800169 | | | ESTs |
| 13580 | K | 1030 | AI178507 | | | ESTs |
| 13581 | E | 1035 | AI178602 | | | ESTs |
| 13634 | A | 1061 | AI179381 | | | ESTs, Highly similar to S26812 |
| | | | | | | transcription factor ATF-4 - mouse |
| | | | | | | [M. musculus] |
| 13640 | E, H | 814 | AI137761 | | | ESTs |
| 13646 | C, D, E | 1509 | X62166 | | | ESTs, Highly similar to RL3_RAT 60S |
| | | | | | | RIBOSOMAL PROTEIN L3 |
| | | | | | | [R. norvegicus] |
| 13684 | A, D, I | 81 | AA818770 | | | Rattus norvegicus serine protease gene, |
| | | | | | | complete cds |
| 13723 | D | 1419 | M55534 | | Crystallin, alpha polypeptide 2 | ESTs, Rat alpha-crystallin B chain |
| | | | | | | mRNA, complete cds |
| 13749 | A | 1089 | AI228540 | | | ESTs |
| 13757 | A | 1094 | AI228676 | | | ESTs |
| 13762 | A, E | 1129 | AI230326 | | | ESTs |
| 13799 | L | 947 | AI175871 | | | ESTs |
| 13812 | R | 1101 | AI229167 | | | ESTs |
| 13838 | R | 1111 | AI229416 | | | ESTs |
| 13874 | C, D | 1117 | AI229832 | | | ESTs, Weakly similar to KIAA0859 |
| | | | | | | protein [H. sapiens] |
| 13895 | M | 1127 | AI230270 | | | ESTs |
| 13918 | E | 569 | AI013832 | | | ESTs |
| 13926 | H | 17 | AA799601 | | | ESTs |
| 13932 | E, H, N | 1142 | AI230988 | | | ESTs |
| 13949 | R | 1149 | AI231193 | | | ESTs, Moderately similar to |
| | | | | | | SEC_HUMAN SEC PROTEIN |
| | | | | | | [H. sapiens] |
| 13963 | A, O | 1154 | AI231388 | | | ESTs |
| 13967 | E | 1155 | AI231439 | | | EST |
| 13992 | Q | 1281 | AI236679 | | | ESTs |
| 14007 | A, E | 1166 | AI231808 | | | ESTs |
| 14016 | F | 489 | AF026505 | | | Rattus norvegicus SH3-containing |
| | | | | | | protein p4015 mRNA, complete cds |
| 14017 | F | 211 | AA891194 | | | Rattus norvegicus SH3-containing |
| | | | | | | protein p4015 mRNA, complete cds |
| 14035 | A | 1177 | AI232328 | Tyrosine | HHs:homogentisate 1,2- | ESTs, Highly similar to homogentisate |
| | | | | metabolism | dioxygenase (homogentisate | 1,2-dioxygenase [M. musculus] |
| | | | | | oxidase) | |
| 14051 | A, C, D | 1183 | AI232489 | | | ESTs, Weakly similar to PIR1 |
| | | | | | | [H. sapiens] |
| 14053 | E | 1243 | AI235046 | | | ESTs, Highly similar to |
| | | | | | | DDX6_MOUSE |
| | | | | | | PROBABLE ATP-DEPENDENT RNA |
| | | | | | | HELICASE P54 [M. musculus] |
| 14074 | A | 1206 | AI233323 | | | ESTs |
| 14081 | P | 1198 | AI233164 | | | ESTs |
| 14083 | A | 1009 | AI177181 | | | ESTs |
| 14095 | A | 1211 | AI233468 | | | ESTs |
| 14103 | A | 1199 | AI233172 | | | ESTs, Weakly similar to AF073727_1 |
| | | | | | | EH domain-binding mitotic |
| | | | | | | phosphoprotein [H. sapiens] |
| 14116 | S | 1207 | AI233361 | | | ESTs |
| 14118 | A | 1208 | AI233367 | | | EST |
| 14126 | E | 1062 | AI179415 | | HHs:neurotrophic | Rattus norvegicus tropomyosin non- |
| | | | | | tyrosine kinase, | muscle isoform NM1 (TPM-gamma) |
| | | | | | receptor, type 1 | mRNA, complete cds, |
| | | | | | | Rattus norvegicus |
| | | | | | | tropomyosin non-muscle isoform NM3 |
| | | | | | | (TPM-gamma) mRNA, complete cds |
| 14139 | H | 175 | AA859700 | Porphyrin and | HMm:protoporphyrinogen | EST, Highly similar to PPOX_MOUSE |
| | | | | chlorophyll | oxidase | PROTOPORPHYRINOGEN OXIDASE |
| | | | | metabolism | | [M. musculus], EST, |
| | | | | | | Moderately similar to PPOX_HUMAN |
| | | | | | | PROTOPORPHYRINOGEN OXIDASE |
| | | | | | | [H. sapiens] |
| 14171 | E | 1024 | AI178073 | | | ESTs, Weakly similar to cDNA EST |
| | | | | | | yk249b3.5 comes from this gene |
| | | | | | | [C. elegans] |
| 14181 | A | 1233 | AI234107 | | | ESTs |
| 14185 | P | 177 | AA859837 | Purine metabolism | HMm:guanine deaminase | Rattus norvegicus guanine |
| | | | | | | aminohydrolase (GAH) mRNA, |
| | | | | | | complete cds |
| 14195 | E | 775 | AI105205 | | | ESTs |
| 14199 | K | 1234 | AI234133 | | | ESTs |
| 14206 | A | 182 | AA859994 | | | ESTs |
| 14208 | A, B | 723 | AI102017 | | | ESTs |
| 14224 | C | 1140 | AI230956 | | | ESTs, Moderately similar to TFG |
| | | | | | | protein [M. musculus] |
| 14242 | C, D | 1086 | AI228197 | | | ESTs |
| 14250 | K | 21 | AA799729 | Purine metabolism | Phosphodiesterase 4B, | ESTs, Phosphodiesterase 4B, cAMP- |
| | | | | | cAMP-specific (dunce | specific (dunce (Drosophila)- |
| | | | | | (Drosophila)-homolog | homolog phosphodiesterase E4) |
| 14258 | C | 1118 | AI229902 | | phosphodiesterase E4) | ESTs |
| 14264 | S | 1181 | AI232409 | | | ESTs, Weakly similar to bK126B4.2 |
| | | | | | | [H. sapiens] |
| 14266 | O | 1366 | H33842 | | | ESTs, Highly similar to phosphoprotein |
| | | | | | | [M. musculus] |
| 14303 | L | 1148 | AI231159 | | | ESTs, Highly similar to KIAA1049 |
| | | | | | | protein [H. sapiens] |
| 14312 | A, E | 1261 | AI236036 | | | ESTs, Moderately similar to UBE-1b |
| | | | | | | [M. musculus] |
| 14330 | P | 233 | AA892146 | | | ESTs |
| 14335 | E | 1006 | AI177115 | | | ESTs |
| 14353 | A | 171 | AA859585 | | | ESTs |
| 14400 | F, M | 858 | AI169620 | | | ESTs |
| 14424 | A, J | 654 | AI070421 | | | ESTs |
| 14449 | E | 1235 | AI234152 | | | ESTs |
| 14458 | C, I | 826 | AI145095 | | | ESTs |
| 14462 | C, D | 703 | AI100871 | | | ESTs |
| 14465 | F | 253 | AA892950 | | | ESTs, Moderately similar to |
| | | | | | | mitochondrial DNA polymerase |
| | | | | | | accessory subunit [M. musculus] |
| 14491 | M | 535 | AI010147 | | | ESTs |
| 14504 | M, P | 25 | AA799804 | | | ESTs |
| 14506 | A | 1359 | H32584 | | | ESTs |
| 14507 | S | 132 | AA850618 | | | ESTs, Highly similar to gp250 |
| | | | | | | precursor [M. musculus] |
| 14512 | A, G | 793 | AI112964 | | | ESTs |
| 14584 | A | 1250 | AI235360 | | | ESTs, Moderately similar to |
| | | | | | | glutathione-S-transferase homolog |
| | | | | | | [M. musculus] |
| 14595 | S | 232 | AA892128 | | | ESTs |
| 14600 | E, R | 38 | AA801076 | | | ESTs |
| 14619 | C, D | 1290 | AI236989 | | | ESTs |
| 14638 | E | 803 | AI137049 | | | ESTs, Moderately similar to Nibrin |
| | | | | | | [M. musculus] |
| 14693 | A, C, D | 1240 | AI234830 | | | ESTs, Weakly similar to ORF |
| | | | | | | YKR081c [S. cerevisiae] |
| 14738 | N, O | 997 | AI176993 | | | ESTs |
| 14746 | A | 1252 | AI235584 | | | ESTs, Moderately similar to KIAA0922 |
| | | | | | | protein [H. sapiens] |
| 14767 | A | 1256 | AI235895 | | | ESTs |
| 14776 | A, E, N | 1258 | AI235950 | | | ESTs |
| 14840 | K | 1301 | AI237698 | | | ESTs |
| 14869 | A | 1264 | AI236089 | | | ESTs, Weakly similar to /prediction |
| 14882 | S | 1324 | D00362 | | Esterase 2 | Esterase 2 |
| 14913 | L, R | 1274 | AI236461 | | | ESTs |
| 14937 | A, E | 1293 | AI237159 | | | ESTs, Highly similar to lipoic acid |
| | | | | | | synthetase [H. sapiens] |
| 14939 | C, D | 1090 | AI228557 | | | ESTs |
| 14958 | N | 105 | AA819744 | | | ESTs |
| 14959 | I | 1444 | U03390 | | | Rattus norvegicus Sprague Dawley |
| | | | | | | protein kinase C receptor mRNA, |
| | | | | | | complete cds |
| 14960 | A, G, O | 897 | AI171319 | | | ESTs, Highly similar to integrase |
| | | | | | | interactor 1a protein |
| | | | | | | [M. musculus, Rattus norvegicus |
| | | | | | | Sprague Dawley protein kinase C |
| | | | | | | receptor mRNA, complete cds |
| 14962 | A, C, D | 845 | AI169171 | | | ESTs, Highly similar to ENHANCER |
| | | | | | | OF RUDIMENTARY HOMOLOG |
| | | | | | | [M. musculus] |
| 14970 | G | 218 | AA891738 | Sulfur metabolism | HHs:sulfite oxidase | Rattus norvegicus sulfite oxidase |
| | | | | | | mRNA, complete cds |
| 14989 | O | 1012 | AI177366 | | Integrin, beta 1 | Integrin, beta 1 |
| 14996 | A, N | 1597 | NM_013059 | Folate biosynthesis, | Tissue-nonspecific ALP alkaline | Tissue-nonspecific ALP alkaline |
| | | | | Glycerolipid metabolism | phosphatase | phosphatase |
| 14997 | A, E, N, | 1597 | NM_013059 | Folate biosynthesis, | Tissue-nonspecific ALP alkaline | Tissue-nonspecific ALP alkaline |
| | O | | | Glycerolipid metabolism | phosphatase | phosphatase |
| 15002 | F | 851 | AI169327 | | | Rattus norvegicus tissue inhibitor of |
| | | | | | | metalloproteinase-1 (TIMP1), mRNA, |
| | | | | | | complete cds |
| 15003 | F | 851 | AI169327 | | | Rattus norvegicus tissue inhibitor of |
| | | | | | | metalloproteinase-1 (TIMP1), mRNA, |
| | | | | | | complete cds |
| 15004 | A | 1244 | AI235224 | | | Rattus norvegicus tissue inhibitor of |
| | | | | | | metalloproteinase-1 (TIMP1), mRNA, |
| | | | | | | complete cds |
| 15015 | S | 961 | AI176363 | | | ESTs |
| 15016 | A | 925 | AI172285 | | | ESTs |
| 15018 | E, S | 430 | AA964688 | | | ESTs |
| 15029 | A, C, D, | 878 | AI170696 | | | ESTs, Weakly similar to development- |
| | E, P | | | | | related protein [R. norvegicus] |
| 15030 | L | 113 | AA848378 | | | ESTs |
| 15032 | A, D | 1576 | NM_012816 | | Methylacyl-CoA racemase alpha | Methylacyl-CoA racemase alpha |
| 15051 | J, R | 1271 | AI236332 | Arginine and proline | Spermidine / spermine N1- | ESTs, Highly similar to |
| | | | | | | ATDA_MOUSE |
| | | | | metabolism | acyltransferase (diamine | DIAMINE ACETYLTRANSFERASE |
| | | | | | acetyltransferase) | [M. musculus |
| 15055 | A | 1463 | U48220 | Fatty acid | HHs:cytochrome P450, | Rattus norvegicus cytochrome P450 |
| | | | | metabolism | subfamily IID (debrisoquine, | 2D18 mRNA, complete cds |
| | | | | Tryptophan | sparteine, etc., -metabolizing), | |
| | | | | metabolism | polypeptide 6 | |
| 15057 | O | 1675 | NM_019291 | Nitrogen | carbonic anhydrase 2 | carbonic anhydrase 2 |
| | | | | metabolism | | |
| 15070 | H | 1081 | AI180442 | Sterol | HHs:farnesyl diphosphate | Rat testis-specific farnesyl |
| | | | | biosynthesis | synthase (farnesyl | pyrophosphate synthetase mRNA, |
| | | | | | pyrophosphate synthetase | complete cds |
| | | | | | dimethylallyltranstransferase, | |
| | | | | | geranyltranstransferase) | |
| 15080 | A | 724 | AI102045 | | | ESTs, Highly similar to OS-4 protein |
| | | | | | | [H. sapiens] |
| 15089 | F | 530 | AI009752 | | | ESTs |
| 15091 | J | 1040 | AI178740 | | YY1 transcription factor | ESTs |
| 15097 | L, O | 1548 | NM_012588 | | Insulin-like growth factor- | Insulin-like growth factor-binding |
| | | | | | binding protein (IGF-BP3) | protein (IGF-BP3) |
| 15113 | A, G | 941 | AI175590 | | | ESTs, Highly similar to dJ1118D24.1c |
| | | | | | | [H. sapiens] |
| 15116 | P | 190 | AA874928 | | | ESTs, Highly similar to sorting nexin 4 |
| | | | | | | [H. sapiens] |
| 15121 | E | 746 | AI103159 | | | Rattus norvegicus interferon-inducible |
| | | | | | | protein 16 mRNA, complete cds |
| 15122 | E | 1176 | AI232303 | | | ESTs, Weakly similar to Sid1669p |
| | | | | | | [M. musculus] |
| 15127 | B, K | 1434 | S56937 | Androgen | UDP-glucuronosyltransferase | Rattus norvegicus UDP- |
| | | | | and estrogen | 1 family, member 1 | glucuronosyltransferase (UGT1.1) gene, |
| | | | | metabolism, | | complete cds, Rattus norvegicus UDP- |
| | | | | Pentose and | | glucuronosyltransferase UGT1A7 |
| | | | | glucuronate | | mRNA, complete cds, UDP- |
| | | | | inter- | | glucuronosyltransferase 1 family, |
| | | | | conversions, | | member 1 |
| | | | | Porphyrin | | |
| | | | | and | | |
| | | | | chlorophyll | | |
| | | | | metabolism, | | |
| | | | | Starch and | | |
| | | | | sucrose | | |
| | | | | metabolism | | |
| 15135 | A, D | 1436 | S71021 | | | R. norvegicus mRNA for ribosomal |
| | | | | | | protein L6 |
| 15136 | A | 20 | AA799672 | | | R. norvegicus mRNA for ribosomal |
| | | | | | | protein L6 |
| 15139 | H | 818 | AI144585 | | | ESTs |
| 15141 | E, F | 1649 | NM_017278 | | proteasome | proteasome (prosome, macropain) |
| | | | | | (prosome, macropain) | subunit, alpha type 1 |
| | | | | | subunit, alpha | |
| | | | | | type 1 | |
| 15149 | R | 164 | AA859327 | | | ESTs |
| 15156 | A, E | 165 | AA859341 | | | ESTs, Highly similar to KIAA0418 |
| | | | | | | [H. sapiens] |
| 15162 | L | 168 | AA859350 | | | ESTs |
| 15170 | A, H, N | 1299 | AI237618 | | | ESTs |
| 15171 | J | 1160 | AI231792 | | | ESTs, Moderately similar to |
| | | | | | | BAG-family molecular chaperone |
| | | | | | | regulator-3 [H. sapiens] |
| 15172 | J | 169 | AA859362 | | | ESTs, Moderately similar to |
| | | | | | | BAG-family molecular chaperone |
| | | | | | | regulator-3 [H. sapiens] |
| 15179 | R | 982 | AI176675 | | | ESTs |
| 15181 | H | 1245 | AI235234 | | | ESTs |
| 15189 | M, N | 1399 | M11794 | | Metallothionein | Metallothionein |
| 15190 | N | 729 | AI102562 | | Metallothionein | Metallothionein |
| 15191 | N | 964 | AI176456 | | Metallothionein | Metallothionein |
| 15197 | A | 778 | AI105444 | | | ESTs |
| 15203 | I | 1389 | L19698 | | | Rat GTP-binding protein |
| | | | | | | (ral A) mRNA, |
| | | | | | | complete cds |
| 15207 | A, B, Q | 147 | AA858448 | | | ESTs |
| 15239 | A | 1619 | NM_016989 | | | R. norvegicus (Sprague Dawley) |
| | | | | | | ribosomal protein L15 mRNA |
| 15240 | A | 609 | AI044241 | | | ESTs, Moderately similar to cell death |
| | | | | | | activator CIDE-B [M. musculus] |
| 15251 | E, L | 1011 | AI177363 | | | ESTs, Highly similar to CSK_RAT |
| | | | | | | TYROSINE-PROTEIN KINASE CSK |
| | | | | | | [R. norvegicus] |
| 15281 | I | 1328 | D13623 | | | ESTs |
| 15282 | D, I, L | 1034 | AI178573 | | | ESTs |
| 15283 | D | 148 | AA858548 | | | ESTs |
| 15291 | J | 780 | AI111401 | | multiple inositol | multiple inositol polyphosphate |
| | | | | | polyphosphate histidine | histidine phosphatase 1 |
| | | | | | phosphatase 1 | |
| 15292 | J | 484 | AF012714 | | multiple inositol | multiple inositol polyphosphate |
| | | | | | polyphosphate histidine | histidine phosphatase 1 |
| | | | | | phosphatase 1 | |
| 15295 | O | 1602 | NM_013102 | | FK506-binding | FK506-binding protein 1 (12kD) |
| | | | | | protein 1 (12kD) | |
| 15299 | A | 1647 | NM_017259 | | B-cell translocation | B-cell translocation gene 2, anti- |
| | | | | | gene 2, anti- | proliferative |
| | | | | | proliferative | |
| 15300 | A, F | 1647 | NM_017259 | | B-cell translocation | B-cell translocation gene 2, anti- |
| | | | | | gene 2, anti- | proliferative |
| | | | | | proliferative | |
| 15301 | A | 1647 | NM_017259 | | B-cell translocation | B-cell translocation gene 2, anti- |
| | | | | | gene 2, anti- | proliferative |
| | | | | | proliferative | |
| 15312 | C, D, I, J | 198 | AA875126 | | | ESTs |
| 15313 | C, D, J | 198 | AA875126 | | | ESTs |
| 15315 | G | 1021 | AI177911 | | calpactin I heavy chain | calpactin I heavy chain |
| 15345 | L | 902 | AI171587 | | | ESTs |
| 15365 | D | 1637 | NM_017147 | | cofilin 1, non-muscle | cofilin 1, non-muscle |
| 15374 | C, D | 1368 | H34186 | | | ESTs, Highly similar to IF39_HUMAN |
| | | | | | | EUKARYOTIC TRANSLATION |
| | | | | | | INITIATION FACTOR 3 SUBUNIT 9 |
| | | | | | | [H. sapiens] |
| 15382 | A, J | 926 | AI172302 | | | ESTs, Weakly similar to S43056 |
| | | | | | | hypothetical protein - mouse |
| | | | | | | [M. musculus] |
| 15391 | K | 534 | AI010083 | | | Rat mRNA for HBP23 (heme-binding |
| | | | | | | protein 23 kDa), complete cds |
| 15398 | C | 1277 | AI236566 | | | ESTs |
| 15433 | L | 1641 | NM_017187 | | high mobility group | high mobility group protein 2 |
| | | | | | protein 2 | |
| 15441 | K | 834 | AI146216 | | | EST |
| 15462 | G | 1447 | U06230 | | | Rattus norvegicus protein S mRNA, |
| | | | | | | partial cds |
| 15467 | H | 1265 | AI236106 | | | ESTs |
| 15480 | F | 201 | AA875362 | | | ESTs |
| 15490 | J | 1107 | AI229253 | | | Rattus norvegicus zinc finger protein |
| | | | | | | (pMLZ-4) mRNA, 3′ untranslated |
| | | | | | | region |
| 15491 | H | 979 | AI176642 | | | ESTs |
| 15500 | K | 1110 | AI229337 | | | ESTs |
| 15503 | P | 1668 | NM_019237 | | procollagen C-proteinase | procollagen C-proteinase enhancer |
| | | | | | enhancer | protein |
| | | | | | protein | |
| 15504 | M, P | 1668 | NM_019237 | | procollagen C-proteinase | procollagen C-proteinase enhancer |
| | | | | | enhancer | protein |
| | | | | | protein | |
| 15519 | A | 1036 | AI178629 | | Proteasome | ESTs, Highly similar to PRCY_RAT |
| | | | | | (prosome, macropain) | PROTEASOME COMPONENT C13 |
| | | | | | subunit, beta type, 8 | PRECURSOR [R. norvegicus] |
| | | | | | (low molecular | |
| | | | | | mass polypeptide 7) | |
| 15534 | O | 955 | AI176266 | | | ESTs |
| 15535 | F | 1653 | NM_017283 | | proteasome | proteasome (prosome, macropain) |
| | | | | | (prosome, macropain) | subunit, alpha type 6 |
| | | | | | subunit, alpha type 6 | |
| 15543 | D, I | 1163 | AI231800 | | | ESTs |
| 15551 | R | 1138 | AI230759 | | | ESTs, Moderately similar to ornithine |
| | | | | | | decarboxylase antizyme 2 |
| | | | | | | [M. musculus] |
| 15558 | J | 204 | AA875537 | | | ESTs |
| 15571 | G | 1413 | M27207 | | procollagen, type I, | R. norvegicus mRNA for collagen |
| | | | | | alpha 1 | alpha 1 type I |
| 15606 | B, N | 356 | AA944401 | | | ESTs |
| 15612 | A | 1618 | NM_016987 | Citrate cycle | ATP citrate lyase | ATP citrate lyase |
| | | | | (TCA cycle) | | |
| 15616 | J | 1562 | NM_012699 | | Microvascular endothelial | Microvascular endothelial |
| | | | | | differentiation | differentiation gene 1 |
| | | | | | gene 1 | |
| 15617 | J | 205 | AA875620 | | | ESTs |
| 15634 | H | 1546 | NM_012576 | | Glucocorticoid receptor | Glucocorticoid receptor |
| 15642 | A | 1016 | AI177503 | | | R. norvegicus mRNA for histone H3.3 |
| 15645 | K | 879 | AI170709 | | | R. norvegicus mRNA for histone H3.3 |
| 15647 | A, J | 488 | AF025424 | Purine | HMm:RNA polymerase | Rattus norvegicus RNA polymerase I |
| | | | | metabolism, | 1-2 (128 kDa | 127 kDa subunit mRNA, complete cds |
| | | | | Pyrimidine | subunit) | |
| | | | | metabolism | | |
| 15655 | I, L | 733 | AI102739 | | | ESTs |
| 15663 | D, R | 940 | AI175566 | | | Rattus norvegicus mRNA for Tctex-1, |
| | | | | | | complete cds |
| 15672 | S | 281 | AA900009 | | | Rat mRNA for 5E5 antigen, complete |
| | | | | | | cds |
| 15673 | G | 921 | AI172107 | | | Rat mRNA for 5E5 antigen, complete |
| | | | | | | cds |
| 15700 | A, D | 479 | AB010466 | | | Rattus norvegicus mRNA for multidrug |
| | | | | | | resistance-associated protein (MRP)-like |
| | | | | | | protein-1 (MLP-1), complete cds |
| 15701 | F, G | 1645 | NM_017220 | | | Rattus norvegicus mRNA for multidrug |
| | | | | | | resistance-associated protein (MRP)-like |
| | | | | | | protein-2 (MLP-2), complete cds |
| 15755 | A, K | 1718 | NM_022960 | | | Rattus norvegicus neutral solute channel |
| | | | | | | aquaporin 9 (AQP9) mRNA, complete |
| | | | | | | cds |
| 15778 | E | 1726 | NM_024163 | | | Rattus norvegicus brain-enriched |
| | | | | | | guanylate kinase-associated protein 1 |
| | | | | | | mRNA, complete cds |
| 15786 | B, Q | 575 | AI013924 | | | ESTs |
| 15834 | B, E | 286 | AA900580 | Oxidative | HHs:NADH | ESTs, Moderately similar to NADH- |
| | | | | phosphorylation, | dehydrogenase | ubiquinone oxidoreductase B14.5B |
| | | | | Ubiquinone | (ubiquinone) 1, | subunit [H. sapiens] |
| | | | | biosynthesis | subcomplex unknown, 2 | |
| | | | | | (14.5kD, B14.5b) | |
| 15860 | D | 738 | AI102868 | | | ESTs, Weakly similar to phosphoserine |
| | | | | | | aminotransferase [H. sapiens] |
| 15861 | C, D | 738 | AI102868 | | | ESTs, Weakly similar to phosphoserine |
| | | | | | | aminotransferase [H. sapiens] |
| 15862 | A, C, D | 1126 | AI230228 | | | ESTs, Weakly similar to phosphoserine |
| | | | | | | aminotransferase [H. sapiens] |
| 15884 | A, Q | 185 | AA866276 | | | ESTs |
| 15888 | K | 199 | AA875225 | | | Rat guanine nucleotide-binding |
| | | | | | | protein G i, alpha subunit mRNA, |
| | | | | | | complete cds |
| 15892 | A, F | 1074 | AI179988 | | | ESTs |
| 15900 | A, C, D | 1202 | AI233262 | | | ESTs |
| 15914 | F | 451 | AA997711 | | | ESTs |
| 15933 | A | 200 | AA875253 | | | R. norvegicus ARL1 mRNA for |
| | | | | | | ARF-like protein 1 |
| 15955 | A, K, L | 1175 | AI232294 | | | ESTs |
| 15959 | E, L | 972 | AI176540 | | | ESTs |
| 15961 | P | 550 | AI012130 | | | ESTs |
| 15980 | H | 186 | AA866426 | | | ESTs |
| 15987 | K | 187 | AA866435 | | | EST |
| 16006 | A, F | 497 | AF062594 | | | Rattus norvegicus nucleosome |
| | | | | | | assembly protein mRNA, complete cds |
| 16023 | G | 225 | AA891872 | Nicotinate and | Nicotinamide nucleotide | ESTs, Highly similar to NAD(P) + |
| | | | | nicotinamide | transhydrogenase | transhydrogenase [M. musculus] |
| | | | | metabolism | (NAD(P) + | |
| | | | | | transhydrogenase) | |
| 16053 | L | 1091 | AI228596 | | | ESTs, Weakly similar to weakly |
| | | | | | | similar to gastrula zinc finger protein |
| | | | | | | [C. elegans] |
| 16080 | A, J, Q | 1547 | NM_012580 | Porphyrin and | Heme oxygenase | Heme oxygenase |
| | | | | chlorophyll | | |
| | | | | metabolism | | |
| 16081 | A, J, Q | 1067 | AI179610 | Porphyrin and | Heme oxygenase | Heme oxygenase |
| | | | | chlorophyll | | |
| | | | | metabolism | | |
| 16085 | A, C, D | 189 | AA874889 | | | ESTs |
| 16087 | L | 1145 | AI231011 | | | ESTs |
| 16124 | K | 994 | AI176963 | | | ESTs, Weakly similar to melanocyte- |
| | | | | | | specific gene 1 protein [R. norvegicus] |
| 16125 | Q | 503 | AF090134 | | | Rattus norvegicus lin-7-Ba mRNA, |
| | | | | | | complete cds |
| 16134 | A, H | 265 | AA893485 | | | Rattus norvegicus clone BB.1.4.1 |
| | | | | | | unknown Glu-Pro dipeptide repeat |
| | | | | | | protein mRNA, complete cds |
| 16167 | E | 191 | AA874941 | | | ESTs, Moderately similar to adipophilin |
| | | | | | | [H. sapiens] |
| 16169 | E | 598 | AI030932 | | | ESTs, Moderately similar to adipophilin |
| | | | | | | [H. sapiens] |
| 16172 | A | 1179 | AI232341 | | | ESTs, Weakly similar to C13B9.2 |
| | | | | | | [C. elegans] |
| 16173 | M, P | 408 | AA957003 | | | Rattus norvegicus intercellular calcium- |
| | | | | | | binding protein (MRP8) mRNA, |
| | | | | | | complete cds |
| 16190 | A, S | 757 | AI104482 | | | ESTs, Weakly similar to ECHM_RAT |
| | | | | | | ENOYL-COA HYDRATASE, |
| | | | | | | MITOCHONDRIAL PRECURSOR |
| | | | | | | [R. norvegicus] |
| 16205 | L | 1488 | X06423 | | | Rat mRNA for ribosomal protein S8 |
| 16215 | H | 192 | AA874999 | | | ESTs, Moderately similar to |
| | | | | | | AF133910_1 ARL-6 interacting |
| | | | | | | protein-3 [M. musculus] |
| 16219 | G | 1557 | NM_012656 | | Secreted acidic | Secreted acidic cystein-rich |
| | | | | | cystein-rich | glycoprotein (osteonectin) |
| | | | | | glycoprotein | |
| | | | | | (osteonectin) | |
| 16240 | M | 166 | AA859342 | | | ESTs, Moderately similar to |
| | | | | | | DHB2_RAT ESTRADIOL 17 BETA- |
| | | | | | | DEHYDROGENASE 2 [R. norvegicus] |
| 16251 | E, Q | 347 | AA944077 | | Solute carrier family | Rat brain glucose-transporter protein |
| | | | | | 2 a 1 (facilitated | mRNA, complete cds |
| | | | | | glucose transporter) | |
| | | | | | brain | |
| 16278 | E, K | 1338 | D38381 | Fatty acid | Hsp:CYTOCHROME | R. norvegicus CYP3 mRNA |
| | | | | metabolism, | P450 3A18 | |
| | | | | Tryptophan | | |
| | | | | metabolism | | |
| 16283 | O | 1667 | NM_019229 | | solute carrier | solute carrier family 12, member 4 |
| | | | | | family 12, member 4 | |
| 16312 | A | 193 | AA875032 | | | ESTs |
| 16314 | A | 167 | AA859348 | | | ESTs |
| 16317 | B | 194 | AA875041 | | | ESTs, Moderately similar to |
| | | | | | | AF123655_1 FEZ1 [H. sapiens] |
| 16318 | J | 174 | AA859648 | | | ESTs, Weakly similar to DnaJ |
| | | | | | | homolog 2 [R. norvegicus] |
| 16319 | K | 195 | AA875047 | | | ESTs, Highly similar to |
| | | | | | | TCPZ_MOUSE T |
| | | | | | | COMPLEX PROTEIN 1, ZETA |
| | | | | | | SUBUNIT [M. musculus] |
| 16321 | C | 1157 | AI231506 | | | ESTs |
| 16323 | S | 184 | AA866240 | | | EST |
| 16324 | A | 722 | AI102009 | | | ESTs |
| 16327 | A, O | 196 | AA875050 | | | ESTs, Weakly similar to |
| | | | | | | choline/ethanolamine kinase |
| | | | | | | [R. norvegicus] |
| 16361 | H | 1442 | U01344 | | Hsp:ARYLAMINE N- | Rattus norvegicus clone A-2 |
| | | | | | ACETYLTRANSFERASE 1 | arylamine N acetyltransferase mRNA, |
| | | | | | complete cds | |
| 16364 | A, H | 235 | AA892251 | | | R. norvegicus mRNA for V1a arginine |
| | | | | | | vasopressin receptor |
| 16366 | P | 250 | AA892888 | | | EST |
| 16367 | P | 250 | AA892888 | | | EST |
| 16408 | F | 145 | AA852027 | | | ESTs |
| 16409 | S | 145 | AA852027 | | | ESTs |
| 16438 | I | 958 | AI176294 | | | ESTs, Highly similar to |
| | | | | | | SMD2_HUMAN SMALL NUCLEAR |
| | | | | | | RIBONUCLEOPROTEIN SM D2 |
| | | | | | | [H. sapiens] |
| 16446 | A | 214 | AA891423 | | | ESTs |
| 16449 | H | 1669 | NM_019238 | Sterol | farnesyl diphosphate | farnesyl diphosphate farnesyl |
| | | | | biosynthesis | farnesyl | transferase 1 |
| | | | | | transferase 1 | |
| 16458 | B, Q | 362 | AA944956 | | | ESTs |
| 16477 | Q | 983 | AI176701 | | | Rat low molecular weight fatty acid |
| | | | | | | binding protein mRNA, complete cds |
| 16513 | C | 118 | AA848782 | | | ESTs, Moderately similar to |
| | | | | | | hypothetical protein [M. musculus] |
| 16518 | D | 973 | AI176546 | | | ESTs, Weakly similar to HS9B_RAT |
| | | | | | | HEAT SHOCK PROTEIN HSP |
| | | | | | | 90-BETA [R. norvegicus] |
| 16519 | P | 1539 | NM_012532 | Porphyrin and | Ceruloplasmin | Ceruloplasmin (ferroxidase) |
| | | | | chlorophyll | (ferroxidase) | |
| | | | | metabolism | | |
| 16524 | H | 1362 | H33219 | | | ESTs |
| 16562 | E, N | 904 | AI171630 | | | Rattus norvegicus p38 mitogen |
| | | | | | | activated protein kinase mRNA, |
| | | | | | | complete cds |
| 16566 | H | 1131 | AI230395 | | | Rattus norvegicus mRNA for TIP120, |
| | | | | | | complete cds |
| 16610 | I | 1333 | D28557 | | | Rattus norvegicus muscle Y-box protein |
| | | | | | | YB2 mRNA, complete cds |
| 16616 | R | 1230 | AI234079 | | | ESTs |
| 16618 | C | 837 | AI168967 | | | ESTs |
| 16623 | E | 1150 | AI231196 | | | ESTs |
| 16649 | I | 1606 | NM_013132 | | Annexin V | Annexin V |
| 16650 | I | 1606 | NM_013132 | | Annexin V | Annexin V |
| 16654 | I | 1522 | X98517 | | | R. norvegicus mRNA for macrophage |
| | | | | | | metalloelastase (MME) |
| 16673 | R | 759 | AI104608 | | | ESTs |
| 16680 | A | 436 | AA965190 | | | ESTs |
| 16683 | I | 1596 | NM_013052 | | Tyrosine | Tyrosine 3-monooxygenase/tryptophan |
| | | | | | 3-monooxygenase/tryptophan | 5-monooxygenase activation protein, eta |
| | | | | | 5-monooxygenase | polypeptide |
| | | | | | activation protein, | |
| | | | | | eta polypeptide | |
| 16684 | I, O | 1596 | NM_013052 | | Tyrosine | Tyrosine 3-monooxygenase/tryptophan |
| | | | | | 3-monooxygenase/tryptophan | 5-monooxygenase activation protein, eta |
| | | | | | 5-monooxygenase | polypeptide |
| | | | | | activation protein, | |
| | | | | | eta polypeptide | |
| 16688 | L | 870 | AI170327 | | | ESTs |
| 16700 | A, E, S | 517 | AI008838 | | | ESTs, Weakly similar to |
| | | | | | | LONN_HUMAN |
| | | | | | | MITOCHONDRIAL LON PROTEASE |
| | | | | | | HOMOLOG PRECURSOR |
| | | | | | | [H. sapiens] |
| 16701 | A | 517 | AI008838 | | | ESTs, Weakly similar to |
| | | | | | | LONN_HUMAN |
| | | | | | | MITOCHONDRIAL LON PROTEASE |
| | | | | | | HOMOLOG PRECURSOR |
| | | | | | | [H. sapiens] |
| 16703 | A, C, O | 1060 | AI179300 | | | ESTs, Weakly similar to |
| | | | | | | LONN_HUMAN |
| | | | | | | MITOCHONDRIAL LON PROTEASE |
| | | | | | | HOMOLOG PRECURSOR |
| | | | | | | [H. sapiens] |
| 16704 | S | 4 | AA686132 | | | ESTs, Weakly similar to |
| | | | | | | LONN_HUMAN |
| | | | | | | MITOCHONDRIAL LON PROTEASE |
| | | | | | | HOMOLOG PRECURSOR |
| | | | | | | [H. sapiens] |
| 16726 | A | 1427 | M86235 | Fructose and | Hsp:KETOHEXOKINASE | Rat ketohexokinase mRNA, complete |
| | | | | mannose | | cds |
| | | | | metabolism | | |
| 16728 | H | 1020 | AI177885 | | | ESTs |
| 16730 | A, I | 23 | AA799766 | | | ESTs, Moderately similar to |
| | | | | | | JTV1_HUMAN JTV-1 PROTEIN |
| | | | | | | [H. sapiens] |
| 16747 | L | 336 | AA943131 | | | ESTs |
| 16756 | C, D | 52 | AA818089 | | | ESTs, Highly similar to glycyl-tRNA |
| | | | | | | synthetase [H. sapiens] |
| 16765 | A | 632 | AI058319 | | | ESTs |
| 16766 | A | 682 | AI072137 | | | ESTs |
| 16768 | N | 1331 | D16478 | Butanoate | HHs:hydroxyacyl-Coenzyme A | Rat mRNA for mitochondrial |
| | | | | metabolism, | dehydrogenase/ | long-chain enoyl-CoA hydratase/ |
| | | | | Fatty | 3-ketoacyl-Coenzyme | 3-hydroxyacyl-CoA dehydrogenase |
| | | | | acid | A thiolase/enoyl-Coenzyme A | alpha-subunit of mitochondrial |
| | | | | biosynthesis | hydratase (trifunctional | trifunctional protein, complete cds |
| | | | | (path 2), | protein), alpha | |
| | | | | Fatty acid | subunit | |
| | | | | metabolism, | | |
| | | | | Lysine | | |
| | | | | degradation, | | |
| | | | | Propanoate | | |
| | | | | metabolism, | | |
| | | | | Tryptophan | | |
| | | | | metabolism, | | |
| | | | | Valine, | | |
| | | | | leucine and | | |
| | | | | isoleucine | | |
| | | | | degradation, beta- | | |
| | | | | Alanine | | |
| | | | | metabolism | | |
| 16780 | E, K | 1510 | X62660 | | | ESTs, Highly similar to glutathione |
| | | | | | | transferase [R. norvegicus] |
| 16783 | L, O | 553 | AI012215 | | | ESTs, Weakly similar to nonmuscle |
| | | | | | | myosin heavy chain-A [R. norvegicus] |
| 16809 | B, O, Q | 1503 | X58828 | | Hsp:PROTEIN-TYROSINE | Rat PTP-S mRNA for protein-tyrosine |
| | | | | | PHOSPHATASE, | phosphatase |
| | | | | | NON-RECEPTOR | |
| | | | | | TYPE 2 | |
| 16825 | J | 245 | AA892602 | | | ESTs |
| 16854 | I | 188 | AA866454 | | | Rat alpha-2(I) promoter |
| 16859 | A, C, N | 1283 | AI236753 | | | ESTs |
| 16871 | H | 1583 | NM_012887 | | Thymopoietin | Thymopoietin (lamina associated |
| | | | | | (lamina associated | polypeptide 2) |
| | | | | | polypeptide 2) | |
| 16879 | A, E, F | 848 | AI169284 | | | ESTs |
| 16883 | A, C, D, I | 446 | AA997345 | | | ESTs, Weakly similar to nitrilase |
| | | | | | | homolog 1 [M. musculus] |
| 16884 | B, E | 754 | AI103758 | Arginine and | HHs:aldehyde | Rattus norvegicus 4- |
| | | | | proline | dehydrogenase 9 | trimethylaminobutyraldehyde |
| | | | | metabolism, | (gamma-aminobutyraldehyde | dehydrogenase (Tmabadh) mRNA, |
| | | | | Ascorbate and | dehydrogenase, | complete cds |
| | | | | aldarate | E3 isozyme) | |
| | | | | metabolism, Bile | | |
| | | | | acid biosynthesis, | | |
| | | | | Butanoate | | |
| | | | | metabolism, | | |
| | | | | Fatty acid | | |
| | | | | metabolism, | | |
| | | | | Glycerolipid | | |
| | | | | metabolism, | | |
| | | | | Histidine | | |
| | | | | metabolism, | | |
| | | | | Lysine | | |
| | | | | degradation, | | |
| | | | | Propanoate | | |
| | | | | metabolism, | | |
| | | | | Pyruvate | | |
| | | | | metabolism, | | |
| | | | | Tryptophan | | |
| | | | | metabolism | | |
| 16885 | A, B, E, Q | 773 | AI105188 | Arginine and | HHs:aldehyde | Rattus norvegicus 4- |
| | | | | proline | dehydrogenase 9 | trimethylaminobutyraldehyde |
| | | | | metabolism, | (gamma-aminobutyraldehyde | dehydrogenase (Tmabadh) mRNA, |
| | | | | Ascorbate and | dehydrogenase, | complete cds |
| | | | | aldarate | E3 isozyme) | |
| | | | | metabolism, Bile | | |
| | | | | acid biosynthesis, | | |
| | | | | Butanoate | | |
| | | | | metabolism, | | |
| | | | | Fatty acid | | |
| | | | | metabolism, | | |
| | | | | Glycerolipid | | |
| | | | | metabolism, | | |
| | | | | Histidine | | |
| | | | | metabolism, | | |
| | | | | Lysine | | |
| | | | | degradation, | | |
| | | | | Propanoate | | |
| | | | | metabolism, | | |
| | | | | Pyruvate | | |
| | | | | metabolism, | | |
| | | | | Tryptophan | | |
| | | | | metabolism | | |
| 16894 | O | 144 | AA852018 | | | ESTs, Moderately similar to |
| | | | | | | AF097362_1 gamma- |
| | | | | | | interferon inducible lysosomal thiol |
| | | | | | | reductase [H. sapiens] |
| 16944 | S | 320 | AA925541 | | | ESTs, Highly similar to protein L |
| | | | | | | [M. musculus] |
| 16945 | S | 320 | AA925541 | | | ESTs, Highly similar to protein L |
| | | | | | | [M. musculus] |
| 16947 | E | 1572 | NM_012793 | Arginine and | Guanidinoacetate | Guanidinoacetate methyltransferase |
| | | | | proline | methyltransferase | |
| | | | | metabolism, | | |
| | | | | Glycine, serine | | |
| | | | | and threonine | | |
| | | | | metabolism, | | |
| | | | | Urea cycle and | | |
| | | | | metabolism of | | |
| | | | | amino groups | | |
| 16958 | G | 92 | AA819021 | | | EST |
| 16961 | P | 1058 | AI179236 | | | ESTs |
| 16982 | A | 1608 | NM_013144 | | Insulin-like growth | Insulin-like growth factor binding |
| | | | | | factor binding protein | protein 1 |
| | | | | | 1 | |
| 16993 | A | 14 | AA799560 | | | ESTs |
| 17027 | A, E | 877 | AI170679 | Galactose | HHs:UDP-glucose | ESTs, Highly similar to |
| | | | | | | UDP1_HUMAN |
| | | | | metabolism, | pyrophosphorylase | UTP-GLUCOSE-1-PHOSPHATE |
| | | | | Nucleotide | 2 | URIDYLYLTRANSFERASE 1 |
| | | | | sugars | | [H. sapiens] |
| | | | | metabolism, | | |
| | | | | Pentose and | | |
| | | | | glucuronate | | |
| | | | | interconversions, | | |
| | | | | Starch and | | |
| | | | | sucrose | | |
| | | | | metabolism | | |
| 17049 | A | 929 | AI172417 | | | ESTs, Weakly similar to Similarity to |
| | | | | | | B. subtilis YQJC protein [C. elegans] |
| 17064 | I | 1660 | NM_019170 | Prostaglandin and | carbonyl reductase | carbonyl reductase |
| | | | | leukotriene | | |
| | | | | metabolism | | |
| 17090 | G, K | 1474 | U73174 | Glutamate | HHs:glutathione reductase | Rattus norvegicus glutathione reductase |
| | | | | metabolism, | | mRNA, complete cds |
| | | | | Glutathione | | |
| | | | | metabolism | | |
| 17091 | G, K | 1474 | U73174 | Glutamate | HHs:glutathione reductase | Rattus norvegicus glutathione reductase |
| | | | | metabolism, | | mRNA, complete cds |
| | | | | Glutathione | | |
| | | | | metabolism | | |
| 17092 | K | 259 | AA893189 | Glutamate | HHs:glutathione reductase | Rattus norvegicus glutathione reductase |
| | | | | metabolism, | | mRNA, complete cds |
| | | | | Glutathione | | |
| | | | | metabolism | | |
| 17107 | E | 1638 | NM_017160 | | ribosomal protein S6 | ribosomal protein S6 |
| 17117 | K | 1085 | AI228042 | | | ESTs, Weakly similar to AC007080_2 |
| | | | | | | NG38 [M. musculus] |
| 17154 | A | 1407 | M15883 | | | Rat clathrin light chain (LCB2) mRNA, |
| | | | | | | complete cds, Rat clathrin light chain |
| | | | | | | (LCB3) mRNA, complete cds |
| 17157 | I | 326 | AA926129 | | | ESTs, Highly similar to AF168795_1 |
| | | | | | | schlafen-4 [R. norvegicus] |
| 17158 | H | 1699 | NM_022298 | | | Rat mRNA encoding alpha-tubulin |
| 17167 | M | 566 | AI013690 | | | ESTs |
| 17175 | A | 1501 | X58389 | | | R. norvegicus ASI mRNA for |
| | | | | | | mammalian equivalent of bacterial large |
| | | | | | | ribosomal subunit protein L22 |
| 17225 | A, I | 215 | AA891553 | | | ESTs, Highly similar to eIF3 p66 |
| | | | | | | [M. musculus] |
| 17256 | A | 219 | AA891739 | | | ESTs, Weakly similar to p60 protein |
| | | | | | | [R. norvegicus] |
| 17257 | E, R | 1568 | NM_012766 | | Cyclin D3 | Cyclin D3 |
| 17258 | P | 1568 | NM_012766 | | Cyclin D3 | Cyclin D3 |
| 17261 | R | 1568 | NM_012766 | | Cyclin D3 | Cyclin D3 |
| 17277 | B, P, Q | 523 | AI009338 | | | Rattus norvegicus glycine-, glutamate-, |
| | | | | | | thienylcyclohexylpiperidine-binding |
| | | | | | | protein mRNA, complete cds |
| 17281 | M, P | 1450 | U10697 | | Hsp:LIVER | R. norvegicus mRNA for |
| | | | | | CARBOXYLESTERASE 4 | pl esterase (ES-4) |
| | | | | | PRECURSOR | |
| 17291 | E | 931 | AI172491 | Citrate cycle | HHs:isocitrate | ESTs, Weakly similar to IDHC_RAT |
| | | | | (TCA cycle), | dehydrogenase 2 | ISOCITRATE DEHYDROGENASE |
| | | | | Glutathione | (NADP+), mitochondrial | [R. norvegicus] |
| | | | | metabolism | | |
| 17324 | A | 1686 | NM_021593 | | | Rattus norvegicus kynurenine 3- |
| | | | | | | hydroxylase mRNA, complete cds |
| 17334 | A | 151 | AA858704 | | | ESTs, Highly similar to responsible for |
| | | | | | | hereditary multiple exotosis |
| | | | | | | [M. musculus] |
| 17335 | A | 732 | AI102634 | | | ESTs, Weakly similar to W06B4.2 |
| | | | | | | [C. elegans] |
| 17337 | J | 472 | AB000717 | Methionine | HHs:methionine | ESTs |
| | | | | metabolism, | adenosyltransferase II, | |
| | | | | Selenoamino | alpha | |
| | | | | acid | | |
| | | | | metabolism | | |
| 17339 | A | 123 | AA849497 | | | ESTs |
| 17340 | A, E | 507 | AI007803 | | | Rattus norvegicus ERM-binding |
| | | | | | | phosphoprotein mRNA, complete cds |
| 17368 | E, R | 284 | AA900548 | | | ESTs |
| 17369 | C, I, P | 812 | AI137572 | | | ESTs |
| 17377 | A | 1491 | X13058 | | Tumor protein | Rat mRNA for nuclear oncoprotein p53 |
| | | | | | p53 (Li-Fraumeni | |
| | | | | | syndrome) | |
| 17393 | A, O | 1377 | J04943 | | Nucleoplasmin-related | Nucleoplasmin-related protein (Nuclear |
| | | | | | protein (Nuclear | protein B23 |
| | | | | | protein B23 | |
| 17400 | E | 744 | AI103097 | | | ESTs, Highly similar to |
| | | | | | | ATPK_MOUSE |
| | | | | | | ATP SYNTHASE F CHAIN, |
| | | | | | | MITOCHONDRIAL [M. musculus] |
| 17401 | A | 1595 | NM_013043 | | Transforming growth | Transforming growth factor beta |
| | | | | | factor beta | stimulated clone 22 |
| | | | | | stimulated clone 22 | |
| 17451 | E | 806 | AI137356 | | | ESTs, Highly similar to |
| | | | | | | DHYS_HUMAN |
| | | | | | | DEOXYHYPUSINE SYNTHASE |
| | | | | | | [H. sapiens] |
| 17479 | R | 827 | AI145385 | | | ESTs |
| 17481 | E | 1529 | Z49761 | | | R. norvegicus mRNA for RT1.Ma |
| 17496 | A | 325 | AA926109 | | | ESTs |
| 17500 | I, P | 1713 | NM_022866 | | | Rattus norvegicus sodium-dependent |
| | | | | | | high-affinity dicarboxylate transporter |
| | | | | | | (NADC3) mRNA, complete cds |
| 17506 | L | 649 | AI070068 | | | ESTs |
| 17516 | O | 1739 | NM_017321 | | iron-responsive | iron-responsive element-binding protein |
| | | | | | element-binding protein | |
| 17524 | A | 539 | AI010568 | | | ESTs |
| 17541 | G, K | 1580 | NM_012844 | | Epoxide hydrolase 1 | Epoxide hydrolase 1 (microsomal |
| | | | | | (microsomal | xenobiotic hydrolase) |
| | | | | | xenobiotic hydrolase) | |
| 17571 | H, I | 1276 | AI236484 | | | Rattus norvegicus mRNA for hnRNP |
| | | | | | | protein, partial |
| 17572 | E | 71 | AA818524 | | | Rattus norvegicus mRNA for hnRNP |
| | | | | | | protein, partial |
| 17589 | A | 248 | AA892851 | | | ESTs |
| 17590 | F | 248 | AA892851 | | | ESTs |
| 17591 | A | 898 | AI171354 | | | ESTs |
| 17613 | O | 10 | AA799511 | | | ESTs |
| 17617 | E | 1269 | AI236301 | | | ESTs, Weakly similar to FKB1_RAT |
| | | | | | | FK506-BINDING PROTEIN |
| | | | | | | [R. norvegicus] |
| 17644 | R | 293 | AA924036 | | | ESTs |
| 17664 | B, Q | 1238 | AI234496 | | | ESTs |
| 17672 | N | 1123 | AI230074 | Oxidative | HMm:NADH ubiquinone | ESTs, Highly similar to |
| | | | | | | NIMM_MOUSE |
| | | | | phosphorylation, | oxidoreductase subunit | NADH-UBIQUINONE |
| | | | | Ubiquinone | MWFE | OXIDOREDUCTASE MWFE |
| | | | | biosynthesis | | SUBUNIT [M. musculus] |
| 17677 | E | 683 | AI072246 | | | ESTs |
| 17683 | N | 700 | AI073257 | | | ESTs |
| 17684 | G | 236 | AA892345 | | | Rat mRNA for dimethylglycine |
| | | | | | | dehydrogenase (EC number 1.5.99.2) |
| 17685 | K | 797 | AI113055 | | | EST |
| 17687 | C | 12 | AA799531 | | | ESTs, Weakly similar to predicted using |
| | | | | | | Genefinder [C. elegans] |
| 17688 | A | 12 | AA799531 | | | ESTs, Weakly similar to predicted using |
| | | | | | | Genefinder [C. elegans] |
| 17695 | N | 1192 | AI232784 | | | ESTs, Weakly similar to putative |
| | | | | | | peroxisomal 2,4-dienoyl-CoA reductase |
| | | | | | | [R. norvegicus] |
| 17699 | O | 135 | AA851233 | | | ESTs, Weakly similar to NG28 |
| | | | | | | [M. musculus] |
| 17709 | A | 1456 | U24489 | | Tenascin X | Tenascin X |
| 17730 | G | 1709 | NM_022697 | | | Rat mRNA for ribosomal protein L28 |
| 17734 | C, D | 466 | AA998683 | | | ESTs, Rattus norvegicus heat shock |
| | | | | | | protein 27 (hsp 27) gene, complete cds |
| 17735 | C, D, J | 981 | AI176658 | | | ESTs, Rattus norvegicus heat shock |
| | | | | | | protein 27 (hsp 27) gene, complete cds |
| 17736 | C, D | 1428 | M86389 | | | ESTs, Rattus norvegicus heat shock |
| | | | | | | protein 27 (hsp 27) gene, complete cds |
| 17747 | E | 1236 | AI234223 | | | ESTs, Highly similar to cellular |
| | | | | | | apoptosis susceptibility protein |
| | | | | | | [H. sapiens] |
| 17753 | J | 748 | AI103246 | | | ESTs, Highly similar to S65568 |
| | | | | | | CCAAT-binding factor CBF2-mouse |
| | | | | | | [M. musculus] |
| 17754 | I | 261 | AA893246 | | | ESTs, Highly similar to vacuolar H- |
| | | | | | | ATPase subunit D [H. sapiens] |
| 17758 | G | 1645 | NM_017220 | Butanoate | HHs:enoyl-Coenzyme A, | Rat peroxisomal enoyl-CoA: |
| | | | | metabolism, | hydratase/3- | hydrotase-3 hydroxyacyl-CoA |
| | | | | Fatty | hydroxyacyl Coenzyme A | bifunctional enzyme mRNA, |
| | | | | acid | dehydrogenase | complete cds |
| | | | | biosynthesis | | |
| | | | | (path 2), | | |
| | | | | Fatty acid | | |
| | | | | metabolism, | | |
| | | | | Lysine | | |
| | | | | degradation, | | |
| | | | | Propanoate | | |
| | | | | metabolism, | | |
| | | | | Tryptophan | | |
| | | | | metabolism, | | |
| | | | | Valine, | | |
| | | | | leucine and | | |
| | | | | isoleucine | | |
| | | | | degradation, beta- | | |
| | | | | Alanine | | |
| | | | | metabolism | | |
| 17768 | B | 774 | AI105196 | | | ESTs |
| 17785 | N | 1534 | NM_012501 | | Apolipoprotein C-III | Apolipoprotein C-III |
| 17788 | K | 271 | AA899045 | | Esterase D/formylglutathione | ESTs, Highly similar to sid478p |
| | | | | | hydrolase | [M. musculus] |
| 17794 | E, N | 772 | AI105184 | Cyanoamino | HHs:serine | ESTS |
| | | | | acid | hydroxymethyltransferase 1 | |
| | | | | metabolism, | (soluble) | |
| | | | | Glycine, | | |
| | | | | serine | | |
| | | | | and threonine | | |
| | | | | metabolism, | | |
| | | | | Lysine | | |
| | | | | degradation, | | |
| | | | | Methane | | |
| | | | | metabolism, | | |
| | | | | One carbon pool | | |
| | | | | by folate | | |
| 17800 | N | 262 | AA893436 | | | ESTs |
| 17809 | B | 5 | AA686461 | | | Rat ribosomal protein L30 mRNA, |
| | | | | | | complete cds |
| 17812 | A, E | 841 | AI169075 | Glutathione | HMm:glutathione | ESTs |
| | | | | metabolism, | transferase zeta 1 | |
| | | | | Tyrosine | (maleylacetoacetate isomerase) | |
| | | | | metabolism | | |
| 17819 | A | 891 | AI171095 | | | ESTs, Highly similar to unknown |
| | | | | | | [H. sapiens] |
| 17844 | A, E | 398 | AA955927 | | | ESTs |
| 17847 | A | 1025 | AI178214 | | | ESTs |
| 17850 | A | 734 | AI102750 | | | ESTs, Weakly similar to |
| | | | | | | TCPA_RAT T- |
| | | | | | | COMPLEX PROTEIN 1, ALPHA |
| | | | | | | SUBUNIT [R. norvegicus] |
| 17854 | Q | 1490 | X13016 | | | Rat mRNA for MRC OX-45 surface |
| | | | | | | antigen |
| 17894 | E, F | 1594 | NM_013027 | | Selenoprotein W muscle 1 | Selenoprotein W muscle 1 |
| 17908 | A, J | 1670 | NM_019242 | | interferon-related | interferon-related developmental |
| | | | | | developmental | regulator 1 |
| | | | | | regulator 1 | |
| 17935 | S | 289 | AA901006 | | | Rattus norvegicus membrane interacting |
| | | | | | | protein of RGS16 (Mir16) mRNA, |
| | | | | | | complete cds |
| 17950 | Q | 1278 | AI236590 | | myeloid differentiation | ESTs |
| | | | | | primary | |
| | | | | | response gene 88 | |
| 17955 | L | 590 | AI030069 | | | ESTs |
| 17956 | I | 427 | AA964379 | | adaptor-related protein | adaptor-related protein complex AP-1, |
| | | | | | complex AP-1, | beta 1 subunit |
| | | | | | beta 1 subunit | |
| 17982 | A | 1727 | NM_017010 | | Glutamate receptor, | Glutamate receptor, ionotropic, |
| | | | | | ionotropic, N- | N-methyl D-aspartate 1, Rat |
| | | | | | N-methyl-D-aspartate | |
| | | | | | methyl D-aspartate 1 | receptor (NMDAR1) gene, first exon |
| 18001 | A | 149 | AA858573 | | | ESTs, Highly similar to SP24_RAT |
| | | | | | | SECRETED PHOSPHOPROTEIN 24 |
| | | | | | | [R. norvegicus], |
| | | | | | | Rattus norvegicus spp-24 |
| | | | | | | precursor mRNA, partial cds |
| 18002 | A, D, E | 600 | AI043655 | | | ESTs, Highly similar to SP24_RAT |
| | | | | | | SECRETED PHOSPHOPROTEIN 24 |
| | | | | | | [R. norvegicus], |
| | | | | | | Rattus norvegicus spp-24 |
| | | | | | | precursor mRNA, partial cds |
| 18028 | G | 1337 | D38062 | | | Rattus norvegicus UDP- |
| | | | | | | glucuronosyltransferase UGT1A7 |
| | | | | | | mRNA, complete cds |
| 18029 | S | 1418 | M38759 | | Sex hormone binding | Sex hormone binding globulin or |
| | | | | | globulin or | androgen-binding protein |
| | | | | | androgen-binding | |
| | | | | | protein | |
| 18043 | J | 487 | AF020618 | | | Rattus norvegicus progression elevated |
| | | | | | | gene 3 protein mRNA, complete cds |
| 18046 | I | 500 | AF072892 | | | Rattus norvegicus versican V0 isoform |
| | | | | | | mRNA, partial cds, Rattus norvegicus |
| | | | | | | versican V3 isoform precursor, mRNA, |
| | | | | | | complete cds |
| 18082 | S | 478 | AB010429 | | | R. norvegicus mRNA for mitochondrial |
| | | | | | | very-long-chain acyl-CoA thioesterase |
| 18083 | S | 1524 | Y09333 | | Hsp:ACYL COENZYME | R. norvegicus mRNA for mitochondrial |
| | | | | | A THIOESTER | very-long-chain acyl-CoA thioesterase |
| | | | | | HYDROLASE, | |
| | | | | | MITOCHONDRIAL | |
| | | | | | PRECURSOR | |
| 18099 | G | 1604 | NM_013119 | | | ESTs, Highly similar to A60054 sodium |
| | | | | | | channel protein IIIb, long form-rat |
| | | | | | | [R. norvegicus] |
| 18107 | I | 1717 | NM_022949 | | | R. norvegicus mRNA for ribosomal |
| | | | | | | protein L14 |
| 18109 | A | 1577 | NM_012823 | | Annexin III | ESTs, Weakly similar to LURT3 |
| | | | | | (Lipocortin III) | annexin III - rat [R. norvegicus] |
| 18115 | A | 31 | AA800339 | | | ESTs |
| 18125 | S | 515 | AI008787 | | | ESTs |
| 18136 | H | 737 | AI102820 | | | ESTs |
| 18141 | O | 1014 | AI177413 | | ATP synthase | ATP synthase subunit d, ESTs, Weakly |
| | | | | | subunit d | similar to myo-inositol-1-phosphate |
| | | | | | | synthase [D. melanogaster] |
| 18203 | P | 1584 | NM_012891 | | | ESTs, Highly similar to ACDV_RAT |
| | | | | | | ACYL-COA DEHYDROGENASE, |
| | | | | | | VERY-LONG-CHAIN SPECIFIC, |
| | | | | | | MITOCHONDRIAL PRECURSOR |
| | | | | | | [R. norvegicus] |
| 18235 | L | 758 | AI104523 | | | ESTs |
| 18237 | Q | 1065 | AI179539 | | | ESTs, Highly similar to CDC45L |
| | | | | | | [M. musculus] |
| 18259 | J | 1280 | AI236601 | | | ESTs |
| 18272 | B | 6 | AA799294 | | | ESTs, Moderately similar to KIAA0740 |
| | | | | | | protein [H. sapiens] |
| 18280 | L | 384 | AA946361 | | | ESTs, Highly similar to Ring3 |
| | | | | | | [M. musculus] |
| 18285 | R | 341 | AA943791 | | | ESTs |
| 18316 | K | 499 | AF072411 | | | Rattus norvegicus FAT mRNA, |
| | | | | | | complete cds |
| 18318 | S | 385 | AA946368 | | | Rattus norvegicus FAT mRNA, |
| | | | | | | complete cds |
| 18323 | E | 556 | AI012498 | | | ESTs |
| 18349 | J | 22 | AA799744 | | | ESTs |
| 18369 | G | 19 | AA799645 | | | Rattus norvegicus phospholemman |
| | | | | | | chloride channel mRNA, complete cds |
| 18389 | A, B, Q | 9 | AA799498 | | Brain natriuretic | Rattus norvegicus brain natriuretic |
| | | | | | factor | peptide (BNP) mRNA, complete cds |
| 18390 | A, E | 128 | AA850038 | | | ESTs |
| 18418 | C | 969 | AI176483 | | | ESTs |
| 18452 | A | 1630 | NM_017074 | Cysteine | CTL target | CTL target antigen |
| | | | | metabolism, | antigen | |
| | | | | Methionine | | |
| | | | | metabolism, | | |
| | | | | Nitrogen | | |
| | | | | metabolism, | | |
| | | | | Selenoamino acid | | |
| | | | | metabolism | | |
| 18453 | A | 1630 | NM_017074 | Cysteine | CTL target | CTL target antigen |
| | | | | metabolism, | antigen | |
| | | | | Methionine | | |
| | | | | metabolism, | | |
| | | | | Nitrogen | | |
| | | | | metabolism, | | |
| | | | | Selenoamino acid | | |
| | | | | metabolism | | |
| 18465 | B, Q | 1077 | AI180187 | | | ESTs |
| 18473 | K | 838 | AI168975 | | | ESTs |
| 18482 | H | 1311 | AI639151 | | | ESTs, Highly similar to pinin |
| | | | | | | [H. sapiens] |
| 18484 | L | 1249 | AI235349 | | | ESTs, Highly similar to KIAA0184 |
| | | | | | | [H. sapiens] |
| 18495 | B | 1307 | AI639042 | | | ESTs |
| 18501 | J | 1414 | M31178 | | | Rat calbindin D28 mRNA, complete cds |
| 18522 | A, E | 830 | AI145870 | | | ESTs |
| 18529 | B, Q | 1136 | AI230716 | | | ESTs |
| 18580 | M, P | 142 | AA851963 | | | ESTs |
| 18584 | H | 216 | AA891694 | | | ESTs |
| 18588 | E | 276 | AA899635 | | | ESTs, Moderately similar to 2020285A |
| | | | | | | BRG1 protein [M. musculus] |
| 18597 | A | 481 | AB013732 | Nucleotide | HMm:UDP-glucose | Rattus norvegicus mRNA for UDP- |
| | | | | sugars | dehydrogenase | glucose dehydrogeanse, complete cds |
| | | | | metabolism, | | |
| | | | | Pentose and | | |
| | | | | glucuronate | | |
| | | | | interconversions, | | |
| | | | | Starch and | | |
| | | | | sucrose | | |
| | | | | metabolism | | |
| 18604 | N | 1292 | AI237124 | | | ESTs |
| 18606 | A | 1497 | X53504 | | | ESTs, Highly similar to |
| | | | | | | RL12_RAT 60S |
| | | | | | | RIBOSOMAL PROTEIN L12 |
| | | | | | | [R. norvegicus] |
| 18612 | E, O | 1092 | AI228624 | | | ESTs, Highly similar to |
| | | | | | | RL23_HUMAN |
| | | | | | | 60S RIBOSOMAL PROTEIN L23 |
| | | | | | | [R. norvegicus] |
| 18647 | E | 1435 | S69316 | | | ESTs, Weakly similar to HS9B_RAT |
| | | | | | | HEAT SHOCK PROTEIN HSP |
| | | | | | | 90-BETA [R. norvegicus] |
| 18660 | A | 894 | AI171262 | | cyclin G2 | ESTs |
| 18661 | A | 376 | AA945751 | | | ESTs |
| 18685 | L | 453 | AA997746 | Fatty acid | dodecenoyl-Coenzyme | dodecenoyl-Coenzyme A delta |
| | | | | metabolism | A delta | isomerase (3,2 trans-enoyl-Coenyme A |
| | | | | | isomerase (3,2 | isomerase) |
| | | | | | trans-enoyl-Coenyme A | |
| | | | | | isomerase) | |
| 18705 | I | 1732 | NM_020103 | | Ly6-C antigen gene | Ly6-C antigen gene |
| 18727 | S | 1685 | NM_021577 | Alanine and | HHs:argininosuccinate | Rat mRNA for argininosuccinate lyase, |
| | | | | aspartate | lyase | complete cds |
| | | | | metabolism, | | |
| | | | | Arginine and | | |
| | | | | proline | | |
| | | | | metabolism, | | |
| | | | | Urea | | |
| | | | | cycle and | | |
| | | | | metabolism of | | |
| | | | | amino groups | | |
| 18742 | O, S | 769 | AI105131 | | | ESTs, Highly similar to AF189764_1 |
| | | | | | | alpha/beta hydrolase-1 [M. musculus] |
| 18746 | S | 900 | AI171506 | Pyruvate | Malic enzyme 1, | Malic enzyme 1, soluble |
| | | | | metabolism | soluble | |
| 18747 | S | 1550 | NM_012600 | Pyruvate | Malic enzyme 1, | Malic enzyme 1, soluble |
| | | | | metabolism | soluble | |
| 18749 | S | 1550 | NM_012600 | Pyruvate | Malic enzyme 1, | Malic enzyme 1, soluble |
| | | | | metabolism | soluble | |
| 18755 | C, D | 1279 | AI236599 | | | ESTs |
| 18783 | N | 1282 | AI236746 | | | ESTs |
| 18792 | A | 662 | AI071177 | | | ESTs |
| 18795 | N | 1483 | U95001 | | | ESTs |
| 18796 | A | 45 | AA817761 | | | ESTs |
| 18829 | H | 84 | AA818796 | | | ESTs |
| 18837 | G | 901 | AI171583 | | | ESTs, Moderately similar to |
| | | | | | | PLTP_MOUSE PHOSPHOLIPID |
| | | | | | | TRANSFER PROTEIN PRECURSOR |
| | | | | | | [M. musculus] |
| 18854 | A | 1300 | AI237636 | | | ESTs, Weakly similar to N-copine |
| | | | | | | [M. musculus] |
| 18860 | A, K | 861 | AI169695 | | | Rattus norvegicus mRNA for |
| | | | | | | hydroxysteroid sulfotransferase subunit, |
| | | | | | | complete cds |
| 18861 | A | 1329 | D14989 | Androgen and | Hsp:ALCOHOL | Rattus norvegicus mRNA for |
| | | | | estrogen | SULFOTRANSFERASE | hydroxysteroid sulfotransferase subunit, |
| | | | | metabolism, | | complete cds |
| | | | | Sulfur | | |
| | | | | metabolism | | |
| 18867 | A | 1348 | D88250 | | | Rattus norvegicus mRNA for serine |
| | | | | | | protease, complete cds |
| 18877 | O | 686 | AI072393 | | | ESTs |
| 18885 | R | 583 | AI029827 | | | ESTs, Highly similar to AF157028_1 |
| | | | | | | protein phosphatase methylesterase-1 |
| | | | | | | [H. sapiens] |
| 18886 | R | 340 | AA943785 | | | ESTs, ESTs, Highly similar to |
| | | | | | | AF157028_1 protein phosphatase |
| | | | | | | methylesterase-1 [H. sapiens] |
| 18890 | B, P, S | 280 | AA899964 | | | ESTs |
| 18891 | B, Q, S | 303 | AA924598 | | | ESTs |
| 18900 | F | 1214 | AI233570 | | | ESTs, Highly similar to |
| | | | | | | PSD8_HUMAN |
| | | | | | | 26S PROTEASOME REGULATORY |
| | | | | | | SUBUNIT S14 [H. sapiens] |
| 18905 | E | 883 | AI170770 | Oxidative | HHs:NADH | ESTs, Highly similar to NADH- |
| | | | | phosphorylation, | dehydrogenase | ubiquinone oxidoreductase NDUFS2 |
| | | | | Ubiquinone | (ubiquinone) Fe—S | subunit [H. sapiens] |
| | | | | biosynthesis | protein 2 (49kD) | |
| | | | | | (NADH-coenzyme Q reductase) | |
| 18906 | A, K | 243 | AA892561 | | | ESTs, Moderately similar to PTD012 |
| | | | | | | [H. sapiens] |
| 18908 | A | 122 | AA849426 | | | ESTs |
| 18909 | A | 122 | AA849426 | | | ESTs |
| 18910 | A | 1182 | AI232419 | | | ESTs |
| 18956 | S | 1631 | NM_017075 | Bile acid | Acetyl-Co A | Acetyl-Co A acetyltransferase 1, |
| | | | | biosynthesis, | acetyltransferase 1, | mitochondrial |
| | | | | Butanoate | mitochondrial | |
| | | | | metabolism, Fatty | | |
| | | | | acid | | |
| | | | | biosynthesis | | |
| | | | | (path 2), | | |
| | | | | Fatty acid | | |
| | | | | metabolism, | | |
| | | | | Lysine | | |
| | | | | degradation, | | |
| | | | | Propanoate | | |
| | | | | metabolism, | | |
| | | | | Pyruvate metabolism, | | |
| | | | | Synthesis and | | |
| | | | | degradation | | |
| | | | | of ketone bodies, | | |
| | | | | Tryptophan | | |
| | | | | metabolism | | |
| 18960 | A | 1004 | AI177103 | | | ESTs |
| 18962 | R | 574 | AI013918 | | | Rattus norvegicus TM6P1 (TM6P1) |
| | | | | | | mRNA, complete cds |
| 18974 | M | 319 | AA925384 | | | EST |
| 18981 | H | 11 | AA799523 | | | ESTs, Moderately similar to hnRNP |
| | | | | | | protein [R. norvegicus] |
| 18990 | G | 1438 | S72506 | Glutathione | Glutathione-S-transferase, | Glutathione-S-transferase, alpha type |
| | | | | metabolism | alpha type | (Yc?) |
| | | | | | (Yc?) | |
| 18996 | N | 1027 | AI178326 | | | ESTs |
| 19012 | J, K | 918 | AI172056 | | | ESTs |
| 19040 | I | 1374 | J03627 | | | Rat S-100 related protein mRNA, |
| | | | | | | complete cds, clone 42C |
| 19043 | F | 130 | AA850378 | | | ESTs, Highly similar to methyl-CpG |
| | | | | | | binding protein MBD2 [M. musculus] |
| 19044 | S | 386 | AA946379 | | | ESTs, Highly similar to methyl-CpG |
| | | | | | | binding protein MBD2 [M. musculus] |
| 19052 | E, R | 1253 | AI235675 | | | ESTs |
| 19053 | K | 1327 | D12770 | | | Rattus norvegicus mRNA for |
| | | | | | | mitochondrial adenine nucleotide |
| | | | | | | translocator |
| 19069 | A, L | 339 | AA943737 | | | ESTs |
| 19073 | F | 34 | AA800576 | | | ESTs |
| 19075 | B, J | 1275 | AI236473 | | | ESTs, Moderately similar to cysteine- |
| | | | | | | rich hydrophobic 1 [M. musculus] |
| 19085 | A, J | 244 | AA892598 | | | ESTs |
| 19086 | A, J | 244 | AA892598 | | | ESTs |
| 19103 | A | 36 | AA800797 | | | ESTs |
| 19105 | E | 162 | AA859230 | | | ESTs, Highly similar to |
| | | | | | | HG14_MOUSE |
| | | | | | | NONHISTONE CHROMOSOMAL |
| | | | | | | PROTEIN HMG-14 [M. musculus] |
| 19121 | P | 608 | AI044101 | | | ESTs |
| 19150 | C | 8 | AA799461 | | | ESTs |
| 19158 | B | 140 | AA851953 | | | ESTs, Moderately similar to |
| | | | | | | hypothetical protein [H. sapiens] |
| 19184 | J | 1022 | AI178025 | | | ESTs, Highly similar to TGIF_MOUSE |
| | | | | | | 5′-TG-3′ INTERACTING FACTOR |
| | | | | | | [M. musculus] |
| 19211 | N | 136 | AA851329 | | | ESTs |
| 19230 | R | 646 | AI059604 | | | ESTs |
| 19241 | I | 1666 | NM_019206 | | Serine/threonine | Serine/threonine kinase 10 |
| | | | | | kinase 10 | |
| 19252 | N | | NM_019382 | | anti-oxidant protein 2 | anti-oxidant protein 2 |
| 19255 | K | 1406 | M15562 | | | Rat (diabetic BB) MHC class II alpha |
| | | | | | | chain RT1.D alpha (u) |
| 19256 | K | 1406 | M15562 | | | Rat (diabetic BB) MHC class II alpha |
| | | | | | | chain RT1.D alpha (u) |
| 19258 | O | 287 | AA900613 | | | ESTs |
| 19261 | O | 741 | AI102943 | | | ESTs |
| 19264 | C, D, R | 743 | AI103078 | | | ESTs |
| 19292 | K | 445 | AA997323 | | | EST |
| 19298 | A, D, I | 1272 | AI236338 | | | ESTs, Weakly similar to NHPX_RAT |
| | | | | | | NHP2/RS6 FAMILY PROTEIN |
| | | | | | | YEL026W HOMOLOG [R. norvegicus] |
| 19315 | E | 1144 | AI231010 | | | EST |
| 19363 | A, F | 954 | AI176247 | | | ESTs, Moderately similar to unnamed |
| | | | | | | protein product [H. sapiens] |
| 19373 | N | 1684 | NM_021266 | | Hyaluronan mediated | Hyaluronan mediated motility receptor |
| | | | | | motility receptor | (RHAMM) |
| | | | | | (RHAMM) | |
| 19377 | I | 180 | AA859971 | | | ESTs, Moderately similar to RL3_RAT |
| | | | | | | 60S RIBOSOMAL PROTEIN L3 |
| | | | | | | [R. norvegicus] |
| 19388 | F | 206 | AA891032 | | | EST |
| 19392 | M | 1592 | NM_012998 | Arginine | Protein disulfide | Protein disulfide isomerase (Prolyl 4- |
| | | | | and | isomerase (Prolyl 4- | hydroxylase, beta polypeptide) |
| | | | | proline | hydroxylase, beta | |
| | | | | metabolism, | polypeptide) | |
| | | | | Biosynthesis | | |
| | | | | and | | |
| | | | | degradation of | | |
| | | | | glycoprotein | | |
| 19410 | B, Q | 268 | AA893667 | | | ESTs, Moderately similar to |
| | | | | | | AC006978_1 supported by human and |
| | | | | | | rodent ESTs [H. sapiens] |
| 19411 | M, P | 268 | AA893667 | | | ESTs, Moderately similar to |
| | | | | | | AC006978_1 supported by human and |
| | | | | | | rodent ESTs [H. sapiens] |
| 19412 | B, Q | 120 | AA849222 | | | ESTs, Moderately similar to |
| | | | | | | AC006978_1 supported by human and |
| | | | | | | rodent ESTs [H. sapiens] |
| 19444 | P | 309 | AA924993 | | | ESTs |
| 19458 | E | 462 | AA998345 | | | EST |
| 19465 | K | 630 | AI045881 | | | EST |
| 19469 | A, P | 231 | AA892112 | | | ESTs, Weakly similar to proline |
| | | | | | | dehydrogenase [M. musculus] |
| 19470 | A | 1203 | AI233266 | | | ESTs, Weakly similar to proline |
| | | | | | | dehydrogenase [M. musculus] |
| 19476 | O | 1188 | AI232612 | | | ESTs |
| 19503 | P | 116 | AA848639 | | | ESTs, Moderately similar to vascular |
| | | | | | | endothelial growth factor D |
| | | | | | | [M. musculus] |
| 19508 | A | 1114 | AI229698 | | | EST |
| 19512 | M | 855 | AI169612 | | | Rattus norvegicus adipocyte |
| | | | | | | lipid-binding protein (ALBP) mRNA, |
| | | | | | | complete cds |
| 19513 | R | 1100 | AI229035 | | | ESTs |
| 19566 | E | 112 | AA819879 | | | ESTs, Highly similar to ATP binding |
| | | | | | | protein [H. sapiens] |
| 19591 | S | 559 | AI012747 | | | ESTs |
| 19605 | E, L | 97 | AA819172 | | | EST |
| 19641 | J | 663 | AI071181 | | | EST |
| 19650 | H | 486 | AF016387 | | | ESTs, Rattus norvegicus retinoid X |
| | | | | | | receptor gamma (RXRgamma) mRNA, |
| | | | | | | partial cds |
| 19669 | R | 1740 | NM_022944 | | | Rattus norvegicus mRNA for SH2- |
| | | | | | | containing inositol phosphatase 2 |
| | | | | | | (SHIP2), complete cds |
| 19671 | B, Q | 1656 | NM_017309 | | protein phospatase 3, | protein phospatase 3, regulatory subunit |
| | | | | | regulatory | B, alpha isoform (calcineurin B, type I) |
| | | | | | subunit B, alpha | |
| | | | | | isoform (calcineurin B, | |
| | | | | | type I) | |
| 19678 | A | 1733 | NM_021653 | | Thyroxine deiodinase, | Rat mRNA for type I thyroxine |
| | | | | | type I | deiodinase |
| 19679 | A | 1733 | NM_021653 | | Thyroxine deiodinase, | Rat mRNA for type I thyroxine |
| | | | | | type I | deiodinase |
| 19715 | M | 1662 | NM_019190 | | membrane cofactor | membrane cofactor protein |
| | | | | | protein | |
| 19728 | O | 872 | AI170394 | | | ESTs |
| 19729 | A | 87 | AA818910 | | | ESTs |
| 19732 | A, G | 1262 | AI236066 | | | ESTs |
| 19762 | R | 272 | AA899113 | | | EST |
| 19768 | I | 237 | AA892373 | | | ESTs |
| 19787 | H | 1304 | AI638994 | | | ESTs |
| 19824 | O | 1688 | NM_021750 | Taurine and | HHs:cysteine sulfinic acid | Rattus norvegicus brain mRNA for |
| | | | | hypotaurine | decarboxylase-related | cysteine-sulfinate decarboxylase |
| | | | | metabolism | protein 2 | |
| 19825 | O | 1688 | NM_021750 | Taurine and | HHs:cysteine sulfinic acid | Rattus norvegicus brain mRNA for |
| | | | | hypotaurine | decarboxylase-related | cysteine-sulfinate decarboxylase |
| | | | | metabolism | protein 2 | |
| 19830 | A | 853 | AI169529 | | | ESTs, Weakly similar to 3O5B_RAT |
| | | | | | | 3-OXO-5-BETA-STEROID 4- |
| | | | | | | DEHYDROGENASE [R. norvegicus] |
| 19843 | A | 1308 | AI639055 | | | EST |
| 19909 | A | 1315 | AI639310 | | | EST |
| 19940 | C | 1254 | AI235689 | | | ESTs, Moderately similar to pescadillo |
| | | | | | | [H. sapiens] |
| 19952 | A | 1310 | AI639108 | | | ESTs |
| 20016 | B | 1312 | AI639158 | | | ESTs, Moderately similar to |
| | | | | | | dJ967N21.3 [H. sapiens] |
| 20035 | A | 1689 | NM_021754 | | | Rattus norvegicus Nopp140 associated |
| | | | | | | protein (NAP65) mRNA, complete cds |
| 20038 | S | 278 | AA899797 | | | EST |
| 20041 | K | 787 | AI112161 | | | ESTs |
| 20063 | E, L | 313 | AA925063 | | | ESTs, Highly similar to R32184_3 |
| | | | | | | [H. sapiens] |
| 20082 | C | 1316 | AI639488 | | | EST, Highly similar to A42772 mdm2 |
| | | | | | | protein - rat [R. norvegicus] |
| 20088 | A | 246 | AA892666 | | | ESTs |
| 20090 | R | 1690 | NM_021757 | | | Rattus norvegicus pleiotropic |
| | | | | | | regulator 1 (PLRG1) mRNA, |
| | | | | | | complete cds |
| 20119 | P | 1033 | AI178533 | | | EST, Moderately similar to |
| | | | | | | TNFC_MOUSE LYMPHOTOXIN- |
| | | | | | | BETA [M. musculus] |
| 20134 | P | 1692 | NM_021852 | | | Rattus norvegicus EH domain binding |
| | | | | | | protein epsin 2 mRNA, complete cds |
| 20161 | A, B | 1691 | NM_021836 | | | R. norvegicus pJunB gene |
| 20200 | M | 1693 | NM_022194 | | | Rat interleukin 1 receptor antagonist |
| | | | | | | gene, complete cds |
| 20282 | H | 1648 | NM_017274 | Glycerolipid metabolism | glycerol-3-phosphate | glycerol-3-phosphate acyltransferase, |
| | | | | | acyltransferase, | mitochondrial |
| | | | | | mitochondrial | |
| 20299 | A, D | 1694 | NM_022220 | | | Rattus norvegicus gene for L-gulono- |
| | | | | | | gamma-lactone oxidase |
| 20350 | L, Q | 1186 | AI232552 | | | EST |
| 20354 | B, N, Q | 1404 | M14369 | | K-kininogen, differential | K-kininogen, differential splicing |
| | | | | | splicing leads to | leads to HMW Kngk |
| | | | | | HMW Kngk | |
| 20380 | E, G | 1330 | D16102 | Glycerolipid metabolism | glycerol kinase | Rattus norvegicus mRNA for ATP- |
| | | | | | | stimulated glucocorticoid-receptor |
| | | | | | | translocaton promoter, complete cds |
| 20397 | A, E | 1151 | AI231226 | | | ESTs, Moderately similar to |
| | | | | | | SYM_HUMAN MENTHIONYL- |
| | | | | | | TRNA SYNTHETASE [H. sapiens] |
| 20449 | A, C, I | 1494 | X17053 | | Small inducible gene JE | Rattus norvegicus JE/MCP-1 mRNA, |
| | | | | | | complete cds |
| 20456 | A, C | 1355 | H31144 | | | ESTs |
| 20502 | A, F | 370 | AA945533 | | | Rattus norvegicus mRNA for organic |
| | | | | | | anion transporting polypeptide 4 |
| | | | | | | (slc21a10 gene) |
| 20503 | A, C, E | 864 | AI169779 | | | Rattus norvegicus mRNA for organic |
| | | | | | | anion transporting polypeptide 4 |
| | | | | | | (slc21a10 gene) |
| 20513 | A | 1554 | NM_012624 | Glycolysis/ | Pyruvate kinase, liver | Pyruvate kinase, liver and RBC |
| | | | | Gluconeogenesis, Purine | and RBC | |
| | | | | metabolism, Pyruvate | | |
| | | | | metabolism | | |
| 20522 | P | 224 | AA891842 | | | ESTS, Moderately similar to |
| | | | | | | podocalyxin [R. norvegicus] |
| 20523 | C, P | 224 | AA891842 | | | ESTS, Moderately similar to |
| | | | | | | podocalyxin [R. norvegicus] |
| 20529 | F, M, P | 1644 | NM_017208 | | lipopolysaccharide | lipopolysaccharide binding protein |
| | | | | | binding protein | |
| 20555 | G | 1458 | U26033 | | | Rattus norvegicus carnitine |
| | | | | | | octanoyltransferase mRNA, complete |
| | | | | | | cds |
| 20579 | O | 1654 | NM_017288 | | sodium channel, voltage- | sodium channel, voltage-gated, type I, |
| | | | | | gated, type I, | beta polypeptide |
| | | | | | beta polypeptide | |
| 20589 | I | 1553 | NM_012618 | | Protein 9 Ka homologous | Protein 9 Ka homologous to calcium- |
| | | | | | to calcium-binding | binding protein |
| | | | | | protein | |
| 20597 | S | 1489 | X12459 | Alanine and aspartate | Arginosuccinate | Arginosuccinate synthetase 1 |
| | | | | metabolism, Arginine and | synthetase 1 | |
| | | | | proline metabolism, Urea | | |
| | | | | cycle and metabolism of | | |
| | | | | amino groups | | |
| 20644 | I | 996 | AI176990 | | | ESTs, Highly similar to |
| | | | | | | SRPR_HUMAN |
| | | | | | | SIGNAL RECOGNITION PARTICLE |
| | | | | | | RECEPTOR ALPHA SUBUNIT |
| | | | | | | [H. sapiens] |
| 20651 | P | 1460 | U36992 | | Cytochrom P450 | Cytochrom P450 |
| 20684 | C | 1361 | H32977 | | | ESTs |
| 20694 | A | 442 | AA997048 | | | ESTs |
| 20698 | N | 1519 | X86561 | | | Rat alpha-fibrinogen mRNA, 3' end |
| 20701 | A, B, F, Q | 197 | AA875097 | | | Rat alpha-fibrinogen mRNA, 3' end |
| 20705 | A, D | 1541 | NM_012541 | Fatty acid | Cytochrome P450, | Cytochrome P450, subfamily I |
| | | | | metabolism, Tryptophan | subfamily I (aromatic | (aromatic compound-inducible), |
| | | | | metabolism | compound-inducible), | member A2 (Q42, form d) |
| | | | | | member A2 (Q42, form d) | |
| 20707 | A, D, K | 1481 | U88036 | | | Rattus norvegicus brain digoxin carrier |
| | | | | | | protein mRNA, complete cds |
| 20708 | C, F | 476 | AB006461 | | | Rattus norvegicus mRNA for |
| | | | | | | NORBIN, complete cds |
| 20711 | E, K | 1622 | NM_016999 | Fatty acid metabolism, | Cytochrome P450, | Cytochrome P450, subfamily IVB, |
| | | | | Tryptophan metabolism | subfamily IVB, | polypeptide 1 |
| | | | | | polypeptide 1 | |
| 20713 | K | 1622 | NM_016999 | Fatty acid metabolism, | Cytochrome P450, | Cytochrome P450, subfamily IVB, |
| | | | | Tryptophan metabolism | subfamily IVB, | polypeptide 1 |
| | | | | | polypeptide 1 | |
| 20714 | K | 1622 | NM_016999 | Fatty acid metabolism, | Cytochrome P450, | Cytochrome P450, subfamily IVB, |
| | | | | Tryptophan metabolism | subfamily IVB, | polypeptide 1 |
| | | | | | polypeptide 1 | |
| 20715 | E, N | 1622 | NM_016999 | Fatty acid metabolism, | Cytochrome P450, | Cytochrome P450, subfamily IVB, |
| | | | | Tryptophan metabolism | subfamily IVB, | polypeptide 1 |
| | | | | | polypeptide 1 | |
| 20734 | A | 1672 | NM_019283 | | antigen identified by | antigen identified by monoclonal |
| | | | | | monoclonal antibodies 4F2 | antibodies 4F2 |
| 20735 | A, C, D | 1672 | NM_019283 | | antigen identified by | antigen identified by monoclonal |
| | | | | | monoclonal antibodies 4F2 | antibodies 4F2 |
| 20741 | F | 502 | AF084186 | | | R. norvegicus mRNA for alpha II |
| | | | | | | spectrin |
| 20744 | K | 1545 | NM_012571 | Alanine and aspartate | Glutamic-oxaloacetic | Glutamic-oxaloacetic transaminase 1, |
| | | | | metabolism, Arginine and | transaminase 1, soluble | soluble (aspartate aminotransferase, |
| | | | | proline metabolism, | (aspartate aminotransferase, | cytosolic) see also D1Mgh12 |
| | | | | Cysteine metabolism, | cytosolic) see also | |
| | | | | Glutamate metabolism, | D1Mgh12 | |
| | | | | Phenylalanine metabolism, | | |
| | | | | Phenylalanine, tyrosine and | | |
| | | | | tryptophan biosynthesis, | | |
| | | | | Tyrosine metabolism | | |
| 20755 | I | 1587 | NM_012923 | | Cyclin G1 | Cyclin G1 |
| 20757 | A | 1587 | NM_012923 | | Cyclin G1 | Cyclin G1 |
| 20772 | A, F | 1468 | U60882 | | | Rattus norvegicus protein arginine N- |
| | | | | | | methyltransferase (PRMT1) mRNA, |
| | | | | | | comptete cds |
| 20795 | J | 355 | AA944397 | | | ESTs, Moderately similar to |
| | | | | | | HS9B_RAT |
| | | | | | | HEAT SHOCK PROTEIN HSP 90- |
| | | | | | | BETA [R. norvegicus] |
| 20799 | H | 1405 | M15428 | egf, epo, il2, il3, il6, | Murine leukemia viral | Murine leukemia viral (v-raf-1) |
| | | | | insulin, interact6-1, ngf, | (v-raf-1) oncogene homolog | oncogene homolog 1 (3611-MSV) |
| | | | | pdgf, tpo | 1 (3611-MSV) | |
| 20801 | A, I | 1723 | NM_024148 | | Apurinic/apyrimidinic | Rattus norvegicus mRNA for APEX |
| | | | | | endonuclease 1 | nuclease, complete cds |
| 20803 | K | 1707 | NM_022592 | Pentose phosphate cycle | HMm:transketolase | Rattus norvegicus Sprague-Dawley |
| | | | | | | transketolase mRNA, complete cds |
| 20804 | K | 1707 | NM_022592 | Pentose phosphate cycle | HMm:transketolase | Rattus norvegicus Sprague-Dawley |
| | | | | | | transketolase mRNA, complete cds |
| 20810 | A | 1493 | X14181 | | | ESTs, Highly similar to |
| | | | | | | RL1X_RAT 60S |
| | | | | | | RIBOSOMAL PROTEIN L18A |
| | | | | | | [R. norvegicus] |
| 20817 | G | 558 | AI012589 | Glutathione metabolism | glutathione S-transferase, | gluathione S-transferase, pi 2 |
| | | | | | pi 2 | |
| 20818 | G | 1458 | X02904 | Glutathione metabolism | glutathione S-transferase, | gluathione S-transferase, pi 2 |
| | | | | | pi 2 | |
| 20843 | C, D | 13 | AA799545 | | | ESTs, Weakly similar to TCPA_RAT |
| | | | | | | T-COMPLEX PROTEIN 1, ALPHA |
| | | | | | | SUBUNIT [R. norvegicus] |
| 20846 | E, N | 1147 | AI231140 | | | ESTs, Highly similar to |
| | | | | | | RL2B_HUMAN |
| | | | | | | 60S RIBOSOMAL PROTEIN L23A |
| | | | | | | [R. norvegicus] |
| 20849 | F, I | 1487 | X05566 | | | Rat mRNA for myosin regulatory light |
| | | | | | | chain (RLC) |
| 20851 | E | 1614 | NM_013214 | | acly-CoA hydrolase | acyl-CoA hydrolase |
| 20855 | S | 1613 | NM_013200 | Fatty acid metabolism, | Carnitine | Carnitine palmitoyltransferase 1 beta, |
| | | | | Glycerolipid metabolism | palmitoyltransferase | muscle isoform |
| | | | | | 1 beta, muscle isoform | |
| 20856 | S | 1613 | NM_013200 | Fatty acid metabolism, | Carnitine | Carnitine palmitoyltransferase 1 beta, |
| | | | | Glycerolipid metabolism | palmitoyltransferase | muscle isoform |
| | | | | | 1 beta, muscle isoform | |
| 20864 | G, K, P | 1615 | NM_013215 | | aflatoxin B1 aldehyde | aflatoxin B1 aldehyde reductase |
| | | | | | reductase | |
| 20873 | G | 1000 | AI177042 | | | ESTs, Highly similar to RS19_RAT |
| | | | | | | 40S RIBOSOMAL PROTEIN S19 |
| | | | | | | [R. norvegicus] |
| 20874 | A | 1116 | AI229789 | | | ESTs, Moderately similar to KIAA0952 |
| | | | | | | protein [H. sapiens] |
| 20879 | I | 1511 | X65296 | | | R. norvegicus mRNA for pl 6.1 esterase |
| | | | | | | (ES-10) |
| 20889 | A | 1563 | NM_012716 | | Solute carrier 16 | Solute carrier 16 (monocarboxylic acid |
| | | | | | (monocarboxylic acid | transporter), member 1 |
| | | | | | transporter), member 1 | |
| 20891 | A, C, I | 852 | AI169337 | | | ESTs, Highly similar to CGI-117 |
| | | | | | | protein [H. sapiens] |
| 20897 | I | 945 | AI175812 | | | ESTs, Highly simialr to Copa protein |
| | | | | | | [M. musculus] |
| 20914 | B | 1412 | M23995 | | Aldehyde dehydrogenase 1 | Aldehyde dehydrogenase 1 |
| | | | | | (phenobarbitol inducible) | (phenobarbitol inducible) |
| 20915 | K, Q | 1730 | NM_017272 | | Aldehyde dehydrogenase 1 | Aldehyde dehydrogenase 1 |
| | | | | | (phenobarbitol inducible) | (phenobarbitol inducible) |
| 20930 | E | 473 | AB004096 | Fatty acid metabolism, | Cytochrom P450 | Cytochrom P450 Lanosterol 14 alpha- |
| | | | | Tryptophan metabolism | Lanosterol 14 | demethylase |
| | | | | | alpha-demethylase | |
| 20950 | I | 7 | AA799323 | | | ESTs, Moderately similar to |
| | | | | | | PLEK_HUMAN PLECKSTRIN |
| | | | | | | [H. sapiens] |
| 20971 | H | 15 | AA799576 | | | ESTs, Weakly similar to nucleolar RNA |
| | | | | | | helicase II/Gu [M. musculus] |
| 20975 | H | 16 | AA799599 | | | ESTs |
| 20980 | E | 18 | AA799633 | | | ESTs |
| 20983 | F | 619 | AI044900 | | Acyl CoA synthetase, | Acyl CoA synthetase, long chain |
| | | | | | long chain | |
| 20986 | G | 260 | AA893242 | | Acyl CoA synthetase, | Acyl CoA synthetase, long chain |
| | | | | | long chain | |
| 20993 | R | 1041 | AI178741 | | | ESTs |
| 20998 | S | 24 | AA799803 | | | ESTs, Weakly similar to serine protease |
| | | | | | | [R. norvegicus] |
| 21010 | S | 318 | AA925306 | Alanine and aspartate | HMm:carnitine | ESTs |
| | | | | metabolism | acetyltransferase | |
| 21014 | P | 1376 | J03914 | Glutathione metabolism | Glutathione-S-transferase, | Glutathione-S-transferase, mu type 2 |
| | | | | | mu type 2 (Yb2) | (Yb2) |
| 21025 | A | 163 | AA859241 | | synaptojanin 2 binding | Rattus norvegicus NPW16 mRNA, |
| | | | | | protein | complete cds |
| 21039 | B | 1373 | J03190 | Glycine, serine and | HHs:aminolevulinate, | Rat 5-aminolevulinate synthase mRNA, |
| | | | | threonine metabolism | delta-, synthase 1 | complete cds |
| 21040 | E | 546 | AI011734 | Glycine, serine and | HHs:aminolevulinate, | Rat 5-aminolevulinate synthase mRNA, |
| | | | | threonine metabolism | delta-, synthase 1 | complete cds |
| 21060 | I | 547 | AI011746 | | | ESTs, Weakly similar to BACR7C10.a |
| | | | | | | [D. melanogaster] |
| 21068 | E | 943 | AI175675 | | | ESTs, Highly similar to |
| | | | | | | RB24_MOUSE |
| | | | | | | RAS-RELATED PROTEIN RAB-24 |
| | | | | | | [M. musculus] |
| 21075 | P | 1706 | NM_022584 | | thioredoxin reductase 2 | Rattus norvegicus thioredoxin reductase |
| | | | | | | (TrxR2) mRNA, nuclear gene encoding |
| | | | | | | mitochondrial protein, complete cds |
| 21078 | K | 1617 | NM_016986 | Fatty acid metabolism, | Acyl-Coenzyme A | Acyl-Coenzyme A dehydrogenase, C-4 |
| | | | | Propanoate metabolism, | dehydrogenase, C-4 | to C-12 straight-chain |
| | | | | Valine, leucine and | to C-12 straight-chain | |
| | | | | isoleucine degradation, | | |
| | | | | beta-Alanine metabolism | | |
| 21088 | A, F | 966 | AI176472 | | | ESTs |
| 21091 | E | 1289 | AI236972 | | | ESTs, Weakly similar to predicted using |
| | | | | | | Genefinder [C. elegans] |
| 21097 | A, H, N | 1400 | M12112 | | Angiotensinogen | Rat angiotensinogen (PAT) gene |
| 21098 | N | 344 | AA943892 | | Angiotensinogen | Rat angiotensinogen (PAT) gene |
| 21125 | A | 114 | AA848437 | | | ESTs |
| 21130 | J | 959 | AI176298 | | | ESTs |
| 21150 | A | 119 | AA848826 | | | ESTs |
| 21157 | A | 383 | AA946189 | | | ESTs |
| 21164 | O, S | 810 | AI137488 | | | ESTs |
| 21175 | H | 768 | AI105113 | | | ESTs |
| 21184 | K | 709 | AI101205 | | | ESTs |
| 21209 | A, E | 913 | AI171772 | | | ESTs |
| 21228 | K, M | 615 | AI044404 | | | ESTs |
| 21238 | K | 1719 | NM_024125 | il6, interact6-1 | Liver activating protein | Rat sfb mRNA for silencer factor B |
| | | | | | (LAP, also NF-IL6, | |
| | | | | | nuclear factor-IL6, | |
| | | | | | previously | |
| | | | | | designated TCF5) | |
| 21256 | Q | 1029 | AI178491 | | | ESTs |
| 21275 | L | 125 | AA849796 | | | ESTs |
| 21281 | B, E, M | 1231 | AI234090 | | | ESTs, Moderately similar to |
| | | | | | | hypothetical protein [H. sapiens] |
| 21285 | P | 126 | AA849898 | | | EST |
| 21305 | G | 258 | AA893082 | | | ESTs |
| 21321 | H | 1227 | AI233902 | | | ESTs |
| 21341 | A, S | 129 | AA850195 | | | ESTs |
| 21354 | S | 277 | AA899721 | | | ESTs |
| 21380 | J | 35 | AA800739 | | | ESTs, Weakly similar to /prediction |
| 21382 | N | 375 | AA945708 | | | ESTs |
| 21396 | A | 1612 | NM_013198 | Arginine and proline | Monoamine oxidase B | Monoamine oxidase B |
| | | | | metabolism, Glycine, serine | | |
| | | | | and threonine | | |
| | | | | metabolism, Histidine | | |
| | | | | metabolism, Phenylalanine | | |
| | | | | metabolism, Tryptophan | | |
| | | | | metabolism, Tyrosine | | |
| | | | | metabolism | | |
| 21414 | P | 1255 | AI235842 | | | ESTs |
| 21416 | I | 37 | AA800962 | | | ESTs, Highly similar to |
| | | | | | | TALI_MOUSE |
| | | | | | | TALIN [M. musculus] |
| 21421 | N | 1664 | NM_019196 | | multiple PDZ domain | multiple PDZ domain protein |
| | | | | | protein | |
| 21443 | P, Q | 1671 | NM_019262 | | complement component | complement component 1, q |
| | | | | | 1, q subcomponent | subcomponent, beta polypeptide |
| | | | | | beta polypeptide | |
| 21444 | Q | 1671 | NM_019262 | | complement component | complement component 1, q |
| | | | | | 1, q subcomponent, | subcomponent, beta polypeptide |
| | | | | | beta polypeptide | |
| 21445 | M, P | 1388 | L18948 | | | Rattus norvegicus intracellular calcium- |
| | | | | | | binding protein (MRP14) mRNA, |
| | | | | | | complete cds |
| 21458 | C | 311 | AA925049 | | | ESTs |
| 21467 | N | 951 | AI176061 | | | ESTs, Weakly similar to tazarotene- |
| | | | | | | induced gene 2 [H. sapiens] |
| 21471 | A | 137 | AA851343 | | | ESTs |
| 21535 | R | 1097 | AI228729 | | | ESTs |
| 21567 | R | 707 | AI101159 | | | ESTs |
| 21570 | B | 762 | AI104683 | | | ESTs |
| 21574 | N | 146 | AA852038 | | | ESTs |
| 21575 | E | 1499 | X55298 | Biosynthesis and | HHs:ribophorin II | Rat ribophorin II mRNA |
| | | | | degradation of glycoprotein | | |
| 21586 | G, I | 1521 | X97772 | | | R. norvegicus mRNA for D-3- |
| | | | | | | phosphoglycerate dehydrogenase |
| 21657 | B | 1507 | X61381 | | | Rattus norvegicus interferon-inducible |
| | | | | | | protein variant 10 mRNA, complete cds |
| 21660 | M | 863 | AI169751 | | | Rattus norvegicus interferon-inducible |
| | | | | | | protein variant 10 mRNA, complete cds |
| 21661 | M | 968 | AI176479 | | | Rattus norvegicus interferon-inducible |
| | | | | | | protein variant 10 mRNA, complete cds |
| 21663 | B | 1635 | NM_017126 | | ferredoxin 1 | ferredoxin 1 |
| 21672 | C | 222 | AA891789 | | | ESTs |
| 21682 | P, Q | 1609 | NM_013154 | | CCAAT/enhancerbinding, | CCAAT/enhancerbinding, protein |
| | | | | | protein (C/EBP) delta | (C/EBP) delta |
| 21683 | P | 1609 | NM_013154 | | CCAAT/enhancerbinding, | CCAAT/enhancerbinding, protein |
| | | | | | protein (C/EBP) delta | (C/EBP) delta |
| 21695 | A, I | 240 | AA892506 | | | ESTs, Weakly similar to coronin-like |
| | | | | | | protein [R. norvegicus] |
| 21696 | C | 1724 | NM_024152 | | | Rattus norvegicus ADP-ribosylation |
| | | | | | | factor 6 mRNA, complete cds |
| 21707 | A, C, E, N | 176 | AA859722 | | | ESTs |
| 21709 | Q | 1334 | D29683 | | Hsp:ENDOTHELIN- | Rat mRNA for endothelin-converting |
| | | | | | CONVERTING | enzyme, complete cds |
| | | | | | ENZYME 1 | |
| 21717 | E | 131 | AA850480 | | | ESTs |
| 21740 | B, M, Q | 986 | AI176810 | | | ESTs |
| 21798 | K | 329 | AA926365 | | | ESTs, Moderately similar to |
| | | | | | | AF151827_1 CGI-69 protein |
| | | | | | | [H. sapiens] |
| 21799 | E | 730 | AI102576 | | | ESTs |
| 21818 | I | 491 | AF036537 | | | Rattus norvegicus homocysteine |
| | | | | | | respondent protein HCYP2 mRNA, |
| | | | | | | complete cds |
| 21823 | E | 1119 | AI229906 | | | ESTs |
| 21893 | E | 1302 | AI237713 | | | ESTs, Moderately similar to |
| | | | | | | Y101_HUMAN HYPOTHETICAL |
| | | | | | | PROTEIN KIAA0101 [H. sapiens] |
| 21909 | H | 210 | AA891161 | | | ESTs |
| 21950 | G | 570 | AI013861 | | | Rattus norvegicus 3-hydroxyiso- |
| | | | | | | butyrate mRNA, 3' end |
| 21976 | R | 379 | AA946011 | | | ESTs |
| 21977 | A, G | 1432 | S46785 | | | Rattus norvegicus insulin-like growth |
| | | | | | | factor binding protein complex acid- |
| | | | | | | labile subunit gene, complete cds |
| 21978 | A, M | 298 | AA924289 | | | Rattus norvegicus insulin-like growth |
| | | | | | | factor binding protein complex acid- |
| | | | | | | labile subunit gene, complete cds |
| 21980 | H | 264 | AA893454 | | | ESTs |
| 22038 | A, C, D | 1297 | AI237609 | | | ESTs |
| 22042 | P | 390 | AA946476 | | | ESTs |
| 22046 | S | 331 | AA942726 | | | ESTs |
| 22051 | E | 275 | AA899498 | | | ESTs, Weakly similar to predicted using |
| | | | | | | Genefinder [C. elegans] |
| 22077 | A | 1003 | AI177099 | | | ESTs, Highly similar to serine protease |
| | | | | | | [H. sapiens] |
| 22099 | A | 727 | AI102258 | | | ESTs, Moderately similar to |
| | | | | | | BI54_MOUSE BRAIN PROTEIN I54 |
| | | | | | | [M. musculus] |
| 22124 | J | 223 | AA891790 | | | ESTs |
| 22135 | R | 887 | AI170821 | | | ESTs, Weakly similar to predicted using |
| | | | | | | Genefinder [C. elegans] |
| 22151 | B, E, Q | 521 | AI009115 | | | ESTs |
| 22177 | J | 753 | AI103730 | | | ESTs |
| 22197 | A, C | 1031 | AI178527 | | | ESTs |
| 22204 | K | 886 | AI170820 | | | ESTs |
| 22212 | A | 1268 | AI236294 | | | ESTs, Highly similar to translation |
| | | | | | | initiation factor eIF6 [M. musculus] |
| 22224 | S | 323 | AA925869 | | | ESTs |
| 22235 | L | 294 | AA924152 | | | ESTs, Moderately similar to |
| | | | | | | AF135422_1 GDP-mannose |
| | | | | | | pyrophosphorylase A [H. sapiens] |
| 22266 | E, K | 373 | AA945601 | | | ESTs |
| 22321 | B, I, M, Q | 1372 | J02962 | | | Rat IgE binding protein mRNA, |
| | | | | | | complete cds |
| 22338 | A | 345 | AA943896 | | | ESTs |
| 22368 | A, Q | 348 | AA944157 | | | ESTs |
| 22370 | S | 349 | AA944158 | | | ESTs |
| 22375 | R | 1121 | AI230046 | | | ESTs |
| 22379 | L | 1156 | AI231448 | Glycolysis/ | Glucose | ESTs, Highly similar to G6PI_MOUSE |
| | | | | Gluconeogenesis, | phosphate | GLUCOSE-6-PHOSPHATE |
| | | | | Pentose | isomerase | ISOMERASE [M. musculus] |
| | | | | phosphate cycle, | | |
| | | | | Starch and | | |
| | | | | sucrose | | |
| | | | | metabolism | | |
| 22392 | S | 351 | AA944269 | | | ESTs, Weakly similar to es 64 |
| | | | | | | [M. musculus] |
| 22395 | A | 352 | AA944289 | | | ESTs |
| 22397 | F | 353 | AA944304 | | | ESTs |
| 22412 | E | 1702 | NM_022392 | | | Rattus norvegicus growth response |
| | | | | | | protein (CL-6) mRNA, complete cds |
| 22416 | S | 354 | AA944380 | | | ESTs |
| 22432 | A, C | 895 | AI171263 | | | ESTs, Highly similar to |
| | | | | | | FBRL_MOUSE |
| | | | | | | FIBRILLARIN [M. musculus] |
| 22443 | J | 1284 | AI236761 | | | ESTs |
| 22457 | A | 358 | AA944572 | | | ESTs, Weakly similar to T2D7_RAT |
| | | | | | | TRANSCRIPTION INITIATION |
| | | | | | | FACTOR TFIID 31 KD SUBUNIT |
| | | | | | | [R. norvegicus] |
| 22487 | A, F, H | 731 | AI102578 | | | ESTs, Highly similar to I49523 Mouse |
| | | | | | | primary response gene B94 mRNA, |
| | | | | | | 3′end - mouse [M. musculus] |
| 22503 | L | 359 | AA944823 | | | ESTs |
| 22512 | M, P | 1531 | NM_012488 | | Alpha-2-macroglobulin | Alpha-2-macroglobulin |
| 22513 | F, M | 1531 | NM_012488 | | Alpha-2-macroglobulin | Alpha-2-macroglobulin |
| 22514 | M, P | 1531 | NM_012488 | | Alpha-2-macroglobulin | Alpha-2-macroglobulin |
| 22515 | M | 1531 | NM_012488 | | Alpha-2-macroglobulin | Alpha-2-macroglobulin |
| 22516 | M, P | 796 | AI113046 | | Alpha-2-macroglobulin | Alpha-2-macroglobulin |
| 22531 | E | 361 | AA944943 | | | ESTs |
| 22534 | E | 310 | AA925045 | | | ESTs |
| 22540 | R | 304 | AA924630 | Glyoxylate and | HHs:glyoxylate | ESTs, Weakly similar to SERA_RAT |
| | | | | dicarboxylate | reductase/ | D-3 PHOSPHOGLYCERATE |
| | | | | metabolism, | hydroxypyruvate | DEHYDROGENASE [R. norvegicus] |
| | | | | Pyruvate | reductase | |
| | | | | metabolism | | |
| 22548 | L | 364 | AA945031 | | | ESTs |
| 22554 | A, E, G, | 366 | AA945076 | | | ESTs |
| | O | | | | | |
| 22558 | A, E | 368 | AA945123 | | Hydroxyacid oxidase 1 | EST |
| | | | | | (glycolate | |
| | | | | | oxidase) | |
| 22559 | A, D | 839 | AI169007 | | | ESTs |
| 22566 | E | 1007 | AI177122 | | | ESTs |
| 22569 | A | 1073 | AI179979 | | | ESTs |
| 22570 | R | 369 | AA945238 | | | ESTs |
| 22582 | A, G | 1605 | NM_013120 | | Glucokinase regulatory | Glucokinase regulatory protein |
| | | | | | protein | |
| 22598 | M | 811 | AI137506 | | | ESTs, Weakly similar to SPI-2 serine |
| | | | | | | protease inhibitor [R. norvegicus] |
| 22603 | E | 494 | AF044574 | | | Rattus norvegicus putative peroxisomal |
| | | | | | | 2,4-dienoyl-CoA reductase (DCR-AKL) |
| | | | | | | mRNA, complete cds |
| 22619 | B, E, Q | 531 | AI009825 | | | ESTs |
| 22620 | S | 316 | AA925258 | | | ESTs |
| 22625 | J | 374 | AA945704 | | | ESTs |
| 22679 | A | 332 | AA942731 | | | ESTs |
| 22681 | J | 357 | AA944413 | | | ESTs |
| 22683 | A | 970 | AI176484 | | | ESTs |
| 22695 | H | 1032 | AI178531 | | | ESTs |
| 22713 | K | 378 | AA945904 | | | ESTs |
| 22717 | L | 1257 | AI235948 | | | ESTs, Highly similar to entactin |
| | | | | | | [R. norvegicus] |
| 22722 | O | 804 | AI137211 | | | ESTs |
| 22725 | Q | 283 | AA900506 | | | ESTs, Highly similar to TS24_MOUSE |
| | | | | | | PROTEIN TSG24 [M. musculus] |
| 22737 | S | 465 | AA998660 | | | ESTs |
| 22770 | A | 387 | AA946428 | | | ESTs |
| 22806 | E, Q | 551 | AI012174 | | | ESTs, Moderately similar to |
| | | | | | | hypothetical protein [H. sapiens] |
| 22835 | L | 1079 | AI180367 | | | Rattus norvegicus small zinc finger-like |
| | | | | | | protein (TIM10) mRNA, complete cds |
| 22840 | N | 528 | AI009676 | | | ESTs |
| 22862 | H | 227 | AA891944 | | | ESTs |
| 22876 | C | 917 | AI172041 | | | ESTs, Moderately similar to CGI-137 |
| | | | | | | protein [H. sapiens] |
| 22877 | A, C, D | 1045 | AI178819 | | | ESTs, Moderately similar to CGI-137 |
| | | | | | | protein [H. sapiens] |
| 22897 | P | 290 | AA901107 | | | ESTs |
| 22898 | L, P | 290 | AA901107 | | | ESTs |
| 22906 | L, N | 944 | AI175790 | | | ESTs |
| 22918 | B, Q | 29 | AA800243 | | | ESTs, Moderately similar to cell death |
| | | | | | | activator CIDE-A [M. musculus] |
| 22928 | A, F | 328 | AA926262 | | | ESTs |
| 22929 | A, L | 670 | AI071578 | | | ESTs |
| 22930 | A | 670 | AI071578 | | | ESTs |
| 22931 | A | 777 | AI105417 | | | ESTs |
| 22957 | R | 764 | AI104897 | | HMm:mitogen activated | ESTs, Moderately similar to |
| | | | | | protein kinase | meningioma expressed antigen 11 |
| | | | | | kinase 3 | [H. sapiens] |
| 22961 | E | 1064 | AI179519 | | | ESTs |
| 22966 | B | 1128 | AI230320 | | | ESTs |
| 23000 | H | 178 | AA859933 | | | ESTs |
| 23005 | F, P | 334 | AA942770 | | | ESTs |
| 23013 | I | 1137 | AI230743 | | | ESTs, Weakly similar to |
| | | | | | | ACTC_HUMAN |
| | | | | | | ACTIN, ALPHA CARDIAC |
| | | | | | | [R. norvegicus] |
| 23030 | L | 305 | AA924763 | | | ESTs |
| 23032 | K | 976 | AI176596 | | | ESTs |
| 23033 | G | 179 | AA859938 | | | ESTs |
| 23043 | N | 1051 | AI178968 | | | ESTs, Weakly similar to URB1_RAT |
| | | | | | | DNA BINDING PROTEIN URE-B1 |
| | | | | | | [R. norvegicus] |
| 23044 | A, H | 490 | AF034218 | | | Rattus norvegicus hyaluronidase |
| | | | | | | (Hyal2) mRNA, complete cds |
| 23047 | H | 230 | AA892027 | | | ESTs |
| 23075 | A | 844 | AI169166 | | | ESTs |
| 23077 | H | 1015 | AI177489 | | | ESTs |
| 23082 | A | 980 | AI176648 | | | ESTs |
| 23099 | C | 789 | AI112365 | | | ESTs, Highly similar to mm-Mago |
| | | | | | | [M. musculus] |
| 23106 | Q, R | 825 | AI145081 | | Mini chromosome | ESTs, Highly similar to cell division |
| | | | | | maintenance | control protein CDC21 [H. sapiens] |
| | | | | | deficient 4 homolog | |
| | | | | | (S. cerevisiae) | |
| 23120 | C, D | 1070 | AI179857 | | | ESTs, Weakly similar to UB5D_RAT |
| | | | | | | UBIQUITIN-CONJUGATING |
| | | | | | | ENZYME E2-17 KD 4 [R. norvegicus] |
| 23125 | B, Q | 1172 | AI232266 | | | ESTs |
| 23128 | E | 561 | AI013011 | | | ESTs |
| 23139 | H | 1076 | AI180040 | | | ESTs |
| 23160 | C, L | 960 | AI176319 | | HMm:nuclear factor | Rattus norvegicus I-kappa-B-beta |
| | | | | | of kappa light chain | mRNA, complete cds |
| | | | | | gene enhancer in | |
| | | | | | B-cells inhibitor, beta | |
| 23170 | E | 850 | AI169317 | | | ESTs, Weakly similar to C43H8.1 |
| | | | | | | [C. elegans] |
| 23173 | I | 312 | AA925057 | | | ESTs, Highly similar to CRIP_MOUSE |
| | | | | | | CYSTEINE-RICH INTESTINAL |
| | | | | | | PROTEIN [R. norvegicus] |
| 23182 | F, N | 1141 | AI230981 | | | ESTs |
| 23183 | O | 819 | AI144586 | | | Rattus norvegicus evectin-1 (EVT1) |
| | | | | | | mRNA, complete cds |
| 23184 | C | 974 | AI176554 | | | ESTs |
| 23220 | O | 1319 | AJ000347 | Sulfur | HMm:bisphosphate | Rattus norvegicus mRNA for 3′(2′), 5′- |
| | | | | metabolism | 3′-nucleotidase 1 | bisphosphate nucleotidase |
| 23229 | C | 1229 | AI234038 | | | ESTs |
| 23230 | A, H, N | 1266 | AI236146 | | | ESTs |
| 23243 | E | 138 | AA851803 | | | ESTs |
| 23245 | Q | 1066 | AI179570 | | | ESTs |
| 23260 | C, D | 856 | AI169617 | | | ESTs, Highly similar to Bop1 |
| | | | | | | [M. musculus] |
| 23261 | A, C, D | 314 | AA925145 | | | ESTs |
| 23299 | C | 989 | AI176839 | | | ESTs |
| 23302 | I, N | 1516 | X78949 | Arginine and | HMm:procollagen-proline, 2- | R. norvegicus mRNA for prolyl 4- |
| | | | | proline | oxoglutarate 4-dioxygenase | hydroxylase alpha subunit |
| | | | | metabolism | (proline 4- | |
| | | | | | hydroxylase), alpha 1 | |
| | | | | | polypeptide | |
| 23304 | E | 1153 | AI231310 | Arginine and | HMm:procollagen-proline, 2- | R. norvegicus mRNA for prolyl 4- |
| | | | | proline | oxoglutarate 4-dioxygenase | hydroxylase alpha subunit |
| | | | | metabolism | (proline 4- | |
| | | | | | hydroxylase), alpha 1 | |
| | | | | | polypeptide | |
| 23315 | E, R | 239 | AA892425 | | | ESTs |
| 23321 | A | 247 | AA892821 | | | Rattus norvegicus aiar mRNA for |
| | | | | | | androgen-inducible aldehyde reductase, |
| | | | | | | complete cds |
| 23322 | A | 247 | AA892821 | | | Rattus norvegicus aiar mRNA for |
| | | | | | | androgen-inducible aldehyde reductase, |
| | | | | | | complete cds |
| 23324 | E | 181 | AA859980 | | | ESTs, Weakly similar to TCPA_RAT |
| | | | | | | T-COMPLEX PROTEIN 1, ALPHA |
| | | | | | | SUBUNIT [R. norvegicus] |
| 23325 | A | 928 | AI172405 | | | ESTs |
| 23331 | J | 1210 | AI233457 | | | ESTs, Highly similar to Mlark |
| | | | | | | [M. musculus] |
| 23337 | E, O | 520 | AI009096 | | | Rattus norvegicus double-stranded RNA |
| | | | | | | binding protein p74 mRNA, |
| | | | | | | complete cds |
| 23362 | O | 1616 | NM_013216 | | Ras homolog enriched | Ras homolog enriched in brain |
| | | | | | in brain | |
| 23380 | A | 141 | AA851961 | | | ESTs |
| 23390 | D, G | 927 | AI172328 | | | ESTs, Weakly similar to TCPA_RAT |
| | | | | | | T-COMPLEX PROTEIN 1, ALPHA |
| | | | | | | SUBUNIT [R. norvegicus] |
| 23435 | C | 1112 | AI229502 | | | ESTs, Highly similar to KIAA0601 |
| | | | | | | protein [H. sapiens] |
| 23437 | A, O | 661 | AI071166 | | | ESTs |
| 23438 | C, J | 745 | AI103101 | | | ESTs, Highly similar to F25965 1 |
| | | | | | | [H. sapiens] |
| 23445 | A, D, F | 1571 | NM_012792 | | Flavin-containing | Flavin-containing monooxygenase 1 |
| | | | | | monooxygenase 1 | |
| 23448 | B | 315 | AA925167 | | | ESTs |
| 23449 | B, Q | 987 | AI176828 | | | ESTs |
| 23491 | H, N, O | 1681 | NM_019359 | | acidic calponin | acidic calponin |
| 23494 | N | 888 | AI170967 | | | ESTs |
| 23499 | A | 393 | AA955249 | | | EST |
| 23500 | A, S | 183 | AA860010 | | | ESTs |
| 23511 | A | 1697 | NM_022294 | | | ESTs |
| 23515 | L | 1063 | AI179498 | | | ESTs, Highly similar to |
| | | | | | | S23B_HUMAN |
| | | | | | | PROTEIN TRANSPORT PROTEIN |
| | | | | | | SEC23 HOMOLOG ISOFORM B |
| | | | | | | [H. sapiens] |
| 23522 | A, F | 1552 | NM_012615 | Arginine and | Ornitine decarboxylase | Ornitine decarboxylase |
| | | | | proline | | |
| | | | | metabolism, | | |
| | | | | Urea cycle and | | |
| | | | | metabolism | | |
| | | | | of amino groups | | |
| 23523 | A | 1552 | NM_012615 | Arginine and | Ornitine decarboxylase | Ornitine decarboxylase |
| | | | | proline | | |
| | | | | metabolism, | | |
| | | | | Urea cycle and | | |
| | | | | metabolism | | |
| | | | | of amino groups | | |
| 23555 | M, P | 394 | AA955443 | | | ESTs |
| 23558 | A | 400 | AA956170 | | | ESTs, Weakly similar to NDKA_RAT |
| | | | | | | NUCLEOSIDE DIPHOSPHATE |
| | | | | | | KINASE A [R. norvegicus] |
| 23567 | J | 1042 | AI178746 | | | ESTs |
| 23584 | A, B | 392 | AA955071 | | | ESTs |
| 23587 | J | 977 | AI176598 | | | ESTs |
| 23606 | H, N | 1714 | NM_022867 | | | Rattus norvegicus microtubule- |
| | | | | | | associated proteins 1A and 1B light |
| | | | | | | chain 3 subunit mRNA, complete cds |
| 23608 | E | 1201 | AI233190 | | | Rattus norvegicus microtubule- |
| | | | | | | associated proteins 1A and 1B light |
| | | | | | | chain 3 subunit mRNA, complete cds |
| 23612 | A | 880 | AI170751 | | | ESTs |
| 23626 | N | 395 | AA955540 | | | ESTs |
| 23627 | S | 628 | AI045624 | | | ESTs, Moderately similar to |
| | | | | | | AF151890_1 CGI-132 protein |
| | | | | | | [H. sapiens] |
| 23633 | A | 706 | AI101130 | | | ESTs |
| 23651 | I | 1582 | NM_012881 | | Sialoprotein (osteopontin) | Sialoprotein (osteopontin) |
| 23656 | R | 616 | AI044533 | | | ESTs |
| 23678 | C | 1674 | NM_019290 | | B-cell translocation | B-cell translocation gene 3 |
| | | | | | gene 3 | |
| 23679 | A, C, D, F | 1674 | NM_019290 | | B-cell translocation | B-cell translocation gene 3 |
| | | | | | gene 3 | |
| 23698 | E | 1532 | NM_012489 | | Acetyl-CoA | Acetyl-CoA acyltransferase, 3-oxo acyl- |
| | | | | | acyltransferase, 3-oxo acyl- | CoA thiolase A, peroxisomal |
| | | | | | CoA thiolase A, | |
| | | | | | peroxisomal | |
| 23709 | H, K | 1603 | NM_013113 | | ATPase Na+/K+ | ATPase Na+/K+ transporting beta 1 |
| | | | | | transporting beta 1 | polypeptide |
| | | | | | polypeptide | |
| 23710 | H | 1135 | AI230614 | | ATPase Na+/K+ | ATPase Na+/K+ transporting beta 1 |
| | | | | | transporting beta 1 | polypeptide |
| | | | | | polypeptide | |
| 23711 | H | 1603 | NM_013113 | | ATPase Na+/K+ | ATPase Na+/K+ transporting beta 1 |
| | | | | | transporting beta 1 | polypeptide |
| | | | | | polypeptide | |
| 23762 | R | 404 | AA956431 | | | ESTs, Highly similar to Lsm5 protein |
| | | | | | | [H. sapiens] |
| 23767 | A | 1295 | AI237207 | | | ESTs |
| 23843 | E, R | 412 | AA957410 | | | ESTs |
| 23847 | B | 405 | AA956723 | | | EST |
| 23854 | G, I | 1514 | X78327 | | | R. norvegicus (Sprague Dawley) |
| | | | | | | ribosomal protein L13 mRNA |
| 23855 | B, C | 1287 | AI236773 | | | ESTs |
| 23868 | F | 1543 | NM_012551 | | Early growth response 1 | Early growth response 1 |
| 23869 | F | 1543 | NM_012551 | | Early growth response 1 | Early growth response 1 |
| 23872 | F | 1543 | NM_012551 | | Early growth response 1 | Early growth response 1 |
| 23884 | A | 1422 | M73714 | Arginine and | aldehyde dehydrogenase | Rat microsomal aldehyde |
| | | | | proline | 4, liver | dehydrogenase mRNA, complete cds |
| | | | | metabolism, | microsomal (class 3) | |
| | | | | Ascorbate and | | |
| | | | | aldarate | | |
| | | | | metabolism, Bile | | |
| | | | | acid biosynthesis, | | |
| | | | | Butanoate | | |
| | | | | metabolism, | | |
| | | | | Fatty acid | | |
| | | | | metabolism, | | |
| | | | | Glycerolipid | | |
| | | | | metabolism, | | |
| | | | | Histidine | | |
| | | | | metabolism, | | |
| | | | | Lysine | | |
| | | | | degradation, | | |
| | | | | Phenylalanine | | |
| | | | | metabolism, | | |
| | | | | Propanoate | | |
| | | | | metabolism, | | |
| | | | | Pyruvate | | |
| | | | | metabolism | | |
| 23885 | E | 866 | AI170007 | | | ESTs |
| 23888 | I | 241 | AA892520 | | | ESTs |
| 23889 | M | 241 | AA892520 | | | ESTs |
| 23890 | B | 406 | AA956864 | | | ESTs |
| 23945 | F | 409 | AA957071 | | | ESTs, Highly similar to Bcl-2- |
| | | | | | | interacting protein beclin [H. sapiens] |
| 23955 | A | 1103 | AI229178 | | | ESTs |
| 23961 | A, D | 1640 | NM_017181 | Tyrosine | fumarylacetoacetate | fumarylacetoacetate hydrolase |
| | | | | metabolism | hydrolase | |
| 23987 | O | 1496 | X51615 | | | ESTs |
| 23989 | B, Q | 1072 | AI179953 | | | ESTs |
| 24012 | M, O | 411 | AA957335 | | | ESTs |
| 24024 | Q | 496 | AF052695 | | | Rattus norvegicus p55CDC |
| | | | | | | mRNA, complete cds |
| 24049 | G | 1010 | AI177341 | | | ESTs, Highly similar to CGI-10 |
| | | | | | | protein [H. sapiens] |
| 24051 | L | 414 | AA957452 | | | EST |
| 24079 | H | 935 | AI175423 | | | ESTs |
| 24112 | O | 514 | AI008773 | | | ESTs |
| 24126 | R | 415 | AA957708 | | | ESTs |
| 24146 | E | 859 | AI169668 | | | ESTs, Weakly similar to |
| | | | | | | hypothetical protein |
| | | | | | | [H. sapiens] |
| 24161 | E | 150 | AA858588 | | | ESTs |
| 24162 | A | 847 | AI169279 | | | ESTs |
| 24200 | N | 555 | AI012356 | | | ESTs |
| 24219 | A | 1395 | L27843 | | protein tyrosine phosphatase 4a1 | Rattus norvegicus tyrosine |
| | | | | | | phosphatase (PRL-1) mRNA, |
| | | | | | | complete cds |
| 24227 | L | 871 | AI170385 | | | ESTs |
| 24228 | M | 30 | AA800318 | | | ESTs, Weakly similar to |
| | | | | | | A1AT_RAT |
| | | | | | | ALPHA-1-ANTIPROTEINASE |
| | | | | | | PRECURSOR [R. norvegicus] |
| 24234 | J | 1469 | U63923 | | | Rattus norvegicus NADPH- |
| | | | | | | dependent thioredoxin reductase |
| | | | | | | (TRR1) mRNA, complete cds |
| 24235 | A, D, J | 213 | AA891286 | | | Rattus norvegicus NADPH- |
| | | | | | | dependent thioredoxin reductase |
| | | | | | | (TRR1) mRNA, complete cds |
| 24236 | C, L | 967 | AI176473 | | | ESTs |
| 24237 | F, M | 44 | AA817726 | | | ESTs |
| 24246 | G | 419 | AA963703 | | | ESTs, Highly similar to cell |
| | | | | | | cycle protein p38-2G4 homolog |
| | | | | | | [H. sapiens] |
| 24264 | A | 1593 | NM_012999 | | Subtilisin - like endoprotease | Subtilisin - like endoprotease |
| 24268 | E | 924 | AI172281 | | | ESTs |
| 24284 | A | 1715 | NM_022869 | | | Rattus norvegicus nucleolar |
| | | | | | | phosphoprotein of 140kD, |
| | | | | | | Nopp140 mRNA, complete cds |
| 24289 | B, Q | 399 | AA955986 | Galactose metabolism | Galactokinase | ESTs, Highly similar to |
| | | | | | | galactokinase [M. musculus] |
| 24296 | E | 1360 | H32867 | | | ESTs, Highly similar to |
| | | | | | | steroidogenic acute regulatory |
| | | | | | | protein [R. norvegicus] |
| 24321 | A, D, G | 1178 | AI232340 | | | ESTs |
| 24323 | P | 763 | AI104798 | | | ESTs, Moderately similar to |
| | | | | | | GTM1_RAT GLUTATHIONE |
| | | | | | | S-TRANSFERASE YB1 |
| | | | | | | [R. norvegicus] |
| 24367 | R | 401 | AA956247 | | | EST |
| 24368 | R | 1080 | AI180392 | | | ESTs, Highly similar to |
| | | | | | | AF114169_1 nucleotide-binding |
| | | | | | | protein short form [M. musculus] |
| 24369 | R | 346 | AA944011 | | | ESTs, Highly similar to |
| | | | | | | AF114169_1 nucleotide-binding |
| | | | | | | protein short form [M. musculus] |
| 24375 | A, D | 766 | AI104979 | | | ESTs, Moderately similar to |
| | | | | | | nucleolar protein p40 |
| | | | | | | [H. sapiens] |
| 24381 | S | 403 | AA956301 | | | ESTs |
| 24388 | C, D, I, R | 1286 | AI236772 | | | ESTs |
| 24434 | A | 1710 | NM_022704 | | | Rat mannose-binding protein C |
| | | | | | | (liver) mRNA, complete cds |
| 24442 | O | 1708 | NM_022667 | | | Rat matrin F/G mRNA, |
| | | | | | | complete cds |
| 24453 | F | 1560 | NM_012690 | | P-glycoprotein 3/multidrug | P-glycoprotein 3/multidrug |
| | | | | | resistance 2, P-glycoprotein/ | resistance 2 |
| | | | | | multidrug resistance 1 | |
| 24458 | A | 1711 | NM_022706 | | | Rat metabotropic glutamate |
| | | | | | | receptor (GLUR4) mRNA, |
| | | | | | | complete cds |
| 24501 | D | 1167 | AI232006 | | | Rattus norvegicus translation |
| | | | | | | elongation factor 1-delta |
| | | | | | | subunit mRNA, partial cds |
| 24508 | E | 1416 | M34643 | | | Rat neutrophin-3 (HDNF/NT-3) |
| | | | | | | mRNA, complete cds |
| 24577 | A | 1498 | X55153 | | | ESTs, Highly similar to |
| | | | | | | RLA2_RAT 60S ACIDIC |
| | | | | | | RIBOSOMAL PROTEIN P2 |
| | | | | | | [R. norvegicus] |
| 24589 | E, P | 1558 | NM_012674 | | Serine protease inhibitor, | Serine protease inhibitor, kanzal |
| | | | | | kanzal type 1/Trypsin inhibitor- | type 1/Trypsin inhibitor-like |
| | | | | | like protein, pancreatic | protein, pancreatic |
| 24597 | C | 1625 | NM_017040 | | Protein phosphatase 2 | Protein phosphatase 2 |
| | | | | | (formerly 2A), catalytic | (formerly 2A), catalytic subunit, |
| | | | | | subunit, beta isoform | beta isoform |
| 24645 | A | 1484 | V01225 | Starch and sucrose | HMm:amylase 2, pancreatic | Rat pancreatic amylase mRNA, |
| | | | | metabolism | | partial coding sequence |
| 24651 | P | 1426 | M83678 | | | Sprague-Dawley (clone LRB10) |
| | | | | | | RAB13 mRNA, 3' end |
| 24654 | E | 100 | AA819333 | | | Sprague-Dawley (clone LRB2) |
| | | | | | | RAB16 mRNA, complete cds |
| 24670 | G | 1642 | NM_017189 | | asialoglycoprotein receptor 2 | asialoglycoprotein receptor 2 |
| 24707 | E, O | 1561 | NM_012693 | Fatty acid metabolism, | Cytochrome P450 IIA2 | Cytochrome P450 IIA2 |
| | | | | Tryptophan metabolism | | |
| 24710 | C | 1430 | M98820 | interact6-1 | Interleukin 1 beta | Rat interleukin 1-beta mRNA, |
| | | | | | | complete cds |
| 24721 | Q | 99 | AA819306 | | | ESTs |
| 24722 | G | 1564 | NM_012725 | | Plasama kallikrein | Plasama kallikrein |
| 24771 | A, G | 1626 | NM_017047 | | Solute carrier family 10 | Solute carrier family 10 |
| | | | | | (sodium/bile acid cotransporter | (sodium/bile acid cotransporter |
| | | | | | family), member 1 | family), member 1 |
| 24779 | F | 1375 | J03863 | Cysteine metabolism, | HHs:serine dehydratase | Rat serine dehydratase (SDH2) |
| | | | | Glycine, serine and | | mRNA, complete cds |
| | | | | threonine metabolism, | | |
| | | | | Oxidative phosphorylation | | |
| 24810 | F, G | 1391 | L22339 | Sulfur metabolism | sulfotransferase, phenol | Rat N-hydroxy-2- |
| | | | | | preferring 2 | acetylaminofluorene (ST1C1) |
| | | | | | | mRNA, complete cds |
| 24811 | G | 1391 | L22339 | Sulfur metabolism | sulfotransferase, phenol | Rat N-hydroxy-2- |
| | | | | | preferring 2 | acetylaminofluorene (ST1C1) |
| | | | | | | mRNA, complete cds |
| 24826 | P | 1421 | M63991 | | | Rat thyroxine-binding globulin |
| | | | | | | (TBG) mRNA, 3' end |
| 24860 | K, S | 1403 | M13506 | Androgen and estrogen | Hsp:UDP- | Rat liver UDP- |
| | | | | metabolism, Pentose and | GLUCURONOSYL- | glucuronosyltransferase, |
| | | | | glucuronate | TRANSFERASE 2B1 | phenobarbital-inducible form |
| | | | | interconversions, Porphyrin | PRECURSOR, MICROSOMAL | mRNA, complete cds |
| | | | | and chlorophyll | | |
| | | | | metabolism, Starch and | | |
| | | | | sucrose metabolism | | |
| 24883 | A | 1677 | NM_019293 | Nitrogen metabolism | carbonic anhydrase 5 | carbonic anhydrase 5 |
| 25024 | F | 1353 | E03229 | | | |
| 25052 | A, F, M, P | 1390 | L22190 | | | |
| 25054 | A | 1396 | L36460 | | | |
| 25055 | K | 1398 | M11251 | | | |
| 25056 | K, L | 1402 | M13234 | | | |
| 25069 | F, G | 1440 | S82820 | | | |
| 25077 | Q | 1453 | U20643 | | | |
| 25083 | P | 1473 | U72632 | Arginine and proline | Hsp:MEMBRANE COPPER | |
| | | | | metabolism, Glycine, serine | AMINE OXIDASE | |
| | | | | and threonine | | |
| | | | | metabolism, Histidine | | |
| | | | | metabolism, Phenylalanine | | |
| | | | | metabolism, Tryptophan | | |
| | | | | metabolism, Tyrosine | | |
| | | | | metabolism, beta-Alanine | | |
| | | | | metabolism | | |
| 25098 | J | 1 | AA108277 | | | |
| 25183 | K | 495 | AF050159 | | insulin receptor substrate 2 | |
| 25198 | J | 1689 | NM_021754 | | | |
| 25203 | E | 501 | AF079873 | | | |
| 25246 | M | 1321 | AJ011607 | | | |
| 25257 | C, I | 1328 | D13623 | | | |
| 25290 | M, O | 1339 | D42148 | | | |
| 25313 | I | 1347 | D87991 | | | |
| 25370 | B, Q | 1387 | L16995 | | | |
| 25379 | Q | 1394 | L26292 | | | |
| 25397 | E | 1401 | M12822 | | | |
| 25409 | E | 1408 | M18527 | | | |
| 25410 | E | 1409 | M18528 | | | |
| 25411 | E | 1410 | M18529 | | | |
| 25413 | E | 1411 | M18531 | | | |
| 25480 | A, G | 1432 | S46785 | | | |
| 25525 | P | 1437 | S72505 | Glutathione metabolism | Hsp:GLUTATHIONE S- | |
| | | | | | TRANSFERASE YC-1 | |
| 25567 | A, J | 1441 | S85184 | | | |
| 25615 | E | 1466 | U58466 | | | |
| 25618 | M | 1470 | U64705 | | | |
| 25619 | M | 1470 | U64705 | | | |
| 25632 | G | 1476 | U75405 | | | |
| 25644 | E | 1479 | U77931 | | | |
| 25675 | A | 1493 | X14181 | | | |
| 25702 | A | 1502 | X58465 | | | |
| 25705 | H | 1504 | X59375 | | | |
| 25706 | L | 1506 | X59608 | | | |
| 25718 | I, O | 1508 | X62145 | | ribosomal protein L8 | |
| 25725 | K | 1510 | X62660 | | | |
| 25747 | A, F | 1518 | X81448 | | | |
| 25768 | Q | 1520 | X94769 | | | |
| 25777 | E | 1523 | Y08355 | | | |
| 25802 | E, I | 1352 | E02315 | | | |
| 25814 | H | 1696 | NM_022268 | | | |
| 25852 | L | 1305 | AI638998 | | | |
| 25892 | G | 1309 | AI639101 | | | |
| 25907 | J | 1313 | AI639167 | | | |
| 25938 | B | 1314 | AI639281 | | | |
| 26088 | E | 291 | AA901152 | | | |
| 26109 | S | 441 | AA997009 | | | |
| 26123 | D | 511 | AI008396 | | | |
| 26133 | M | 532 | AI009950 | | | |
| 26147 | E | 563 | AI013387 | | | |
| 26152 | N | 576 | AI028938 | | | |
| 26190 | E, R | 688 | AI072578 | | | |
| 26280 | Q | 1082 | AI227562 | | | |
| 26288 | E | 1134 | AI230577 | | | |
| 26320 | M | 1242 | AI234927 | | | |
| 26368 | E | 1367 | H34047 | | | |
| 26369 | C, D | 1369 | H34687 | | | |

TABLE 2
Document Number 1650775

| Comparison | Comparison Code |
|---|---|
| General Toxicity: Amitryptiline, ANIT, APAP, | A |
| CCI4, Diclofenac, Indomethacin, Valproate, | |
| Untreated Rats, Various Vehicles, WY-14643, | |
| Cyproterone Acetate, and Estradiol | |
| Hepatitis-inducing and NSAIDS: Diclofenac and | B |
| Indomethacin | |
| Necrosis and Fatty Liver: Carbon Tetrachloride | C |
| and Valproate | |
| Necrosis With and Without Fatty Liver: Carbon | D |
| Tetrachloride, Valproate, and Acetaminophen | |
| Protein Adduct Formers: Valproate and | E |
| Diclofenac | |
| ANIT | F |
| Late Acetaminophen | G |
| Early Acetaminophen | H |
| Late Carbon Tetrachloride | I |
| Early Carbon Tetrachloride | J |
| Late Cyproterone Acetate | K |
| Early Cyproterone Acetate | L |
| Late Diclofenac | M |
| Early Diclofenac | N |
| Estradiol | O |
| Late Indomethacin | P |
| Early Indomethacin | Q |
| Valproate | R |
| WY-14643 | S |

TABLE 3A
General Toxicity

Document Number 1650775

| | | | | Non Tox | Non Tox |
|---|---|---|---|---|---|
| GLGC ID | Tox Mean | Tox Stdev | LDA Score | Mean | Stdev |
| 21471 | 30.43 | 93.54 | 75 | −42.67 | 24.83 |
| 13203 | 35.33 | 61.64 | 74 | −31.14 | 29.79 |
| 19909 | 22.08 | 33.51 | 73 | −15.41 | 29.38 |
| 4553 | 13.83 | 18.08 | 72 | 1.43 | 6.49 |
| 15301 | 124.27 | 140.5 | 77 | 5.51 | 36.16 |
| 20456 | 42.5 | 31.85 | 70 | 7.46 | 20.45 |
| 23679 | 57.12 | 66.55 | 72 | 8.07 | 7.49 |
| 14693 | 37.57 | 38.27 | 72 | 9.49 | 11.63 |
| 12471 | 26.73 | 25.33 | 73 | 9.55 | 21.73 |
| 923 | 60.74 | 80.74 | 71 | 9.6 | 6.57 |
| 15647 | 49.51 | 40.73 | 72 | 10.9 | 23.58 |
| 6322 | 45.84 | 55.48 | 70 | 12.42 | 10.76 |
| 16314 | 48.7 | 48.51 | 70 | 12.45 | 16.75 |
| 25052 | 90.08 | 154.89 | 70 | 14.05 | 18.5 |
| 2164 | 57.65 | 53.74 | 73 | 14.96 | 17.31 |
| 16006 | 58.93 | 36.27 | 80 | 15.18 | 19.39 |
| 25054 | 45.65 | 42.59 | 72 | 15.37 | 40.01 |
| 6410 | 4.65 | 23.5 | 70 | 15.8 | 61.49 |
| 23500 | 39.03 | 35.28 | 70 | 16.65 | 11.6 |
| 16312 | 39.06 | 24.35 | 75 | 17.24 | 10.59 |
| 19843 | 2.55 | 18.74 | 74 | 17.7 | 10.31 |
| 14996 | 58.1 | 47.71 | 71 | 20.43 | 22.52 |
| 16085 | 60.79 | 45.9 | 70 | 21.59 | 14.6 |
| 17982 | 49.3 | 27.48 | 70 | 23.22 | 18.41 |
| 6226 | 46.81 | 36.97 | 71 | 23.54 | 10.28 |
| 9326 | 6.05 | 16.52 | 70 | 24.18 | 25.4 |
| 15055 | −7.1 | 34.32 | 70 | 24.3 | 26.9 |
| 351 | 94.58 | 92.7 | 71 | 26.37 | 19.43 |
| 1126 | 48.74 | 21.68 | 72 | 26.96 | 14.06 |
| 20161 | 87.17 | 88.37 | 76 | 27.44 | 26.92 |
| 8766 | −14.3 | 48.76 | 75 | 27.97 | 35.81 |
| 23511 | 12.84 | 20.12 | 72 | 29.05 | 16 |
| 5461 | 77.51 | 74.15 | 71 | 29.28 | 16.66 |
| 12216 | −22.58 | 61.28 | 71 | 29.83 | 80.65 |
| 5384 | 100.6 | 91.07 | 76 | 30.03 | 29.52 |
| 18389 | 43.98 | 46.66 | 74 | 31.53 | 26.82 |
| 21695 | 45.44 | 55.44 | 72 | 31.53 | 16.62 |
| 11357 | 17.28 | 18.76 | 73 | 31.76 | 16.7 |
| 14424 | 567.82 | 812.48 | 70 | 32.4 | 34.02 |
| 9331 | 60.44 | 27.33 | 70 | 33.81 | 15.06 |
| 23767 | 23.85 | 17.49 | 71 | 34.2 | 50.3 |
| 15862 | 62.08 | 31.33 | 71 | 34.72 | 12.31 |
| 20449 | 117.61 | 143.09 | 71 | 35.82 | 9.2 |
| 10248 | 68.54 | 26.33 | 77 | 36.88 | 16.24 |
| 23082 | 23.23 | 17.75 | 71 | 37.04 | 12.65 |
| 9425 | 17.36 | 27.44 | 71 | 37.87 | 17.12 |
| 16730 | 73.58 | 39.38 | 73 | 39.09 | 20.24 |
| 9583 | 161.94 | 162.1 | 73 | 39.37 | 25.85 |
| 11563 | 71.92 | 56.8 | 70 | 39.98 | 27.02 |
| 352 | 130.52 | 119.67 | 76 | 40.04 | 18.99 |
| 6604 | 24.19 | 16.7 | 74 | 41.3 | 15.53 |
| 7243 | 91.87 | 50.42 | 74 | 41.4 | 14.59 |
| 17709 | 71.49 | 47.04 | 70 | 41.77 | 28.89 |
| 1583 | 62.93 | 26.33 | 71 | 41.81 | 9.01 |
| 761 | 28.63 | 19.45 | 70 | 43.38 | 21.32 |
| 3849 | 81.84 | 39.76 | 71 | 43.61 | 16.59 |
| 24284 | 65.8 | 20.86 | 74 | 45.29 | 13.2 |
| 3207 | 25.59 | 109.41 | 70 | 45.31 | 54.06 |
| 21707 | 108.81 | 66.66 | 72 | 45.32 | 39.4 |
| 17589 | 85.64 | 50.71 | 71 | 46.93 | 27.53 |
| 22212 | 112.59 | 77.44 | 70 | 47.96 | 21.25 |
| 5175 | 72.78 | 115.19 | 71 | 48.48 | 31.56 |
| 7299 | 220.49 | 225.32 | 77 | 49.33 | 34.75 |
| 19678 | 3.58 | 46.62 | 75 | 49.59 | 34.93 |
| 21088 | 58.85 | 18.82 | 72 | 51.63 | 11.12 |
| 15892 | 152 | 118.78 | 75 | 52.52 | 42.58 |
| 14353 | 84.25 | 29.24 | 74 | 53.47 | 12.39 |
| 11527 | 119.25 | 79.46 | 70 | 54.98 | 27.79 |
| 13749 | 38.3 | 29.23 | 73 | 55.43 | 20.89 |
| 4281 | 38.95 | 21.16 | 70 | 57.15 | 17.8 |
| 353 | 194.24 | 177.12 | 76 | 57.46 | 26.37 |
| 14206 | 41.14 | 16.67 | 73 | 57.71 | 14.34 |
| 16080 | 207.65 | 183.99 | 77 | 58.82 | 28.68 |
| 6682 | 53.78 | 37.44 | 70 | 59.02 | 19.46 |
| 825 | 42.12 | 20.91 | 71 | 59.35 | 17.09 |
| 7918 | 90.4 | 45.57 | 71 | 60.65 | 23.06 |
| 21150 | 138.34 | 101.42 | 71 | 64.19 | 46.67 |
| 7531 | 57.13 | 26.96 | 70 | 64.99 | 18.47 |
| 22487 | 81.97 | 69.8 | 71 | 66.94 | 27.76 |
| 24264 | 112.04 | 51.05 | 72 | 67.41 | 29.12 |
| 22077 | 46.19 | 26.57 | 70 | 67.77 | 24.16 |
| 21209 | 174.43 | 157.48 | 73 | 70.46 | 46.49 |
| 20772 | 102.74 | 37.31 | 72 | 70.49 | 15.59 |
| 8600 | 33.46 | 36.07 | 72 | 71.84 | 38.68 |
| 9826 | 49.36 | 28.75 | 70 | 72 | 22.77 |
| 17688 | 108.65 | 39.15 | 70 | 72.62 | 19.69 |
| 6640 | 40.46 | 39.18 | 74 | 73.64 | 29.52 |
| 3074 | 75.98 | 91.66 | 70 | 73.84 | 44.71 |
| 4473 | 54.98 | 25.48 | 70 | 74.37 | 21.06 |
| 354 | 227.5 | 203.23 | 77 | 74.89 | 23.89 |
| 23522 | 107.75 | 42.24 | 73 | 74.91 | 18.29 |
| 15299 | 176.87 | 143.39 | 75 | 75.35 | 20.66 |
| 13166 | 145.19 | 92.31 | 71 | 75.39 | 33.67 |
| 7936 | 59.06 | 21.73 | 70 | 76.33 | 18.71 |
| 17819 | 57.46 | 25.12 | 71 | 76.84 | 20.15 |
| 17908 | 191.58 | 159.91 | 71 | 77.06 | 30.42 |
| 7681 | 125.85 | 57.35 | 71 | 77.88 | 39.68 |
| 23633 | 66.31 | 40.72 | 70 | 78.12 | 28.98 |
| 19508 | 49.65 | 31.49 | 70 | 78.53 | 32.19 |
| 9541 | 166.47 | 123.33 | 72 | 79.59 | 34.68 |
| 16446 | 58.49 | 21.61 | 71 | 80.2 | 20.86 |
| 17377 | 119.83 | 80.06 | 72 | 82.65 | 37.63 |
| 20801 | 136.04 | 60.94 | 71 | 83 | 38.58 |
| 7352 | 164.48 | 94.53 | 70 | 83.91 | 38.34 |
| 2901 | 63.21 | 31.06 | 71 | 84.9 | 24.78 |
| 15156 | 85.12 | 43.67 | 71 | 85.31 | 23.45 |
| 22877 | 140.94 | 62.91 | 71 | 85.66 | 25.88 |
| 15207 | 112.17 | 89.27 | 73 | 85.8 | 32.15 |
| 9627 | 65.98 | 37.05 | 73 | 86.7 | 25.5 |
| 4017 | 71.08 | 40.29 | 70 | 86.72 | 27.99 |
| 4944 | 252.32 | 217.46 | 76 | 86.84 | 38.34 |
| 3073 | 78.22 | 126.03 | 72 | 87.19 | 58.64 |
| 5046 | 99.33 | 75.05 | 70 | 91.34 | 37.3 |
| 3713 | 66.05 | 38.37 | 71 | 91.52 | 27.81 |
| 11576 | 56.54 | 27.2 | 75 | 92.19 | 28.07 |
| 1246 | 57.52 | 28.55 | 70 | 92.34 | 25.09 |
| 15382 | 699.61 | 884.63 | 73 | 92.89 | 30.78 |
| 18109 | 105.09 | 108.04 | 71 | 93.58 | 44.98 |
| 18906 | 66.76 | 34.6 | 72 | 93.87 | 22.06 |
| 16324 | 65.53 | 39.09 | 72 | 94.25 | 27.97 |
| 7903 | 31.76 | 35.55 | 72 | 94.94 | 65.97 |
| 7063 | 179.3 | 93.83 | 74 | 95.16 | 22.48 |
| 9053 | 60.23 | 42.49 | 72 | 97.12 | 25.77 |
| 5813 | 67.41 | 28.11 | 70 | 97.48 | 35.73 |
| 9245 | 39.62 | 45.11 | 73 | 97.55 | 55.74 |
| 16081 | 293.48 | 225.5 | 78 | 97.81 | 34.89 |
| 19085 | 146.97 | 54.5 | 71 | 98.39 | 27.86 |
| 3189 | 48.18 | 30.77 | 70 | 99.15 | 55.31 |
| 12655 | 74.53 | 78.23 | 70 | 99.85 | 45.15 |
| 5219 | 54.76 | 44.93 | 70 | 100.79 | 47.29 |
| 7062 | 157.19 | 68.98 | 70 | 101.14 | 24.11 |
| 6820 | 132.9 | 40.9 | 71 | 101.15 | 18.57 |
| 21025 | 52.78 | 49.73 | 75 | 102 | 38.88 |
| 14746 | 72.12 | 42.89 | 70 | 102.6 | 35.3 |
| 11745 | 127.84 | 29.61 | 71 | 102.7 | 19.78 |
| 20035 | 330.62 | 323.46 | 73 | 105.65 | 47.24 |
| 12587 | 72.78 | 43.64 | 72 | 105.95 | 35.48 |
| 2372 | 89.09 | 42.56 | 70 | 107.07 | 30.91 |
| 2383 | 87.59 | 39.36 | 72 | 108.56 | 32.43 |
| 2532 | 28.55 | 57.57 | 72 | 109.2 | 73.94 |
| 11959 | 91.5 | 26.27 | 70 | 109.84 | 20.36 |
| 24375 | 200.33 | 108.66 | 72 | 110.42 | 32.85 |
| 15884 | 135.81 | 86.11 | 70 | 111.91 | 36.88 |
| 2576 | 81.51 | 44.81 | 71 | 112.47 | 36.08 |
| 23955 | 98.48 | 60.26 | 72 | 113.59 | 36.89 |
| 5008 | 152.54 | 61.16 | 71 | 113.65 | 24.98 |
| 20891 | 174.25 | 85.84 | 72 | 114.45 | 35.06 |
| 18390 | 78.44 | 44.36 | 70 | 116.93 | 42.8 |
| 1844 | 172.33 | 73.68 | 70 | 117.06 | 23.94 |
| 17591 | 177.66 | 76.44 | 70 | 119.35 | 26.88 |
| 22038 | 178.88 | 77.12 | 70 | 119.93 | 32.92 |
| 20874 | 102.83 | 26.99 | 76 | 120.76 | 19.57 |
| 17844 | 225.91 | 107.09 | 73 | 120.8 | 50.32 |
| 11691 | 80.29 | 49.49 | 73 | 124.21 | 42.81 |
| 19086 | 192.42 | 71.46 | 72 | 124.7 | 32.65 |
| 14937 | 93.31 | 50.67 | 75 | 125.88 | 34.64 |
| 20513 | 76.12 | 59.17 | 72 | 127.29 | 74 |
| 6037 | 90.3 | 39.56 | 73 | 127.31 | 44.99 |
| 12332 | 24.75 | 72.13 | 73 | 128.95 | 100.98 |
| 17335 | 99.84 | 36.82 | 73 | 129.97 | 30.57 |
| 134 | 71.14 | 58.38 | 77 | 133.41 | 39.47 |
| 7784 | 109.76 | 36.32 | 70 | 134.08 | 25.84 |
| 25567 | 222.63 | 133.25 | 70 | 134.17 | 40.36 |
| 4951 | 296.48 | 152.65 | 74 | 135.21 | 102.87 |
| 13351 | 87.72 | 56.78 | 76 | 135.45 | 45.49 |
| 22432 | 207.69 | 93.56 | 71 | 137.45 | 35.3 |
| 3075 | 134.78 | 146.57 | 74 | 138.67 | 65.46 |
| 16134 | 88.41 | 44.61 | 74 | 139.59 | 36.27 |
| 18660 | 99.04 | 62.72 | 74 | 141.07 | 60.13 |
| 17225 | 208.62 | 72.16 | 71 | 141.32 | 36.37 |
| 10509 | 91.25 | 50 | 70 | 142.42 | 48.95 |
| 6190 | 108.44 | 39.25 | 71 | 142.68 | 30.93 |
| 17393 | 216.6 | 101.01 | 70 | 144.48 | 27.96 |
| 22197 | 295.18 | 157.65 | 75 | 144.6 | 54.77 |
| 19952 | 98.31 | 43.39 | 75 | 145.63 | 36.13 |
| 1690 | 206.44 | 90.45 | 70 | 147.21 | 36.46 |
| 23044 | 188.12 | 53.18 | 74 | 148 | 23.7 |
| 22931 | 50.06 | 64.25 | 72 | 148.05 | 101.64 |
| 14776 | 103.46 | 45.74 | 74 | 148.29 | 40.54 |
| 14051 | 218.89 | 97.53 | 70 | 149.85 | 36.11 |
| 22569 | 103.93 | 53.65 | 76 | 150.14 | 42.57 |
| 11403 | 485.69 | 353.08 | 74 | 150.23 | 94.34 |
| 13762 | 105.01 | 72.99 | 71 | 151.26 | 47.6 |
| 14074 | 72.32 | 60.1 | 74 | 153.35 | 74.91 |
| 18960 | 120.13 | 59.4 | 71 | 156.6 | 44.43 |
| 20889 | 193.77 | 86.18 | 70 | 156.83 | 37.64 |
| 4084 | 127.09 | 64.08 | 71 | 158.37 | 49.57 |
| 18854 | 124.79 | 56.31 | 70 | 158.52 | 38.36 |
| 20735 | 294.63 | 147.51 | 80 | 164.19 | 33.2 |
| 14181 | 117.28 | 41.72 | 73 | 165.97 | 41.05 |
| 24883 | 122.66 | 51.37 | 75 | 165.99 | 38.66 |
| 15933 | 192.2 | 65.93 | 70 | 166.13 | 35.32 |
| 18792 | 112.37 | 55.57 | 73 | 167.2 | 48.33 |
| 10544 | 240.01 | 60.23 | 77 | 167.22 | 32.41 |
| 14208 | 98.76 | 46.96 | 77 | 167.76 | 48.04 |
| 20734 | 292.65 | 126.84 . | 78 | 169.42 | 39.52 |
| 17334 | 283.45 | 131.16 | 76 | 170.46 | 50.64 |
| 22457 | 319.78 | 159.2 | 71 | 170.89 | 83.07 |
| 21978 | 127.23 | 34.44 | 75 | 172 | 37.41 |
| 20088 | 138.87 | 33.78 | 75 | 173.08 | 29.79 |
| 15300 | 301.38 | 143.25 | 73 | 174 | 53.02 |
| 16364 | 109.25 | 72.42 | 74 | 174.33 | 56.68 |
| 8829 | 280.85 | 107.19 | 74 | 174.35 | 39.95 |
| 1007 | 71.78 | 95.85 | 73 | 174.52 | 94.52 |
| 6443 | 130.76 | 76.39 | 77 | 174.54 | 46.87 |
| 17154 | 237.49 | 69.3 | 73 | 174.79 | 36.28 |
| 6473 | 107.85 | 42.8 | 72 | 175.56 | 60.84 |
| 2335 | 121.97 | 52.51 | 71 | 175.91 | 56.34 |
| 12450 | 90.03 | 92.4 | 75 | 181.36 | 63.89 |
| 16700 | 116.46 | 131.83 | 75 | 181.51 | 86.73 |
| 15955 | 105.87 | 86.17 | 73 | 183.02 | 74.51 |
| 23523 | 254.3 | 77.51 | 75 | 184.72 | 39.26 |
| 15900 | 300.11 | 139.69 | 72 | 184.95 | 58.44 |
| 10545 | 272.15 | 72.91 | 74 | 188.26 | 35.42 |
| 16982 | 503.02 | 283.02 | 72 | 188.67 | 203.36 |
| 12848 | 147.36 | 47.97 | 70 | 188.99 | 42.1 |
| 5749 | 219.23 | 62.17 | 70 | 189.76 | 42.51 |
| 15004 | 289.65 | 146.93 | 71 | 189.87 | 51.07 |
| 23075 | 307.83 | 118.82 | 72 | 190.09 | 58.23 |
| 23584 | 123.89 | 91.92 | 73 | 190.24 | 73.31 |
| 14997 | 311.34 | 155.46 | 77 | 193.29 | 31.96 |
| 7617 | 133.32 | 123.53 | 70 | 193.38 | 108.54 |
| 11404 | 425.93 | 237.07 | 74 | 193.8 | 75.57 |
| 14095 | 145.71 | 64.97 | 77 | 194.48 | 44.06 |
| 16766 | 128.68 | 62.34 | 72 | 197.3 | 64.57 |
| 13757 | 132.12 | 63.33 | 72 | 197.76 | 47.88 |
| 3981 | 165.72 | 126.27 | 71 | 199.27 | 79.29 |
| 6632 | 374.92 | 164.24 | 76 | 199.58 | 56.28 |
| 22770 | 344.97 | 196.08 | 74 | 199.66 | 52.17 |
| 1099 | 159.6 | 51.35 | 71 | 200.56 | 47.88 |
| 15170 | 132.07 | 62.08 | 79 | 201.16 | 44.18 |
| 21125 | 104.89 | 85.5 | 74 | 205.52 | 74.23 |
| 23499 | 149 | 73.65 | 71 | 206.76 | 68.16 |
| 16765 | 131.63 | 64.51 | 74 | 208.95 | 60.5 |
| 23321 | 173.83 | 57.63 | 71 | 209.49 | 31.61 |
| 18908 | 94.04 | 112.32 | 72 | 209.75 | 126.49 |
| 4360 | 159.27 | 76.32 | 72 | 212.18 | 102.53 |
| 5027 | 165.48 | 78.52 | 73 | 212.59 | 52.82 |
| 14007 | 147.14 | 73.93 | 77 | 213.84 | 62.97 |
| 4719 | 153.89 | 88.13 | 74 | 216.28 | 70.99 |
| 9754 | 78.35 | 97.33 | 75 | 218.88 | 111.68 |
| 5867 | 342.61 | 167.79 | 70 | 219.32 | 57.15 |
| 16859 | 374.28 | 189.12 | 73 | 220.43 | 60.14 |
| 24434 | 132.32 | 69.32 | 71 | 226.73 | 56.25 |
| 22683 | 206.07 | 65.39 | 71 | 228.15 | 41.78 |
| 13963 | 218.82 | 179.67 | 72 | 228.18 | 75.69 |
| 11179 | 165.79 | 72.22 | 70 | 230.16 | 61.5 |
| 23445 | 110.29 | 87.9 | 82 | 231.61 | 62.42 |
| 18115 | 174.03 | 108.43 | 71 | 231.75 | 102.05 |
| 11429 | 189.45 | 42.84 | 72 | 232.42 | 40.03 |
| 11520 | 175.16 | 127.89 | 72 | 233.8 | 92.23 |
| 7927 | 202.04 | 106.05 | 70 | 234.79 | 57.37 |
| 22099 | 137.03 | 97.01 | 71 | 235.76 | 97.02 |
| 7888 | 376.09 | 171.23 | 72 | 236.43 | 56.75 |
| 17496 | 75.49 | 73.53 | 76 | 239.51 | 173.47 |
| 11742 | 161.82 | 79.25 | 71 | 239.68 | 82.64 |
| 6855 | 194.24 | 59.54 | 71 | 245.57 | 58.27 |
| 22928 | 87.17 | 110.53 | 70 | 245.88 | 162.18 |
| 7064 | 397.22 | 140.47 | 77 | 247.28 | 40.15 |
| 10879 | 202.31 | 103.86 | 70 | 248.56 | 66.82 |
| 20757 | 401.81 | 200.88 | 71 | 249.74 | 57.1 |
| 7113 | 200.31 | 111.11 | 74 | 250.23 | 78.75 |
| 11635 | 186.84 | 60.17 | 75 | 254.75 | 47.63 |
| 135 | 174.94 | 73.25 | 78 | 256.19 | 65.78 |
| 24235 | 390.14 | 159.67 | 70 | 259.52 | 50.47 |
| 1479 | 205.28 | 61.98 | 72 | 261.61 | 51.03 |
| 5923 | 172.52 | 80.09 | 78 | 262.06 | 70.65 |
| 15642 | 368.73 | 123.22 | 77 | 262.87 | 41.31 |
| 9336 | 140.36 | 75.51 | 72 | 264.38 | 147.6 |
| 23325 | 326.83 | 125.56 | 70 | 265.55 | 63.28 |
| 9063 | 214.94 | 71.54 | 74 | 266.92 | 47.88 |
| 23612 | 382.82 | 255.62 | 72 | 267.25 | 92.93 |
| 912 | 326.5 | 67.38 | 73 | 268 | 33.47 |
| 14506 | 208.78 | 65.03 | 70 | 272.49 | 69.62 |
| 5748 | 328.41 | 66.67 | 70 | 274.63 | 44.97 |
| 8477 | 399.36 | 174.12 | 71 | 275.64 | 90.8 |
| 11021 | 177.75 | 93.53 | 73 | 275.95 | 97.97 |
| 8630 | 206.38 | 87.63 | 72 | 276.18 | 71.7 |
| 12331 | 142.97 | 91.35 | 73 | 276.42 | 113.01 |
| 12694 | 196.38 | 106.12 | 70 | 280.6 | 91.59 |
| 23380 | 201.35 | 91.04 | 71 | 280.63 | 98.56 |
| 25747 | 406.23 | 174.62 | 79 | 281.96 | 48.12 |
| 3418 | 416.76 | 178.28 | 75 | 282.48 | 51.77 |
| 19298 | 475.37 | 243.42 | 71 | 283.29 | 78.74 |
| 23558 | 187.58 | 94.53 | 72 | 284.57 | 75.57 |
| 6366 | 365.38 | 251.12 | 70 | 289.81 | 76.83 |
| 14103 | 153.89 | 84.24 | 76 | 291.22 | 113.41 |
| 24219 | 410.88 | 138.62 | 75 | 297.66 | 69 |
| 1929 | 232.96 | 81.98 | 71 | 298.56 | 77.17 |
| 5863 | 225.48 | 130.42 | 75 | 299.73 | 84.35 |
| 3504 | 395.85 | 157.69 | 70 | 301.1 | 58.36 |
| 4868 | 220.65 | 100.78 | 75 | 301.7 | 70.8 |
| 1753 | 235.94 | 62.13 | 72 | 304.05 | 74.62 |
| 22679 | 185.35 | 110.73 | 72 | 304.26 | 119.66 |
| 23230 | 431.68 | 274.8 | 77 | 305.51 | 73.66 |
| 17401 | 211.41 | 101.33 | 70 | 308.15 | 101.7 |
| 4179 | 444.58 | 228.79 | 73 | 308.58 | 63.03 |
| 24645 | 228.44 | 65.97 | 73 | 308.66 | 90.32 |
| 19679 | 212.7 | 94.25 | 74 | 309.08 | 79.13 |
| 8387 | 209.62 | 77.78 | 74 | 309.81 | 64.43 |
| 17324 | 236.31 | 65.13 | 73 | 311.13 | 52.23 |
| 1501 | 434.85 | 171.45 | 79 | 314.29 | 63.39 |
| 22582 | 224.5 | 87.58 | 71 | 316.36 | 75.3 |
| 25702 | 423.41 | 113.7 | 72 | 320.39 | 51.32 |
| 9399 | 222.67 | 63.69 | 76 | 320.67 | 86.48 |
| 3131 | 228.57 | 86.2 | 72 | 321.25 | 92.07 |
| 812 | 231.65 | 67.37 | 76 | 321.96 | 51.58 |
| 15519 | 303.98 | 284.36 | 71 | 322.04 | 142.67 |
| 1409 | 258.93 | 68.93 | 72 | 323.5 | 60.85 |
| 17049 | 207.81 | 93.01 | 77 | 324.1 | 63.71 |
| 7003 | 213.89 | 133.94 | 75 | 328.74 | 101.01 |
| 15612 | 208.41 | 106.4 | 71 | 329.06 | 202.57 |
| 851 | 259.03 | 53.32 | 76 | 331.68 | 47.82 |
| 4291 | 203.94 | 139.04 | 77 | 334.29 | 127.4 |
| 1478 | 262.27 | 68.1 | 74 | 334.41 | 51.89 |
| 7868 | 201.78 | 131.72 | 80 | 338.05 | 94.52 |
| 19469 | 284.04 | 59.16 | 72 | 342.98 | 50.36 |
| 15700 | 259.03 | 65.96 | 77 | 345.34 | 50.31 |
| 15197 | 263 | 83.78 | 70 | 348.89 | 85.31 |
| 2484 | 152.64 | 144.08 | 75 | 349.45 | 189.22 |
| 21396 | 274.52 | 76.97 | 73 | 354.24 | 57.86 |
| 15032 | 262.98 | 104.76 | 72 | 354.96 | 94.2 |
| 6825 | 321.55 | 146.79 | 71 | 355.67 | 98.41 |
| 14767 | 212.27 | 97.6 | 80 | 359.19 | 95.6 |
| 15136 | 482.9 | 133.86 | 71 | 361.06 | 68.44 |
| 2993 | 498.11 | 173.18 | 73 | 362.5 | 53.1 |
| 1175 | 211.25 | 155.83 | 72 | 367.03 | 107.25 |
| 16680 | 296.57 | 157.31 | 71 | 368.4 | 135.7 |
| 961 | 300.69 | 83.8 | 73 | 370.86 | 65.28 |
| 2696 | 463.19 | 111.26 | 71 | 371.94 | 59.78 |
| 17256 | 266.11 | 96.28 | 72 | 373.05 | 70.36 |
| 4937 | 305.59 | 112.68 | 74 | 375.59 | 89.26 |
| 18860 | 314.98 | 128.88 | 70 | 375.92 | 92.09 |
| 23884 | 312.54 | 72.12 | 70 | 379.68 | 59.35 |
| 17850 | 516.17 | 220.77 | 70 | 383.69 | 72.82 |
| 17175 | 504.94 | 132.64 | 72 | 384.43 | 64.15 |
| 12946 | 275.06 | 103.13 | 74 | 384.61 | 80.84 |
| 23322 | 308.64 | 91 .46 | 73 | 385.69 | 58.02 |
| 16327 | 318.14 | 112.83 | 72 | 386.27 | 63.57 |
| 6824 | 820.68 | 540.91 | 70 | 386.87 | 102.09 |
| 1900 | 230.35 | 153.17 | 72 | 387.22 | 135.44 |
| 14869 | 290.26 | 114.01 | 70 | 388.39 | 93.33 |
| 15239 | 472.89 | 104.14 | 70 | 393.48 | 56.96 |
| 20694 | 256 | 155.8 | 75 | 396.34 | 127.36 |
| 6321 | 661.68 | 352.96 | 71 | 397.84 | 101.24 |
| 21157 | 628.44 | 255.63 | 70 | 401.01 | 132.71 |
| 1529 | 316.33 | 75.8 | 73 | 401.61 | 56.86 |
| 5934 | 166.87 | 133.41 | 76 | 401.67 | 162.84 |
| 18597 | 452.56 | 154.66 | 72 | 402.92 | 64.14 |
| 6801 | 284.93 | 123.62 | 70 | 403.58 | 114.82 |
| 8317 | 302.02 | 115.59 | 71 | 403.7 | 92.47 |
| 3959 | 651.41 | 284.48 | 73 | 404.94 | 125.39 |
| 6017 | 218.37 | 162.51 | 71 | 408.35 | 157.64 |
| 7785 | 309.16 | 154.16 | 71 | 411.11 | 92.69 |
| 18453 | 272.77 | 135.91 | 72 | 412.12 | 103.91 |
| 11157 | 347.22 | 111.72 | 73 | 412.71 | 76.32 |
| 2799 | 186.49 | 165.24 | 73 | 413.66 | 193.94 |
| 18606 | 551.54 | 140.45 | 71 | 415.6 | 65.98 |
| 25480 | 298.56 | 93.25 | 80 | 417.76 | 62.1 |
| 6554 | 327.78 | 86.42 | 75 | 418.15 | 72.16 |
| 22395 | 337.48 | 106 | 70 | 424.15 | 101.1 |
| 18861 | 353.52 | 146.94 | 71 | 431.18 | 96.34 |
| 556 | 363.95 | 72.87 | 72 | 431.39 | 47.74 |
| 15016 | 614.84 | 191.45 | 72 | 431.42 | 106 |
| 20707 | 297.52 | 182.87 | 72 | 432.6 | 110.59 |
| 6615 | 313.91 | 151.88 | 70 | 435.29 | 105.91 |
| 25675 | 559.03 | 149.18 | 71 | 435.84 | 78.46 |
| 24458 | 391.59 | 66.22 | 70 | 440.47 | 58.22 |
| 2264 | 348.28 | 114.55 | 70 | 442.01 | 101.65 |
| 811 | 339.77 | 83.76 | 80 | 442.46 | 54.75 |
| 14962 | 595.24 | 186.44 | 71 | 443.26 | 86.3 |
| 9905 | 351.99 | 86.2 | 73 | 443.66 | 62.13 |
| 4670 | 1011.12 | 757.17 | 70 | 449.34 | 279.51 |
| 15135 | 572.07 | 128.52 | 72 | 452.98 | 71.41 |
| 1877 | 381.72 | 99.89 | 72 | 455.58 | 70.01 |
| 2905 | 368.76 | 236.61 | 74 | 455.99 | 171.06 |
| 10176 | 362.61 | 131.62 | 73 | 458.21 | 78.68 |
| 8880 | 270.36 | 150.83 | 71 | 461.94 | 178.82 |
| 21977 | 333.82 | 102.68 | 78 | 464.63 | 71.57 |
| 19103 | 373.87 | 152.27 | 72 | 466.17 | 87.18 |
| 2505 | 361.86 | 109.11 | 73 | 466.31 | 72.15 |
| 7582 | 256.38 | 164.17 | 72 | 466.34 | 223.76 |
| 18001 | 369.81 | 89.98 | 72 | 467.77 | 75.36 |
| 15755 | 405.73 | 112.28 | 71 | 473.79 | 67.48 |
| 24577 | 583.7 | 137.54 | 73 | 474.11 | 65.9 |
| 20299 | 326.39 | 113.27 | 76 | 477.33 | 90.93 |
| 7697 | 273.75 | 100.92 | 83 | 481.09 | 117.81 |
| 18867 | 425.79 | 164.92 | 71 | 486.56 | 85.09 |
| 16726 | 386.57 | 78.35 | 71 | 489.29 | 90.61 |
| 18522 | 338.66 | 110.39 | 78 | 493.05 | 127.44 |
| 794 | 364.93 | 131.6 | 73 | 493.86 | 73.31 |
| 21097 | 596.6 | 213.78 | 72 | 494.87 | 76.63 |
| 11166 | 392.77 | 163.68 | 74 | 496.16 | 102.35 |
| 3823 | 819.94 | 253.21 | 84 | 496.62 | 131.46 |
| 20701 | 546.93 | 267.9 | 71 | 497.17 | 122.04 |
| 13283 | 374.45 | 137.36 | 71 | 498.65 | 90.97 |
| 14312 | 379.02 | 130.24 | 70 | 498.8 | 162.03 |
| 1561 | 489.56 | 192.41 | 70 | 503.1 | 74.48 |
| 11693 | 280.1 | 210.45 | 74 | 504.39 | 202.02 |
| 19470 | 355.43 | 120.62 | 75 | 507.23 | 102.75 |
| 20705 | 406.75 | 228.32 | 72 | 520.73 | 125.68 |
| 6060 | 377.46 | 110.54 | 75 | 524.04 | 95.02 |
| 4143 | 411.36 | 153.04 | 70 | 526.83 | 142.72 |
| 573 | 397.93 | 141.77 | 74 | 527.31 | 101.53 |
| 2111 | 431.14 | 135.97 | 70 | 535.18 | 95.74 |
| 6132 | 389.97 | 132.3 | 70 | 536.05 | 116.38 |
| 1531 | 432.89 | 99.85 | 74 | 537.37 | 84.23 |
| 13684 | 732.21 | 234.57 | 71 | 538.64 | 123.03 |
| 4914 | 320.44 | 176.4 | 77 | 542.57 | 159.28 |
| 16172 | 384.09 | 149.87 | 71 | 543.43 | 107 |
| 18661 | 375.83 | 155.78 | 71 | 546.25 | 136.03 |
| 14035 | 354.4 | 185.79 | 72 | 546.44 | 215.25 |
| 18452 | 376.32 | 156.49 | 75 | 548.91 | 124.57 |
| 10109 | 683.1 | 154.88 | 71 | 554.69 | 60.26 |
| 15113 | 422.52 | 185.06 | 72 | 557.21 | 136.1 |
| 12087 | 426.39 | 140.52 | 70 | 558.91 | 91.57 |
| 11492 | 398.17 | 152.29 | 73 | 559.08 | 143.79 |
| 14083 | 400.42 | 184.48 | 74 | 569.39 | 131.38 |
| 23961 | 487.24 | 102.51 | 71 | 571.23 | 72.66 |
| 6761 | 734.58 | 239.42 | 73 | 572.66 | 144.55 |
| 16993 | 402.56 | 131.25 | 80 | 574.27 | 86.25 |
| 11536 | 347.49 | 123.19 | 77 | 575.39 | 198.99 |
| 12312 | 415.93 | 131.04 | 75 | 579.26 | 98.18 |
| 20810 | 686.37 | 181.4 | 70 | 589.89 | 79.84 |
| 24771 | 441.44 | 127.76 | 75 | 592.18 | 94.5 |
| 6007 | 477.65 | 139.01 | 76 | 592.68 | 113.45 |
| 3145 | 432.3 | 212.79 | 72 | 610.87 | 178.16 |
| 12064 | 392.31 | 195.73 | 78 | 611.49 | 148.58 |
| 15080 | 468.83 | 133 | 74 | 613.82 | 131.38 |
| 22338 | 858.3 | 334.36 | 70 | 633.42 | 176.07 |
| 23437 | 417.21 | 173.85 | 75 | 633.59 | 238.89 |
| 20397 | 775.65 | 145.47 | 74 | 638.29 | 86.47 |
| 22930 | 206.34 | 282.8 | 72 | 638.83 | 389.14 |
| 5943 | 365.28 | 277.04 | 78 | 658.15 | 266.99 |
| 13088 | 440.35 | 191.07 | 72 | 659.11 | 130.73 |
| 3969 | 461.16 | 167.2 | 73 | 671.43 | 138.26 |
| 2536 | 229.18 | 164.07 | 75 | 680.76 | 402.5 |
| 8946 | 488.94 | 198.29 | 74 | 698.4 | 191.02 |
| 1173 | 454.86 | 255.52 | 73 | 701.71 | 147.85 |
| 6613 | 475.14 | 319.24 | 71 | 703.21 | 206.38 |
| 17847 | 587.34 | 146.42 | 73 | 728.57 | 116.89 |
| 19069 | 401.65 | 251.38 | 70 | 736.55 | 312.13 |
| 3121 | 582.17 | 314.22 | 75 | 743.82 | 177.43 |
| 2762 | 549.37 | 222.1 | 73 | 744.04 | 144.72 |
| 9191 | 353.85 | 236.51 | 80 | 747.6 | 226.01 |
| 17339 | 394.82 | 309.4 | 71 | 757.04 | 450.78 |
| 3365 | 465.6 | 196.26 | 75 | 759.09 | 201.02 |
| 5622 | 781.85 | 245.85 | 70 | 761.19 | 118.25 |
| 19729 | 390.13 | 332.32 | 78 | 764.27 | 355.89 |
| 9012 | 363.63 | 210.98 | 77 | 764.48 | 253.76 |
| 4193 | 592.69 | 173.22 | 72 | 771.85 | 108.77 |
| 8549 | 428.57 | 212.41 | 77 | 776.74 | 195.59 |
| 16190 | 633.77 | 300.61 | 71 | 788.33 | 198.05 |
| 6143 | 563.65 | 311.9 | 76 | 807.95 | 145.12 |
| 11228 | 611.37 | 254.64 | 71 | 817.25 | 249.82 |
| 19830 | 639.79 | 218.85 | 75 | 827.94 | 161.07 |
| 11504 | 659.77 | 278.75 | 70 | 831.93 | 222.74 |
| 2569 | 457.34 | 317.75 | 82 | 855.43 | 152.77 |
| 12160 | 812.82 | 573.26 | 70 | 864.88 | 230.19 |
| 21341 | 583.63 | 407.72 | 73 | 869.75 | 255.69 |
| 24321 | 471.3 | 256.45 | 83 | 871.6 | 204.88 |
| 14584 | 778.69 | 204.76 | 72 | 899.51 | 154.36 |
| 4440 | 592.51 | 190.31 | 81 | 903.2 | 141.99 |
| 17340 | 1192.58 | 780.31 | 70 | 918.51 | 258.08 |
| 2196 | 676.58 | 230.37 | 76 | 961.23 | 265.77 |
| 16879 | 875.19 | 424.83 | 74 | 998.63 | 195.4 |
| 14118 | 716.41 | 266.36 | 72 | 1006.89 | 263.75 |
| 20503 | 598.26 | 362.91 | 74 | 1021.64 | 320.28 |
| 12306 | 1122.58 | 844.77 | 71 | 1023.1 | 338.53 |
| 2911 | 675.36 | 278.69 | 72 | 1039.76 | 290.7 |
| 18796 | 825.55 | 557.51 | 70 | 1043.22 | 369.63 |
| 19732 | 639.42 | 377.16 | 74 | 1044.68 | 344.85 |
| 11205 | 763.23 | 299.36 | 72 | 1062.45 | 233.92 |
| 13634 | 1541.83 | 591.67 | 70 | 1065.68 | 230.26 |
| 8692 | 729.45 | 328.96 | 71 | 1075.69 | 284.09 |
| 22559 | 707.2 | 351.3 | 74 | 1078.43 | 298.05 |
| 9475 | 633.07 | 305.29 | 76 | 1091.11 | 321.49 |
| 6033 | 695.09 | 293.08 | 78 | 1093.71 | 230.15 |
| 7893 | 681.36 | 341.8 | 72 | 1123.77 | 299.15 |
| 3822 | 1790.91 | 546.55 | 78 | 1156.91 | 279.92 |
| 18910 | 691.91 | 316.7 | 77 | 1158.26 | 375.48 |
| 16703 | 811.27 | 347.36 | 78 | 1176.58 | 244.51 |
| 10984 | 769.03 | 347.66 | 74 | 1177.95 | 295.11 |
| 24162 | 935.19 | 218.55 | 71 | 1183.5 | 254.36 |
| 14960 | 1815.81 | 619.16 | 72 | 1189.85 | 282.97 |
| 22368 | 809.54 | 304.72 | 78 | 1204.44 | 255.44 |
| 14512 | 758.14 | 344.89 | 75 | 1207.73 | 316.98 |
| 22929 | 345.04 | 524.79 | 76 | 1263.79 | 749.31 |
| 6633 | 1158.38 | 523.64 | 70 | 1282.41 | 230.42 |
| 5899 | 868.41 | 419.97 | 75 | 1320.55 | 275.91 |
| 17027 | 885.56 | 416.43 | 74 | 1334.54 | 460.45 |
| 633 | 1120.93 | 302.27 | 71 | 1460.55 | 215.38 |
| 15240 | 1096.17 | 411.07 | 71 | 1507.99 | 426.62 |
| 3916 | 981.26 | 439.68 | 78 | 1583.55 | 340.89 |
| 22554 | 987.76 | 444.02 | 77 | 1595.12 | 393.47 |
| 3995 | 1025.02 | 387.98 | 75 | 1611.33 | 356.12 |
| 16885 | 1112.24 | 354.14 | 71 | 1613.71 | 341.53 |
| 9889 | 981.18 | 477.47 | 73 | 1620.07 | 396.24 |
| 15029 | 925.54 | 487.41 | 79 | 1688.81 | 378.2 |
| 6015 | 1123.82 | 384.91 | 78 | 1698.32 | 346 |
| 4330 | 991.16 | 483.62 | 84 | 1718.02 | 326.97 |
| 18909 | 1097.68 | 570.79 | 73 | 1735.42 | 607.51 |
| 3934 | 1109.15 | 552.14 | 74 | 1739.43 | 460.08 |
| 19363 | 867.12 | 620.13 | 74 | 1779.39 | 738.12 |
| 18002 | 1288.49 | 485.23 | 71 | 1800.22 | 448.73 |
| 4933 | 1364.86 | 630.42 | 74 | 1830.55 | 501.46 |
| 6380 | 1372.29 | 707.55 | 71 | 1841.36 | 514.23 |
| 16883 | 1363.62 | 527.7 | 78 | 2010.57 | 420.12 |
| 6072 | 1574.16 | 580.37 | 71 | 2013.52 | 377.64 |
| 17812 | 1417.56 | 569.56 | 70 | 2054.51 | 507.28 |
| 16701 | 1417.08 | 583.17 | 75 | 2071.93 | 447.2 |
| 6016 | 1345.93 | 620.12 | 75 | 2194.85 | 585.99 |
| 23261 | 1440.1 | 757.17 | 76 | 2245.13 | 579.05 |
| 9016 | 1484.15 | 791.38 | 72 | 2570.48 | 765.58 |
| 17524 | 1867.91 | 789.56 | 72 | 2578.07 | 684.86 |
| 22558 | 2228.15 | 660.37 | 73 | 3099.17 | 679.05 |
| 20502 | 2254.47 | 1019.37 | 72 | 3293.47 | 799.82 |

TABLE 3B
Hepatitis-inducing and NSAIDS

Document Number 1650775

| | Group | Group | LDA | Non Group | Non Group |
|---|---|---|---|---|---|
| GLGC ID | Mean | Stdev | Score | Mean | Stdev |
| 1661 | 41.81 | 18.92 | 85% | 1.48 | 29.99 |
| 16317 | 30.67 | 11.58 | 80% | 8.6 | 15.46 |
| 11893 | 54.33 | 34.89 | 85% | 10.78 | 84.99 |
| 1507 | 46.98 | 9 | 89% | 15.22 | 15.58 |
| 22966 | 36.69 | 8.83 | 81% | 19.74 | 17.28 |
| 19671 | 37.69 | 7.44 | 85% | 22.27 | 14.65 |
| 20016 | 36 | 8.96 | 81% | 22.47 | 17.54 |
| 18495 | 49.47 | 12.55 | 87% | 26.89 | 16.39 |
| 671 | 1.28 | 14.77 | 83% | 29.18 | 22.7 |
| 1221 | 443.26 | 150.05 | 94% | 31.23 | 89.26 |
| 25938 | 56.45 | 7.66 | 83% | 32.22 | 17.92 |
| 18389 | 86.77 | 18.28 | 87% | 33.41 | 32.92 |
| 11974 | −0.81 | 15.18 | 84% | 37.19 | 30.74 |
| 15834 | −27.94 | 45.21 | 80% | 40.53 | 65.46 |
| 20161 | 128.51 | 48.18 | 89% | 43.77 | 57.9 |
| 17809 | 73.73 | 16.32 | 83% | 46.32 | 27.65 |
| 7056 | 3.07 | 13.95 | 81% | 47.6 | 27.96 |
| 5384 | 140.18 | 41.23 | 89% | 47.78 | 62.23 |
| 16809 | 124.52 | 30.87 | 89% | 53.12 | 26.62 |
| 11423 | 97.3 | 21.17 | 90% | 54.32 | 20.04 |
| 22918 | 25.37 | 5.71 | 92% | 57.72 | 29.27 |
| 20354 | 223.3 | 84.74 | 94% | 65.21 | 49.13 |
| 18529 | 131.4 | 33.67 | 86% | 68.42 | 53.24 |
| 1514 | 90.15 | 14.51 | 83% | 70.26 | 23.25 |
| 8079 | −4.51 | 23.75 | 93% | 71.3 | 43.24 |
| 23847 | 116.7 | 16.84 | 84% | 72.04 | 35.87 |
| 9712 | 23.03 | 12.25 | 88% | 77.04 | 28.42 |
| 3660 | 16.83 | 21.57 | 82% | 79.66 | 62.38 |
| 11904 | 167.34 | 25.7 | 93% | 81.27 | 36.83 |
| 19158 | 45.35 | 20.66 | 81% | 83.61 | 36.03 |
| 3710 | −36.33 | 22.78 | 94% | 85.53 | 112.55 |
| 15207 | 201.4 | 59.51 | 87% | 87.46 | 53.13 |
| 18272 | 60.07 | 14.42 | 82% | 88.02 | 33.03 |
| 353 | 141.35 | 40.91 | 85% | 91.87 | 108.42 |
| 19410 | 151.13 | 23.55 | 87% | 95.16 | 23.41 |
| 22321 | 170.96 | 42.18 | 92% | 100.6 | 89.13 |
| 17277 | 197.62 | 54.02 | 87% | 107.61 | 40.04 |
| 8597 | 164.65 | 22.23 | 88% | 114.16 | 40.18 |
| 22151 | 53.9 | 21.51 | 85% | 114.65 | 59.1 |
| 8274 | 76.86 | 17.29 | 87% | 123.17 | 47.02 |
| 6532 | 271.93 | 51.51 | 94% | 134.9 | 41.19 |
| 21570 | 190.77 | 30.4 | 81% | 139.02 | 39.64 |
| 2555 | 331.4 | 107.66 | 92% | 140.78 | 56.13 |
| 25370 | 84.18 | 22.52 | 80% | 142.29 | 76.05 |
| 14208 | 94.74 | 20.59 | 84% | 147.42 | 57.13 |
| 4250 | 206.6 | 31.57 | 81% | 151.25 | 44.71 |
| 1521 | 259.23 | 49.47 | 85% | 156.72 | 61.63 |
| 19075 | 223.09 | 35.39 | 81% | 163.86 | 101.01 |
| 23584 | 77.34 | 44.36 | 81% | 169.97 | 88.21 |
| 23855 | 348.59 | 60.39 | 85% | 174.64 | 78.04 |
| 9595 | 340.35 | 75.95 | 82% | 175.69 | 67.44 |
| 13332 | 103.75 | 23.14 | 88% | 187.8 | 61.54 |
| 10544 | 215.74 | 17.73 | 83% | 188.96 | 55.01 |
| 20914 | 95.15 | 42 | 80% | 195.52 | 132.48 |
| 1796 | 121.33 | 29.79 | 82% | 209 | 97.51 |
| 21039 | 106.61 | 32.3 | 84% | 211.38 | 102.32 |
| 18891 | 79.72 | 50.3 | 84% | 246.65 | 190.37 |
| 5464 | 135.66 | 32.82 | 82% | 247.44 | 149.05 |
| 15786 | 143.55 | 47.13 | 84% | 247.54 | 88.85 |
| 22619 | 538.26 | 124.75 | 87% | 252.1 | 119.33 |
| 2655 | 82.89 | 32.9 | 90% | 258.6 | 179.08 |
| 12156 | 181.92 | 29.95 | 83% | 278.7 | 159.97 |
| 17664 | 741.68 | 141.39 | 92% | 307.07 | 186.68 |
| 3504 | 500.63 | 92.33 | 90% | 315.63 | 104.18 |
| 21281 | 205.42 | 64.7 | 81% | 330.89 | 91.63 |
| 23890 | 215.59 | 58.3 | 82% | 335.94 | 112.79 |
| 21663 | 239 | 51.32 | 81% | 340.75 | 88.67 |
| 1795 | 160.6 | 58.49 | 90% | 341.81 | 148.58 |
| 6825 | 186.43 | 50.61 | 90% | 343.11 | 120.89 |
| 1900 | 172.64 | 60.15 | 81% | 346.3 | 165.46 |
| 18465 | 620.04 | 89.19 | 89% | 351.76 | 235.3 |
| 19412 | 785.76 | 148.65 | 93% | 362.14 | 121.09 |
| 4026 | 890.4 | 293.19 | 94% | 365.48 | 125.1 |
| 9148 | 247.98 | 44.83 | 82% | 370.2 | 91.6 |
| 12928 | 537.35 | 88.04 | 83% | 411.28 | 98.02 |
| 2905 | 272.3 | 68.62 | 83% | 428.13 | 203.06 |
| 21657 | 770.91 | 200.72 | 85% | 465.93 | 129.71 |
| 15127 | 328.43 | 46.16 | 84% | 473.84 | 141.3 |
| 20701 | 957.82 | 322.59 | 85% | 491.66 | 156.52 |
| 23125 | 211.15 | 54.99 | 87% | 522.67 | 517.03 |
| 15606 | 391.12 | 82.13 | 80% | 555.3 | 143.44 |
| 13557 | 380.72 | 110.05 | 84% | 601.18 | 180.33 |
| 3365 | 412.07 | 116.59 | 83% | 652.4 | 245.48 |
| 18890 | 249.81 | 125.41 | 88% | 681.61 | 362.92 |
| 21740 | 1634.89 | 574.14 | 94% | 692.6 | 269.8 |
| 3121 | 283.35 | 133.91 | 89% | 701.53 | 256.63 |
| 16458 | 914 | 77.34 | 87% | 721.93 | 196.36 |
| 11720 | 1413.34 | 300.55 | 94% | 727.31 | 251.26 |
| 11504 | 489.83 | 118.52 | 82% | 806.57 | 268.81 |
| 17768 | 607.41 | 128.96 | 82% | 831.34 | 168.24 |
| 13093 | 311.95 | 133.36 | 85% | 873.19 | 562.27 |
| 6236 | 496.56 | 151.3 | 84% | 902.06 | 432.96 |
| 23449 | 168.69 | 130.37 | 84% | 927.26 | 659.99 |
| 23989 | 1753.97 | 311.2 | 89% | 1058.6 | 400.01 |
| 23448 | 180.53 | 167.78 | 84% | 1073.75 | 757.46 |
| 24289 | 653.83 | 137.29 | 88% | 1100.08 | 340.79 |
| 16885 | 781.13 | 224.04 | 92% | 1490.2 | 403.55 |
| 3917 | 948.73 | 233.94 | 87% | 1606.37 | 494.39 |
| 6072 | 1216.55 | 290.18 | 86% | 1863.45 | 506.08 |
| 9016 | 1131.05 | 452.13 | 84% | 2271.36 | 942.23 |
| 6189 | 1001.77 | 624.81 | 84% | 2994.32 | 1665.75 |
| 16884 | 1730.22 | 430.96 | 83% | 3305.32 | 4446.34 |

TABLE 3C
Necrosis and Fatty Liver

Document Number 1650775

| | Group | Group | | Non Group | Non Group |
|---|---|---|---|---|---|
| GLGC ID | Mean | Stdev | LDA Score | Mean | Stdev |
| 7271 | 47.32 | 123.63 | 82% | −98.96 | 40.35 |
| 1727 | 109.71 | 134.11 | 80% | −50.93 | 105.7 |
| 5780 | 186.95 | 173.5 | 86% | −46.09 | 31.81 |
| 13203 | 59.69 | 60.36 | 82% | −17.7 | 44.77 |
| 16513 | 26.79 | 31.17 | 82% | −17.26 | 20.41 |
| 14619 | 43.31 | 34.51 | 85% | 2.15 | 12.76 |
| 4553 | 26.34 | 19.46 | 83% | 3.22 | 9.94 |
| 13458 | 45.73 | 26.41 | 89% | 5.65 | 18.85 |
| 1610 | 44.15 | 19.04 | 83% | 12.68 | 16.79 |
| 14693 | 74.3 | 48.25 | 83% | 13.17 | 17.15 |
| 23679 | 133.75 | 76.1 | 90% | 13.54 | 19.85 |
| 20456 | 59.55 | 30.52 | 86% | 15.2 | 27.25 |
| 5733 | 152.59 | 121.24 | 80% | 16.96 | 49.09 |
| 23435 | 130.84 | 87.29 | 81% | 21.19 | 45.23 |
| 15312 | 97.29 | 57.4 | 83% | 23.69 | 24.18 |
| 23678 | 101.95 | 55.99 | 89% | 23.69 | 13.19 |
| 15861 | 71.17 | 46.83 | 82% | 24.47 | 42.1 |
| 9181 | 83.64 | 43.77 | 86% | 24.64 | 15.48 |
| 1598 | 201.08 | 146.9 | 80% | 25.42 | 45.83 |
| 19940 | 83.79 | 44.07 | 83% | 25.73 | 17.82 |
| 9796 | 72.8 | 40.14 | 82% | 25.76 | 21.99 |
| 16085 | 106.34 | 47.32 | 89% | 28.48 | 22.62 |
| 13467 | 155.47 | 95.96 | 86% | 30.98 | 34.92 |
| 16618 | 94.85 | 58.13 | 80% | 33.73 | 25.67 |
| 24710 | 86.03 | 43.14 | 83% | 33.9 | 21 |
| 23260 | 157.52 | 100.81 | 83% | 37.65 | 37.29 |
| 22876 | 70.57 | 22.75 | 82% | 37.66 | 16.34 |
| 9331 | 80.05 | 31.38 | 80% | 38.03 | 18.65 |
| 12614 | 139.71 | 71.97 | 88% | 39.91 | 23.39 |
| 3280 | 81.33 | 28.39 | 81% | 40.1 | 20.81 |
| 13874 | 88.42 | 37.45 | 84% | 40.85 | 22.09 |
| 15862 | 84.57 | 34.63 | 80% | 42.44 | 41.06 |
| 5926 | 80.04 | 27.03 | 83% | 42.65 | 20.36 |
| 20449 | 254.92 | 200.63 | 82% | 44.06 | 38.62 |
| 15313 | 148.78 | 79.95 | 82% | 44.12 | 32.74 |
| 2897 | 110.58 | 50.4 | 86% | 47.14 | 25.32 |
| 10549 | 203.78 | 148.01 | 82% | 49.51 | 39.18 |
| 7243 | 132.31 | 62.02 | 80% | 50.65 | 27.72 |
| 14939 | 115.22 | 49.92 | 83% | 53.09 | 45.97 |
| 14242 | 118.61 | 49.19 | 85% | 53.41 | 25.56 |
| 7161 | 136.07 | 72.13 | 81% | 53.54 | 28.94 |
| 20708 | 91.32 | 26.75 | 86% | 53.6 | 18.5 |
| 3831 | 104.66 | 45.67 | 83% | 54.97 | 24.3 |
| 21707 | 135.19 | 53.83 | 81% | 55.69 | 51.38 |
| 19264 | 117.33 | 44.24 | 83% | 59.31 | 20.88 |
| 19150 | 109.31 | 32.72 | 86% | 60.72 | 15.98 |
| 17687 | 99.1 | 21.62 | 85% | 61.04 | 15.35 |
| 14462 | 156.22 | 62.83 | 84% | 62.47 | 36.02 |
| 7036 | 131.87 | 57.57 | 81% | 62.54 | 25.28 |
| 11527 | 177.9 | 80.35 | 84% | 62.69 | 44.14 |
| 20082 | 124.7 | 51.02 | 84% | 63.08 | 42.14 |
| 17736 | 432.83 | 313.35 | 81% | 65.71 | 142.15 |
| 1841 | 136.63 | 50.08 | 81% | 67.1 | 44.8 |
| 20523 | 102.48 | 38.3 | 83% | 67.66 | 66.06 |
| 12965 | 169.8 | 78.23 | 83% | 71.26 | 51.46 |
| 6085 | 208.53 | 104.4 | 83% | 72.61 | 45.7 |
| 14458 | 330.83 | 217.41 | 83% | 73.29 | 65.46 |
| 24236 | 184.01 | 75.75 | 85% | 73.32 | 33.88 |
| 23160 | 176.55 | 75.81 | 83% | 73.36 | 35.73 |
| 13251 | 323.03 | 180.5 | 84% | 75.07 | 50.76 |
| 9784 | 153.22 | 64.68 | 82% | 79.16 | 35.89 |
| 15398 | 239.17 | 147.09 | 84% | 79.65 | 55.81 |
| 353 | 280.56 | 162.02 | 81% | 80.59 | 90.86 |
| 20684 | 131.06 | 32.29 | 86% | 86.62 | 20.64 |
| 14258 | 198.53 | 76.19 | 81% | 87.06 | 38.11 |
| 22877 | 194.7 | 70.48 | 86% | 93.61 | 36.71 |
| 1411 | 202.73 | 82.72 | 81% | 98.83 | 39.17 |
| 11660 | 170.21 | 44.78 | 84% | 99.62 | 34.3 |
| 23099 | 201.64 | 75.74 | 81% | 104.62 | 41.86 |
| 23438 | 195.84 | 62.14 | 85% | 104.93 | 43.18 |
| 17734 | 614.42 | 397.11 | 81% | 110.47 | 174.81 |
| 7063 | 256.37 | 132.72 | 84% | 114.31 | 69.93 |
| 1399 | 215.1 | 91.12 | 82% | 116.84 | 76.67 |
| 5008 | 201.49 | 60.1 | 84% | 118.38 | 36.13 |
| 11331 | 223.98 | 89.07 | 83% | 120.5 | 40.92 |
| 25257 | 274.45 | 132.38 | 80% | 121.28 | 48.13 |
| 16321 | 210.67 | 63.57 | 83% | 124.13 | 43.97 |
| 20891 | 244.46 | 85.07 | 84% | 125.01 | 52.71 |
| 2938 | 92.66 | 29.87 | 81% | 127.24 | 29.13 |
| 22038 | 251.93 | 88.6 | 85% | 127.34 | 44.31 |
| 17369 | 207.5 | 75.1 | 82% | 129.13 | 60.27 |
| 5794 | 226.31 | 75.22 | 81% | 130.44 | 40.81 |
| 5489 | 273.17 | 111.54 | 82% | 136.39 | 59.55 |
| 20843 | 213.04 | 53.39 | 82% | 136.57 | 33.06 |
| 2555 | 219.93 | 71.85 | 81% | 139.38 | 59 |
| 15374 | 243.38 | 59.14 | 83% | 141.32 | 44.16 |
| 24388 | 624.21 | 327.48 | 89% | 143.82 | 68.72 |
| 22432 | 292.49 | 109.98 | 83% | 146.05 | 50.66 |
| 18418 | 239.91 | 82.99 | 83% | 146.58 | 40.53 |
| 12999 | 347.57 | 138.68 | 83% | 153.73 | 65.66 |
| 26369 | 308.75 | 109.91 | 81% | 154.12 | 55.73 |
| 14051 | 299.77 | 104 | 82% | 156.87 | 52.25 |
| 4592 | 257.24 | 62.73 | 86% | 157.37 | 38.03 |
| 4952 | 684.4 | 441.82 | 80% | 158.99 | 145.89 |
| 23184 | 332.9 | 137.24 | 81% | 159.3 | 52.72 |
| 7887 | 338.64 | 115.83 | 86% | 162.05 | 60.73 |
| 18755 | 279.19 | 80.05 | 83% | 163.56 | 53.86 |
| 17735 | 512.06 | 294.56 | 82% | 167.32 | 151.69 |
| 4781 | 344.83 | 111.41 | 85% | 169.37 | 65.78 |
| 22197 | 414.63 | 204.11 | 83% | 169.48 | 88.02 |
| 23855 | 282.27 | 93.29 | 80% | 171.07 | 75.56 |
| 14224 | 333.11 | 104.73 | 83% | 174.8 | 67.56 |
| 6796 | 410.28 | 172.66 | 86% | 185.7 | 72.52 |
| 20735 | 408.72 | 201.02 | 82% | 185.89 | 74.3 |
| 21696 | 297.51 | 89.84 | 81% | 186.09 | 42.02 |
| 11561 | 362.43 | 142.46 | 82% | 188.78 | 64.86 |
| 3203 | 308.57 | 101.34 | 81% | 194.76 | 46.19 |
| 7414 | 535.61 | 335.02 | 83% | 197.35 | 92.11 |
| 15900 | 420.93 | 177.15 | 81% | 202.45 | 80.18 |
| 23299 | 835.51 | 456.01 | 87% | 214.06 | 131.12 |
| 2615 | 386.6 | 100.97 | 86% | 217.6 | 65.98 |
| 5867 | 511.55 | 202.2 | 82% | 233.57 | 78.63 |
| 24597 | 382.02 | 100.07 | 86% | 233.91 | 54.34 |
| 11404 | 578.06 | 245.72 | 83% | 238.77 | 146.51 |
| 1460 | 401.14 | 112.53 | 84% | 244.96 | 91.82 |
| 498 | 416.48 | 120.92 | 83% | 249.32 | 96.83 |
| 16859 | 472.45 | 162.72 | 81% | 251.02 | 122.56 |
| 7888 | 537.76 | 182.29 | 85% | 257.15 | 89.71 |
| 16756 | 553.61 | 229.09 | 83% | 281.56 | 137.56 |
| 7064 | 502.34 | 176.81 | 85% | 282.57 | 116.55 |
| 3418 | 612.35 | 201.12 | 86% | 297.77 | 79.32 |
| 21458 | 1369.61 | 969.19 | 80% | 306.95 | 224.17 |
| 2818 | 499.79 | 119.08 | 85% | 321.5 | 81.64 |
| 23120 | 466.17 | 110.7 | 82% | 322.94 | 76.21 |
| 4179 | 559.24 | 157.01 | 86% | 323.2 | 127.86 |
| 21672 | 477.65 | 79.51 | 85% | 327.31 | 77.78 |
| 23229 | 626.51 | 235.94 | 81% | 338.12 | 95.94 |
| 1501 | 526.15 | 137.21 | 81% | 342.01 | 115.25 |
| 7785 | 234.09 | 120.53 | 83% | 402.39 | 211.3 |
| 6824 | 1330.86 | 651 | 84% | 457.47 | 265.81 |
| 14962 | 735.07 | 188.78 | 85% | 460.88 | 120.76 |
| 13646 | 647.84 | 120.93 | 81% | 469.35 | 113.75 |
| 11693 | 194.51 | 110.15 | 81% | 475.41 | 349.8 |
| 6132 | 303.54 | 124.75 | 81% | 496.77 | 136.48 |
| 7935 | 319.95 | 130.18 | 81% | 539.48 | 150.81 |
| 4193 | 471.49 | 196.67 | 86% | 732.69 | 138.33 |
| 2569 | 363.05 | 288.34 | 84% | 741.53 | 276.55 |
| 6143 | 440.17 | 239.99 | 82% | 761.21 | 219.76 |
| 20503 | 406.67 | 194.67 | 86% | 913.12 | 368.79 |
| 16703 | 657.32 | 260.25 | 82% | 1074.26 | 319.63 |
| 7403 | 747.37 | 603.65 | 82% | 1275.15 | 420.96 |
| 7199 | 888.57 | 501.29 | 81% | 1460.27 | 432.28 |
| 15029 | 731.54 | 467.45 | 85% | 1526.56 | 513.26 |
| 4330 | 744.46 | 374.66 | 83% | 1547.62 | 486.62 |
| 6380 | 907.19 | 397.41 | 84% | 1723.63 | 601.93 |
| 16883 | 1078.56 | 580.73 | 82% | 1877.14 | 516.54 |
| 6016 | 1048.32 | 457.34 | 84% | 2002.18 | 710.82 |
| 23261 | 1133.22 | 790.5 | 81% | 2083.71 | 702.84 |
| 9016 | 1179.45 | 473.8 | 81% | 2319.89 | 929.08 |

TABLE 3D
Necrosis With or Without Fatty Liver

Document Number 1650775

| | Group | Group | | Non Group | Non Group |
|---|---|---|---|---|---|
| GLGC ID | Mean | Stdev | LDA Score | Mean | Stdev |
| 5780 | 149.44 | 174.82 | 83% | −46.61 | 31.66 |
| 14619 | 39.67 | 32.26 | 81% | 1.81 | 12.49 |
| 5504 | 40.54 | 56.94 | 82% | 4.45 | 12.06 |
| 13458 | 39.01 | 28.21 | 82% | 5.58 | 18.92 |
| 15860 | 31.78 | 22.42 | 81% | 6.3 | 24.49 |
| 14693 | 68.27 | 45.68 | 82% | 12.72 | 16.78 |
| 23679 | 113.2 | 81.03 | 82% | 13.37 | 19.88 |
| 15312 | 89.9 | 55.01 | 81% | 23.16 | 23.77 |
| 15861 | 75.5 | 43.95 | 86% | 23.4 | 41.45 |
| 9181 | 78.27 | 41.53 | 85% | 24.18 | 14.99 |
| 16085 | 90.49 | 54.22 | 81% | 28.58 | 22.73 |
| 13723 | 125.68 | 115.97 | 84% | 29.26 | 45.67 |
| 23260 | 150.76 | 92.71 | 85% | 36.36 | 35.87 |
| 9331 | 78.82 | 28.75 | 82% | 37.48 | 18.21 |
| 12614 | 122.76 | 74.47 | 81% | 39.76 | 23.36 |
| 13874 | 91.42 | 39.76 | 85% | 39.87 | 20 |
| 15862 | 87.12 | 32.75 | 83% | 41.59 | 40.71 |
| 2838 | 145.55 | 92.3 | 83% | 42.77 | 33.6 |
| 15313 | 138.73 | 76.22 | 81% | 43.33 | 32.1 |
| 2897 | 102.26 | 48.95 | 80% | 46.84 | 25.34 |
| 10549 | 187.81 | 138.33 | 82% | 48.44 | 38.17 |
| 14939 | 109.91 | 48.48 | 81% | 52.56 | 45.94 |
| 14242 | 115.77 | 46.52 | 85% | 52.64 | 24.7 |
| 17736 | 447.8 | 300.15 | 85% | 58.86 | 128.94 |
| 19264 | 110.15 | 43.15 | 81% | 59.01 | 20.79 |
| 14462 | 146.65 | 60.75 | 83% | 61.81 | 35.78 |
| 15663 | 150.74 | 81.27 | 81% | 61.88 | 28.94 |
| 13251 | 296.06 | 174.05 | 83% | 73.46 | 48.79 |
| 6012 | 176.64 | 72.48 | 83% | 84.55 | 40.71 |
| 22877 | 181.18 | 70.29 | 80% | 93.15 | 36.67 |
| 1411 | 191.96 | 79.06 | 80% | 98.12 | 38.82 |
| 11660 | 165 | 42.53 | 82% | 98.96 | 34.06 |
| 17734 | 628.16 | 382.62 | 85% | 101.62 | 156.16 |
| 6820 | 162.7 | 43.24 | 81% | 105.26 | 24.87 |
| 1399 | 254.19 | 123.38 | 83% | 112.16 | 66.1 |
| 7063 | 246.94 | 123.92 | 84% | 112.9 | 69.1 |
| 24375 | 284.9 | 130.19 | 82% | 122.22 | 50.94 |
| 22038 | 242.92 | 82.73 | 85% | 126.16 | 43.47 |
| 15282 | 345.28 | 174.2 | 83% | 133.39 | 77.83 |
| 20843 | 205.85 | 51.68 | 80% | 135.98 | 32.8 |
| 11235 | 307.17 | 131.67 | 83% | 138.32 | 42.12 |
| 15374 | 245.25 | 54.33 | 85% | 139.6 | 42.14 |
| 8886 | 258.45 | 90.02 | 82% | 140.07 | 40.87 |
| 24388 | 550.6 | 333.76 | 85% | 142.43 | 67.72 |
| 6039 | 298.35 | 118.74 | 82% | 149.78 | 54.28 |
| 26369 | 303.77 | 102.86 | 83% | 152.16 | 53.29 |
| 14051 | 288.38 | 98.7 | 81% | 155.61 | 51.3 |
| 4592 | 241.58 | 65.95 | 80% | 157.11 | 38.16 |
| 17735 | 549.36 | 298.48 | 85% | 159 | 133.2 |
| 7887 | 321.75 | 114.32 | 83% | 160.72 | 59.56 |
| 18755 | 284.26 | 77.14 | 85% | 161.37 | 50.75 |
| 4781 | 337.58 | 103.44 | 85% | 167.27 | 63.76 |
| 20735 | 413.37 | 184.38 | 86% | 182.1 | 67.45 |
| 7414 | 505.45 | 309.7 | 84% | 194.61 | 89.53 |
| 11403 | 734.85 | 335.38 | 87% | 196.39 | 177.82 |
| 15900 | 425.49 | 161.92 | 84% | 198.73 | 74.48 |
| 15543 | 413.52 | 162.64 | 83% | 212.02 | 73.08 |
| 23445 | 63.7 | 78.02 | 82% | 213.22 | 89.74 |
| 6911 | 135.77 | 67.21 | 81% | 214.68 | 51.49 |
| 11404 | 616.53 | 242.57 | 86% | 230.44 | 130.03 |
| 5867 | 485.57 | 189.97 | 84% | 231.42 | 77.22 |
| 1460 | 416.34 | 113.77 | 87% | 241.33 | 86.89 |
| 7888 | 525.74 | 174.65 | 87% | 253.82 | 84.82 |
| 26123 | 592.58 | 263.62 | 81% | 267.76 | 130.29 |
| 16756 | 536.74 | 209.62 | 86% | 278.76 | 136.63 |
| 24235 | 489.44 | 179.4 | 82% | 280.21 | 94.54 |
| 3418 | 575.64 | 197.63 | 85% | 295.93 | 78.26 |
| 19298 | 630.43 | 229.07 | 82% | 317.49 | 143.34 |
| 23120 | 479.07 | 107.1 | 84% | 319.7 | 71.63 |
| 2818 | 482.71 | 116.97 | 82% | 320.15 | 81.06 |
| 15700 | 230.09 | 67.32 | 81% | 324.4 | 64.93 |
| 228 | 236.54 | 61.87 | 80% | 334.29 | 69.66 |
| 15032 | 205.99 | 56.82 | 80% | 339.35 | 104.9 |
| 13294 | 644.35 | 170.98 | 82% | 387.09 | 129.3 |
| 20707 | 228.73 | 113.6 | 81% | 399.4 | 144.8 |
| 20299 | 283.13 | 98.83 | 81% | 438.73 | 122.19 |
| 6824 | 1346.97 | 605.91 | 87% | 442.76 | 235.61 |
| 14962 | 719.5 | 177.74 | 85% | 457.94 | 118.72 |
| 794 | 301.18 | 105.82 | 81% | 460.38 | 105.58 |
| 13646 | 650.4 | 113.01 | 84% | 466.4 | 111.75 |
| 15135 | 628.19 | 146.12 | 81% | 475.33 | 93.64 |
| 11693 | 181.61 | 105.42 | 82% | 480.77 | 349.7 |
| 23390 | 900.94 | 286.52 | 82% | 482.87 | 204.25 |
| 6132 | 287.11 | 119.69 | 84% | 501.07 | 132.83 |
| 20705 | 268.91 | 129.82 | 81% | 501.83 | 170.59 |
| 16518 | 745.69 | 208.61 | 80% | 522.4 | 147.11 |
| 24501 | 924.14 | 324.29 | 81% | 549.2 | 118.31 |
| 13684 | 940.24 | 251.12 | 84% | 561.02 | 160.11 |
| 23961 | 413.97 | 100.86 | 81% | 563.48 | 84.42 |
| 2350 | 914.43 | 280.02 | 83% | 566.27 | 157.14 |
| 7262 | 1171.93 | 460.29 | 82% | 616.91 | 222.19 |
| 15283 | 1210.53 | 436.26 | 84% | 630.12 | 224.34 |
| 4193 | 484.87 | 182.86 | 85% | 735.61 | 136.93 |
| 15365 | 1249.48 | 437.43 | 82% | 780.82 | 1098.83 |
| 24321 | 376.06 | 230.84 | 83% | 789.46 | 268.88 |
| 22559 | 540.14 | 342.39 | 81% | 1011.15 | 343.11 |
| 5899 | 694.24 | 374.16 | 80% | 1263.41 | 404.09 |
| 7403 | 704.59 | 553.96 | 83% | 1286.73 | 413.15 |
| 7199 | 835.65 | 469.87 | 84% | 1473.34 | 421.86 |
| 15029 | 702.04 | 429.52 | 87% | 1541.16 | 503.02 |
| 4330 | 675.9 | 370.63 | 85% | 1565.51 | 467.91 |
| 18002 | 948.21 | 459.72 | 81% | 1684.6 | 511.86 |
| 6380 | 882.65 | 369.95 | 86% | 1738.14 | 594.45 |
| 16883 | 1007.86 | 547.7 | 85% | 1895.14 | 498.99 |
| 6016 | 963.32 | 454.45 | 86% | 2023.72 | 694.11 |
| 23261 | 1077.62 | 726.72 | 85% | 2102.8 | 690.37 |
| 9016 | 1096.76 | 480.03 | 84% | 2344.1 | 914.36 |
| 3062 | 1684.88 | 888.35 | 81% | 2819.77 | 870.18 |

TABLE 3E
Protein Adduct Formers

Document Number 1650775

| | Group | Group | | Non Group | Non Group |
|---|---|---|---|---|---|
| GLGC ID | Mean | Stdev | LDA Score | Mean | Stdev |
| 26190 | 48.28 | 140.35 | 73% | −116.76 | 71.12 |
| 8700 | 49.85 | 77.95 | 72% | −12.19 | 36.84 |
| 1661 | 36.36 | 40.61 | 72% | 1.43 | 29.6 |
| 18323 | 56.4 | 33.89 | 74% | 6.38 | 36.18 |
| 4348 | 50.39 | 34.87 | 73% | 11.17 | 31.72 |
| 17481 | 36.46 | 27.96 | 72% | 13.35 | 33.51 |
| 5434 | 29.26 | 14.26 | 76% | 13.66 | 16.78 |
| 5930 | 23.92 | 9.03 | 70% | 17.21 | 18.45 |
| 15778 | 24.37 | 10.62 | 70% | 18.73 | 13.8 |
| 16251 | 28.52 | 7.89 | 78% | 20.02 | 13.7 |
| 23315 | 33.84 | 16.8 | 71% | 20.08 | 11.03 |
| 23843 | 65.54 | 53.1 | 73% | 20.76 | 16.77 |
| 24268 | 31.94 | 6.01 | 72% | 20.84 | 19.94 |
| 12185 | 40.45 | 26.74 | 73% | 21.92 | 18.47 |
| 6026 | 60.83 | 27.25 | 80% | 21.94 | 33.9 |
| 9603 | 38.75 | 22.25 | 71% | 21.97 | 31.16 |
| 17747 | 8.38 | 6.53 | 74% | 22.43 | 16.15 |
| 21799 | −5.84 | 13.09 | 81% | 23.01 | 22.31 |
| 14195 | 36.74 | 19.21 | 73% | 23.09 | 19.24 |
| 3976 | 17.49 | 10.74 | 71% | 23.34 | 30.4 |
| 6533 | 32.77 | 10.84 | 73% | 23.83 | 29.19 |
| 9166 | 69.93 | 53.74 | 72% | 26.99 | 17.75 |
| 4610 | 63.26 | 38.33 | 71% | 31.07 | 36.11 |
| 16167 | 26.11 | 7.76 | 73% | 34.04 | 13.5 |
| 13967 | 69.09 | 21.43 | 77% | 35.02 | 22.23 |
| 17677 | −27.82 | 68.69 | 74% | 36.4 | 69.93 |
| 14449 | 56.08 | 25.32 | 70% | 37.77 | 22.83 |
| 11700 | 55.37 | 19.55 | 71% | 38.12 | 21.59 |
| 1538 | 7.74 | 23.48 | 75% | 38.59 | 30.39 |
| 14053 | 24.71 | 9.07 | 76% | 39.07 | 22.35 |
| 6804 | 17.85 | 7.18 | 72% | 40.39 | 128.09 |
| 15834 | −16.44 | 51.96 | 73% | 40.56 | 65.53 |
| 23170 | 43.49 | 9.26 | 75% | 40.79 | 23.99 |
| 21823 | 40.81 | 9.62 | 70% | 41.44 | 26.15 |
| 11485 | 76.43 | 21.72 | 79% | 41.78 | 31.48 |
| 26288 | 55.27 | 10.43 | 70% | 42.31 | 15.42 |
| 25409 | 8.36 | 31.39 | 76% | 43.05 | 24.65 |
| 15251 | 38.39 | 9.43 | 76% | 46.23 | 24.25 |
| 8124 | 57.68 | 9.64 | 72% | 46.93 | 19.16 |
| 14126 | 34.95 | 11.94 | 71% | 47.89 | 50.38 |
| 25203 | 29.38 | 13.58 | 73% | 47.94 | 21.85 |
| 9432 | 100.75 | 48.6 | 73% | 48.25 | 28.18 |
| 2153 | 74.75 | 38.6 | 74% | 49.01 | 17.57 |
| 11127 | 51.39 | 6.96 | 73% | 50.24 | 17.35 |
| 2933 | 50.64 | 8.95 | 72% | 51.06 | 22.58 |
| 25615 | 71.69 | 18.81 | 70% | 52.1 | 17.72 |
| 24654 | 81.41 | 24.85 | 75% | 52.19 | 24.88 |
| 15018 | 84.77 | 83.88 | 71% | 52.26 | 40.53 |
| 21707 | 126.24 | 73.39 | 70% | 59.01 | 53.51 |
| 13918 | 98.73 | 44.7 | 74% | 59.06 | 31.3 |
| 10549 | 42.34 | 9.93 | 70% | 59.31 | 64.81 |
| 22566 | 92.71 | 49.39 | 70% | 60.91 | 42.33 |
| 23304 | 84.45 | 28.37 | 70% | 61.03 | 41.36 |
| 25413 | 37.94 | 16.74 | 79% | 61.59 | 20.66 |
| 25410 | 30.99 | 21.26 | 78% | 62.85 | 30.41 |
| 25411 | 27.66 | 23.64 | 80% | 62.98 | 33.69 |
| 13581 | 83.19 | 33.57 | 71% | 63.07 | 26.31 |
| 13932 | −7.5 | 82.93 | 71% | 63.9 | 55.62 |
| 14171 | 74.42 | 21.1 | 71% | 64.55 | 37.62 |
| 90 | 36.07 | 18.79 | 70% | 65.79 | 40.02 |
| 17257 | 114.03 | 67.46 | 70% | 67.08 | 34.52 |
| 7537 | 58.32 | 14.12 | 77% | 67.47 | 33.14 |
| 25397 | 33.74 | 21.21 | 73% | 68.15 | 31.21 |
| 17894 | 82.35 | 13.84 | 78% | 68.79 | 26.36 |
| 6814 | 89.6 | 32.08 | 73% | 69.88 | 23.93 |
| 21893 | 44.34 | 8.05 | 72% | 71.05 | 72.75 |
| 11438 | 111.77 | 49.88 | 74% | 71.31 | 27.16 |
| 23324 | 87.26 | 41.21 | 73% | 73.64 | 76.07 |
| 4168 | 104.37 | 21.68 | 75% | 75.31 | 30.27 |
| 7903 | 30.15 | 21.43 | 74% | 75.81 | 76.12 |
| 14335 | 83.34 | 14.3 | 71% | 76.03 | 33.52 |
| 24589 | 112.98 | 48.88 | 76% | 76.16 | 48.86 |
| 9712 | 59.65 | 43.73 | 73% | 76.42 | 28.63 |
| 20980 | 95.23 | 16.77 | 71% | 79.04 | 22.6 |
| 6003 | 97.63 | 17.55 | 73% | 80.11 | 26.51 |
| 13175 | 132.4 | 51.99 | 72% | 81.55 | 39.28 |
| 19315 | 140.15 | 42.44 | 84% | 81.73 | 41.23 |
| 15156 | 110.09 | 19.69 | 72% | 81.74 | 31.08 |
| 1169 | 63.7 | 12.97 | 72% | 82.79 | 31.48 |
| 6032 | 51.63 | 16.54 | 72% | 83.57 | 48.94 |
| 17400 | 145.45 | 66.75 | 71% | 85.87 | 52.06 |
| 2006 | 25.42 | 45.67 | 71% | 86.52 | 90.27 |
| 21068 | 264.69 | 160.27 | 72% | 87.31 | 146.99 |
| 11215 | −7.35 | 163.64 | 72% | 87.87 | 83.21 |
| 3074 | 54.49 | 18.32 | 70% | 88.91 | 83.5 |
| 22961 | 111.83 | 20.67 | 72% | 89.09 | 31.98 |
| 2506 | 141.66 | 97.88 | 71% | 91.9 | 70.92 |
| 6409 | 148.77 | 36.6 | 74% | 92.24 | 57.46 |
| 22531 | 91.66 | 12.53 | 73% | 93.27 | 36.37 |
| 21209 | 227.02 | 212.22 | 71% | 95.2 | 92.15 |
| 2383 | 83.79 | 16.73 | 73% | 102.14 | 37.31 |
| 11174 | 184.12 | 65.2 | 77% | 102.16 | 98.46 |
| 17368 | 171.8 | 96.78 | 71% | 103.87 | 47.72 |
| 20851 | 137.3 | 28.16 | 71% | 104.02 | 55.43 |
| 3091 | 153.51 | 67.82 | 75% | 104.92 | 90.83 |
| 18390 | 78.71 | 19.55 | 74% | 106.46 | 50.88 |
| 3073 | 52.19 | 23.11 | 73% | 106.62 | 118.05 |
| 6798 | 135.78 | 43.18 | 74% | 106.64 | 46.11 |
| 14600 | 214.24 | 98.46 | 78% | 109.92 | 74.91 |
| 17617 | 99.3 | 12.59 | 72% | 110.02 | 31.44 |
| 14638 | 87.23 | 22.1 | 77% | 111.45 | 74.07 |
| 10184 | 123.58 | 33.76 | 72% | 112.37 | 55.43 |
| 9170 | 183.59 | 55.27 | 70% | 114.2 | 52.72 |
| 22151 | 79.59 | 31.13 | 71% | 114.31 | 59.46 |
| 12880 | 139.94 | 22.05 | 75% | 114.56 | 32.47 |
| 14937 | 131.42 | 66.88 | 72% | 114.75 | 41.55 |
| 2342 | 166.44 | 44.77 | 70% | 115.31 | 58.59 |
| 18612 | 131.39 | 23.5 | 75% | 116.94 | 56.6 |
| 11691 | 62.73 | 41.24 | 71% | 118 | 79.85 |
| 17451 | 101.96 | 15.77 | 72% | 120.36 | 30.67 |
| 19566 | 145.76 | 30.8 | 71% | 120.45 | 44.75 |
| 24508 | 154.79 | 40.91 | 71% | 123.72 | 32.09 |
| 1641 | 165.12 | 40.83 | 70% | 128.2 | 35.55 |
| 23885 | 161.49 | 29.33 | 72% | 129.48 | 47.42 |
| 20930 | 134.38 | 23.9 | 71% | 130.09 | 61.62 |
| 5795 | 132.03 | 27.82 | 71% | 130.17 | 53.46 |
| 22051 | 101.35 | 28.02 | 72% | 130.68 | 67.38 |
| 26368 | 145.81 | 51.6 | 71% | 132.19 | 91.73 |
| 19605 | 113.2 | 19.79 | 72% | 133.82 | 51.82 |
| 21040 | −18.07 | 52.54 | 71% | 133.85 | 229.8 |
| 14776 | 102.58 | 34.94 | 70% | 134.24 | 48.08 |
| 1223 | 182.79 | 51.88 | 71% | 136.08 | 48.54 |
| 13762 | 158.63 | 98.43 | 77% | 138.6 | 59.12 |
| 11048 | 119.54 | 22.24 | 73% | 142.6 | 56.03 |
| 2292 | 84.06 | 42.12 | 70% | 143.71 | 71.66 |
| 17844 | 277.9 | 176.64 | 73% | 144.36 | 79.81 |
| 12215 | 204 | 107.83 | 71% | 146.76 | 116.15 |
| 2043 | 179.12 | 22.45 | 78% | 147.6 | 36.11 |
| 4157 | 177.19 | 33.3 | 74% | 147.73 | 62.63 |
| 20711 | 228.01 | 78.2 | 72% | 150.83 | 116.07 |
| 26088 | 145.54 | 50.27 | 74% | 156.38 | 187.59 |
| 17572 | 159.65 | 44.25 | 71% | 158.21 | 87.38 |
| 1690 | 229.65 | 95.98 | 71% | 160.28 | 60.57 |
| 15141 | 173.57 | 16.39 | 73% | 162.21 | 36.81 |
| 16700 | 83.29 | 55.96 | 71% | 162.48 | 108.7 |
| 20380 | 146.38 | 29.01 | 71% | 163.02 | 57.5 |
| 15959 | 167.27 | 18.31 | 73% | 166.48 | 70.66 |
| 9598 | 288.09 | 95.08 | 73% | 168.1 | 93.9 |
| 11590 | 190.23 | 28.5 | 74% | 168.24 | 68.73 |
| 22806 | 131.95 | 29.2 | 75% | 169.43 | 77.82 |
| 18588 | 206.23 | 40.15 | 73% | 170.98 | 65.63 |
| 1141 | 203.77 | 31.9 | 74% | 172.68 | 35.21 |
| 9595 | 271.77 | 94.28 | 73% | 176.57 | 69.08 |
| 24146 | 216.8 | 34.19 | 71% | 177.31 | 65.74 |
| 17291 | 239.96 | 109.02 | 74% | 177.33 | 137.8 |
| 21717 | 206.89 | 32.09 | 71% | 189.62 | 69.87 |
| 13640 | 218.18 | 27.37 | 72% | 190.6 | 71.83 |
| 14007 | 153.67 | 25.25 | 74% | 191.38 | 72.77 |
| 16562 | 238.09 | 59.35 | 70% | 194.57 | 50.93 |
| 10187 | 223.84 | 49.38 | 72% | 198.22 | 88 |
| 25802 | 244.19 | 49.71 | 70% | 214.98 | 65.34 |
| 11742 | 217.52 | 133.21 | 72% | 216.12 | 86.16 |
| 5020 | 191.66 | 26.95 | 72% | 222.98 | 53.97 |
| 22603 | 221.37 | 90.45 | 71% | 229.9 | 65.5 |
| 1728 | 238.87 | 23.07 | 75% | 230.92 | 67.51 |
| 13534 | 182.27 | 33.55 | 75% | 232.74 | 85.78 |
| 2868 | 286.73 | 53.61 | 71% | 234.2 | 69.67 |
| 14997 | 375.7 | 196 | 72% | 235.84 | 152.48 |
| 5111 | 393.78 | 167.65 | 73% | 236.27 | 143.66 |
| 20063 | 181.07 | 59.31 | 70% | 236.39 | 97.14 |
| 16780 | 267.07 | 94.4 | 75% | 242.2 | 64.47 |
| 23337 | 207.26 | 31.63 | 70% | 243.84 | 91.24 |
| 19052 | 433.77 | 178.35 | 77% | 253.21 | 91.88 |
| 22619 | 416.09 | 190.68 | 70% | 253.69 | 121.24 |
| 6821 | 297.59 | 92.7 | 71% | 255.52 | 167.53 |
| 17794 | 256.5 | 47.37 | 72% | 259.54 | 87.89 |
| 5110 | 444.91 | 212.14 | 72% | 270.46 | 106.82 |
| 4929 | 215.55 | 43.79 | 71% | 270.62 | 101.5 |
| 23698 | 318.89 | 170.39 | 75% | 278.46 | 123.55 |
| 10594 | 382.41 | 57.15 | 78% | 291.69 | 58.26 |
| 6366 | 466.38 | 163.71 | 75% | 301.16 | 141.67 |
| 5091 | 204.8 | 54.15 | 76% | 305.72 | 121.65 |
| 12317 | 489.39 | 140.01 | 77% | 306.86 | 86.66 |
| 15122 | 284.14 | 30.38 | 70% | 308.23 | 65.78 |
| 2763 | 390 | 85.38 | 73% | 308.26 | 88.64 |
| 20715 | 439.32 | 105.47 | 74% | 310.12 | 180.07 |
| 25644 | 345.9 | 39.5 | 71% | 314.7 | 121.98 |
| 1175 | 204.91 | 111.96 | 71% | 321.32 | 143.78 |
| 24161 | 356.93 | 42.23 | 71% | 327.71 | 79.09 |
| 18647 | 397.22 | 64.9 | 73% | 330.24 | 91.79 |
| 21281 | 233.54 | 99.86 | 71% | 330.78 | 91.46 |
| 4179 | 625.2 | 324.6 | 71% | 330.92 | 127.34 |
| 43 | 237.61 | 86.82 | 75% | 341.37 | 75.07 |
| 19458 | 364 | 43.15 | 72% | 346.08 | 133.08 |
| 23128 | 313.06 | 51.91 | 71% | 349.02 | 136.57 |
| 22412 | 366.89 | 96.19 | 71% | 351.91 | 164.5 |
| 3143 | 483.63 | 141.06 | 72% | 352.34 | 102.15 |
| 6801 | 355 | 56.71 | 70% | 360.03 | 142.03 |
| 6066 | 431.59 | 75.6 | 72% | 368.47 | 141.78 |
| 21575 | 432.67 | 63.41 | 73% | 374.58 | 82.96 |
| 8317 | 421.43 | 158.85 | 72% | 379.92 | 111.94 |
| 4371 | 507.88 | 124.44 | 71% | 394.01 | 171.93 |
| 11157 | 373.15 | 134.06 | 70% | 394.37 | 101.64 |
| 24296 | 481.18 | 92.3 | 72% | 403.62 | 139.39 |
| 556 | 373.54 | 45.1 | 71% | 408.23 | 71.6 |
| 13055 | 482.08 | 75.69 | 75% | 411.9 | 164.09 |
| 8173 | 519.73 | 67.84 | 74% | 419.47 | 110.06 |
| 3219 | 317.14 | 59.47 | 73% | 426.13 | 99.03 |
| 16278 | 309.41 | 102.23 | 78% | 429.92 | 164.15 |
| 23608 | 566.48 | 164.2 | 70% | 431.27 | 241.18 |
| 25777 | 330.46 | 55.36 | 76% | 441.54 | 130.73 |
| 18522 | 334.4 | 99.2 | 70% | 443.31 | 151.76 |
| 6188 | 512.63 | 55.77 | 74% | 448.02 | 139.04 |
| 794 | 333.35 | 131.81 | 72% | 451.08 | 111.83 |
| 11693 | 254.85 | 149.73 | 72% | 463 | 348.51 |
| 14312 | 397.8 | 81.06 | 71% | 466.35 | 160.88 |
| 5339 | 852.55 | 606.3 | 72% | 468.96 | 257.55 |
| 13646 | 546.37 | 100.3 | 71% | 478.7 | 121.95 |
| 22534 | 444.69 | 49.89 | 76% | 478.75 | 159.7 |
| 15121 | 635.12 | 147.29 | 73% | 513.19 | 224.34 |
| 5038 | 398.62 | 86.39 | 71% | 513.52 | 201.59 |
| 7916 | 483.75 | 53.88 | 76% | 515.32 | 200.18 |
| 4759 | 421.47 | 104.72 | 71% | 536.6 | 127.07 |
| 2339 | 519.32 | 64.43 | 73% | 536.85 | 137.81 |
| 16947 | 444.15 | 113.82 | 74% | 564.09 | 119.37 |
| 24707 | 469.06 | 76.22 | 77% | 596.18 | 184.62 |
| 13557 | 472.83 | 125.45 | 74% | 600 | 181.83 |
| 11322 | 781.82 | 176.95 | 71% | 605.26 | 189.58 |
| 16623 | 815.06 | 113.69 | 75% | 643.07 | 187.67 |
| 20397 | 756.19 | 106.73 | 71% | 670.62 | 123.59 |
| 3121 | 513.81 | 224.23 | 72% | 698 | 260.45 |
| 6673 | 697.31 | 124.67 | 71% | 713.3 | 302.28 |
| 4193 | 655.24 | 191.97 | 71% | 718.19 | 154.45 |
| 7552 | 709.86 | 131.78 | 73% | 813.29 | 320.57 |
| 820 | 636.5 | 127.73 | 71% | 821.94 | 204.55 |
| 19105 | 924.47 | 159.69 | 70% | 829.48 | 236.56 |
| 16169 | 456.68 | 219.61 | 72% | 862.69 | 796.4 |
| 20503 | 559 | 204.67 | 80% | 889.74 | 380.31 |
| 6236 | 529.47 | 148.78 | 79% | 903.06 | 433.66 |
| 16879 | 841.82 | 418.27 | 71% | 946.87 | 285.04 |
| 17340 | 1644.38 | 815.75 | 74% | 997.68 | 474.22 |
| 7451 | 1340.55 | 383.41 | 73% | 1014.34 | 341.2 |
| 12306 | 1456.43 | 258.06 | 79% | 1024.68 | 517.58 |
| 18905 | 880.62 | 169.73 | 78% | 1175.6 | 278.99 |
| 17027 | 844.61 | 248.1 | 71% | 1257.61 | 538.33 |
| 22554 | 997.94 | 184.01 | 86% | 1359.91 | 523.26 |
| 26147 | 1510.64 | 528.64 | 72% | 1410.78 | 338.29 |
| 9192 | 941.24 | 221.51 | 74% | 1413.17 | 565.76 |
| 23243 | 872.48 | 380.03 | 72% | 1417.04 | 675.7 |
| 16885 | 1012.98 | 320.39 | 72% | 1487.91 | 407.92 |
| 15029 | 1042.74 | 622.16 | 70% | 1488.18 | 539.06 |
| 4330 | 1083.48 | 398.15 | 72% | 1508.27 | 516.11 |
| 22266 | 1415.56 | 499.05 | 71% | 1514.02 | 441.93 |
| 18002 | 1259.73 | 300.25 | 77% | 1637.82 | 545.26 |
| 4933 | 1137.93 | 526.28 | 71% | 1700.05 | 608.74 |
| 21091 | 1307.31 | 329.46 | 70% | 1706.98 | 564.25 |
| 6072 | 1518.7 | 338.39 | 72% | 1859.25 | 511.2 |
| 17812 | 1406.92 | 373.38 | 70% | 1884.53 | 608.25 |
| 17107 | 1929.94 | 1307.4 | 71% | 2218.38 | 823.7 |
| 9016 | 1497.78 | 482.54 | 71% | 2267.81 | 949.1 |
| 20846 | 2090.67 | 1066.14 | 76% | 2478.45 | 898.34 |
| 22558 | 2580.09 | 1019.35 | 72% | 2867.4 | 846.53 |
| 6189 | 1470.69 | 763.08 | 73% | 2992.11 | 1673.91 |
| 11623 | 2359.03 | 1401.37 | 73% | 3039.92 | 2772.61 |
| 16884 | 1876.68 | 541.26 | 76% | 3308.78 | 4455.6 |
| 6018 | 1795.01 | 783.44 | 73% | 3626.1 | 3303 |

TABLE 3F
ANIT

Document Number 1650775

| | Group | Group | | Non Group | Non Group |
|---|---|---|---|---|---|
| GLGC ID | Mean | Stdev | LDA Score | Mean | Stdev |
| 22513 | 633.15 | 232.37 | 98% | −132.38 | 329.17 |
| 19388 | 29.83 | 17.06 | 91% | −25.03 | 31.57 |
| 72 | 49.9 | 30.74 | 90% | −17.96 | 34.45 |
| 489 | 86.15 | 31.02 | 99% | −11.18 | 21.72 |
| 11645 | 46.52 | 22.15 | 95% | −10.46 | 29.11 |
| 15003 | 103.65 | 34.94 | 91% | 5.13 | 35.34 |
| 4318 | 23.26 | 6.71 | 91% | 7.08 | 9.22 |
| 372 | 43.1 | 11.62 | 90% | 10.4 | 12.2 |
| 14400 | 115.49 | 28.78 | 96% | 12.11 | 47.49 |
| 15480 | 45.43 | 16.54 | 92% | 12.38 | 8.62 |
| 22397 | 98.15 | 29.08 | 90% | 18.38 | 61.47 |
| 23679 | 58.03 | 21.94 | 92% | 20.39 | 39.25 |
| 10790 | −79.79 | 34.37 | 91% | 24 | 51.35 |
| 16006 | 71.89 | 13.1 | 93% | 26.66 | 31.65 |
| 15701 | 115.07 | 45.82 | 92% | 29.52 | 22.06 |
| 25052 | 170.78 | 53.79 | 98% | 31.24 | 82.74 |
| 1221 | 221.03 | 65.82 | 92% | 36.47 | 104.6 |
| 23945 | 98.4 | 22.42 | 91% | 37.09 | 29.06 |
| 11608 | 68.37 | 11.81 | 92% | 39.75 | 16.9 |
| 20741 | 140.96 | 42.97 | 91% | 47.33 | 36.73 |
| 5384 | 110.15 | 33.33 | 91% | 48.7 | 63.05 |
| 1809 | 660.39 | 204.87 | 91% | 51.86 | 210.98 |
| 21088 | 88.49 | 15.38 | 90% | 52.62 | 15.58 |
| 488 | 302.77 | 84.83 | 99% | 55.29 | 40.85 |
| 20708 | 69.43 | 8.17 | 90% | 55.72 | 21.17 |
| 11940 | 79.89 | 7.9 | 90% | 56.21 | 16.71 |
| 6585 | 124.92 | 40.67 | 93% | 56.76 | 84.64 |
| 15914 | 167.68 | 28.59 | 98% | 58.06 | 29.32 |
| 1279 | 124.99 | 36.23 | 92% | 60.16 | 22.09 |
| 22487 | 203.14 | 70.64 | 92% | 66.54 | 38.82 |
| 17894 | 123.11 | 19.61 | 91% | 68.4 | 25.56 |
| 2801 | 158.72 | 27.08 | 95% | 68.44 | 49.17 |
| 14465 | 5.28 | 16.66 | 90% | 70.62 | 29.14 |
| 15892 | 279.1 | 77.25 | 95% | 73.2 | 79.81 |
| 7903 | 9.08 | 6.85 | 90% | 75.62 | 75.73 |
| 20772 | 127.51 | 24.47 | 94% | 79.34 | 26.84 |
| 11904 | 152.49 | 15.73 | 96% | 81.95 | 37.81 |
| 23522 | 149.93 | 28.04 | 91% | 84.93 | 35.96 |
| 14017 | 168.86 | 47.57 | 91% | 94.1 | 25.48 |
| 23869 | 219.91 | 36.9 | 95% | 98.3 | 110.47 |
| 14016 | 172.79 | 34.4 | 91% | 101.88 | 27.02 |
| 23005 | 231.25 | 60.04 | 96% | 102.75 | 100.99 |
| 24453 | 296.76 | 77.39 | 97% | 107.86 | 52.64 |
| 23872 | 208.24 | 51.83 | 93% | 110.93 | 125.84 |
| 10016 | 224.63 | 64.84 | 91% | 116.67 | 48.65 |
| 17590 | 228.93 | 49.97 | 90% | 127.17 | 38.31 |
| 4944 | 218.13 | 56.11 | 93% | 129.57 | 134.8 |
| 15002 | 208.14 | 35.44 | 90% | 134.25 | 36.07 |
| 20529 | 372.92 | 69.59 | 93% | 138.52 | 121.65 |
| 20849 | 259.34 | 55.56 | 91% | 150.94 | 38.19 |
| 15141 | 216.05 | 18.73 | 91% | 161.78 | 36.17 |
| 15089 | 428.71 | 94.42 | 90% | 164.31 | 111.52 |
| 24779 | −119.55 | 53.79 | 90% | 169.39 | 275.44 |
| 7665 | 325.89 | 51.47 | 94% | 171.6 | 94 |
| 12577 | 530.07 | 99.18 | 92% | 176.81 | 126.07 |
| 3253 | 242.21 | 21.26 | 92% | 177.78 | 42.54 |
| 25069 | 384.72 | 63.15 | 96% | 181.27 | 147.24 |
| 23182 | 70.96 | 27.02 | 90% | 182.67 | 82.66 |
| 19043 | 461.37 | 93.08 | 91% | 184.16 | 86.52 |
| 23445 | 44.92 | 13.64 | 96% | 204.01 | 96.17 |
| 22928 | 18.25 | 13.42 | 90% | 205.31 | 168.08 |
| 15300 | 301.52 | 31.01 | 95% | 208.5 | 106.84 |
| 19073 | 357.79 | 55.66 | 90% | 215.38 | 51.37 |
| 24237 | 602.69 | 44.81 | 99% | 219.11 | 138.4 |
| 1447 | 293.32 | 18.87 | 94% | 221.41 | 41.58 |
| 16408 | 151.08 | 35.06 | 90% | 254.15 | 84.03 |
| 23868 | 529.77 | 129.48 | 90% | 266.34 | 657.93 |
| 24810 | 103 | 36.24 | 90% | 273.16 | 90.15 |
| 5235 | 460.06 | 75.16 | 90% | 286.43 | 79.01 |
| 2802 | 498.79 | 58.22 | 95% | 287.5 | 90.87 |
| 25747 | 698.21 | 163.03 | 91% | 318.26 | 115.19 |
| 2818 | 510.22 | 88.82 | 94% | 330.07 | 92.39 |
| 5934 | 42.22 | 26 | 94% | 342.34 | 187.09 |
| 1501 | 711.93 | 121.22 | 96% | 348.6 | 117.83 |
| 15535 | 499.6 | 40.24 | 91% | 391.06 | 75.12 |
| 5437 | 327.15 | 25.07 | 90% | 409.5 | 102.21 |
| 12928 | 607.12 | 43.69 | 97% | 411.1 | 97.29 |
| 4207 | 611.82 | 98.48 | 90% | 440.38 | 323.23 |
| 20701 | 762.37 | 110.98 | 94% | 496.87 | 170.59 |
| 1562 | 360.31 | 37.96 | 90% | 504.85 | 111.39 |
| 6824 | 806.51 | 180.29 | 90% | 506.91 | 368.25 |
| 20983 | 343.07 | 66.3 | 93% | 516.16 | 120.95 |
| 13088 | 199.67 | 54 | 96% | 593.92 | 183.67 |
| 6613 | 320.2 | 65.66 | 92% | 626.43 | 272.37 |
| 25024 | 451.39 | 46.56 | 91% | 661.12 | 185.97 |
| 8549 | 262.14 | 62.15 | 93% | 665.65 | 258.33 |
| 4193 | 484.74 | 47.1 | 95% | 719.76 | 154.17 |
| 2569 | 257.19 | 110.15 | 91% | 724.41 | 288.37 |
| 7892 | 1166.36 | 244.14 | 92% | 809.73 | 244.53 |
| 18900 | 1202.22 | 137.08 | 92% | 830.76 | 217.68 |
| 16879 | 540.35 | 100.54 | 93% | 949.72 | 286.7 |
| 475 | 635.1 | 94.59 | 92% | 976.05 | 230.62 |
| 5899 | 704.5 | 125.15 | 92% | 1227.29 | 427.31 |
| 3916 | 883.71 | 181.1 | 91% | 1427.83 | 464.67 |
| 10378 | 2563.09 | 466.04 | 90% | 1469.47 | 449.7 |
| 19363 | 372.52 | 212.88 | 90% | 1539.84 | 830.44 |
| 6072 | 1270.16 | 177.57 | 91% | 1859.03 | 508.9 |
| 20502 | 1504.84 | 383.84 | 91% | 3017.48 | 1038.48 |

TABLE 3G
Late Acetaminophen

Document Number 1650775

| | Group | Group | | Non Group | Non Group |
|---|---|---|---|---|---|
| GLGC ID | Mean | Stdev | LDA Score | Mean | Stdev |
| 18028 | 62.86 | 12.89 | 98% | 11.46 | 17.68 |
| 6151 | 41.98 | 5.06 | 97% | 11.63 | 19.32 |
| 1394 | 46.55 | 7.94 | 98% | 13.22 | 8.97 |
| 15701 | 104.85 | 30.26 | 98% | 29.54 | 22.64 |
| 21586 | 129.12 | 22.29 | 98% | 37.42 | 35.11 |
| 18099 | 74.54 | 10.03 | 98% | 37.77 | 12.82 |
| 18990 | 191.58 | 50.21 | 98% | 37.78 | 56 |
| 5492 | 154.99 | 36.3 | 98% | 42.55 | 45.33 |
| 16958 | 152.1 | 24.97 | 99% | 48.17 | 21.95 |
| 25892 | 5.84 | 14.89 | 97% | 52.01 | 13.92 |
| 4281 | 8.04 | 4.69 | 97% | 52.71 | 20.31 |
| 20817 | 552.74 | 204.49 | 99% | 56.23 | 83.19 |
| 494 | −58.87 | 15.28 | 99% | 57.66 | 57 |
| 17091 | 221.12 | 37.22 | 99% | 64.55 | 35.7 |
| 5493 | 201.07 | 32.69 | 98% | 68.52 | 42.64 |
| 4650 | 257.12 | 41.99 | 98% | 74.24 | 55.94 |
| 20818 | 387.65 | 157.18 | 99% | 81.37 | 42.47 |
| 8356 | 191.89 | 39.3 | 98% | 81.94 | 31.64 |
| 17090 | 166.91 | 23.91 | 98% | 82.55 | 25.23 |
| 6153 | 47.01 | 7.23 | 98% | 89.68 | 30.74 |
| 1399 | 422.27 | 102.52 | 97% | 118.53 | 72.23 |
| 18369 | 14.78 | 33.12 | 98% | 154.92 | 43.99 |
| 8107 | 82.52 | 12.58 | 99% | 157.67 | 30.22 |
| 21305 | 78.03 | 11.47 | 97% | 162.22 | 42.69 |
| 16219 | 91.23 | 10.22 | 97% | 162.24 | 35.05 |
| 20380 | 51.46 | 16.74 | 97% | 164.24 | 55.84 |
| 14970 | 64.35 | 7.2 | 98% | 165.35 | 37.88 |
| 11039 | 22.92 | 14.76 | 98% | 165.75 | 75.12 |
| 1644 | 69.04 | 14.22 | 99% | 166.93 | 43.07 |
| 25632 | 23.75 | 9.64 | 100% | 170.77 | 437.48 |
| 25069 | 648.62 | 107.28 | 98% | 177.18 | 137.77 |
| 12848 | 77.84 | 12.22 | 98% | 178.82 | 51.97 |
| 15571 | 37.5 | 7.71 | 100% | 182.36 | 613.17 |
| 5998 | 82.64 | 16 | 98% | 198.22 | 47.74 |
| 1542 | 75.63 | 15.75 | 97% | 201.9 | 67.93 |
| 11429 | 113.75 | 15.07 | 97% | 220.8 | 45.17 |
| 11635 | 84.37 | 10.31 | 100% | 235.11 | 58.7 |
| 24246 | 680.67 | 154.62 | 97% | 235.68 | 110.38 |
| 17684 | 115.68 | 11.83 | 97% | 243.52 | 58.44 |
| 1479 | 111.19 | 13.1 | 98% | 246.79 | 62.43 |
| 16023 | 118.74 | 16.82 | 97% | 262.5 | 67.56 |
| 20986 | 100.65 | 16.03 | 98% | 269.03 | 97.64 |
| 23033 | 164.75 | 20.5 | 97% | 269.22 | 53.32 |
| 24810 | 78 | 27.42 | 97% | 273.76 | 89.28 |
| 8592 | 97.92 | 12.74 | 99% | 275.69 | 78.69 |
| 12156 | 66.84 | 25.24 | 99% | 279.94 | 158.15 |
| 20555 | 74.21 | 32.18 | 97% | 280.75 | 96.14 |
| 18837 | 70.96 | 24.35 | 98% | 281.18 | 112.85 |
| 17758 | 47.9 | 17.49 | 98% | 283.74 | 151.83 |
| 11152 | 89.81 | 23.98 | 98% | 284.55 | 88.62 |
| 22582 | 97.84 | 15.79 | 98% | 290.41 | 88.62 |
| 6155 | 86.76 | 17.03 | 100% | 302.82 | 149.97 |
| 10093 | 894.21 | 296.81 | 97% | 307.41 | 125.35 |
| 23854 | 518.98 | 43.24 | 97% | 317.71 | 83.8 |
| 4314 | 161.66 | 22.27 | 99% | 325.66 | 70.88 |
| 20864 | 896.29 | 162.64 | 98% | 340.85 | 169.02 |
| 9072 | 134.11 | 29.83 | 97% | 372.6 | 132.4 |
| 15462 | 187.89 | 20.53 | 99% | 377.51 | 69.64 |
| 3023 | 74.88 | 27.06 | 99% | 377.75 | 123.14 |
| 1529 | 196.76 | 20.46 | 97% | 378.11 | 72.49 |
| 24670 | 211.91 | 19.4 | 98% | 380.22 | 75.72 |
| 25480 | 139.68 | 36.79 | 97% | 384.92 | 88.4 |
| 4224 | 217.33 | 27.1 | 98% | 385.39 | 68.02 |
| 1653 | 161.77 | 30.91 | 99% | 413.84 | 133.06 |
| 9905 | 215.17 | 33.74 | 97% | 417.78 | 81.53 |
| 11153 | 184.99 | 26.78 | 98% | 424.64 | 112.76 |
| 21977 | 167.03 | 43.78 | 97% | 425.7 | 100.74 |
| 21950 | 225.05 | 28.55 | 97% | 431.25 | 83.14 |
| 2505 | 181.37 | 17.8 | 99% | 437.97 | 99.3 |
| 794 | 185.22 | 23.41 | 98% | 452.2 | 109.84 |
| 5920 | 1687.13 | 555.96 | 99% | 456.93 | 241.47 |
| 2667 | 266.65 | 38.11 | 98% | 472.54 | 95.54 |
| 24722 | 177.21 | 38.39 | 99% | 491.55 | 112.03 |
| 23390 | 1178.14 | 133.27 | 98% | 504.75 | 225.74 |
| 1562 | 261.12 | 32.84 | 98% | 506.49 | 108.81 |
| 15113 | 155.11 | 52.14 | 98% | 515.14 | 163.96 |
| 4199 | 289.55 | 26.97 | 98% | 519.47 | 108.02 |
| 8872 | 1732.12 | 253.22 | 99% | 539.58 | 281.13 |
| 24771 | 204.77 | 35.86 | 99% | 548.56 | 123.7 |
| 13088 | 127.47 | 50.84 | 97% | 595.53 | 180.73 |
| 17541 | 1185.11 | 145.34 | 98% | 686.63 | 152.47 |
| 24811 | 244.05 | 55.21 | 98% | 713.37 | 236.19 |
| 24321 | 133.15 | 53.97 | 98% | 767.37 | 279.51 |
| 7552 | 180.78 | 39.85 | 98% | 820.01 | 310.92 |
| 19732 | 145.53 | 28.91 | 98% | 918.79 | 410.43 |
| 11205 | 330.78 | 77.32 | 97% | 976.22 | 280.85 |
| 15673 | 1721.01 | 183.17 | 98% | 1022.66 | 229.71 |
| 14512 | 230.44 | 36.6 | 99% | 1088.1 | 390.72 |
| 11850 | 2429.93 | 244.48 | 98% | 1189.68 | 370.45 |
| 633 | 647.11 | 128.95 | 97% | 1346.47 | 304.28 |
| 14960 | 3443.82 | 469.79 | 99% | 1352.48 | 446.55 |
| 22554 | 383.07 | 75.73 | 98% | 1365.63 | 511.2 |
| 24049 | 4317.73 | 1756.71 | 97% | 1441.54 | 440.22 |
| 2587 | 661.56 | 121.75 | 98% | 1598.85 | 493.87 |
| 12314 | 743.43 | 156.24 | 98% | 2014.22 | 647.46 |
| 15315 | 4723.83 | 784.41 | 97% | 2482.27 | 635.01 |
| 17730 | 6017.72 | 1076.55 | 98% | 2933.25 | 821.08 |
| 6189 | 422.42 | 136.09 | 97% | 2994.06 | 1657.8 |
| 20873 | 5487.66 | 1292.77 | 97% | 3014.46 | 6409.47 |

TABLE 3H
Early Acetominophen

Document Number 1650775

| | Group | Group | | Non Group | Non Group |
|---|---|---|---|---|---|
| GLGC ID | Mean | Stdev | LDA Score | Mean | Stdev |
| 21175 | 8.2 | 4.71 | 94% | 28.82 | 12.57 |
| 7528 | 8.32 | 4.93 | 95% | 34.66 | 16.43 |
| 20282 | −15.7 | 9.27 | 92% | 36.02 | 33.93 |
| 5966 | −2.42 | 11.53 | 95% | 36.31 | 21.84 |
| 22695 | 10.13 | 6.89 | 92% | 38.79 | 17.51 |
| 15634 | 1.39 | 5.65 | 94% | 39.68 | 19.47 |
| 1520 | 15.99 | 5.3 | 94% | 47.93 | 19.37 |
| 16524 | 20.02 | 6.63 | 94% | 48.44 | 13.24 |
| 18482 | 16.24 | 5.44 | 95% | 48.47 | 17.05 |
| 2280 | 19.83 | 5.96 | 93% | 49.02 | 23.16 |
| 19787 | 15.18 | 6.28 | 94% | 50.55 | 15.04 |
| 18584 | 6.53 | 10.13 | 95% | 51.53 | 23.14 |
| 13926 | 21.46 | 6.96 | 92% | 52.65 | 14.76 |
| 11423 | 15.02 | 8.15 | 94% | 56.28 | 19.95 |
| 11940 | 21.79 | 9.2 | 93% | 57.53 | 15.9 |
| 23000 | 22.53 | 12.08 | 93% | 57.77 | 15.01 |
| 3080 | −6.92 | 14.95 | 93% | 58.31 | 48.7 |
| 23710 | 158.41 | 53.72 | 92% | 58.38 | 71.02 |
| 23047 | 15.29 | 11.17 | 95% | 58.49 | 16.56 |
| 16566 | 17.77 | 6.03 | 98% | 58.51 | 15.69 |
| 19650 | −70.3 | 47.02 | 93% | 61.72 | 44.09 |
| 15467 | 11.36 | 7.01 | 95% | 62.46 | 46.17 |
| 16728 | 14.72 | 12.75 | 92% | 64.03 | 32.75 |
| 13568 | 28.12 | 10.02 | 94% | 67.08 | 17.03 |
| 13932 | −112.44 | 63.3 | 94% | 67.38 | 48.47 |
| 15139 | 21.25 | 9.99 | 96% | 68.11 | 25.84 |
| 24079 | 25.3 | 8.6 | 95% | 69.08 | 26.17 |
| 22487 | 6.73 | 8.7 | 98% | 70.08 | 41.42 |
| 14139 | 19.82 | 7.55 | 95% | 71.65 | 22.54 |
| 15181 | 26.59 | 10.69 | 94% | 79.78 | 30.61 |
| 23077 | 38.94 | 17.17 | 92% | 81.22 | 21.14 |
| 17158 | 17.52 | 10.77 | 94% | 83.01 | 45.36 |
| 20971 | 43.32 | 10.04 | 92% | 83.29 | 21.37 |
| 1169 | 27.52 | 12.64 | 92% | 83.96 | 30.23 |
| 16871 | 19.55 | 12.49 | 93% | 85.46 | 26.85 |
| 9164 | 27.2 | 10.23 | 95% | 85.81 | 27.4 |
| 15980 | 26.43 | 18.24 | 93% | 86.7 | 23.87 |
| 16361 | 43.56 | 12.22 | 92% | 91.15 | 25.64 |
| 21321 | 27.09 | 14.56 | 93% | 105.32 | 56.02 |
| 3486 | 34.72 | 10.49 | 97% | 107.9 | 41.25 |
| 2727 | 45.87 | 10.75 | 92% | 110.53 | 48.76 |
| 8597 | 69.34 | 16.36 | 93% | 116.43 | 40.21 |
| 574 | 65.57 | 6.51 | 93% | 117.45 | 179.89 |
| 8730 | 45.4 | 17.81 | 92% | 119.22 | 42.05 |
| 13351 | 36.93 | 12.29 | 95% | 122.54 | 50.81 |
| 6330 | 28.64 | 17.18 | 98% | 123.06 | 58.01 |
| 18829 | 33.89 | 17.14 | 94% | 128.07 | 58.85 |
| 16134 | 18.36 | 24.36 | 94% | 128.31 | 40.65 |
| 20975 | 70.64 | 13.75 | 93% | 135.77 | 31.44 |
| 64 | 64.42 | 13.23 | 93% | 141.31 | 35.51 |
| 11426 | 36.73 | 16.99 | 94% | 143.85 | 61.64 |
| 4127 | 42.82 | 25.2 | 92% | 147.26 | 55.78 |
| 2043 | 94.32 | 14.17 | 93% | 149.89 | 35.38 |
| 25814 | 49.58 | 15.47 | 93% | 150.18 | 60.26 |
| 23044 | 256.5 | 54.33 | 94% | 154.34 | 33.61 |
| 23491 | 80.29 | 14.78 | 92% | 156.45 | 57.06 |
| 21909 | 77.01 | 15.95 | 92% | 157.72 | 48.89 |
| 16364 | 54.12 | 18.74 | 92% | 161.04 | 68.62 |
| 6861 | 53.34 | 24.76 | 95% | 173.75 | 47.49 |
| 23709 | 365.56 | 102.97 | 92% | 174.65 | 139.26 |
| 18981 | 80.53 | 12.18 | 98% | 180 | 124.54 |
| 18136 | 92.28 | 22.73 | 96% | 180.63 | 44.47 |
| 15170 | 63.67 | 31 | 93% | 182.69 | 57.04 |
| 15491 | 50.3 | 18.75 | 94% | 184.71 | 62.38 |
| 13640 | 81.51 | 25.5 | 94% | 194.43 | 69.6 |
| 1542 | 110.94 | 15.7 | 93% | 202.72 | 68.33 |
| 23711 | 965.1 | 437.75 | 93% | 203.15 | 366.12 |
| 3549 | 100.08 | 20.01 | 93% | 203.26 | 64.36 |
| 5749 | 105.17 | 17.76 | 96% | 203.46 | 50.97 |
| 1921 | 469.15 | 75.54 | 94% | 203.88 | 88.71 |
| 5953 | 1395.67 | 589.94 | 92% | 204.16 | 203.2 |
| 11179 | 51.98 | 16.53 | 97% | 213.56 | 68.01 |
| 17571 | 121.22 | 22.36 | 91% | 215.28 | 47.28 |
| 1919 | 540.5 | 142.58 | 94% | 224.99 | 91 |
| 16449 | −17.52 | 49.15 | 92% | 225.71 | 118.83 |
| 7927 | 58.81 | 47.71 | 94% | 235.03 | 77.05 |
| 8735 | 104.51 | 40.55 | 92% | 260.2 | 118.96 |
| 15070 | 64.72 | 20.64 | 92% | 276.22 | 127.77 |
| 23606 | 645.68 | 142.54 | 92% | 308.45 | 97.73 |
| 4291 | 55.74 | 33.3 | 95% | 309.48 | 143.72 |
| 6366 | 132.6 | 38.47 | 93% | 309.95 | 143.06 |
| 22862 | 102.99 | 68.89 | 92% | 331.29 | 84.1 |
| 1920 | 699.35 | 125.66 | 94% | 334.22 | 116.2 |
| 23230 | 101.11 | 53.57 | 94% | 347.39 | 161.95 |
| 1802 | 68.01 | 68.24 | 93% | 348.21 | 129.62 |
| 1501 | 135.65 | 55.72 | 93% | 359.59 | 120.35 |
| 3143 | 180.22 | 37.55 | 93% | 360.43 | 101.81 |
| 20799 | 195.78 | 28.73 | 95% | 368.39 | 68.29 |
| 21980 | 205.1 | 26.69 | 96% | 380.01 | 105.72 |
| 4234 | 728.11 | 88.4 | 91% | 441.47 | 146.01 |
| 16215 | 277.82 | 31.3 | 92% | 468.47 | 103.74 |
| 25705 | 303.85 | 36.79 | 95% | 471.16 | 88.31 |
| 164 | 290.9 | 32.23 | 97% | 476.12 | 84.6 |
| 21097 | 844.93 | 124.78 | 93% | 521.05 | 142.52 |
| 23139 | 297.32 | 105.82 | 94% | 614.3 | 226.46 |
| 8549 | 197.64 | 79.57 | 92% | 674.01 | 251.68 |
| 9190 | 372.68 | 47.07 | 94% | 1016.16 | 415.34 |
| 6291 | 552.9 | 84.63 | 97% | 1091 | 307.85 |

TABLE 3I
Late Carbon Tetrachloride

Document Number 1650775

| | Group | Group | | Non Group | Non Group |
|---|---|---|---|---|---|
| GLGC ID | Mean | Stdev | LDA Score | Mean | Stdev |
| 17064 | 50.24 | 16.97 | 96% | −4.18 | 20 |
| 1625 | 114.41 | 34.24 | 99% | 0.07 | 12.89 |
| 5885 | 38.36 | 18.29 | 97% | 1.99 | 9.82 |
| 18046 | 46.73 | 12.92 | 99% | 2.71 | 14.04 |
| 16649 | 220.02 | 92.9 | 99% | 3.43 | 37.53 |
| 1554 | 47.01 | 20.46 | 98% | 4.33 | 6.64 |
| 20950 | 54.4 | 13.02 | 98% | 6.19 | 12 |
| 13458 | 58.51 | 18.25 | 97% | 6.84 | 20.17 |
| 6879 | 53.86 | 20.46 | 98% | 10.45 | 8.61 |
| 2065 | 77.67 | 43.56 | 98% | 14.07 | 10.39 |
| 16654 | 153.26 | 64.25 | 99% | 14.11 | 9.91 |
| 23651 | 330.28 | 228.17 | 97% | 21.42 | 37.58 |
| 15312 | 116.71 | 36.41 | 96% | 25.99 | 29.2 |
| 21818 | 119.6 | 30.36 | 97% | 26.66 | 21.99 |
| 4048 | 1573.97 | 2042.27 | 100% | 28.72 | 92.76 |
| 21695 | 174.77 | 50.28 | 99% | 30.87 | 22.35 |
| 1126 | 93.96 | 18.28 | 98% | 31.78 | 16.86 |
| 17157 | 116.08 | 34.36 | 98% | 33.37 | 18.38 |
| 21586 | 155.13 | 41.01 | 98% | 35.85 | 31.46 |
| 4097 | 202.62 | 143.18 | 96% | 36.77 | 20.82 |
| 20589 | 204.58 | 80.85 | 99% | 39.66 | 14.51 |
| 4856 | 195.72 | 58.45 | 98% | 44.87 | 22.87 |
| 17500 | 1.65 | 7.49 | 96% | 45.77 | 44.45 |
| 16730 | 154.98 | 38.01 | 97% | 46.39 | 26.25 |
| 20449 | 440.43 | 164.04 | 98% | 47.45 | 46.4 |
| 15655 | 237.45 | 149.71 | 98% | 48.19 | 26.25 |
| 19040 | 396.02 | 114.12 | 99% | 54.95 | 29.77 |
| 1037 | 191.13 | 61.49 | 99% | 55.16 | 22.83 |
| 4178 | 263.2 | 73.51 | 99% | 58.46 | 46.4 |
| 23302 | 134 | 32.72 | 97% | 60.71 | 24.04 |
| 21060 | 195.49 | 44.63 | 99% | 66.73 | 22.3 |
| 2781 | 300.75 | 90.51 | 100% | 67.08 | 21.7 |
| 1571 | 306.34 | 84.06 | 98% | 69.24 | 44.27 |
| 1258 | 201.18 | 53.89 | 99% | 69.76 | 26.45 |
| 20755 | 315.54 | 99.4 | 98% | 70.92 | 37.08 |
| 21416 | 180.67 | 33.54 | 98% | 71.26 | 32.81 |
| 4327 | 209.63 | 44.69 | 97% | 73.46 | 30.98 |
| 2853 | 243.76 | 74.49 | 99% | 79.5 | 27.62 |
| 14458 | 462.45 | 169.29 | 97% | 79.77 | 81.9 |
| 17956 | 135.44 | 24.53 | 96% | 80.41 | 19.61 |
| 16650 | 335.98 | 95.22 | 99% | 82.71 | 42.71 |
| 8152 | 184.75 | 44.1 | 98% | 84.34 | 21.12 |
| 22321 | 565.88 | 166.7 | 98% | 90.43 | 44.8 |
| 20801 | 244.26 | 53.66 | 97% | 93.54 | 45.27 |
| 15203 | 217.53 | 41.56 | 99% | 94.08 | 22.2 |
| 16683 | 214.61 | 51.64 | 98% | 96.97 | 26.38 |
| 7690 | 485.59 | 136.48 | 97% | 98.07 | 100.2 |
| 18705 | 230.49 | 55.83 | 99% | 103.84 | 19.16 |
| 574 | 566.67 | 151.26 | 99% | 104.84 | 163.13 |
| 20644 | 284.09 | 69.38 | 96% | 104.86 | 53.3 |
| 12613 | 385.02 | 81.17 | 98% | 105.74 | 49.08 |
| 23173 | 527.13 | 156.81 | 99% | 112.95 | 62.38 |
| 10016 | 305.83 | 117.64 | 98% | 113.41 | 37.12 |
| 25257 | 401.37 | 69.21 | 98% | 123.93 | 52.05 |
| 19377 | 245.39 | 39.45 | 98% | 124.66 | 31.89 |
| 25313 | 368.62 | 55.36 | 99% | 125.11 | 47.2 |
| 23888 | 323.47 | 71.72 | 99% | 127.05 | 34.78 |
| 17754 | 280.21 | 65.27 | 98% | 127.56 | 39.49 |
| 20891 | 284.25 | 57.73 | 96% | 128.54 | 57.37 |
| 19241 | 305.11 | 61.55 | 99% | 128.91 | 25.25 |
| 17369 | 251.93 | 28.1 | 96% | 130.99 | 61.88 |
| 4049 | 1800.21 | 615.67 | 99% | 131.28 | 173.33 |
| 4426 | 226.63 | 33.81 | 98% | 134.21 | 26.79 |
| 15282 | 495.77 | 127.65 | 97% | 140.76 | 88.42 |
| 20849 | 288.07 | 45.99 | 98% | 148.97 | 33.86 |
| 17225 | 314.55 | 56.91 | 96% | 156.73 | 51.3 |
| 24388 | 756.8 | 218.92 | 98% | 158.69 | 122.1 |
| 16854 | 274.55 | 32.55 | 98% | 161.83 | 29.13 |
| 16610 | 376.93 | 79.48 | 97% | 165.18 | 49.27 |
| 6193 | 447.67 | 59.78 | 99% | 194.57 | 54.15 |
| 3549 | 368.01 | 54.43 | 97% | 196.19 | 60.45 |
| 2744 | 487.89 | 65.94 | 98% | 202.98 | 55.42 |
| 15281 | 509.13 | 65.19 | 98% | 207.9 | 69.15 |
| 17571 | 337.5 | 57.58 | 97% | 209.52 | 44.91 |
| 8928 | 323.46 | 31.08 | 98% | 210.05 | 36.77 |
| 25802 | 411.96 | 57.18 | 98% | 210.79 | 57.41 |
| 12551 | 48.43 | 13.62 | 98% | 212.69 | 71.68 |
| 7602 | 453.04 | 80.74 | 97% | 213.06 | 62.29 |
| 15543 | 555.28 | 110.77 | 97% | 219.06 | 83.33 |
| 958 | 492.73 | 90.77 | 98% | 234.42 | 59.68 |
| 2854 | 520.08 | 129.87 | 99% | 239.21 | 54.99 |
| 5331 | 517.46 | 66.57 | 99% | 253.08 | 62.49 |
| 23013 | 631.62 | 255.14 | 98% | 253.69 | 77.98 |
| 19768 | 497.6 | 88.61 | 97% | 258.31 | 86.39 |
| 18107 | 475.79 | 86.06 | 98% | 270.37 | 50.73 |
| 10306 | 537.72 | 79 | 97% | 270.7 | 72.51 |
| 3138 | 773.53 | 129.57 | 99% | 280.59 | 128.8 |
| 16684 | 591.01 | 105.06 | 98% | 303.32 | 77.67 |
| 23854 | 563.93 | 104.51 | 97% | 314.55 | 77.09 |
| 20897 | 602.65 | 120.81 | 96% | 315.7 | 85.83 |
| 19298 | 835.39 | 188.74 | 97% | 328.8 | 152.97 |
| 25718 | 579.2 | 77.87 | 98% | 328.95 | 68.42 |
| 14959 | 676.74 | 116.99 | 97% | 377.46 | 94.35 |
| 20879 | 73.93 | 55.35 | 98% | 390.34 | 126.05 |
| 6824 | 1794.5 | 585.37 | 97% | 479.02 | 298.25 |
| 13684 | 1052.78 | 207.71 | 96% | 578.09 | 181.33 |
| 16438 | 1299.24 | 155.02 | 99% | 582.93 | 144.92 |
| 4193 | 332.28 | 95.67 | 96% | 726.26 | 144.3 |
| 7552 | 163.75 | 89.31 | 97% | 826.93 | 304.52 |
| 16883 | 681.46 | 275.09 | 96% | 1856.78 | 528.87 |

TABLE 3J
Early Carbon Tetrachloride

Document Number 1650775

| | Group | Group | | Non Group | Non Group |
|---|---|---|---|---|---|
| GLGC ID | Mean | Stdev | LDA Score | Mean | Stdev |
| 8663 | 721.93 | 225.97 | 97% | −87.65 | 146.96 |
| 8662 | 653.64 | 143.71 | 99% | −66.58 | 95.42 |
| 1727 | 348.89 | 185.42 | 95% | −57.26 | 75.16 |
| 11493 | 129.55 | 67.26 | 96% | −32.97 | 39.87 |
| 2628 | 251.75 | 147.92 | 96% | 8.65 | 34 |
| 15647 | 109.5 | 26.81 | 94% | 11.25 | 155.64 |
| 13265 | 78.29 | 37.64 | 97% | 12.05 | 9.28 |
| 923 | 199.22 | 94.23 | 95% | 15.81 | 23.49 |
| 8661 | 614.42 | 215.98 | 99% | 16.84 | 60.47 |
| 7301 | 187.05 | 149.7 | 95% | 19.02 | 15.94 |
| 15312 | 129.52 | 34.52 | 94% | 23.98 | 24.69 |
| 1305 | 159.8 | 80 | 94% | 27.12 | 24.91 |
| 1598 | 232.56 | 58.02 | 96% | 28.01 | 58.64 |
| 23567 | 918.41 | 595.26 | 94% | 30.79 | 97.73 |
| 25198 | 145.62 | 46.46 | 97% | 31.18 | 21.37 |
| 22443 | 413.57 | 187.24 | 96% | 32.31 | 38.97 |
| 809 | 170.72 | 83.79 | 94% | 33 | 26.32 |
| 18043 | 157.01 | 66.2 | 95% | 35.05 | 27.16 |
| 16825 | 86.21 | 14.87 | 95% | 36.95 | 15.49 |
| 11494 | 365.78 | 87.61 | 98% | 39.57 | 52.58 |
| 12969 | 315.69 | 145.09 | 97% | 39.62 | 30.17 |
| 347 | 94.32 | 20.45 | 94% | 44.31 | 19.5 |
| 15313 | 188.23 | 47.79 | 95% | 44.81 | 34.49 |
| 25907 | 196.63 | 51.46 | 96% | 45.95 | 29.69 |
| 2629 | 258.22 | 130.51 | 94% | 47.27 | 31.18 |
| 4119 | 172.99 | 53.46 | 96% | 49.1 | 27.57 |
| 15617 | 131.28 | 26.96 | 94% | 49.13 | 28.01 |
| 11483 | 356.15 | 129.53 | 95% | 49.85 | 57.22 |
| 25098 | 263.21 | 101.83 | 95% | 51.71 | 35.09 |
| 8664 | 685.72 | 187.22 | 98% | 51.77 | 117.57 |
| 7806 | 173.92 | 56.36 | 95% | 51.78 | 24.26 |
| 5932 | 142.26 | 26.26 | 94% | 51.91 | 24.37 |
| 18501 | 128.83 | 31.95 | 94% | 53.7 | 17.47 |
| 352 | 306.66 | 117.09 | 94% | 53.93 | 48.46 |
| 3831 | 120.45 | 24.02 | 95% | 55.42 | 25.76 |
| 651 | 234.03 | 95.8 | 96% | 55.88 | 31.26 |
| 650 | 252.68 | 84.65 | 96% | 57.08 | 37.09 |
| 17337 | 140.87 | 38.01 | 95% | 60.97 | 56.3 |
| 7036 | 176.78 | 42.65 | 98% | 62.22 | 22.87 |
| 22124 | 125.04 | 23.89 | 94% | 64.53 | 17.38 |
| 23587 | 208.43 | 60.7 | 94% | 66.37 | 32.19 |
| 21130 | 369.23 | 131.33 | 98% | 72.63 | 40.41 |
| 353 | 475.4 | 152.81 | 94% | 76.96 | 69.6 |
| 1183 | 426.68 | 140.86 | 99% | 78.14 | 33.96 |
| 16080 | 464.2 | 128.58 | 94% | 81.55 | 87.93 |
| 18349 | 210.66 | 61.07 | 98% | 82.84 | 26.6 |
| 19184 | 623.72 | 284.24 | 97% | 83.93 | 71.71 |
| 2788 | 214.08 | 67.37 | 95% | 87.98 | 29.5 |
| 15291 | 225.71 | 67.73 | 96% | 89.73 | 24.64 |
| 21380 | 195.27 | 36.2 | 95% | 90.84 | 24.55 |
| 17908 | 489.98 | 67.94 | 99% | 91.5 | 64.42 |
| 1475 | 764.62 | 270.51 | 94% | 95.88 | 162.38 |
| 354 | 549.22 | 181.76 | 94% | 96.35 | 76.24 |
| 14424 | 1887.85 | 604.98 | 95% | 104.46 | 294.14 |
| 23438 | 233.78 | 45.73 | 94% | 105.37 | 42.63 |
| 19085 | 235.47 | 46.91 | 96% | 105.97 | 34.08 |
| 16318 | 569.79 | 137.14 | 98% | 106.93 | 68.65 |
| 19641 | 354.6 | 119.72 | 94% | 111.15 | 52.02 |
| 2049 | 351.74 | 96.17 | 96% | 113.35 | 54.16 |
| 22625 | 588.59 | 137.7 | 98% | 119.99 | 73.04 |
| 15616 | 363.79 | 100.12 | 94% | 126.33 | 57.91 |
| 16081 | 590.52 | 148.03 | 94% | 131.04 | 114.9 |
| 1306 | 354.57 | 112.94 | 96% | 131.39 | 47.78 |
| 5489 | 361.63 | 79.95 | 96% | 135.76 | 55.44 |
| 19086 | 312.97 | 47.23 | 96% | 137.05 | 43.97 |
| 22681 | 1733.5 | 1045.76 | 94% | 138.8 | 233.99 |
| 25567 | 440.46 | 120.5 | 94% | 146.39 | 68.31 |
| 5820 | 392.73 | 112.42 | 94% | 148.03 | 58.75 |
| 19075 | 541.95 | 182.12 | 95% | 149.36 | 55.34 |
| 8314 | 4119.47 | 2769.99 | 98% | 151.41 | 501.27 |
| 24234 | 520.49 | 130.96 | 97% | 152.5 | 60.67 |
| 15490 | 337.2 | 71.58 | 94% | 153.12 | 62.58 |
| 18259 | 558.61 | 152.63 | 96% | 160.23 | 83.57 |
| 4952 | 867.67 | 202.68 | 94% | 163.05 | 167.45 |
| 20795 | 498.26 | 84.68 | 97% | 165.95 | 99.22 |
| 15292 | 331.21 | 64.99 | 94% | 168.13 | 43.41 |
| 17735 | 616.97 | 206.23 | 95% | 170.62 | 159.27 |
| 15382 | 2086.55 | 655.12 | 96% | 179.06 | 342.56 |
| 6892 | 472.18 | 95.02 | 96% | 185.03 | 58.03 |
| 10019 | 573.47 | 205.58 | 98% | 186.54 | 69.46 |
| 8984 | 284.45 | 40.11 | 94% | 186.61 | 41.02 |
| 3587 | 1589.64 | 832.55 | 95% | 189.25 | 164.29 |
| 23331 | 343.71 | 75.44 | 96% | 197.53 | 41.31 |
| 17753 | 422.58 | 107.22 | 94% | 199.72 | 55.6 |
| 3430 | 482.45 | 99.02 | 96% | 205.47 | 61.75 |
| 5937 | 398.98 | 79.16 | 95% | 210.95 | 55.18 |
| 15091 | 457.85 | 75.14 | 94% | 214.95 | 79.48 |
| 2615 | 475.24 | 65.04 | 95% | 217.68 | 61.55 |
| 22177 | 437.19 | 83.23 | 94% | 220.99 | 76.02 |
| 15558 | 421.96 | 49.45 | 96% | 261.21 | 89.18 |
| 15171 | 2476.94 | 637.89 | 99% | 267.37 | 221.89 |
| 24235 | 651.38 | 135.2 | 94% | 281.24 | 89.88 |
| 15172 | 1130.82 | 386.63 | 99% | 294.17 | 160.06 |
| 8665 | 2451.27 | 808.98 | 94% | 320.3 | 582.92 |
| 3816 | 941.08 | 189.07 | 97% | 375.12 | 97.06 |
| 15051 | 1917.64 | 600.05 | 97% | 421.84 | 274.9 |
| 6321 | 1227.19 | 294.21 | 96% | 436.54 | 171.1 |
| 11495 | 1157.08 | 222.69 | 95% | 479.89 | 170.9 |
| 19012 | 1131.9 | 195.46 | 95% | 491.44 | 164.34 |
| 3139 | 3078.65 | 1586.03 | 96% | 683.5 | 401.95 |

TABLE 3K
Late Cyproterone Acetate

Document Number 1650775

| | Group | Group | | Non Group | Non Group |
|---|---|---|---|---|---|
| GLGC ID | Mean | Stdev | LDA Score | Mean | Stdev |
| 25183 | 57.99 | 11.18 | 99% | −65.21 | 41.14 |
| 9969 | 66.32 | 43.47 | 97% | −28.99 | 30.94 |
| 19292 | 39.25 | 15.99 | 99% | −0.31 | 8.76 |
| 1749 | 36.95 | 4.96 | 97% | 6.56 | 12.85 |
| 9697 | 56.57 | 15.67 | 98% | 10.84 | 13.14 |
| 19465 | 72.95 | 28.72 | 97% | 20.05 | 13.1 |
| 15441 | 57.11 | 16.22 | 98% | 20.18 | 10.67 |
| 15987 | 363.79 | 45.36 | 100% | 34.51 | 32.07 |
| 13580 | 0.18 | 7.99 | 96% | 36.01 | 21.03 |
| 16319 | 89.11 | 16.96 | 97% | 40.72 | 16.75 |
| 3510 | 7.29 | 10.94 | 97% | 41.17 | 13.42 |
| 906 | 86.53 | 14.25 | 98% | 49.56 | 12.1 |
| 19053 | 13.57 | 5.47 | 95% | 50.36 | 50.88 |
| 5824 | 209.96 | 52.5 | 99% | 54.58 | 27.78 |
| 17685 | 17.67 | 8.55 | 98% | 59.93 | 29.82 |
| 4588 | 22.45 | 6.38 | 97% | 60.62 | 24.09 |
| 14250 | 25.11 | 4.35 | 96% | 61.29 | 33.6 |
| 17091 | 228.81 | 44.44 | 99% | 65.14 | 36.75 |
| 4312 | 458.51 | 102.72 | 98% | 74.88 | 65.39 |
| 6667 | 35.58 | 7.42 | 95% | 79.42 | 27.4 |
| 9668 | 25.68 | 7.88 | 95% | 82.74 | 43.74 |
| 17090 | 174.43 | 31.41 | 98% | 82.84 | 25.5 |
| 14840 | 25.84 | 4.54 | 97% | 84.25 | 56.66 |
| 18906 | 165.1 | 25.73 | 97% | 86.57 | 33.68 |
| 21184 | 24.35 | 7.77 | 96% | 88.84 | 44.65 |
| 11960 | −21.76 | 29.8 | 98% | 91.47 | 36.61 |
| 17092 | 282.98 | 55.61 | 99% | 100.94 | 37.11 |
| 18316 | 41.41 | 4.56 | 96% | 101.42 | 51.02 |
| 11724 | 26.29 | 6.1 | 97% | 107.83 | 53.24 |
| 21238 | 29.51 | 14.62 | 96% | 107.94 | 65.27 |
| 9015 | 50.88 | 4.22 | 97% | 111.21 | 39.72 |
| 22204 | 31.75 | 11.16 | 96% | 111.85 | 67.38 |
| 21228 | 60.32 | 10.12 | 95% | 127.7 | 59.24 |
| 25725 | 303.56 | 97.38 | 99% | 127.99 | 39.22 |
| 3381 | 215.51 | 15.65 | 98% | 129.07 | 31.01 |
| 14199 | 49.89 | 11.18 | 96% | 129.55 | 63.16 |
| 12158 | 539.59 | 79.37 | 98% | 149.3 | 94.76 |
| 20711 | 15.4 | 13.95 | 97% | 153.96 | 115.63 |
| 25055 | 543.96 | 83.34 | 98% | 160.37 | 97.11 |
| 15955 | 401.03 | 64.61 | 97% | 167.69 | 104.75 |
| 10002 | 79.22 | 8.3 | 96% | 169.5 | 85.35 |
| 15888 | 103.8 | 7.37 | 96% | 174.62 | 107.57 |
| 23709 | 91.99 | 7.53 | 96% | 180.95 | 142.33 |
| 19255 | 96.69 | 11.59 | 96% | 191.17 | 81.51 |
| 16124 | 59.91 | 18.31 | 97% | 198.11 | 129.25 |
| 8053 | 55.5 | 21.16 | 95% | 199.73 | 121.49 |
| 1796 | 713.84 | 124.8 | 99% | 202.3 | 82.74 |
| 6431 | 44.99 | 10.12 | 99% | 211.22 | 232.8 |
| 4576 | 60.8 | 23.4 | 95% | 213.43 | 78.15 |
| 22713 | 83.58 | 18.05 | 96% | 218.87 | 74.81 |
| 20803 | 489.88 | 37.25 | 100% | 230.7 | 84.72 |
| 8905 | 129.45 | 13.33 | 96% | 236.42 | 105.34 |
| 16780 | 482.97 | 115.87 | 98% | 240.36 | 60.06 |
| 1479 | 143.4 | 14.02 | 96% | 245.89 | 63.54 |
| 12156 | 947.53 | 169.32 | 98% | 270.19 | 144.04 |
| 24860 | 762.67 | 137.57 | 99% | 271.87 | 106.81 |
| 20744 | 131.35 | 9.57 | 96% | 277.11 | 153.4 |
| 12157 | 890.46 | 241.3 | 96% | 295.84 | 176.52 |
| 19256 | 169.36 | 16.84 | 97% | 300.56 | 93.48 |
| 12155 | 849.1 | 121.68 | 98% | 328.83 | 112.43 |
| 1795 | 886.32 | 169.03 | 98% | 332.97 | 138.76 |
| 20864 | 838.11 | 192.14 | 98% | 343.82 | 174.37 |
| 23032 | 174.66 | 35.02 | 96% | 348.75 | 98.36 |
| 18860 | 658.47 | 93.14 | 97% | 352.87 | 102.72 |
| 6801 | 167.82 | 26.32 | 95% | 361.85 | 140 |
| 20915 | 707.08 | 113.27 | 95% | 376.44 | 136.93 |
| 20707 | 836.46 | 117.26 | 98% | 382.05 | 142.91 |
| 18473 | 830.53 | 86.28 | 99% | 405.69 | 223.02 |
| 16278 | 872.29 | 116.7 | 98% | 422.72 | 158.18 |
| 20041 | 189.58 | 32.85 | 98% | 435.36 | 136.08 |
| 25056 | 1055.84 | 195.39 | 98% | 435.67 | 129.34 |
| 20714 | 148.21 | 41.46 | 96% | 438.15 | 637.41 |
| 15500 | 239.22 | 24.81 | 97% | 456.63 | 119.52 |
| 15755 | 214.37 | 34.27 | 99% | 457.32 | 99.49 |
| 11693 | 37.65 | 37.02 | 96% | 462.5 | 345.74 |
| 15127 | 911.94 | 86.23 | 98% | 466.74 | 134.84 |
| 21078 | 321.33 | 18.18 | 96% | 470.87 | 98.57 |
| 19012 | 218.63 | 26.43 | 98% | 519.87 | 206.37 |
| 20713 | 192.33 | 64.34 | 97% | 523.9 | 200.74 |
| 8872 | 2206.69 | 222.08 | 99% | 539.95 | 267.56 |
| 1551 | 300.22 | 24.52 | 98% | 540.56 | 133.08 |
| 15391 | 748.88 | 48.29 | 98% | 555.42 | 79.76 |
| 17541 | 1121.82 | 231.52 | 96% | 689.41 | 156.88 |
| 2569 | 1283.55 | 169.03 | 96% | 712.78 | 286.97 |
| 20804 | 2441.26 | 676.23 | 98% | 723.52 | 393.32 |
| 12160 | 2592.66 | 403.1 | 99% | 826.97 | 370.84 |
| 11644 | 421.94 | 97.8 | 96% | 834 | 240.59 |
| 17788 | 2318.81 | 523.51 | 98% | 909.78 | 263.72 |
| 17117 | 1568.35 | 191.58 | 96% | 1006.34 | 230.44 |
| 15645 | 474.3 | 53.72 | 99% | 1085.08 | 601.13 |
| 6479 | 446.51 | 75.83 | 98% | 1215.32 | 472.08 |
| 22266 | 2441.41 | 319.93 | 97% | 1502.46 | 434.41 |
| 21798 | 2671.47 | 378.77 | 98% | 1532.27 | 351.77 |
| 1957 | 451.84 | 140.88 | 95% | 1533.47 | 786.6 |

TABLE 3L
Early Cyproterone Acetate

Document Number 1650775

| | Group | Group | | Non Group | Non Group |
|---|---|---|---|---|---|
| GLGC ID | Mean | Stdev | LDA Score | Mean | Stdev |
| 12375 | 39.55 | 6.91 | 93% | 6.16 | 21.17 |
| 2803 | 101.95 | 30.32 | 98% | 12.74 | 30.67 |
| 18685 | 55.02 | 18.44 | 95% | 16.95 | 33.49 |
| 15162 | 38.84 | 5.14 | 93% | 19.37 | 14.99 |
| 10200 | 71.52 | 14.25 | 98% | 21.52 | 18.12 |
| 11619 | 40.76 | 5.29 | 93% | 24.39 | 9.81 |
| 5018 | 43.56 | 9.08 | 93% | 25.12 | 11.36 |
| 11125 | 95.81 | 17.05 | 97% | 28.28 | 20.68 |
| 25706 | 108.93 | 17.96 | 98% | 28.74 | 24.94 |
| 17506 | 202.1 | 34.4 | 99% | 28.98 | 70.24 |
| 25852 | 57.42 | 8.81 | 96% | 29.52 | 10.16 |
| 16783 | 107.34 | 24.04 | 95% | 33.35 | 33.97 |
| 4725 | 93.9 | 10.69 | 96% | 40.84 | 123.37 |
| 15097 | 97.88 | 13.08 | 95% | 42.76 | 28.79 |
| 2594 | 115.78 | 19.67 | 97% | 43.16 | 28.35 |
| 18484 | 139.66 | 35.48 | 98% | 43.46 | 17.72 |
| 7967 | 80.61 | 8.41 | 93% | 45.01 | 25.09 |
| 15251 | 113.13 | 7.4 | 98% | 45.58 | 23.44 |
| 14913 | 104.39 | 13.3 | 94% | 51.71 | 28.53 |
| 15655 | 103.19 | 9.18 | 98% | 52.4 | 44.96 |
| 5740 | 98.42 | 10.02 | 93% | 54.17 | 22.49 |
| 15433 | 88.27 | 7.53 | 96% | 55.12 | 26.88 |
| 6676 | 81.6 | 7.48 | 94% | 55.36 | 26.6 |
| 12203 | 284.85 | 67.35 | 98% | 57.37 | 50.59 |
| 11876 | 164.99 | 37.72 | 97% | 59.91 | 38.15 |
| 24051 | 156.13 | 27.52 | 97% | 60.29 | 28.94 |
| 24227 | 159.76 | 22.26 | 98% | 64.47 | 29.99 |
| 23160 | 140.18 | 19.33 | 94% | 79.22 | 46.25 |
| 24236 | 118.22 | 13 | 94% | 79.8 | 46.11 |
| 5754 | 354.87 | 77.25 | 99% | 82.05 | 52.7 |
| 5046 | 201.39 | 29.93 | 96% | 91.8 | 52.22 |
| 4679 | 155.83 | 15.02 | 94% | 93.09 | 39.05 |
| 2372 | 227.9 | 45.92 | 97% | 99.62 | 37.53 |
| 466 | 147.74 | 16.09 | 93% | 100.97 | 24.77 |
| 9128 | 497.34 | 121.83 | 99% | 101.85 | 43.69 |
| 16087 | 72.43 | 6.68 | 96% | 105.7 | 17.95 |
| 22898 | 203.84 | 9.33 | 98% | 107.87 | 73.23 |
| 22717 | 160.84 | 13.59 | 94% | 114.08 | 91.92 |
| 9775 | 472.31 | 82.29 | 98% | 118.73 | 84.58 |
| 19605 | 335.27 | 35.78 | 99% | 131.91 | 48.58 |
| 22503 | 297.45 | 72.36 | 96% | 134.1 | 70.26 |
| 1903 | 323.28 | 80.7 | 97% | 134.88 | 55.57 |
| 6582 | 298.97 | 43.04 | 96% | 137.13 | 83.58 |
| 15030 | 175.94 | 7.66 | 94% | 138.35 | 50.24 |
| 18235 | 287.07 | 66.63 | 97% | 138.94 | 38.25 |
| 15282 | 203.3 | 21.11 | 94% | 148.94 | 105 |
| 13799 | 391.75 | 74.97 | 99% | 152.36 | 52.97 |
| 17955 | 257.17 | 57.57 | 93% | 154.46 | 62.37 |
| 6272 | 415.31 | 82.23 | 98% | 157.51 | 61.87 |
| 3266 | 238.25 | 22.7 | 93% | 160.5 | 50.15 |
| 15959 | 389.2 | 63.99 | 97% | 164.9 | 67.38 |
| 1884 | 191.9 | 7.86 | 93% | 166.42 | 45.16 |
| 15955 | 294.4 | 26.85 | 95% | 169.12 | 106.78 |
| 9486 | 468.68 | 91.29 | 94% | 177.99 | 126.67 |
| 21275 | 349.64 | 80.81 | 96% | 178.44 | 97.42 |
| 16053 | 311.13 | 32.05 | 96% | 206.21 | 223.6 |
| 16747 | 445.78 | 87.8 | 96% | 210.09 | 78.61 |
| 20350 | 393.34 | 72.05 | 94% | 217.18 | 69.07 |
| 6855 | 290.54 | 8.31 | 95% | 227.55 | 64.59 |
| 2326 | 437.32 | 39.57 | 98% | 229.27 | 188.62 |
| 20063 | 579.31 | 78.7 | 98% | 232.67 | 92.42 |
| 11403 | 386.09 | 85.89 | 93% | 235.8 | 240.72 |
| 14303 | 381.51 | 38.02 | 94% | 240.55 | 89.2 |
| 5696 | 167.33 | 17.35 | 93% | 246.96 | 110.75 |
| 7586 | 568.83 | 104.54 | 95% | 247.96 | 137.64 |
| 6821 | 667.02 | 106.37 | 96% | 253.55 | 163 |
| 12956 | 525.48 | 76.44 | 96% | 256.59 | 86.57 |
| 11404 | 487.51 | 32.83 | 97% | 257.84 | 173.77 |
| 4092 | 428.51 | 31.72 | 96% | 269.02 | 120.09 |
| 20 | 182.6 | 13.17 | 93% | 280.26 | 77.1 |
| 7003 | 480.07 | 48.06 | 93% | 299.91 | 136.85 |
| 22835 | 515.95 | 104.87 | 95% | 316.8 | 87.86 |
| 22235 | 511.17 | 15.69 | 98% | 321.64 | 119.46 |
| 1900 | 909.26 | 49.41 | 99% | 339.05 | 159.22 |
| 9674 | 997.96 | 198.11 | 93% | 345.29 | 332.5 |
| 2757 | 553.61 | 62.46 | 93% | 349.8 | 112.21 |
| 3233 | 469.14 | 29.71 | 94% | 350.16 | 111.19 |
| 4937 | 644.14 | 96.95 | 97% | 351.09 | 99.81 |
| 16688 | 485.77 | 14.98 | 95% | 367.52 | 115.86 |
| 8215 | 528.57 | 63.29 | 95% | 395.11 | 169.02 |
| 23515 | 527.7 | 47.35 | 94% | 399.57 | 182.28 |
| 22548 | 1110.25 | 157.18 | 97% | 429.36 | 198.23 |
| 25056 | 701.5 | 107.45 | 94% | 439.98 | 142.37 |
| 23030 | 298.12 | 25.05 | 94% | 443.27 | 320.1 |
| 1930 | 795.75 | 79.48 | 96% | 488.29 | 180.53 |
| 22379 | 987.52 | 105.4 | 98% | 497.46 | 281.53 |
| 18280 | 625.22 | 42.6 | 95% | 500.51 | 355.18 |
| 13557 | 431.55 | 35.49 | 94% | 598.3 | 181.76 |
| 1901 | 1382.54 | 291.7 | 97% | 621.54 | 268.35 |
| 16205 | 433.92 | 33.39 | 96% | 622.45 | 128.79 |
| 19069 | 172.52 | 18.28 | 97% | 622.95 | 345.06 |
| 22906 | 1189.14 | 110.88 | 96% | 633 | 508.28 |
| 7262 | 974.62 | 93.19 | 94% | 656.38 | 287.35 |
| 2354 | 1225.56 | 104.8 | 96% | 666.98 | 252.59 |
| 7362 | 563.59 | 37.8 | 94% | 816.77 | 299.68 |
| 15345 | 1802.55 | 235.04 | 95% | 907.53 | 318.35 |
| 3803 | 1252.52 | 61.21 | 95% | 914.67 | 209.78 |
| 22929 | 620.51 | 53.83 | 95% | 1008.19 | 813.54 |

TABLE 3M
Late Diclofenac

Document Number 1650775

| | Group | Group | | Non Group | Non Group |
|---|---|---|---|---|---|
| GLGC ID | Mean | Stdev | LDA Score | Mean | Stdev |
| 22513 | 2558.9 | 1121.55 | 99% | −137.91 | 262.53 |
| 19512 | 46.17 | 16.3 | 99% | −20.41 | 27.06 |
| 8700 | 150.91 | 57.74 | 98% | −11.7 | 37.23 |
| 19715 | 70.75 | 11.06 | 98% | −11.14 | 18.14 |
| 11645 | 79.3 | 16.37 | 99% | −10.24 | 29 |
| 20200 | 64.31 | 15.52 | 98% | −7.94 | 37.09 |
| 7858 | 64.65 | 32.07 | 99% | −1.01 | 21.41 |
| 22516 | 230.66 | 81.61 | 99% | 0.06 | 50.52 |
| 18974 | 52.85 | 14.89 | 98% | 1.86 | 14 |
| 5291 | 56.16 | 15.92 | 98% | 7.46 | 12.49 |
| 9977 | 33.87 | 1.2 | 99% | 9.6 | 16.15 |
| 372 | 53.19 | 3.15 | 99% | 10.58 | 12.35 |
| 14400 | 168.71 | 36.04 | 98% | 12.55 | 47.33 |
| 955 | 44.09 | 5.41 | 98% | 13.21 | 12.09 |
| 26320 | 148.57 | 67.07 | 98% | 20.83 | 30.04 |
| 23555 | 177.11 | 52.37 | 99% | 22.61 | 21.13 |
| 10790 | −147.58 | 11.69 | 99% | 23.65 | 51 |
| 21445 | 152.54 | 38.45 | 99% | 24.94 | 41.96 |
| 16173 | 102.32 | 21.29 | 99% | 25.18 | 32.39 |
| 25052 | 653.33 | 363.97 | 98% | 29.48 | 65.56 |
| 3452 | 158.59 | 24.76 | 99% | 29.79 | 27.82 |
| 12277 | 126.55 | 32.95 | 98% | 30.14 | 31.31 |
| 16240 | −1.46 | 1.38 | 98% | 31.65 | 28.31 |
| 22512 | 280.38 | 149.23 | 99% | 44.34 | 59.45 |
| 7056 | −11.07 | 4.54 | 99% | 47.11 | 28.14 |
| 19411 | 117.91 | 13.87 | 98% | 47.27 | 27.38 |
| 6198 | 184.84 | 21.67 | 99% | 47.55 | 71.13 |
| 25246 | 17.4 | 2.21 | 98% | 50.19 | 18.57 |
| 15504 | 223.77 | 86.68 | 98% | 54.96 | 108.78 |
| 22514 | 404.55 | 221.07 | 99% | 61.23 | 63.25 |
| 13045 | −1.13 | 17.95 | 98% | 64.8 | 29.82 |
| 9826 | −2.67 | 5.61 | 99% | 66.89 | 26.12 |
| 8079 | −12.12 | 4.26 | 99% | 70.37 | 43.83 |
| 2310 | 520.93 | 356.23 | 98% | 71.67 | 85.7 |
| 25290 | 159.42 | 12.09 | 98% | 74.09 | 78.6 |
| 1430 | −67.02 | 9.22 | 98% | 76.13 | 70.5 |
| 13895 | 199.32 | 16.84 | 98% | 81.85 | 53.19 |
| 11904 | 162.22 | 8.31 | 98% | 82.4 | 38.06 |
| 11596 | 208.15 | 21.91 | 98% | 92.32 | 36.27 |
| 22515 | 1549.73 | 711.86 | 98% | 100.85 | 133.92 |
| 22321 | 175.23 | 33.28 | 98% | 101.48 | 89.03 |
| 8522 | 399.56 | 124.51 | 99% | 108.85 | 69.48 |
| 14491 | 261.16 | 27.37 | 98% | 115.78 | 52.28 |
| 21228 | 330.87 | 20.94 | 99% | 125.87 | 57.45 |
| 20529 | 887 | 406.86 | 98% | 137.26 | 107.43 |
| 3250 | 366.5 | 30.94 | 99% | 144.45 | 58.3 |
| 14504 | 691.37 | 422.61 | 99% | 151.43 | 95.9 |
| 26133 | 549.15 | 106.67 | 98% | 153.02 | 280.02 |
| 21978 | 81 | 5.94 | 98% | 160.08 | 42.54 |
| 3708 | 397.54 | 42.39 | 98% | 161.72 | 77.01 |
| 396 | 355.91 | 58.85 | 98% | 172.48 | 57.78 |
| 23889 | 72.55 | 12 | 99% | 175.14 | 49.66 |
| 12577 | 1097.35 | 411.24 | 98% | 176.09 | 109.22 |
| 18580 | 822.77 | 189.24 | 98% | 201.23 | 172.81 |
| 24237 | 928.14 | 321.39 | 98% | 219.99 | 132.72 |
| 25618 | 180.02 | 2.6 | 98% | 245.62 | 81.24 |
| 4969 | 1833.13 | 949.96 | 98% | 265.19 | 240.61 |
| 5110 | 738.94 | 147.68 | 98% | 271.77 | 107.36 |
| 25619 | 193.88 | 2.98 | 98% | 274.38 | 108.29 |
| 13353 | 101.42 | 6.77 | 99% | 275.78 | 68.9 |
| 7225 | 610.95 | 103.39 | 98% | 276.52 | 112.14 |
| 1175 | 89.72 | 12.52 | 98% | 319.98 | 143.49 |
| 4314 | 199.22 | 16.19 | 98% | 324.04 | 72.64 |
| 21281 | 119 | 14.89 | 99% | 329.77 | 91.62 |
| 699 | 744.08 | 166.35 | 98% | 385.87 | 84.98 |
| 17281 | 191.29 | 11.48 | 99% | 407.86 | 108.78 |
| 7697 | 126.05 | 9.16 | 99% | 418.46 | 147.54 |
| 24012 | 650.52 | 28.61 | 99% | 423.59 | 476.52 |
| 5339 | 1561.45 | 746.53 | 98% | 471.48 | 259.27 |
| 1561 | 1103.42 | 310.4 | 98% | 483.63 | 109.78 |
| 24228 | 1037.63 | 336.37 | 98% | 510.12 | 105.18 |
| 5616 | 1252.37 | 399.53 | 98% | 617.19 | 131.84 |
| 15189 | 2393.48 | 562.64 | 98% | 642.89 | 398.85 |
| 563 | 1286.12 | 293.65 | 98% | 647.49 | 154.22 |
| 19392 | 1380.71 | 448.01 | 98% | 669.42 | 123.39 |
| 21740 | 2258.4 | 588.09 | 98% | 701.14 | 280.06 |
| 1854 | 2250.76 | 618.07 | 99% | 730.54 | 265.59 |
| 3292 | 2871.21 | 931.15 | 99% | 892.15 | 311.65 |
| 22598 | 2831.24 | 966.7 | 98% | 1051.05 | 357.55 |
| 21661 | 2797.22 | 982.49 | 98% | 1087.36 | 376.19 |
| 21660 | 4837.56 | 1684.22 | 98% | 1692.71 | 582.02 |
| 17167 | 4555.27 | 1157.69 | 98% | 2481.92 | 715.65 |

TABLE 3N
Early Diclofenac

Document Number 1650775

| | Group | Group | LDA | Non Group | Non Group |
|---|---|---|---|---|---|
| GLGC ID | Mean | Stdev | Score | Mean | Stdev |
| 10667 | 411.83 | 248.79 | 97% | 13.74 | 165.12 |
| 17695 | 47.26 | 305.83 | 96% | 15.36 | 60.09 |
| 3452 | 91.31 | 23.32 | 97% | 29.73 | 28.67 |
| 21421 | 5.58 | 8.51 | 95% | 31.49 | 16.56 |
| 6222 | −12.72 | 9.64 | 95% | 32.02 | 30.46 |
| 14996 | 180.85 | 117.09 | 98% | 32.69 | 45.29 |
| 12844 | −11.84 | 8.74 | 96% | 39.54 | 27.67 |
| 1843 | 88.96 | 20.57 | 96% | 48.67 | 17.77 |
| 9635 | −9.83 | 19.06 | 95% | 48.68 | 40.62 |
| 21707 | 169.82 | 64.58 | 95% | 59.13 | 53.37 |
| 23302 | 37.52 | 28.79 | 96% | 62.8 | 26.58 |
| 13932 | −63.25 | 79.49 | 95% | 63.9 | 55.2 |
| 18604 | 24.17 | 7.4 | 97% | 65.08 | 25.49 |
| 20354 | 220.66 | 86.86 | 98% | 66.15 | 50.9 |
| 1841 | 188.63 | 53.81 | 95% | 69.83 | 46.13 |
| 355 | 149.37 | 52.24 | 97% | 71.24 | 34.86 |
| 17683 | 40.01 | 12.49 | 96% | 77.75 | 25.92 |
| 2359 | 17.87 | 8.17 | 98% | 86.55 | 44.73 |
| 3713 | 168.44 | 419.14 | 97% | 89.98 | 96.34 |
| 11840 | 51.82 | 10.03 | 96% | 100.7 | 37.97 |
| 19211 | 88.71 | 85.04 | 96% | 108.71 | 56.23 |
| 17800 | 70.19 | 39.86 | 98% | 118.7 | 28.58 |
| 1844 | 277.5 | 69.37 | 96% | 129.25 | 44.39 |
| 356 | 249.59 | 82.38 | 98% | 129.82 | 46.84 |
| 23494 | 49.03 | 10.06 | 96% | 131.42 | 50.45 |
| 14776 | 49.01 | 22.62 | 97% | 134.61 | 47.31 |
| 23626 | 251.41 | 69.01 | 97% | 141.32 | 90.59 |
| 23491 | 85.95 | 100.32 | 96% | 155.17 | 56.53 |
| 21382 | 60.1 | 10.48 | 95% | 162.86 | 70.74 |
| 6213 | 75.91 | 24.03 | 97% | 177.43 | 53.8 |
| 15170 | 66.01 | 17.61 | 95% | 180.78 | 58.76 |
| 23182 | 47.61 | 14.34 | 95% | 182.97 | 82.24 |
| 14958 | 77.51 | 24.88 | 99% | 192.52 | 57.74 |
| 16562 | 315.91 | 84.36 | 96% | 194 | 49.14 |
| 23043 | 116.23 | 50.3 | 97% | 200.45 | 58.35 |
| 18996 | 115.11 | 26.79 | 96% | 211.48 | 69.45 |
| 14997 | 807.1 | 529.54 | 98% | 231.67 | 129.71 |
| 10879 | 84.17 | 41 | 95% | 235.09 | 83.29 |
| 11021 | 90.03 | 69.2 | 95% | 247.67 | 106.37 |
| 2655 | 43.2 | 16.5 | 97% | 258.1 | 178.54 |
| 16859 | 704.09 | 252.4 | 97% | 258.84 | 124.37 |
| 17794 | 130.88 | 63.44 | 97% | 261.13 | 86.21 |
| 6919 | 1235.49 | 468.87 | 99% | 269.17 | 229.63 |
| 13353 | 151.45 | 114.9 | 97% | 276.39 | 67.85 |
| 20 | 432.75 | 81.44 | 97% | 277.59 | 75.26 |
| 12964 | 106.32 | 32.26 | 95% | 288.44 | 95.46 |
| 3722 | 585.01 | 101.14 | 97% | 295.66 | 101.48 |
| 20715 | 308.31 | 50.21 | 96% | 313.11 | 180.79 |
| 23606 | 668.08 | 172.75 | 97% | 313.49 | 105.76 |
| 23230 | 176.98 | 99.78 | 98% | 342.52 | 164.69 |
| 12946 | 142.18 | 31.13 | 97% | 349.51 | 100.28 |
| 24200 | 1265.26 | 395.08 | 97% | 369.8 | 208.75 |
| 16768 | 264.62 | 55.65 | 95% | 376.13 | 78.38 |
| 12857 | 231.61 | 293.1 | 96% | 392.81 | 143.31 |
| 18795 | 726.51 | 149.33 | 97% | 395.27 | 107.88 |
| 19 | 654.92 | 135.45 | 97% | 397.11 | 105.29 |
| 18783 | 716.54 | 157.61 | 95% | 402.03 | 119.63 |
| 19252 | 288.39 | 79.84 | 95% | 410.59 | 104.1 |
| 1114 | 645.09 | 101.99 | 96% | 427.86 | 137.39 |
| 20698 | 914.65 | 381.61 | 97% | 479.92 | 178.44 |
| 21098 | 1119.71 | 394.89 | 99% | 521.35 | 157.69 |
| 21097 | 883.9 | 345.03 | 98% | 525.66 | 142.61 |
| 15191 | 1868.16 | 232.88 | 99% | 528.3 | 355.46 |
| 19373 | 957.63 | 171.61 | 96% | 529.59 | 254.13 |
| 9424 | 1020 | 141.63 | 96% | 537.58 | 150.22 |
| 15606 | 331.04 | 100.93 | 95% | 555.14 | 142.5 |
| 4670 | 2609.57 | 936.24 | 97% | 576.03 | 466.99 |
| 402 | 1115.89 | 448.86 | 99% | 596.85 | 131.13 |
| 13557 | 267.85 | 27.9 | 96% | 601.37 | 178.89 |
| 2368 | 429.73 | 38.72 | 96% | 606.25 | 88.63 |
| 22906 | 2134.54 | 974.52 | 97% | 617.58 | 470.92 |
| 15189 | 1986.69 | 445.74 | 98% | 635.58 | 391.8 |
| 15190 | 2159.12 | 392.22 | 99% | 661.42 | 378.72 |
| 1995 | 1259.5 | 439.49 | 98% | 684.23 | 244.32 |
| 11830 | 1983.61 | 566.45 | 98% | 692.89 | 304.27 |
| 1805 | 1229.6 | 164.21 | 97% | 703.35 | 218.45 |
| 1174 | 1340.59 | 440.4 | 96% | 726.33 | 411.01 |
| 6013 | 1139.77 | 436.67 | 96% | 749.39 | 184.56 |
| 17785 | 1846.83 | 672.05 | 97% | 752.99 | 445.33 |
| 22840 | 1352.3 | 529.97 | 95% | 755.78 | 273.45 |
| 8515 | 346.51 | 83 | 96% | 765.99 | 292.49 |
| 21574 | 391.95 | 100 | 97% | 817.75 | 226.02 |
| 6477 | 1367.6 | 542.86 | 97% | 857.33 | 304.69 |
| 3292 | 1879.44 | 784.97 | 98% | 890.76 | 323.1 |
| 12306 | 3293.83 | 1170.7 | 99% | 1005.26 | 433.69 |
| 7451 | 1583.77 | 483.79 | 96% | 1014.48 | 337.6 |
| 6295 | 2775.87 | 1040.34 | 99% | 1068.45 | 493.12 |
| 21467 | 2391.61 | 1040.88 | 96% | 1118.01 | 516.67 |
| 6633 | 2355.01 | 832.32 | 99% | 1206.88 | 312.71 |
| 14738 | 2426.79 | 883.37 | 99% | 1231.22 | 312.92 |
| 3730 | 2978.69 | 1180.6 | 98% | 1232.87 | 586.1 |
| 3617 | 2869.63 | 1011.46 | 98% | 1268.73 | 398.2 |
| 8715 | 3069.61 | 1101.03 | 99% | 1353.63 | 759.44 |
| 17672 | 2889.9 | 351.84 | 96% | 1930.21 | 397.38 |
| 26152 | 5392.56 | 2027.73 | 98% | 1991.62 | 852.89 |
| 20846 | 4030.03 | 570.84 | 96% | 2449.47 | 889.44 |
| 6018 | 11859.37 | 4320.03 | 98% | 3477.55 | 3126.6 |

TABLE 3O
Estradiol

Document Number 1650775

| | Group | Group | | Non Group | Non Group |
|---|---|---|---|---|---|
| GLGC ID | Mean | Stdev | LDA Score | Mean | Stdev |
| 19476 | 221.25 | 108.8 | 94% | −58.59 | 73.88 |
| 20579 | 65.59 | 26.23 | 87% | −13.8 | 30.61 |
| 4520 | 74.3 | 35.09 | 90% | −1.56 | 34.15 |
| 55 | 34.69 | 14.89 | 86% | 4.7 | 13.41 |
| 384 | 44.98 | 13.2 | 86% | 5.76 | 28.49 |
| 22722 | 566.51 | 262.91 | 96% | 19.66 | 47.88 |
| 12120 | 291.19 | 164.4 | 93% | 20.32 | 48.27 |
| 16283 | 59.56 | 11.97 | 91% | 25.04 | 15.43 |
| 10611 | 78.35 | 19.48 | 91% | 26.01 | 28.58 |
| 3570 | 1203.99 | 486.89 | 96% | 27.26 | 139.67 |
| 3929 | 66.1 | 15.81 | 88% | 32.04 | 17.87 |
| 16783 | 94.16 | 35.66 | 86% | 32.29 | 33.01 |
| 6604 | 9.87 | 7.84 | 88% | 36.24 | 17.57 |
| 10540 | 70.62 | 15.26 | 85% | 39.69 | 19.11 |
| 3846 | 63.36 | 11.22 | 85% | 40.64 | 15.95 |
| 14266 | 463.56 | 161.4 | 95% | 42 | 79.9 |
| 15097 | −4.06 | 20.79 | 88% | 44.39 | 28.23 |
| 16809 | 77.26 | 7.57 | 89% | 53.84 | 28.46 |
| 672 | 185.2 | 45.2 | 92% | 57.01 | 48.59 |
| 25290 | 322.26 | 83.7 | 94% | 68.08 | 67.25 |
| 5493 | 104.13 | 22.09 | 86% | 69.51 | 45.42 |
| 17699 | 379.25 | 121.82 | 95% | 77.01 | 64.08 |
| 15057 | 178.76 | 62.35 | 89% | 80.64 | 61.88 |
| 4082 | 137.71 | 29.22 | 87% | 81.24 | 39.54 |
| 3074 | 305.3 | 91.43 | 94% | 82.44 | 74.5 |
| 12655 | 222.74 | 65.14 | 88% | 90.1 | 61.41 |
| 3073 | 404.03 | 113.1 | 94% | 97.56 | 106.47 |
| 23220 | 158.44 | 34.05 | 86% | 104.71 | 23.6 |
| 18612 | 214.55 | 48.01 | 88% | 114.72 | 54.02 |
| 24442 | 253.1 | 51.52 | 95% | 119.28 | 39.27 |
| 19258 | 345.84 | 102.07 | 91% | 119.63 | 94.13 |
| 6789 | 266.72 | 63.61 | 88% | 130.61 | 57.1 |
| 11465 | 687.63 | 230.97 | 94% | 136.61 | 114.55 |
| 23491 | 259.04 | 44.02 | 89% | 151.54 | 55.44 |
| 3075 | 515.63 | 145.3 | 94% | 159.61 | 267.05 |
| 19261 | 291.37 | 82.45 | 86% | 163.74 | 57.85 |
| 17393 | 223.13 | 34.27 | 86% | 164.98 | 67.02 |
| 23987 | 254.16 | 41.43 | 86% | 168.68 | 53.84 |
| 13229 | 314.84 | 68.95 | 90% | 184.84 | 61.96 |
| 15295 | 252.4 | 28.26 | 85% | 191.1 | 52.8 |
| 23183 | 91.05 | 26.84 | 85% | 192.16 | 88.8 |
| 6549 | 522.38 | 151.13 | 89% | 204.39 | 114.46 |
| 13092 | 440.75 | 124.27 | 92% | 206.68 | 86.61 |
| 9402 | 278.52 | 27.55 | 85% | 207.63 | 69.5 |
| 23362 | 362.98 | 58.85 | 92% | 209.03 | 55.26 |
| 729 | 141.14 | 32.05 | 85% | 209.19 | 55.66 |
| 13963 | 572.36 | 193.21 | 91% | 220.12 | 112.51 |
| 17516 | 287.34 | 30.47 | 85% | 223.48 | 56.14 |
| 7927 | 368.05 | 56.64 | 86% | 226.41 | 79.19 |
| 14989 | 306.39 | 34.48 | 90% | 229.8 | 59.41 |
| 5464 | 608.63 | 139.88 | 93% | 235.86 | 136.35 |
| 14997 | 313.77 | 45.38 | 92% | 237.05 | 156.21 |
| 23337 | 388.86 | 61.57 | 87% | 239.19 | 87.95 |
| 6541 | 835.22 | 410.07 | 90% | 240.86 | 107.93 |
| 9621 | 349.89 | 41.41 | 91% | 242.89 | 62.26 |
| 18877 | 1770.96 | 536.63 | 95% | 251.02 | 323.54 |
| 19825 | 76.2 | 82.83 | 85% | 256.34 | 107.9 |
| 291 | 413.96 | 84.34 | 85% | 256.37 | 66.6 |
| 17613 | 349.67 | 47.08 | 86% | 259.18 | 106.99 |
| 19824 | 83.21 | 81.92 | 87% | 260.01 | 99.57 |
| 7684 | 577.91 | 188.77 | 85% | 279.08 | 126.11 |
| 2373 | 634.92 | 150.17 | 92% | 285.8 | 133.51 |
| 2484 | 57.67 | 44.88 | 86% | 289.53 | 213.13 |
| 16684 | 447.2 | 65.17 | 88% | 306.67 | 87.7 |
| 6975 | 700.83 | 228.78 | 86% | 312.49 | 161.5 |
| 18141 | 1086.32 | 372.55 | 88% | 330.82 | 216.89 |
| 25718 | 464.33 | 56.04 | 91% | 331.59 | 76.26 |
| 18742 | 172.88 | 37.74 | 87% | 352.25 | 190.08 |
| 12361 | 1014.46 | 256.68 | 94% | 354.09 | 232.49 |
| 16327 | 558.02 | 61.36 | 88% | 369.06 | 94.06 |
| 21164 | 169.42 | 47.37 | 86% | 370.17 | 185.53 |
| 24012 | 2053.62 | 525.68 | 94% | 382.21 | 392.09 |
| 4674 | 167.98 | 66.36 | 88% | 452.2 | 224.88 |
| 6060 | 310.86 | 53.86 | 86% | 477.05 | 121.08 |
| 1561 | 310.14 | 86.6 | 90% | 491.78 | 117.97 |
| 11227 | 841.6 | 140.02 | 86% | 496.07 | 212.99 |
| 19728 | 229.27 | 93.53 | 88% | 501.97 | 174.65 |
| 12746 | 759.81 | 83.64 | 93% | 520.3 | 104.48 |
| 12585 | 909.57 | 150.85 | 86% | 542.79 | 178.84 |
| 23437 | 271.75 | 62.16 | 86% | 558.17 | 246.21 |
| 11821 | 1051.26 | 228.29 | 86% | 574.09 | 309.97 |
| 24707 | 407.68 | 85.92 | 85% | 598.16 | 183.22 |
| 16894 | 1105.64 | 177.51 | 91% | 731.2 | 332.55 |
| 11720 | 397.65 | 148.44 | 88% | 748.93 | 265 |
| 4440 | 398.17 | 156.94 | 89% | 804.73 | 210.24 |
| 7584 | 2336.91 | 636.07 | 91% | 819.41 | 712.46 |
| 13093 | 2287.36 | 766.73 | 90% | 825.52 | 505.38 |
| 11644 | 485.11 | 142.46 | 86% | 838.95 | 238.55 |
| 9475 | 422.84 | 219.9 | 86% | 958.81 | 372.8 |
| 24112 | 1879.78 | 259.59 | 90% | 1026.22 | 630.45 |
| 16703 | 714.02 | 96.32 | 86% | 1057.6 | 331.01 |
| 15534 | 1418.23 | 154.26 | 88% | 1104.88 | 261.78 |
| 14738 | 862.34 | 156.54 | 85% | 1256.55 | 349.62 |
| 14960 | 1831.5 | 294.22 | 85% | 1370.37 | 509.8 |
| 22554 | 609.46 | 270.71 | 86% | 1371.14 | 511.54 |
| 6015 | 707.01 | 273.93 | 89% | 1539.98 | 455.17 |
| 7497 | 1136.4 | 136.44 | 87% | 1691.66 | 329.88 |

TABLE 3P
Late indomethacin

Document Number 1650775

| | Group | Group | | Non Group | Non Group |
|---|---|---|---|---|---|
| GLGC ID | Mean | Stdev | LDA Score | Mean | Stdev |
| 21075 | 56.56 | 18.08 | 99% | −101.64 | 72.06 |
| 3626 | 270.02 | 126.67 | 99% | −91.68 | 41.85 |
| 20522 | 88.79 | 62.74 | 99% | −86.26 | 44.12 |
| 18203 | 28.03 | 7.89 | 100% | −59.65 | 26.67 |
| 21682 | 139.83 | 65.11 | 99% | −56.8 | 31.49 |
| 20119 | 75.13 | 51.9 | 99% | −51.89 | 22.95 |
| 945 | 164.01 | 44.63 | 98% | −32.43 | 36.01 |
| 8017 | 40.5 | 7.12 | 99% | −4.91 | 18.36 |
| 22516 | 427.71 | 48.74 | 100% | −3.53 | 27.61 |
| 7858 | 133.46 | 131.64 | 99% | −2.18 | 10.32 |
| 11731 | 57.13 | 15.61 | 99% | −1.13 | 13.51 |
| 2011 | 88.53 | 22.86 | 99% | 5.7 | 10.46 |
| 19121 | 104.23 | 50.09 | 99% | 16.77 | 12.76 |
| 24826 | 218.27 | 46.71 | 99% | 17.2 | 179.73 |
| 23555 | 133.19 | 49.37 | 99% | 22.23 | 20.8 |
| 21445 | 313.48 | 71.78 | 99% | 22.36 | 29.24 |
| 1777 | 117.77 | 21.2 | 99% | 22.67 | 16.4 |
| 16173 | 249.12 | 60.67 | 99% | 23.05 | 21.76 |
| 21683 | 179.43 | 48.48 | 99% | 24.37 | 26.58 |
| 19503 | 106.66 | 42.52 | 99% | 24.54 | 12.74 |
| 19444 | 479 | 225.49 | 99% | 26.17 | 29.3 |
| 20651 | 252.93 | 78.27 | 99% | 26.84 | 24.52 |
| 11172 | 108.09 | 14.64 | 99% | 27.38 | 25.08 |
| 7196 | 70.2 | 6.99 | 99% | 27.5 | 18.37 |
| 8864 | 168.51 | 38.98 | 98% | 28.16 | 40.98 |
| 25052 | 413.35 | 149.76 | 98% | 28.65 | 72.19 |
| 12277 | 188.8 | 30.97 | 99% | 28.87 | 27.27 |
| 20134 | 115.79 | 25.97 | 99% | 31.07 | 21.72 |
| 15961 | 155.48 | 44.33 | 99% | 31.59 | 27.65 |
| 22897 | 135.13 | 41.74 | 99% | 33.43 | 19.08 |
| 1893 | 250.46 | 53.73 | 99% | 40.37 | 21.42 |
| 22512 | 493.75 | 186.61 | 99% | 40.54 | 35.84 |
| 14081 | 1307.16 | 578.37 | 99% | 40.73 | 109.27 |
| 25083 | 96.77 | 17.16 | 99% | 41.1 | 19.54 |
| 17500 | 182.9 | 29.18 | 100% | 43.12 | 42.04 |
| 2013 | 191.84 | 31.9 | 99% | 44.55 | 23.34 |
| 8273 | 410.92 | 194.88 | 99% | 45.89 | 30.96 |
| 19411 | 184.69 | 32.53 | 99% | 46.1 | 23.55 |
| 15504 | 896.04 | 321.22 | 99% | 46.28 | 53.42 |
| 22514 | 543.21 | 150.84 | 99% | 57.67 | 44.72 |
| 155 | 187.91 | 27.8 | 99% | 62.07 | 21.49 |
| 20523 | 337.44 | 89.8 | 98% | 66.71 | 58.22 |
| 16961 | 225.29 | 41.42 | 99% | 71.58 | 40.53 |
| 24589 | 412.43 | 149.59 | 98% | 73.14 | 30.15 |
| 21285 | 903.94 | 338.62 | 99% | 73.28 | 108.74 |
| 15503 | 519.54 | 109.49 | 100% | 74.61 | 27.28 |
| 6200 | 1572.18 | 522.18 | 99% | 78 | 145.78 |
| 7743 | 288.96 | 85.4 | 98% | 83.77 | 52.71 |
| 2012 | 357.34 | 70.02 | 99% | 84.87 | 34.39 |
| 3749 | −48.1 | 12.54 | 99% | 87.36 | 48.17 |
| 4892 | 2121.77 | 1018.81 | 99% | 97.96 | 339.86 |
| 24651 | 168.51 | 30.23 | 98% | 98.36 | 20.05 |
| 23005 | 536.62 | 86.56 | 99% | 99.43 | 90.49 |
| 1700 | 273.11 | 39.16 | 99% | 102.11 | 30.56 |
| 22898 | 507.42 | 174.82 | 99% | 103.97 | 57.4 |
| 8522 | 552.47 | 146.35 | 99% | 105.43 | 54.02 |
| 12714 | 0.7 | 18.22 | 98% | 106.47 | 34.92 |
| 15116 | 243.85 | 52.64 | 98% | 107.4 | 25.94 |
| 17277 | 239.1 | 35.46 | 99% | 107.78 | 39.78 |
| 22042 | 21.05 | 10.38 | 98% | 109.25 | 91.56 |
| 21414 | 1412.18 | 189.99 | 99% | 116.04 | 143.33 |
| 17258 | 235.7 | 32.66 | 99% | 120.39 | 25.05 |
| 682 | 555.72 | 137.48 | 99% | 126.28 | 58.1 |
| 17369 | 441.37 | 64.2 | 99% | 130.38 | 54.83 |
| 20529 | 790.13 | 186.87 | 99% | 134.07 | 101.45 |
| 14504 | 773.65 | 116.14 | 99% | 147.38 | 84.22 |
| 154 | 347.17 | 63.6 | 99% | 154.37 | 37.49 |
| 12450 | −60.33 | 24.42 | 99% | 154.48 | 84.94 |
| 6431 | 1828.3 | 421.64 | 99% | 190.99 | 149.33 |
| 18580 | 1167.73 | 411.76 | 99% | 193.7 | 141.11 |
| 8310 | 107.35 | 13.86 | 99% | 204.96 | 44.79 |
| 14330 | 633.28 | 126.05 | 99% | 225.12 | 77.1 |
| 5687 | 48.78 | 22.59 | 99% | 227.66 | 79.73 |
| 14185 | 760.34 | 170.85 | 99% | 253.08 | 93.43 |
| 21443 | 569.4 | 110.65 | 99% | 256.7 | 61.78 |
| 16519 | 807.19 | 191.58 | 98% | 273.02 | 117.31 |
| 9079 | 820.52 | 184.52 | 98% | 316.54 | 112.19 |
| 19469 | 162.04 | 26.75 | 99% | 325.82 | 57.22 |
| 373 | 115.43 | 31.34 | 99% | 334.03 | 85.91 |
| 43 | 156.53 | 22.34 | 99% | 341.11 | 74.71 |
| 20864 | 37.65 | 12.15 | 100% | 352.3 | 179.09 |
| 699 | 762.57 | 112.9 | 99% | 383.6 | 79.72 |
| 24323 | 230.34 | 24.71 | 99% | 398.78 | 95.09 |
| 17281 | 100.34 | 30.42 | 99% | 410.15 | 105.21 |
| 16366 | 113.72 | 34.12 | 99% | 439.22 | 103.99 |
| 21014 | 188.22 | 42.97 | 99% | 572.37 | 137.02 |
| 16367 | 166.59 | 86.34 | 99% | 612.27 | 144.06 |
| 25525 | 264.07 | 72.58 | 99% | 645.12 | 117.62 |
| 635 | 308.38 | 68.87 | 99% | 672.17 | 126.74 |
| 18890 | 126.36 | 42.96 | 99% | 679.93 | 361.87 |
| 634 | 355.69 | 72.95 | 99% | 705.77 | 125.16 |
| 6236 | 227.28 | 73.91 | 98% | 902.24 | 429.28 |
| 10984 | 135.85 | 78.66 | 99% | 1092.48 | 362.92 |
| 15029 | 181.72 | 50.19 | 99% | 1492.95 | 529.6 |
| 4933 | 357.28 | 114.44 | 99% | 1702.56 | 598.89 |

TABLE 3Q
Early Indomethacin

Document Number 1650775

| | Group | Group | | Non Group | Non Group |
|---|---|---|---|---|---|
| GLGC ID | Mean | Stdev | LDA Score | Mean | Stdev |
| 21682 | 85.12 | 87.03 | 93% | −56.37 | 33.66 |
| 1510 | 75.53 | 7.54 | 96% | −13.1 | 65.66 |
| 26280 | 109.21 | 31.74 | 89% | −10.05 | 85.78 |
| 11422 | 60.74 | 22.85 | 91% | 13.75 | 11.38 |
| 1507 | 46.96 | 9.51 | 87% | 15.4 | 15.74 |
| 16251 | 34.42 | 5.87 | 90% | 20.02 | 13.62 |
| 19671 | 39.81 | 7.46 | 90% | 22.33 | 14.64 |
| 23106 | 48.6 | 11.99 | 93% | 28.28 | 33.85 |
| 2736 | 49.82 | 5.14 | 93% | 29.89 | 18.47 |
| 25077 | 111.99 | 30.35 | 88% | 30.69 | 73.6 |
| 1221 | 445.47 | 178.19 | 92% | 33.57 | 94.3 |
| 18389 | 94.31 | 16.02 | 94% | 33.62 | 32.95 |
| 3972 | −24.58 | 15.09 | 94% | 34.18 | 35.89 |
| 18237 | 63.23 | 7.16 | 91% | 36.35 | 20.91 |
| 22725 | 4.84 | 8.57 | 88% | 36.54 | 24.3 |
| 17854 | 94.21 | 22.12 | 90% | 48.6 | 21.13 |
| 25379 | 64.97 | 7.1 | 91% | 48.71 | 16.47 |
| 1843 | 85.73 | 19.01 | 94% | 48.71 | 17.88 |
| 4504 | 96.84 | 28.13 | 90% | 48.77 | 77.49 |
| 24024 | 75.74 | 15.08 | 90% | 50.05 | 33.85 |
| 16809 | 117.87 | 32.17 | 90% | 53.62 | 27.39 |
| 11423 | 102.73 | 23.05 | 89% | 54.5 | 20.13 |
| 2042 | 92.88 | 5.97 | 96% | 54.98 | 50.98 |
| 13992 | 110.02 | 45.53 | 90% | 55.81 | 24.86 |
| 22918 | 27.24 | 5.2 | 92% | 57.51 | 29.32 |
| 5059 | 222.71 | 98.2 | 92% | 61.9 | 61.99 |
| 20354 | 194.32 | 79.46 | 91% | 66.49 | 51.97 |
| 18529 | 139.38 | 36.52 | 88% | 68.68 | 53.21 |
| 8079 | −1.13 | 28.24 | 91% | 70.82 | 43.57 |
| 7176 | 83.8 | 6.04 | 89% | 71.68 | 21.23 |
| 24721 | 116.01 | 17.12 | 91% | 75.35 | 29.71 |
| 11904 | 169.62 | 30.75 | 91% | 81.73 | 37.23 |
| 3710 | −40.52 | 24.79 | 89% | 84.89 | 112.56 |
| 1271 | 127.09 | 19.36 | 88% | 87.87 | 22.54 |
| 15207 | 207.84 | 67.65 | 90% | 88.03 | 53.57 |
| 21256 | 150.53 | 29.3 | 87% | 90.66 | 43.12 |
| 1572 | 134.45 | 17.05 | 87% | 92.3 | 26.58 |
| 19410 | 154.21 | 25.11 | 89% | 95.44 | 23.68 |
| 16080 | 172.16 | 50.03 | 89% | 95.77 | 117.15 |
| 17950 | 134.99 | 16.51 | 87% | 96.23 | 39.64 |
| 22321 | 169.07 | 47.34 | 95% | 101.03 | 89.08 |
| 9223 | 166.07 | 27.83 | 88% | 106.75 | 43.32 |
| 17277 | 186.86 | 45.28 | 88% | 108.27 | 41.12 |
| 16125 | 212.34 | 60.78 | 90% | 109.55 | 34.54 |
| 354 | 156.92 | 39.75 | 88% | 113.78 | 121.78 |
| 22151 | 49.94 | 21.66 | 90% | 114.35 | 59.07 |
| 16477 | 205.91 | 47.02 | 87% | 118.16 | 42.37 |
| 15884 | 197.78 | 19.66 | 96% | 119.51 | 58.67 |
| 25768 | 189 | 17.68 | 94% | 128.02 | 30.12 |
| 6532 | 275.04 | 58.08 | 92% | 135.65 | 42.31 |
| 2555 | 342.38 | 116.88 | 91% | 141.73 | 57.69 |
| 25370 | 95.55 | 12.34 | 87% | 141.81 | 76.1 |
| 1426 | 186.05 | 11.71 | 91% | 141.89 | 28.02 |
| 16081 | 293.29 | 79.31 | 90% | 147.43 | 146.68 |
| 154 | 240.39 | 32.25 | 90% | 155.47 | 42.04 |
| 1521 | 271.17 | 53.27 | 87% | 157.16 | 61.75 |
| 22806 | 82.54 | 19.97 | 89% | 169.69 | 77.1 |
| 1141 | 221.49 | 23.61 | 89% | 172.77 | 35.13 |
| 9595 | 369.54 | 72.63 | 90% | 176.26 | 67.68 |
| 21709 | 240.64 | 11.92 | 95% | 179.9 | 33.86 |
| 13332 | 111.82 | 16.97 | 88% | 187.21 | 61.88 |
| 21444 | 292.61 | 40.73 | 91% | 204.56 | 58.9 |
| 20350 | 333.21 | 45.66 | 91% | 216.95 | 69.67 |
| 3776 | 316.54 | 58.6 | 88% | 226.04 | 54.29 |
| 958 | 283.88 | 16 | 89% | 240.09 | 72.64 |
| 18891 | 63.95 | 40.8 | 91% | 245.89 | 190.12 |
| 15786 | 130.41 | 48.25 | 89% | 247.11 | 88.8 |
| 22619 | 509.69 | 128.09 | 87% | 254.11 | 122.09 |
| 2655 | 76.89 | 36.89 | 90% | 257.67 | 178.99 |
| 21443 | 408.93 | 75.59 | 90% | 258.32 | 68.58 |
| 17664 | 718.76 | 159.35 | 90% | 309.86 | 189.82 |
| 1795 | 179.95 | 54.13 | 87% | 340.51 | 149.15 |
| 6825 | 188.01 | 57.66 | 89% | 342.19 | 121.17 |
| 18465 | 583.12 | 68.3 | 93% | 353.78 | 236.17 |
| 19412 | 798.48 | 156.59 | 91% | 364.41 | 124.75 |
| 4026 | 854.17 | 324.83 | 92% | 368.96 | 133.71 |
| 20915 | 208.25 | 51.68 | 88% | 381.94 | 139.96 |
| 12463 | 631.37 | 114.76 | 89% | 391.56 | 105.49 |
| 7122 | 778.65 | 154.65 | 89% | 421.1 | 129.61 |
| 23245 | 695.04 | 100.61 | 88% | 453.5 | 126.98 |
| 20701 | 818.5 | 138.91 | 89% | 496.14 | 169.1 |
| 23125 | 203.3 | 56.02 | 88% | 520.99 | 516.04 |
| 21740 | 1357.78 | 289.81 | 91% | 701.6 | 296.47 |
| 16458 | 933.78 | 80.79 | 89% | 722.78 | 196.14 |
| 11720 | 1393.76 | 333.85 | 92% | 731.5 | 257.06 |
| 23449 | 166.05 | 104.49 | 89% | 922.94 | 660.67 |
| 23989 | 1702.06 | 285.92 | 87% | 1063.27 | 404.32 |
| 22368 | 637.02 | 202.48 | 88% | 1081.65 | 343.44 |
| 24289 | 672.7 | 120.08 | 88% | 1097.27 | 342.03 |
| 16885 | 837.41 | 195.77 | 91% | 1485.4 | 407.68 |
| 9267 | 809.11 | 323.93 | 92% | 1667.39 | 543.29 |

TABLE 3R
Valproate

Document Number 1650775

| | Group | Group | | Non Group | Non Group |
|---|---|---|---|---|---|
| GLGC ID | Mean | Stdev | LDA Score | Mean | Stdev |
| 26190 | 239.04 | 44.21 | 99% | −115.53 | 71.46 |
| 2154 | 26.52 | 22.45 | 98% | −34 | 15.98 |
| 12625 | 129.76 | 35.25 | 98% | −7.97 | 79.74 |
| 4231 | 160.07 | 13.84 | 100% | −6.47 | 34.51 |
| 360 | 42.77 | 15.77 | 97% | −5.58 | 16.63 |
| 24126 | 127.21 | 24.22 | 97% | 6.68 | 31.59 |
| 8993 | 64.31 | 7.77 | 99% | 8.92 | 10.71 |
| 19762 | 168.43 | 71.93 | 99% | 9.69 | 24.52 |
| 11336 | 60.09 | 15.29 | 99% | 12.42 | 10.72 |
| 20993 | 73.86 | 17.79 | 98% | 12.51 | 23.49 |
| 330 | 76.9 | 11.84 | 98% | 13.5 | 26.03 |
| 12058 | 48.89 | 5.96 | 98% | 16.85 | 15.53 |
| 1579 | 75.5 | 19.78 | 98% | 16.86 | 13.09 |
| 5993 | 49.43 | 5.91 | 97% | 17.56 | 13.02 |
| 8054 | 63.83 | 11.7 | 97% | 17.56 | 15.18 |
| 23315 | 53.08 | 6.14 | 98% | 20.16 | 11.05 |
| 23843 | 102.85 | 21.92 | 99% | 21.2 | 18.22 |
| 11315 | 170.88 | 30.14 | 98% | 22.9 | 42.27 |
| 13812 | 138.26 | 33.46 | 99% | 26.62 | 22.64 |
| 23106 | 97.66 | 12.04 | 99% | 28.05 | 33.33 |
| 11625 | 70.95 | 9.83 | 97% | 28.43 | 16.22 |
| 9374 | 155.52 | 11.78 | 99% | 30.44 | 41.52 |
| 10394 | 210.39 | 57.19 | 99% | 35.12 | 29.91 |
| 6101 | 146.33 | 49.53 | 97% | 38.17 | 25.87 |
| 2117 | 107.64 | 17.82 | 97% | 43.75 | 19.24 |
| 12614 | 113.54 | 14.75 | 98% | 45.51 | 37.01 |
| 9766 | 130.53 | 51.66 | 98% | 47.22 | 33.17 |
| 2932 | 256.87 | 86.84 | 98% | 48.26 | 30.66 |
| 13501 | 145.64 | 35.69 | 98% | 48.87 | 22.87 |
| 14913 | 145.2 | 21.59 | 98% | 51.42 | 27.75 |
| 16673 | 133.08 | 23.07 | 98% | 53.6 | 21.07 |
| 2042 | 183.57 | 50.07 | 98% | 54.55 | 49.7 |
| 2915 | 150.2 | 35.95 | 98% | 55.29 | 23.13 |
| 19669 | 192.83 | 28.28 | 99% | 60.25 | 31.79 |
| 19264 | 145.96 | 13.12 | 98% | 62.26 | 25.95 |
| 17257 | 197.58 | 17.21 | 99% | 67.22 | 34.6 |
| 15663 | 157.22 | 12.55 | 98% | 67.92 | 42.04 |
| 11527 | 186.56 | 12.56 | 97% | 68.89 | 53.83 |
| 22375 | 201.22 | 32.17 | 99% | 75.66 | 28.1 |
| 5754 | 289.15 | 110.18 | 98% | 82.52 | 54.48 |
| 12198 | 157.09 | 5.38 | 99% | 83.53 | 37.27 |
| 18885 | 179.92 | 14.06 | 99% | 85.54 | 27.13 |
| 13166 | 392.55 | 98.9 | 98% | 89.27 | 56.47 |
| 13251 | 155.07 | 11.85 | 97% | 89.73 | 88.96 |
| 8728 | 346.01 | 114.17 | 98% | 90.12 | 40.25 |
| 2216 | 234.47 | 28.59 | 99% | 94.87 | 37.16 |
| 21535 | 197.23 | 12.53 | 98% | 96.15 | 38.42 |
| 21567 | 509.19 | 66.46 | 98% | 97.9 | 104.57 |
| 10593 | 328.02 | 63.73 | 99% | 101.91 | 43.97 |
| 17368 | 241.72 | 37.58 | 97% | 104.44 | 49.02 |
| 9800 | 366.46 | 11.6 | 99% | 105.66 | 68.67 |
| 17479 | 261.87 | 40.08 | 99% | 106.14 | 33.44 |
| 21976 | 256.5 | 24.3 | 98% | 106.4 | 45.51 |
| 14600 | 242.39 | 40.76 | 98% | 111.36 | 76.44 |
| 22570 | 241.74 | 26.13 | 97% | 111.56 | 44.08 |
| 23656 | 273.7 | 31.03 | 98% | 112.56 | 52.23 |
| 15179 | 255.98 | 37.97 | 98% | 112.9 | 41.1 |
| 16616 | 304.19 | 58.02 | 98% | 115.37 | 49.86 |
| 5608 | 233.3 | 11.25 | 97% | 122.33 | 53.28 |
| 20090 | 263.76 | 45.31 | 98% | 126.59 | 32.66 |
| 17644 | 333.21 | 52.99 | 98% | 128.35 | 68.07 |
| 15149 | 345.13 | 64.29 | 97% | 128.59 | 59.92 |
| 6789 | 283.91 | 53.49 | 99% | 133.02 | 59.87 |
| 6686 | 369.2 | 41.65 | 99% | 139.06 | 46.36 |
| 19230 | 391.37 | 57.35 | 98% | 149.61 | 84.83 |
| 13949 | 47.22 | 6.84 | 99% | 151.24 | 58.29 |
| 11280 | 287.5 | 36.75 | 98% | 159.37 | 38.65 |
| 19513 | 345.16 | 59.75 | 97% | 163.49 | 60.93 |
| 23762 | 321.28 | 26.82 | 97% | 164.97 | 66.22 |
| 13838 | 437.29 | 30.14 | 99% | 166.7 | 55.87 |
| 2691 | 316.24 | 12.09 | 98% | 168.14 | 70.13 |
| 9572 | 409.53 | 66.85 | 99% | 168.33 | 60.29 |
| 6861 | 397.87 | 34.78 | 100% | 168.71 | 47.4 |
| 22135 | 361.16 | 95.89 | 98% | 170.63 | 47.21 |
| 24388 | 283.3 | 44.23 | 98% | 172.33 | 155.38 |
| 18886 | 403.05 | 74.14 | 98% | 175.49 | 63.14 |
| 24368 | 602.67 | 63.22 | 99% | 183.22 | 79.82 |
| 5381 | 356.13 | 13.85 | 99% | 191.57 | 49.01 |
| 9402 | 342.47 | 21.74 | 97% | 208.49 | 68.96 |
| 17261 | 546.81 | 71.98 | 99% | 219.95 | 72.35 |
| 2101 | 430.5 | 35.07 | 99% | 224.81 | 67.09 |
| 24369 | 546.78 | 56.44 | 97% | 228.98 | 103.39 |
| 11354 | 530 | 66.53 | 99% | 229.49 | 68.24 |
| 8709 | 90.79 | 24.72 | 98% | 233.09 | 61.98 |
| 24367 | 400.74 | 12.79 | 99% | 245.59 | 55.58 |
| 19052 | 646.73 | 83.13 | 98% | 254.53 | 92.68 |
| 22957 | 665.35 | 87.82 | 98% | 274.44 | 208.86 |
| 15551 | 493.87 | 26.61 | 99% | 304.36 | 63.07 |
| 12317 | 639.88 | 73.89 | 99% | 308.65 | 88.02 |
| 4179 | 845.91 | 78.29 | 98% | 333.97 | 135.14 |
| 6440 | 961.78 | 166.32 | 97% | 351.53 | 186.44 |
| 7111 | 553.56 | 43.59 | 98% | 353.19 | 75.73 |
| 18285 | 707.67 | 76.76 | 99% | 357.46 | 132.75 |
| 12928 | 791.23 | 86.89 | 98% | 410.91 | 94.08 |
| 15051 | 1110.61 | 136.73 | 97% | 476.75 | 412.42 |
| 2569 | 338.95 | 14.84 | 98% | 721.15 | 290.78 |
| 3803 | 499.92 | 74.41 | 97% | 920.04 | 208.7 |
| 18962 | 573.38 | 98.13 | 99% | 1606.33 | 624.84 |
| 5052 | 906.23 | 65.55 | 99% | 1930.67 | 442.76 |
| 22540 | 1108.89 | 178.44 | 97% | 2311.11 | 657.83 |

TABLE 3S
WY-14643

Document Number 1650775

| | Group | Group | | Non Group | Non Group |
|---|---|---|---|---|---|
| GLGC ID | Mean | Stdev | LDA Score | Mean | Stdev |
| 3175 | 81.67 | 38.5 | 98% | −24.57 | 20 |
| 2051 | 31.61 | 16.91 | 98% | −19.67 | 25.77 |
| 23627 | 40.97 | 4.93 | 98% | −14.82 | 37.36 |
| 16409 | 95.86 | 23.34 | 97% | −8.25 | 35.38 |
| 14116 | 38.83 | 17.55 | 99% | −7.83 | 5.25 |
| 18029 | 208.84 | 94.33 | 98% | −7.23 | 21.53 |
| 6677 | 32.1 | 15.65 | 98% | −6.62 | 9.95 |
| 20856 | 275.88 | 94.5 | 99% | −5.26 | 14.41 |
| 5565 | 221.64 | 85.1 | 97% | 17.46 | 47.37 |
| 12467 | 216.39 | 65.04 | 99% | 20.32 | 20.78 |
| 23500 | 148.59 | 59.24 | 99% | 22.05 | 17.54 |
| 1858 | 529 | 114.56 | 99% | 23.94 | 68.23 |
| 8820 | 81.06 | 9.86 | 99% | 28.61 | 31.53 |
| 18082 | 128.62 | 31.47 | 99% | 29.7 | 16.97 |
| 4931 | 135.4 | 29.63 | 97% | 33.8 | 32.95 |
| 9925 | 117.26 | 29.18 | 98% | 42.43 | 17 |
| 24381 | 97.68 | 12.7 | 98% | 43.65 | 17.97 |
| 6292 | 96.5 | 10.27 | 98% | 43.76 | 16.97 |
| 5518 | −34.55 | 15.68 | 100% | 44.56 | 14.44 |
| 18083 | 370.91 | 74.26 | 98% | 45.23 | 60.06 |
| 4272 | 590.58 | 82.76 | 100% | 47.77 | 61.51 |
| 7295 | 114.22 | 11.36 | 98% | 48.54 | 27.07 |
| 8315 | 251.82 | 52.39 | 98% | 50.52 | 44.35 |
| 20855 | 205.89 | 56.89 | 100% | 51.41 | 13.97 |
| 15018 | 153.93 | 12.99 | 97% | 51.69 | 40.82 |
| 22046 | 173.79 | 36.81 | 97% | 52.05 | 35.05 |
| 4438 | −53.05 | 31.71 | 99% | 53.83 | 12.81 |
| 18956 | 233.24 | 49.47 | 99% | 57.47 | 28.38 |
| 3631 | 135.16 | 24.43 | 97% | 62.18 | 23.06 |
| 4271 | 1146.85 | 102.6 | 100% | 63.33 | 94.28 |
| 6553 | 215.81 | 43.91 | 97% | 64.81 | 42.7 |
| 3558 | 192.81 | 32.74 | 98% | 65.12 | 31.67 |
| 20038 | 306.38 | 66.25 | 98% | 68.41 | 50.76 |
| 7517 | 190.58 | 26.66 | 98% | 71.67 | 32.59 |
| 3743 | 185.35 | 31.74 | 99% | 71.95 | 25.24 |
| 14507 | 291.71 | 54.52 | 98% | 74.57 | 66.85 |
| 18749 | 288.03 | 90.54 | 98% | 77.94 | 40.13 |
| 4290 | 293.68 | 45.21 | 98% | 87.32 | 46.32 |
| 14595 | 321.16 | 55.3 | 98% | 89.33 | 56.57 |
| 14264 | 331.35 | 82.51 | 98% | 91.8 | 58.3 |
| 397 | 232.66 | 39.79 | 99% | 91.99 | 32.22 |
| 18746 | 280.52 | 43.35 | 98% | 93.45 | 48.78 |
| 3439 | 244.57 | 26.7 | 99% | 100.37 | 28.67 |
| 2190 | 164.79 | 17.03 | 97% | 100.78 | 189.02 |
| 18318 | 279.93 | 40.82 | 98% | 111.57 | 48.48 |
| 5887 | 1076.32 | 275.72 | 99% | 111.64 | 139.98 |
| 3513 | 212.58 | 33.36 | 98% | 114.18 | 27.84 |
| 22416 | 1001.99 | 170.33 | 99% | 121.52 | 83.97 |
| 22224 | 487.47 | 76.85 | 99% | 124.54 | 72.09 |
| 12215 | 632.99 | 209.38 | 98% | 141.79 | 100.45 |
| 9373 | 419.3 | 49.02 | 98% | 144.86 | 76.23 |
| 15672 | 378.23 | 65.03 | 98% | 151.17 | 68.05 |
| 3260 | 508.28 | 175.97 | 98% | 153.29 | 72.65 |
| 16700 | 596.39 | 103.44 | 99% | 155.05 | 96.4 |
| 18747 | 457.04 | 82.08 | 97% | 155.98 | 76.29 |
| 26109 | 1286.05 | 121.59 | 99% | 156.58 | 201.4 |
| 22737 | 685.5 | 206.71 | 99% | 168.28 | 96.83 |
| 3720 | 315.08 | 30.72 | 98% | 179.69 | 49.62 |
| 2113 | 410.43 | 34.36 | 99% | 185.32 | 58.03 |
| 15015 | 374.26 | 31.51 | 99% | 192.11 | 63.36 |
| 6439 | 425.56 | 74.96 | 97% | 196.56 | 74.01 |
| 22370 | 945.85 | 62.98 | 100% | 216.15 | 108.38 |
| 2457 | 1132.75 | 158.6 | 99% | 227.31 | 140.2 |
| 1728 | 477.23 | 66.78 | 98% | 227.92 | 60.65 |
| 18891 | 1245.42 | 225.38 | 99% | 230.61 | 151.12 |
| 22620 | 386.56 | 21.42 | 98% | 235.22 | 68.77 |
| 19591 | 567.11 | 40.94 | 99% | 237.04 | 108.52 |
| 5602 | 1404.36 | 215.76 | 99% | 242.82 | 212.8 |
| 24860 | 67.15 | 34.2 | 97% | 279.45 | 115.83 |
| 22392 | 598.76 | 55.66 | 99% | 296.04 | 67.51 |
| 18742 | 1303.27 | 263.5 | 99% | 335.32 | 154.05 |
| 6825 | 626.39 | 47.06 | 98% | 336.52 | 118 |
| 21164 | 991.37 | 155.11 | 99% | 356.95 | 172.12 |
| 9372 | 1244.96 | 107.3 | 99% | 368.29 | 225.64 |
| 8177 | 121.78 | 23.64 | 97% | 389.45 | 423.88 |
| 17935 | 1404.15 | 220.52 | 97% | 416.54 | 273.3 |
| 10533 | 1054.36 | 147.32 | 98% | 421.36 | 212.4 |
| 16944 | 747.42 | 72.2 | 98% | 422.41 | 133.98 |
| 21354 | 2186.83 | 317.02 | 98% | 437.51 | 348.77 |
| 16323 | 223.57 | 44.79 | 99% | 465.4 | 220.36 |
| 9423 | 273.32 | 30.42 | 98% | 486.76 | 134.12 |
| 19044 | 814.58 | 45.86 | 97% | 502.31 | 184.58 |
| 18727 | 206.23 | 25.52 | 99% | 516.82 | 179.53 |
| 18125 | 1062.51 | 80.83 | 99% | 529.14 | 174.32 |
| 16704 | 1486.63 | 221.63 | 97% | 565.52 | 242.61 |
| 3099 | 922.46 | 83.44 | 97% | 599.33 | 119.33 |
| 2813 | 1250.39 | 172.69 | 98% | 603.02 | 185.25 |
| 20998 | 325.2 | 72.5 | 97% | 606.04 | 134.27 |
| 21010 | 1699.76 | 218.74 | 98% | 606.25 | 249.41 |
| 14882 | 377.63 | 34.39 | 97% | 607.89 | 168.14 |
| 5616 | 386.99 | 47.15 | 97% | 623.82 | 140.57 |
| 16945 | 1098.96 | 98.19 | 98% | 628.67 | 192.67 |
| 7420 | 1415.94 | 79.85 | 97% | 655.69 | 311.93 |
| 18890 | 1900.82 | 258.12 | 99% | 657.78 | 337.82 |
| 3279 | 1571.19 | 374.24 | 98% | 708.13 | 199.08 |
| 16190 | 1581.05 | 206.33 | 98% | 716.2 | 226.42 |
| 20597 | 378.94 | 48.6 | 98% | 742.21 | 189.37 |
| 21341 | 1797.23 | 203.99 | 98% | 768.53 | 328.94 |
| 4940 | 623.22 | 140.4 | 98% | 1632.44 | 469.8 |

We claim:

1. A method of predicting at least one toxic effect of a compound, comprising:
(a) detecting the level of expression in a tissue or cell sample exposed to the compound of two or more genes from Tables 1-3; wherein differential expression of the genes in Tables 1-3 is indicative of at least one toxic effect.

2. A method of predicting the progression of a toxic effect of a compound, comprising:
(a) detecting the level of expression in a tissue or cell sample exposed to the compound of two or more genes from Tables 1-3; wherein differential expression of the genes in Tables 1-3 is indicative of toxicity progression.

3. A method of predicting the hepatotoxicity of a compound, comprising:
(a) detecting the level of expression in a tissue or cell sample exposed to the compound of two or more genes from Tables 1-3; wherein differential expression of the genes in Tables 1-3 is indicative of hepatotoxicity.

4. A method of identifying an agent that modulates the onset or progression of a toxic response, comprising:
(a) exposing a cell to the agent and a known toxin; and
(b) detecting the expression level of two or more genes from Tables 1-3; wherein differential expression of the genes in Tables 1-3 is indicative of toxicity.

5. A method of predicting the cellular pathways that a compound modulates in a cell, comprising:
(a) detecting the level of expression in a tissue or cell sample exposed to the compound of two or more genes from Tables 1-3; wherein differential expression of the genes in Tables 1-3 is associated the modulation of at least one cellular pathway.

6. The method of any one of claims 1-5, wherein the expression levels of at least 3 genes are detected.

7. The method of any one of claims 1-5, wherein the expression levels of at least 4 genes are detected.

8. The method of any one of claims 1-5, wherein the expression levels of at least 5 genes are detected.

9. The method of any one of claims 1-5, wherein the expression levels of at least 6 genes are detected.

10. The method of any one of claims 1-5, wherein the expression levels of at least 7 genes are detected.

11. The method of any one of claims 1-5, wherein the expression levels of at least 8 genes are detected.

12. The method of any one of claims 1-5, wherein the expression levels of at least 9 genes are detected.

13. The method of any one of claims 1-5, wherein the expression levels of at least 10 genes are detected.

14. A method of claim 1 or 2, wherein the effect is selected from the group consisting of hepatitis, liver necrosis, protein adduct formation and fatty liver.

15. A method of claim 3, wherein the hepatotoxicity is associated with at least one liver disease pathology selected from the group consisting of hepatitis, liver necrosis, protein adduct formation and fatty liver.

16. A method of claim 5, wherein the cellular pathway is modulated by a toxin selected from the group consisting of amitryptiline, ANIT, acetaminophen, carbon tetrachloride, cyproterone acetate, diclofenac, estradiol, indomethacin, valproate, and WY-14643.

17. A set of at least two probes, wherein each of the probes comprises a sequence that specifically hybridizes to a gene in Tables 1-3.

18. A set of probes according to claim 17, wherein the set comprises probes that hybridize to at least 3 genes.

19. A set of probes according to claim 17, wherein the set comprises probes that hybridize to at least 5 genes.

20. A set of probes according to claim 17, wherein the set comprises probes that hybridize to at least 7 genes.

21. A set of probes according to claim 17, wherein the set comprises probes that hybridize to at least 10 genes.

22. A set of probes according to any one of claims 17-21, wherein the probes are attached to a solid support.

23. A set of probes according to claim 22, wherein the solid support is selected from the group consisting of a membrane, a glass support and a silicon support.

24. A solid support comprising at least two probes, wherein each of the probes comprises a sequence that specifically hybridizes to a gene in Tables 1-3.

25. A solid support of claim 24, wherein the solid support is an array comprising at least 10 different oligonucleotides in discrete locations per square centimeter.

26. A solid support of claim 25, wherein the array comprises at least 100 different oligonucleotides in discrete locations per square centimeter.

27. A solid support of claim 25, wherein the array comprises at least 1000 different oligonucleotides in discrete locations per square centimeter.

28. A solid support of claim 25, wherein the array comprises at least 10,000 different oligonucleotides in discrete locations per square centimeter.

29. A computer system comprising:
(a) a database containing information identifying the expression level in a tissue or cell sample exposed to a hepatotoxin of a set of genes comprising at least two genes in Tables 1-3; and
(b) a user interface to view the information.

30. A computer system of claim 29, wherein the database further comprises sequence information for the genes.

31. A computer system of claim 29, wherein the database further comprises information identifying the expression level for the set of genes in the tissue or cell sample before exposure to a hepatotoxin.

32. A computer system of claim 29, wherein the database further comprises information identifying the expression level of the set of genes in a tissue or cell sample exposed to at least a second hepatotoxin.

33. A computer system of any of claims 29-32, further comprising records including descriptive information from an external database, which information correlates said genes to records in the external database.

34. A computer system of claim 33, wherein the external database is GenBank.

35. A method of using a computer system of any one of claims 29-32 to present information identifying the expression level in a tissue or cell of at least one gene in Tables 1-3, comprising:
(a) comparing the expression level of at least one gene in Tables 1-3 in a tissue or cell exposed to a test agent to the level of expression of the gene in the database.

36. A method of claim 35, wherein the expression levels of at least two genes are compared.

37. A method of claim 35, wherein the expression levels of at least five genes are compared.

38. A method of claim 35, wherein the expression levels of at least ten genes are compared.

39. A method of claim 35, further comprising the step of displaying the level of expression of at least one gene in the tissue or cell sample compared to the expression level when exposed to a toxin.

40. A method of claim 4, wherein the known toxin is a hepatotoxin.

41. A method of claim 37, wherein the hepatotoxin is selected from the group consisting of ANIT, acetaminophen, carbon tetrachloride, cyproterone acetate, diclofenac, estradiol, indomethacin, valproate, and WY-14643.

42. A method of any one of claims 1-5, wherein nearly all of the genes in Tables 1-3 are detected.

43. A method of claim 42, wherein all of the genes in any one of Tables 3A-3S are detected.

44. A kit comprising at least one solid support of any one of claims 24-28 packaged with gene expression information for said genes.

45. A kit of claim 44, wherein the gene expression information comprises gene expression levels in a tissue or cell sample exposed to a hepatotoxin.

46. A kit of claim 45, wherein the gene expression information is in an electronic format.

47. A method of any one of claims 1-5, wherein the compound exposure is in vivo or in vitro.

48. A method of any one of claims 1-5, wherein the level of expression is detected by an amplification or hybridization assay.

49. A method of claim 48, wherein the amplification assay is quantitative or semi-quantitative PCR.

50 A method of claim 48, wherein the hybridization assay is selected from the group consisting of Northern blot, dot or slot blot, nuclease protection and microarray assays.

51. A method of identifying an agent that modulates at least one activity of a protein encoded by a gene in Tables 1-3 comprising:
(a) exposing the protein to the agent; and
(b) assaying at least one activity of said protein.

52. A method of claim 51 wherein the agent is exposed to a cell expressing the protein.

53. A method of claim 52 wherein the cell is exposed to a known toxin.

54. A method of claim 53 wherein the toxin modulates the expression of the protein.
